(19) United States
(12) Patent Application Publication
(10) Pub. No.: US 2012/0021936 A1
(43) Pub. Date: Jan. 26, 2012

(54) BIOMARKERS FOR DENGUE

(75) Inventors: Brian Ward, Montreal (CA); Momar Ndao, Brossard (CA); Takol Chareonsirisuthikul, Bangkok (TH); Sukathida Ubol, Bangkok (TH)

(73) Assignees: Mahidol University, Nakhonpathom (TH); The Royal Institution for the Advancement of Learning/McGill University, Montreal, QC (CA)

(21) Appl. No.: 13/124,362

(22) PCT Filed: Oct. 14, 2009
(86) PCT No.: PCT/IB2009/007358

§ 371 Date: Oct. 11, 2011

Related U.S. Application Data
(60) Provisional application No. 61/105,381, filed on Oct. 14, 2008.

(51) Int. Cl. C12Q 1/70; C40B 30/04

(52) U.S. Cl. 506/9; 435/5

(57) ABSTRACT

The present invention provides protein-based biomarkers and biomarker combinations that are useful in qualifying dengue status in a patient. In particular, the biomarkers of this invention are useful to classify a subject sample as infected with dengue or not infected with dengue. The biomarkers can be detected by SELDI mass spectrometry.

### FIELD

The invention relates generally to clinical diagnostics and prognostics for infection.

### BACKGROUND

“Break-bone fever”, or dengue fever (DF), was first spread worldwide in the tropics during the 18^{th} and 19^{th} century following the expansion of the commerce and shipping industry. The Aedes aegypti, main mosquito vector, was introduced, along with the dengue virus (DENV), in the new regions chartered by the industry. During the last decade, dengue was able to spread due to an increase in air travel, unprecedented population growth, unplanned and uncontrolled urbanization, and the lack of mosquito control among other things (Rigau-Perez, J., et al., 1998, Lancet 352:971-977). Today it is estimated that 2.5 billion people are at risk of DENV infection in more than 100 countries in the Americas, Southeast Asia, western Pacific, Africa and the eastern Mediterranean. There is an estimated 50 million cases of dengue infection each year with 500,000 cases of dengue hemorrhagic fever (the more severe case of the disease) and at least 12,000 deaths, mostly in children (DengueNet, 2002, Weekly Epidemiological Record 77:300-304).

Dengue virus belongs to the Flavivirus genus that also includes yellow fever, West Nile, tick-borne encephalitis (TBEV), and Japanese encephalitis viruses. There are 4 primary serotypes that exist which can cause different degrees of disease severity ranging from the mildest form of dengue fever (DF), to dengue hemorrhagic fever (DHF), and the most severe form of dengue shock syndrome (DSS). DENV possesses an icosahedral core of 40-50 nm in diameter, containing one of the 3 structural proteins, the C protein. It encapsulates the 10,700 nucleotide plus-sense RNA genome. Surrounding the core is a smooth lipid bilayer composed of the other 2 structural proteins, the membrane (M) protein, and the envelope glycoprotein (E) (Kuhn, R. J., et al., 2002, Cell 108:717-725). The main biological properties of the virus come from the E protein where it allows for receptor binding, haemagglutination of erythrocytes, neutralizing antibody induction, and protective immune response (Chang, G. J. 1997, p. 175-198. In D. J. Gubler and G. Kuno (ed.), CAB International, New York). It also possesses 7 non-structural proteins (NS1, NS2a, NS2b, NS3, NS4a, NS4b, NS5), of which two, NS1 and NS3, are believed to be the most important ones involved in the pathogenesis. Upon primary infection with DENV, antibodies against the surface E, NS1, and NS3 proteins are generated (Green, S. and A. Rothman, 2006, Current Opinion in Infectious Diseases 19:429-436.). Therefore serotypes can be distinguished by virus-neutralizing antibodies, but non-neutralizing antibodies against the E protein and non-structural proteins NS1 and NS3 are cross-reactive. A life-long immunity against the infective serotype ensues, but protection against others is only for a short period of time. During a second infection by a different serotype, the presence of neutralizing antibodies can reduce the severity of the disease. However, if the levels of these antibodies drop under the neutralizing amount, the heterotypic IgG antibodies form complexes with dengue viruses that can bind to the FcyR resulting in an augmentation of the virus infection. This model is called the antibody dependent enhancement (ADE) (Green, S. and A. Rothman, 2006, Current Opinion in Infectious Diseases 19:429-436; Guzman, M. G. and G. Kouri. 2002, The Lancet Infectious Diseases 2:33-42; Kliks, S. C., et al., 1989, American Journal of Tropical Medicine & Hygiene 40:444-451; Oishi, K., et al., 2003, Journal of Medical Virology 71:259-264; and Stephenson, J. R., 2005, Bulletin of the World Health Organization 83:308-314). To further support this model, it has been observed that the incidence of DHF/DSS in children occurs at two distinct peaks in their lives. The first occurs when the child is 6-9 months old. This is the age at which the maternal antibodies are still present in the circulation. If the child gets infected by a different heterotypic DENV than the mother, DHF/DSS ensues since the levels of maternal antibodies have fallen below the protective levels (Simmons, C. P., et al., Journal of Infectious Diseases 196:416-424). The other peak occurs in young children infected for a second time. ADE supports the fact that DHF/DSS is 15-80 times more likely in secondary infections. However, this can not explain the whole pathogenesis of dengue virus and many other factors still to be studied might play a role such as the strain's virulence and the serotype, and the host susceptibility and the specific role of T cells (Chaturvedi, U., et al., 2006, FEMS Immunology & Medical Microbiology 47:155-166, Fink, J., et al., 2006, Reviews in Medical Virology 16:263-275). All these factors need to be considered in the design of a vaccine (Stephenson, J. R., 2005, Bulletin of the World Health Organization 83:308-314).

Once one is bitten by an infected mosquito, there is an incubation period of up to 2 weeks. Most infections are asymptomatic, especially in children under 15 years of age, but can cause a range of symptoms and even lead to death. Population-based studies have shown that the severity of the disease increases with the patient's age (Burke, D. S., 1988, American Journal of Tropical Medicine & Hygiene 38:172-80, Cobra, C., et al., 1995, American Journal of Epidemiology 142:1204-1211, Dietz, V., et al., 1996. Puerto Rico Health Sciences Journal 15:201-210; and Kuberski, T., et al., 1977, American Journal of Tropical Medicine & Hygiene 26:775-783). DF is an acute febrile disease often characterized by frontal headache, retroocular pain, muscle and joint pain, nausea, vomiting, and rash (Kalayanarooj, S., et al., 1997, Journal of Infectious Diseases 176:313-321). The febrile period usually terminates between 5-7 days after the onset of symptoms, often correlating with the disappearance of the virus from the circulation. In Southeast Asia, DHF is mostly seen in children, but it is seen in all age groups in the tropical Americas. This suggests the involvement of race or strain virulence as risk factors. DHF is an acute febrile illness, typically with bleeding, thrombocytopenia, elevated haematocrit, pleural effusions, and hypoproteinaemia. It begins as DF with a sudden onset of fever, and then develops into DHF around 3-7 days of illness (around the time of defervescence for DF) and continues for about 2-7 days. The main pathophysiological difference between DF and DHF is plasma leakage. Dengue shock syndrome (DSS) is the most severe form of the disease characterized by circulatory failure and a narrowing pulse range. Once shock begins, the fatality rate can be as high as 44% if the proper precautions are not taken (Oishi, K., et al., 2003, Journal of Medical Virology 71:259-264). There are no antiviral drugs administered nor are any drugs known to be useful in limiting the plasma leakage. Dengue treatment is only supportive where analgesics and antipyretics (but not aspirin) are given and fluid management is applied. Only when the molecular biology of DHF is understood will we able to treat it (Lei, H. Y., et al., 2001, Journal of Biomedical Science 8:377-388; and Rigau-Perez, J., et al., 1998, Lancet 352:971-977). This is why the diagnostic of a dengue infection needs to be given early in the disease progression so to maximize the patient's chance of survival. However, clinical findings alone are often not very helpful in distinguishing DF from other febrile illnesses (OFIs) such as the chikungunya, measles, leptospirosis, yellow fever, influenza, West Nile, Japanese, and St Louis encephalitis (Rigau-Perez, J., et al., 1998, Lancet 352:971-977; Senanayake, S., 2006, Australian Family Physician 35:609-612; and Teles, F. R., D. M. Prazeres, and J. L. Lima-Filho, 2005, Reviews in Medical Virology 15:287-302).

During a primary infection, IgM antibodies are developed after 5-6 days and are present in the circulation for up to 2-3 months after infection, while IgG antibodies become present after only 7-10 days. On the other hand, a secondary infection occurs when an individual has been previously infected or immunized with a flavivirus. IgM levels are lower if not absent but IgG levels are very high, even during the acute phase of the infection. Therefore, IgM is a sign of an early infection while high levels of IgG reveal a secondary infection (Guzman, M. G. and G. Kouri, 2002, The Lancet Infectious Diseases 2:33-42). Viable DENV particles are detectable in the circulation for up to 5 days after the symptoms but then rapidly disappear upon the appearance of DENV-specific antibodies (Kao, C. L., et al., 2005, Journal of Microbiology, Immunology & Infection 38:5-16).

Enzyme immunoassay (EIA) is used to detect IgM and IgG antibodies to dengue. This method can distinguish a primary infection from a secondary infection by determining the IgM/IgG ratio; if the ratio in convalescent sera exceeds 1.5, it reveals a primary infection. The World Health Organization (WHO) recommends the use of the dengue monoclonal antibody (IgM)-capture EIA (MAC-EIA) which is inexpensive, simple, fast, and only requires one blood sample. However, IgM antibodies can only be detected at least 5 days after infection since this is the time needed for the body to produce anti-dengue antibodies. Moreover, some false-positives can occur due to the persistence of IgM in the blood even after a few months (Teles, F. R., D. M. Prazeres, and J. L. Lima-Filho, 2005, Reviews in Medical Virology 15:287-302).

The haemagglutination-inhibition (HI) is slightly more sensitive than the EIA test. On the other hand, chemical treatment of the samples is needed to remove non-specific inhibitor of heamagglutination as well as non-specific agglutinins Moreover, this test does not differentiate between closely related flavivirus infections or different DENV serotypes. Paired sera are needed and so the results can take weeks.

There exists also the neutralization test which is more sensitive than the HI-test but employs live virus and so Biosafety Level 3 Laboratories are needed. It also encounters the same difficulties as the HI-test in terms of specificity in addition to the extra cost, time, and technical difficulty associated with the neutralizing test (Teles, F. R., D. M. Prazeres, and J. L. Lima-Filho, 2005, Reviews in Medical Virology 15:287-302).

The complement fixation (CF) test is a good marker of recent infection compared to the detection of IgM dengue specific antibodies due to their short persistence in the blood. However, the CF antibody appears only 7-14 days after the onset of symptoms. Also, it is the least sensitive of the serological tests.

Due to some cross-reactivity in flaviviruses, any serologic test must include as controls the four dengue serotypes, another serotype, a non-flavivirus and an uninfected control for it to be a confirmatory diagnosis (Teles, F. R., D. M. Prazeres, and J. L. Lima-Filho, 2005, Reviews in Medical Virology 15:287-302). Also, the high rate of IgG positive results for people in the tropics indicate that paired acute and convalescent serum samples are often critical for the significance of the tests (Rigau-Perez, J., et al., 1998, Lancet 352:971-977).

Inoculation of clinical specimens into mosquito cells, larvae or adult mosquitoes is the most sensitive approach. Specific detection and identification of the virus by immunofluorescence assays with serotype-specific anti-dengue monoclonal antibodies makes this technique able to determine the serotype of DENV. This test is convenient since the samples are relatively suitable for 2 weeks and the test does not require special facilities or special training (Teles, F. R., D. M. Prazeres, and J. L. Lima-Filho, 2005, Reviews in Medical Virology 15:287-302). However, days to weeks are necessary for virus isolation and the cost of equipment and laboratory maintenance is high (Kao, C. L., et al., 2005, Journal of Microbiology, Immunology & Infection 38:5-16).

RNA viral genome can be detected by PCR-based techniques, e.g., RT-PCR. It is a technique that is just as expensive as the virus culture technique with higher contamination risks associated with sample manipulation, but only takes a few hours to perform and is much more sensitive. By using 4 serotype-specific oligonucleotide primers, it is also possible to detect the serotype of the given DENV (Teles, F. R., D. M. Prazeres, and J. L. Lima-Filho, 2005, Reviews in Medical Virology 15:287-302).

Thus a need exists for the identification of biomarkers that could simplify the diagnosis and/or prognosis of dengue and its symptoms at, e.g., reduced costs. The present invention provides for these and other advantages, as described below.

### SUMMARY

The present invention provides, inter alia, biomarkers that are differentially present in subjects with dengue. In addition, the present invention provides methods of using the biomarkers to qualify dengue in a subject or in a biological sample taken from a subject, including a sample of serum, blood, or other donated tissue. As such, the invention provides biomarkers that represent full length proteins or fragments of proteins expressed in infected individuals by a member of the Flaviviridae family, the pathogen responsible for dengue.

The biomarkers can be used, inter alia, to qualify dengue status, determine the course of dengue, monitor the response to treatment by a drug used to treat dengue, and/or determine a treatment regimen for dengue. The dengue can be caused by members of the Flaviviridae family.

In one aspect, the present invention provides a method for qualifying dengue status in a subject, the method including: (a) measuring at least one biomarker in a biological sample from the subject, wherein the at least one biomarker is selected from the group consisting of the biomarkers of Tables 1-5, 17, 21, and 24; and (b) correlating the measurement with dengue status. In one aspect, the biological sample is a serum sample.

The at least one biomarker can be selected from the group consisting of biomarkers of molecular masses of about 2.5, 2.6, 2.7, 2.8, 3.0, 3.2, 3.4, 3.5, 3.8, 3.9, 4.0, 4.1, 4.2, 4.3, 4.4, 4.5, 4.6, 5.0, 5.1, 5.2, 5.3, 5.5, 5.6, 5.7, 6.0, 6.5, 6.6, 6.7, 6.8, 6.9, 7.0, 7.4, 7.5, 7.6, 8.2, 8.8, 9.0, 9.3, 9.5, 9.6, 10.0, 10.1, 10.2, 10.3, 10.4, 10.5, 10.6, 10.7, 10.8, 10.9, 11.0, 11.1, 11.3, 11.5, 11.7, 11.8, 11.9, 12.1, 12.2, 12.4, 12.5, 12.6, 12.7, 12.9, 13.0, 13.1, 13.2, 13.3, 13.4, 13.5, 13.8, 14.0, 14.1, 14.2, 14.3, 14.4, 15.3, 17.4, 17.7, 17.8, 18.0, 21.5, 22.4, 23.1, 23.3, 23.6, 23.7, 23.8, 25.4, 25.6, 26.5, 26.7, 28.2, 28.4, 29.0, 30.3, 30.8, 31.2, 32.3, 33.5, 33.6, 34.0, 34.2, 34.5, 34.7, 36.5, 36.6, 38.7, 39.5, 39.9, 42.3, 43.5, 44.0, 44.6, 44.7, 45.0, 45.4, 45.5, 45.6, 46.2, 46.6, 46.7, 49.7, 50.5, 51.7, 52.4, 52.6, 53.4, 53.6, 54.3, 54.4, 54.6, 54.8, 55.1, 55.3, 55.8, 56.6, 59.4, 59.5, 61.4, 63.1, 66.6, 66.7, 66.8, 67.1, 69.0, 70.9, 71.3, 71.5, 75.1, 75.2, 75.3, 77.1, 79.1, 79.3, 88.3, 95.7, 109.0, 111.3, 117.2, 123.0, 125.4, 133.4, 133.7, 150.1, 164.6, 188.6, 194.2, and 198.3 kDa and any combination thereof.

The at least one biomarker can be selected from the group consisting of biomarkers of molecular masses of about 2.5, 2.6, 2.7, 2.8, 3.0, 3.2, 3.4, 3.5, 3.8, 3.9, 4.0, 4.1, 4.2, 4.3, 4.4, 4.5, 4.6, 5.0, 5.1, 5.2, 5.3, 5.5, 5.6, 5.7, 6.0, 6.5, 6.6, 6.7, 6.8, 6.9, 7.0, 7.4, 7.5, 7.6, 8.2, 8.8, 9.0, 9.3, 9.5, 9.6, 10.0, 10.1, 10.2, 10.3, 10.4, 10.5, 10.6, 10.7, 10.8, 10.9, 11.0, 11.1, 11.3, 11.5, 11.7, 11.8, 11.9, 12.1, 12.2, 12.4, 12.5, 12.6, 12.7, 12.9, 13.0, 13.1, 13.2, 13.3, 13.4, 13.5, 13.8, 14.0, 14.1, 14.2, 14.3, 14.4, 15.3, 17.4, 17.7, 17.8, 18.0, 21.5, 22.4, 23.1, 23.3, 23.6, 23.7, 23.8, and 25.4 kDa and any combination thereof. The at least one biomarker can be selected from the group consisting of biomarkers of molecular masses of about 25.6, 26.5, 26.7, 28.2, 28.4, 29.0, 30.3, 30.8, 31.2, 32.3, 33.5, 33.6, 34.0, 34.2, 34.5, 34.7, 36.5, 36.6, 38.7, 39.5, 39.9, 42.3, 43.5, 44.0, 44.6, 44.7, 45.0, 45.4, 45.5, 45.6, 46.2, 46.6, 46.7, 49.7, 50.5, 51.7, 52.4, 52.6, 53.4, 53.6, 54.3, 54.4, 54.6, 54.8, 55.1, 55.3, 55.8, 56.6, 59.4, 59.5, 61.4, 63.1, 66.6, 66.7, 66.8, 67.1, 69.0, 70.9, 71.3, 71.5, 75.1, 75.2, 75.3, 77.1, 79.1, 79.3, 88.3, 95.7, 109.0, 111.3, 117.2, 123.0, 125.4, 133.4, 133.7, 150.1, 164.6, 188.6, 194.2, and 198.3 kDa and any combination thereof.

The at least one biomarker can be selected from the group consisting of biomarkers of molecular masses of about 3.4, 3.8, 3.9, 4.0, 4.6, 6.6, 6.7, 7.0, 7.6, 10.6, 11.1, 11.7, 12.5, 12.7, 12.9, 13.1, 13.2, 13.3, 13.4, 14.4, 23.1, 23.3, 23.6, 23.8, 25.4, 34.2, 44.7, 45.6, 46.2, 46.4, 56.6, 117.2, 133.4, 133.7, 198.3 kDa and any combination thereof. The at least one biomarker can be selected from the group consisting of biomarkers of molecular masses of about 3.4, 3.8, 3.9, 4.0, 4.6, 6.6, 6.7, 7.0, 7.6, 10.6, 11.1, 11.7, and 12.5 kDa and any combination thereof. The at least one biomarker can be selected from the group consisting of biomarkers of molecular masses of about 12.7, 12.9, 13.1, 13.2, 13.3, 13.4, 14.4, 23.1, and 23.3 kDa. The at least one biomarker can be selected from the group consisting of biomarkers of molecular masses of about 23.6, 23.8, 25.4, 34.2, and 44.7 kDa and any combination thereof. The at least one biomarker can be selected from the group consisting of biomarkers of molecular masses of about 45.6, 46.2, 46.4, 56.6, 117.2, 133.4, 133.7, and 198.3 kDa and any combination thereof. The at least one biomarker can be selected from the group consisting of biomarkers of molecular masses of about 3.4, 3.8, 3.9, 4.0, 4.6, 6.6, 6.7, 133.4, 133.7, and 198.3 kDa and any combination thereof. The at least one biomarker can be selected from the group consisting of biomarkers of molecular masses of about 3.4, 3.8, 3.9, 25.4, 34.2, 44.7, 45.6, 46.2, and 46.4 kDa and any combination thereof. The at least one biomarker can be selected from the group consisting of biomarkers of molecular masses of about 4.6, 25.4, 34.2, and 44.7 kDa and any combination thereof. The at least one biomarker can be selected from the group consisting of biomarkers of molecular masses of about 7.0, 7.6, 10.6, 11.1, 11.7, 12.5, 12.7, 12.9, 13.1, 13.2, 13.3, 13.4, 14.4, 23.8, 25.4, 34.2, 44.7, and 45.6 kDa and any combination thereof. The at least one biomarker can be selected from the group consisting of biomarkers of molecular masses of about 3.4, 3.8, 3.9, 4.0, 4.6, 6.6, 6.7, 117.2, 133.4, 133.7, and 198.3 kDa and any combination thereof. The at least one biomarker can be selected from the group consisting of biomarkers of molecular masses of about 12.7, 12.9, 13.1, 13.2, and 13.3 kDa and any combination thereof.

The at least one biomarker can be selected from the group consisting of biomarkers of molecular masses of about 2.6, 2.7, 2.8, 3.5, 4.2, 5.1, 5.2, 5.3, 5.5, 5.6, 5.7, 6.7, 6.8, 6.9, 7.0, 7.4, 8.8, 9.0, 9.3, 9.5, 9.6, 10.3, 10.9, 11.3, 11.5, 11.7, 11.8, 11.9, 12.4, 12.6, 12.9, 13.5, 13.8, 25.6, 32.3, 39.9, 42.3, 44.0, 44.6, 45.0, 46.6, 46.7, 49.7, 53.6, 54.4, 55.8, 63.1, 67.1, 75.3, 88.3, 111.3, and 150.1 kDa and any combination thereof. The at least one biomarker can be selected from the group consisting of biomarkers of molecular masses of about 2.6, 2.7, 2.8, 3.5, 4.2, 5.1, 5.2, 5.3, 5.5, 5.6, 5.7, 6.7, and 6.8 kDa and any combination thereof. The at least one biomarker can be selected from the group consisting of biomarkers of molecular masses of about 6.9, 7.0, 7.4, 8.8, 9.0, 9.3, 9.5, 9.6, 10.3, 10.9, 11.3, 11.5, 11.7, 11.8, 11.9, and 12.4 kDa and any combination thereof. It will be understood that any combination of the biomarkers described herein can be measured using the methods described herein.

In some aspects, the at least one biomarker is selected from the group consisting of biomarkers of molecular masses of about 12.6, 12.9, 13.5, 13.8, 25.6, 32.3, 39.9, 42.3, 44.0, 44.6, and 45.0 kDa and any combination thereof. In some aspects, the at least one biomarker is selected from the group consisting of biomarkers of molecular masses of about 46.6, 46.7, 49.7, 53.6, 54.4, 55.8, 63.1, 67.1, 75.3, 88.3, 111.3, and 150.1 kDa and any combination thereof. In some aspects, the at least one biomarker is selected from the group consisting of biomarkers of molecular masses of about 2.6, 2.7, 2.8, 3.5, 4.2, 5.1, 5.2, 5.3, 67.1, 75.3, 88.3, 111.3, and 150.1 kDa and any combination thereof. In some aspects, each of the biomarkers having a molecular mass of about 75.3, 88.3, 111.3, and 150.1 kDa is measured.

In some aspects, the at least one biomarker is selected from the group consisting of biomarkers of molecular masses of about 2.6, 2.7, 11.7, 11.8, 11.9, 12.4, 67.1, 75.3, 88.3, 111.3, and 150.1 kDa and any combination thereof. In some aspects, the at least one biomarker is selected from the group consisting of biomarkers of molecular masses of about 7.4, 8.8, 9.0, 9.3, 9.5, 9.6, 10.3, 10.9, 12.4, 12.6, 12.9, 13.5, 13.8, 25.6, and 32.3 kDa and any combination thereof. In some aspects, the at least one biomarker is selected from the group consisting of biomarkers of molecular masses of about 11.5, 25.6, and 32.3 kDa and any combination thereof.

In some aspects, the at least one biomarker is a protein or fragment thereof as provided in Table 5. In certain aspects, the at least one biomarker is represented by at least one of the accession numbers provided in Table 5.

In one aspect, the at least one biomarker is measured by capturing the biomarker on an adsorbent of a SELDI probe and detecting the captured biomarkers by laser desorption-ionization mass spectrometry. In certain aspects, the adsorbent is a cation exchange adsorbent, whereas in other aspects, the adsorbent is a metal chelation adsorbent. In another aspect, the at least one biomarker is measured by immunoassay.

In another aspect, the correlating is performed by a software classification algorithm. In a further aspect, dengue status is selected from chronically infected versus uninfected. In yet other aspects, dengue status is selected from chronically infected status versus acutely infected disease status, chronically infected asymptomatic status versus chronically affected with symptoms, or acutely infected status versus healthy uninfected status. In still another aspect, dengue status is selected from dengue versus healthy. In yet other aspects, dengue status is selected from dengue fever (DF), dengue hemorrhagic fever (DHF), and dengue shock syndrome (DSS). In other aspects, the biomarkers of the present invention can be used to predict the effectiveness of a dengue vaccine. In other aspects, dengue status is selected from primary infection and secondary infection.

In yet another aspect, the method further comprises managing subject treatment based on the status. If the measurement correlates with dengue, then managing subject treatment comprises administering to a patient drugs selected from a group consisting of, but not necessarily limited to, drugs such as paracetamol, antipyretics, and combinations thereof.

In a further aspect, the method further comprises measuring the at least one biomarker after subject management.

In another aspect, the present invention provides a method comprising measuring at least one biomarker in a sample from a subject, wherein the at least one biomarker is selected from the group consisting of the biomarkers set forth in Tables 1-5, 17, 21, and 24. In one aspect, the sample is a serum sample.

In still another aspect, the present invention provides a kit comprising: (a) a solid support comprising at least one capture reagent attached thereto, wherein the capture reagent binds at least one biomarker from a first group consisting of the biomarkers set forth in Table 1, Table 2, Table 3, Table 4, Table 5, Table 17, Table 21, and Table 24; and (b) instructions for using the solid support to detect the at least one biomarker set forth in Table 1, Table 2, Table 3, Table 4, Table 5, Table 17, Table 21, and Table 24.

In other aspects, the kit additionally comprises (c) a container containing at least one of the biomarkers of Table 1, Table 2, Table 3, Table 4, Table 5, Table 17, Table 21, and Table 24.

In yet a further aspect, the present invention provides a software product, the software product comprising: (a) code that accesses data attributed to a sample, the data comprising measurement of at least one biomarker in the sample, the biomarker selected from the group consisting of the biomarkers of Table 1, Table 2, Table 3, Table 4, Table 5, Table 17, Table 21, and Table 24; and (b) code that executes a classification algorithm that classifies dengue status of the sample as a function of the measurement.

In one aspect, the classification algorithm classifies dengue status of the sample as a function of the measurement of a biomarker selected from the biomarkers of Tables 1-5, 17, 21, and 24.

In other aspects, the present invention provides purified biomolecules selected from the biomarkers set forth in Table 1, Table 2, Table 3, Table 4, Table 5, Table 17, Table 21, and Table 24 and, additionally, methods comprising detecting a biomarker set forth in Table 1, Table 2, Table 3, Table 4, Table 5, Table 17, Table 21, and Table 24 by mass spectrometry or immunoassay.

In yet another aspect, the method further comprises testing and qualifying stocks of blood based on the status of blood which has been tested according to the methods described herein. If the measurements taken from blood samples correlate with dengue, then the management of blood stocks comprises decontamination of the infected blood by treatment of the infected blood with purification agents available to one skilled in the art. Alternatively, the infected blood can be discarded or destroyed and only stocks of blood which have not tested positively for dengue are retained.

In one aspect, the present invention provides a method for qualifying dengue status in a subject in comparison to the status of a different viral infection, the method comprising: (a) measuring at least one biomarker in a biological sample from the subject, wherein the at least one biomarker specifically indicates the presence of dengue and does not indicate the presence of a different infection; and (b) correlating the measurement with dengue status in comparison to the status of a different infection. In one aspect, the biological sample is a serum sample. In a preferred aspect of this method, the at least one biomarker is selected from the group of biomarkers of Tables 1-5, 17, 21, and 24. In still another preferred aspect, the infection includes, but is not limited to other febrile illnesses (OFIs).

In another aspect, the present invention provides a method for monitoring the course of progression of dengue in a patient comprising: (a) measuring at least one biomarker in a first biological sample from the patient, wherein the at least one biomarker specifically indicates the presence of dengue; (b) measuring the at least one biomarker in a second biological sample from the subject, wherein the second biological sample was obtained from the subject after the first biological sample; and (c) correlating the measurements with the progression or regression of dengue in the subject. In one aspect, the at least one biomarker is selected from the group consisting of the biomarkers of Tables 1-5, 17, 21, and 24.

Other features, objects and advantages of the invention and its preferred aspects will become apparent from the detailed description, examples and claims that follow.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows Biomarker Pattern Software analysis results for fraction F1CSL (fraction 1 using CM10 at low laser intensity). Using the indicated splitters, 100.000% sensitivity and 94.737% specificity was achieved.

FIG. 2 shows a graphical representation from CiphergenExpress of 3 candidate dengue diagnostic biomarkers of the F1CSL fraction. (A) Predicted MW of 4580 Da. (B) Predicted MW of 3957 Da. (C) Predicted MW of 3870 Da. Each dot represents a sample. The relative intensity of each protein is represented on the y-axis.

FIG. 3 shows a graphical representation from CiphergenExpress of a candidate dengue diagnostic biomarker with predicted MW of 4292 Da in the FISL fraction. (A) CE graphical representation of control and DHF at t2. (B) CE graphical representation of control and DF at t2. Each dot represents a sample. The relative intensity of each protein is represented on the y-axis.

FIG. 4 shows Biomarker Pattern Software analysis results for fraction FlISH (fraction 1 using IMAC at high laser intensity). Using the indicated splitters, 92.592% sensitivity and 100.000% specificity was achieved.

FIG. 5 shows a graphical representation from CiphergenExpress of a candidate dengue diagnostic biomarker with predicted MW of 23105 Da in the F1ISH fraction. Each dot represents a sample. The relative intensity of each protein is represented on the y-axis.

FIG. 6 shows Biomarker Pattern Software analysis results for fraction F5CSH (fraction 5 using CM10 at high laser intensity). Using the indicated splitters, 100.000% sensitivity and 100.000% specificity was achieved.

FIG. 7 shows a graphical representation from CiphergenExpress 2 candidate dengue diagnostic biomarker with predicted MW of 4292 Da in the FISL fraction. (A) CE graphical representation of control and DHF at t1 of candidate biomarker with predicted MW of 12919 Da. (B) CE graphical representation of control and DHF at t2 of candidate biomarker with predicted MW of 13092 Da. Each dot represents a sample. The relative intensity of each protein is represented on the y-axis.

FIG. 8 shows a graphical representation from CiphergenExpress of a candidate dengue diagnostic biomarker with predicted MW of 12650 Da in the F6CSL fraction. Each dot represents a sample. The relative intensity of each protein is represented on the y-axis.

FIG. 9 shows a graphical representation from CiphergenExpress of a candidate dengue diagnostic biomarker with predicted MW of 3437 Da in the F61SL fraction. Each dot represents a sample. The relative intensity of each protein is represented on the y-axis.

FIG. 10 shows Biomarker Pattern Software analysis results for fraction F6ISH (fraction 6 using IMAC at high laser intensity). Using the indicated splitters, 93.333% sensitivity and 100.00% specificity was achieved.

FIG. 11 shows a graphical representation from CiphergenExpress of a candidate dengue diagnostic biomarker with predicted MW of 13317 Da in the F6ISH fraction. Each dot represents a sample. The relative intensity of each protein is represented on the y-axis.

FIG. 12 shows a 4-12% Bis-Tris NuPAGE Gel #1 of pooled controls (C) at time 1 and 2 compared to pooled dengue samples of DF and DHF at time 1 and 2 (D). ZOOM Fractionated and desalted (200 μl). Lane 1 and 10, Marker 12 MW (invitrogen). Each C or D sample was desalted and ZOOM Fractionated using specific pI ranges corresponding to the pH indicated on the figure. The boxes indicate the potential diagnostic biomarkers sent for sequencing.

FIG. 13 shows a graphical representation of the differential signal intensity of the AMBP protein precursor biomarker in control, DF, and DHF groups. The biomarker is characterized by its mass-to-charge ratio as determined by mass spectrometry and is represented in daltons.

FIG. 14 shows a graphical representation of the differential signal intensity of the Apolipoprotein A-I precursor biomarker in control, DF, and DHF groups. The biomarker is characterized by its mass-to-charge ratio as determined by mass spectrometry and is represented in daltons.

FIG. 15 shows a graphical representation of the differential signal intensity of the Apolipoprotein D precursor biomarker in control, DF, and DHF groups. The biomarker is characterized by its mass-to-charge ratio as determined by mass spectrometry and is represented in daltons.

FIG. 16 shows a graphical representation of the differential signal intensity of the C4b-binding protein a chain precursor biomarker in control, DF, and DHF groups. The biomarker is characterized by its mass-to-charge ratio as determined by mass spectrometry and is represented in daltons.

FIG. 17 shows a graphical representation of the differential signal intensity of the Carboxypeptidase N subunit 2 precursor biomarker in control, DF, and DHF groups. The biomarker is characterized by its mass-to-charge ratio as determined by mass spectrometry and is represented in daltons.

FIG. 18 shows a graphical representation of the differential signal intensity of the Ceruloplasmin precursor biomarker in control, DF, and DHF groups. The biomarker is characterized by its mass-to-charge ratio as determined by mass spectrometry and is represented in daltons.

FIG. 19 shows a graphical representation of the differential signal intensity of the Complement Clq subcomponent subunit B precursor biomarker in control, DF, and DHF groups. The biomarker is characterized by its mass-to-charge ratio as determined by mass spectrometry and is represented in daltons.

FIG. 20 shows a graphical representation of the differential signal intensity of the Hemoglobin subunit a biomarker in control, DF, and DHF groups. The biomarker is characterized by its mass-to-charge ratio as determined by mass spectrometry and is represented in daltons.

FIG. 21 shows a graphical representation of the differential signal intensity of the Hemopexin precursor biomarker in control, DF, and DHF groups. The biomarker is characterized by its mass-to-charge ratio as determined by mass spectrometry and is represented in daltons.

FIG. 22 shows a graphical representation of the differential signal intensity of the Insulin-like growth factor-binding protein complex acid labile chain biomarker in control, DF, and DHF groups. The biomarker is characterized by its mass-to-charge ratio as determined by mass spectrometry and is represented in daltons.

FIG. 23 shows a graphical representation of the differential signal intensity of the Plasma protease Cl inhibitor precursor biomarker in control, DF, and DHF groups. The biomarker is characterized by its mass-to-charge ratio as determined by mass spectrometry and is represented in daltons.

FIG. 24 shows a graphical representation of the differential signal intensity of the Sertransferrin precursor biomarker in control, DF, and DHF groups. The biomarker is characterized by its mass-to-charge ratio as determined by mass spectrometry and is represented in daltons.

FIG. 25 shows a graphical representation of the differential signal intensity of the Vitamin K-dependent protein S precursor biomarker in control, DF, and DHF groups. The biomarker is characterized by its mass-to-charge ratio as determined by mass spectrometry and is represented in daltons.

FIG. 26 shows a graphical representation of the differential signal intensity of the Vitronectin precursor biomarker in control, DF, and DHF groups. The biomarker is characterized by its mass-to-charge ratio as determined by mass spectrometry and is represented in daltons.

FIG. 27 shows a graphical representation of the differential signal intensity of the alpha1B-glycoprotein precursor biomarker in control, DF, and DHF groups. The biomarker is characterized by its mass-to-charge ratio as determined by mass spectrometry and is represented in daltons.

FIG. 28 shows a graphical representation of the differential signal intensity of the 3806 and 4596 DA biomarkers in control, DF, and DHF groups. The biomarker is characterized by its mass-to-charge ratio as determined by mass spectrometry and is represented in daltons.

FIG. 29 shows a graphical representation of the differential signal intensity of the 23,260 DA biomarker in control, DF, and DHF groups. The biomarker is characterized by its mass-to-charge ratio as determined by mass spectrometry and is represented in daltons.

FIG. 30 shows a graphical representation of the differential signal intensity of the 12,662 DA biomarker in control, DF, and DHF groups. The biomarker is characterized by its mass-to-charge ratio as determined by mass spectrometry and is represented in daltons.

FIG. 31 shows a graphical representation of the differential signal intensity of the 13,295 DA biomarker in control, DF, and DHF groups. The biomarker is characterized by its mass-to-charge ratio as determined by mass spectrometry and is represented in daltons.

FIG. 32 shows a graphical representation of the differential signal intensity of the 12,650 DA biomarker in control, DF, and DHF groups. The biomarker is characterized by its mass-to-charge ratio as determined by mass spectrometry and is represented in daltons.

FIG. 33 shows a graphical representation of the differential signal intensity of the 7,625 DA biomarker in control, DF, and DHF groups. The biomarker is characterized by its mass-to-charge ratio as determined by mass spectrometry and is represented in daltons.

FIG. 34 shows a graphical representation of the differential signal intensity of the 13,317 DA biomarker in control, DF, and DHF groups. The biomarker is characterized by its mass-to-charge ratio as determined by mass spectrometry and is represented in daltons.

### DETAILED DESCRIPTION

### Introduction

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the expression level of the biomarker (e.g., as indicated by the mean, median, or other measure) in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics), drug toxicity, and the like.

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a biomarker” includes a combination of two or more biomarkers, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

As used herein, the term “residue” refers to amino acids or analogs thereof.

As used herein, the term “peptide” refers to peptides, proteins, fragments of proteins, peptidomimetics, and the like that are comprised of more than one amino acid residue or similar molecule.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., 1990, J. Mol. Biol. 215:403-410. Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics), prognostics, and drug toxicity.

The term “chronic” refers to a disease or condition that is long-lasting or recurrent. The term chronic describes the course of the disease, or its rate of onset and development. A chronic course is distinguished from a recurrent course; recurrent diseases or conditions relapse repeatedly, with periods of remission in between.

The term “acute” means an exacerbated event or attack, of short course, followed by a period of remission.

Biomarkers for Dengue

This invention provides, inter alia, polypeptide-based biomarkers that are differentially present in subjects having dengue, in particular, and particularly that are differentially expressed in subjects infected with dengue versus non uninfected individuals (e.g., control, healthy, benign condition or other disease state). The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomolecules and not process limitations in the manner in which the biomolecules are discriminated. In one aspect, this invention provides these biomarkers in isolated form.

The biomarkers of Tables 3-4 were discovered using SELDI technology employing ProteinChip® arrays from Ciphergen Biosystems, Inc. (Fremont, Calif.) (“Ciphergen”). Serum samples were collected from subjects diagnosed with dengue and subjects diagnosed as healthy as well as subjects diagnosed with other febrile illnesses (OFIs). “Other febrile illnesses” are defined as cases with no evidence of dengue infection and no obvious bacterial, rickettsial or protozoan etiology, including, without limitation, chikungunya, measles, leptospirosis, yellow fever, influenza, West Nile, Japanese, and St Louis encephalitis. The samples were fractionated by anion exchange chromatography. Fractionated samples were applied to SELDI biochips and spectra of polypeptides in the samples were generated by time-of-flight mass spectrometry on a Ciphergen PBS IIc mass spectrometer. The spectra thus obtained were analyzed by Ciphergen Express™ Data Manager Software with Biomarker Wizard and Biomarker Pattern Software from Ciphergen Biosystems, Inc. The mass spectra for each group were subjected to scatter plot analysis. A Mann-Whitney test analysis was employed to compare dengue and control groups for each protein cluster in the scatter plot, and proteins were selected that differed significantly (p<0.05) between the two groups. This method is described in more gel electrophoresis followed by protein identification by matrix-assisted laser desorption/ionization mass spectrometry (DIGE and MALDI-TOFMS). This method is described in more detail in the Examples.

The biomarkers thus discovered are presented in Tables 1-4 (the protocol for the data obtained is further described below in the Examples).

TABLE 1
Biomarkers identified using differential SDS-PAGE gel followed by protein
identification by matrix-assisted laser desorption/ionization mass spectroscopy.
Samples were fractionated using ZOOM IEF Fractionator (Invitrogen).

| | | Approximate | | Predicted | |
|---|---|---|---|---|---|
| | | position on gel | | molecular | Calculated |
| Fraction | Band # | (kDa) | ID | weight (Da) | pI value |
| F1 (pH | 2.3 | 116.3-200.00 | α2-macroglobulin | 164600 | 6.00 |
| 3.0-4.6) | 4.5 | 66.3-97.4 | plasma protease C1 | 55347 | 6.09 |
| | | | inhibitor precursor | | |
| | | | carboxypeptidase N | 61431 | 5.63 |
| | | | subunit 2 precursor | | |
| | | | α1-acid glycoprotein | 23725 | 4.93 |
| | | | 1 precursor | | |
| | | | serotransferrin | 79280 | 6.81 |
| | | | precursor | | |
| | | | lumican precursor | 38747 | 6.16 |
| | 6.7 | 55.4-66.3 | lumican precursor | 38747 | 6.16 |
| | | | hemopexin | 52385 | 6.55 |
| | | | precursor | | |
| | 8.9 | 31.0-36.5 | apolipoprotein D | 21547 | 5.06 |
| | | | precursor | | |
| | 10.11 | 10-14.4 | complement C4 A | 194247 | 6.65 |
| | | | precursor | | |
| F2 (pH | 12.13 | 116.3-200.00 | α2-macroglobulin | 164600 | 6.00 |
| 4.6-5.4) | | | AMBP protein | 39886 | 5.95 |
| | | | precursor | | |
| | 14.15 | 97.4-116.3 | ceruloplasmin | 122983 | 5.44 |
| | | | precursor | | |
| | | | apolipoprotein A-I | 30759 | 5.56 |
| | | | precursor | | |
| | | | complement C4 A | 194247 | 6.65 |
| | | | precursor | | |
| | | | plasma protease C1 | 55347 | 6.09 |
| | | | inhibitor precursor | | |
| | | | hemopexin | 52385 | 6.55 |
| | | | precursor | | |
| | | | apolipoprotein B-100 | 516666 | 6.61 |
| | 16.17 | 66.3-97.4 | prothrombin | 71475 | 5.64 |
| | | | precursor | | |
| | | | lumican precursor | 38747 | 6.16 |
| | | | insulin-like growth | 66735 | 6.33 |
| | | | factor-binding | | |
| | | | protein complex acid | | |
| | | | labile chain | | |
| | | | precursor | | |
| | | | α1B-glycoprotein | 54809 | 5.58 |
| | | | precursor | | |
| | | | apolipoprotein A-I | 30759 | 5.56 |
| | | | precursor | | |
| | | | afamin precursor | 70963 | 5.64 |
| | | | haptoglobin-related | 39496 | 6.42 |
| | | | protein precursor | | |
| | | | vitamin K-dependent | 77127 | 5.48 |
| | | | protein S precursor | | |
| | | | complement C4 A | 194247 | 6.65 |
| | | | precursor | | |
| | | | apolipoprotein A-IV | 45371 | 5.28 |
| | | | precursor | | |
| | | | hemopexin | 52385 | 6.55 |
| | | | precursor | | |
| | | | vitronectin precursor | 55069 | 5.55 |
| | 18.19 | 14.4-21.5 | apolipoprotein A-I | 30759 | 5.56 |
| | | | precursor | | |
| | | | haptoglobin-related | 39496 | 6.42 |
| | | | protein precursor | | |
| F3 (pH | 20.21 | 31.0-36.5 | mannose binding | 26526 | 5.39 |
| 5.4-7.0) | | | protein C precursor | | |
| | | | complement C4 A | 194247 | 6.65 |
| | | | precursor | | |
| | | | prothrombin | 71475 | 5.64 |
| | | | precursor | | |
| | | | haptoglobin-related | 39496 | 6.42 |
| | | | protein precursor | | |
| | | | fibrinogen α chain | 95656 | 5.70 |
| | | | precursor | | |
| | | | complement C3 | 188569 | 6.02 |
| | | | precursor | | |
| F4 (pH | 22.23 | ~66.3 | complement C3 | 188569 | 6.02 |
| 7.0-9.1) | | | precursor | | |
| | | | complement C4 A | 194247 | 6.65 |
| | | | precursor | | |
| | | | serotransferrin | 79280 | 6.81 |
| | | | precursor | | |
| | | | fibrinogen α chain | 95656 | 5.70 |
| | | | precursor | | |
| | | | C4b-binding protein | 69042 | |
| | | | α chain precursor | | |
| | 24.25 | ~36.5 | complement C1q | 26670 | 8.83 |
| | | | subcomponent | | |
| | | | subunit B precursor | | |
| | | | serotransferrin | 79280 | 6.81 |
| | | | precursor | | |
| | | | complement C4 A | 194247 | 6.65 |
| | | | precursor | | |
| | | | prothrombin | 71475 | 5.64 |
| | | | precursor | | |
| | | | complement C3 | 188569 | 6.02 |
| | | | precursor | | |
| | | | fibrinogen α chain | 95656 | 5.70 |
| | | | precursor | | |
| | 26.27 | 14.4-21.5 | haptoglobin-related | 39496 | 6.42 |
| | | | protein precursor | | |
| | | | complement | 22435 | 8.49 |
| | | | component C8 γ | | |
| | | | chain precursor | | |
| F5 (pH | 28.29 | 116.3-200.00 | | | |
| 9.1- 10.0) | 30.31 | ~116.3 | tRNA(Ile)-lysine | 50497 | 9.57 |
| | | | synthase- | | |
| | | | Streptococcus | | |
| | | | mutans | | |
| | 32.33 | 55.4-66.3 | complement C3 | 188569 | 6.02 |
| | | | precursor | | |
| | | | complement C4 A | 194247 | 6.65 |
| | | | precursor | | |
| | 34.35 | 36.5-55.4 | | | |

TABLE 2
TABLE 2. Biomarkers identified employing differential SDS-PAGE gel followed by
matrix-assisted laser desorption/ionization mass spectroscopy, their presence/absence in
sample and their obtained scores using MASCOTT search engine.

| | | Approximate | | Predicted | | | | | |
|---|---|---|---|---|---|---|---|---|---|
| | | position on | | molecular | Calculated | Coverage | | | |
| Fraction | Band # | gel (kDa) | ID | weight (Da) | pI value | (%) | Ct | DV | Score |
| F1 (pH | 2.3 | 116.3-200.00 | α2-macroglobulin | 164600 | 6.00 | 4 | | x | 390 |
| 3.0-4.6) | 4.5 | 66.3-97.4 | plasma protease C1 | 55347 | 6.09 | 11 | x | | 311 |
| | | | inhibitor precursor | | | | | | |
| | | | carboxypeptidase N | 61431 | 5.63 | 9 | x | xx | 220 |
| | | | subunit 2 precursor | | | | | | |
| | | | α1-acid glycoprotein | 23725 | 4.93 | 3 | x | | 52 |
| | | | 1 precursor | | | | | | |
| | | | serotransferrin | 79280 | 6.81 | 3 | | x | 99 |
| | | | precursor | | | | | | |
| | | | lumican precursor | 38747 | 6.16 | 5 | | x | 60 |
| | 6.7 | 55.4-66.3 | Serum albumin | 71317 | 5.92 | 32 | x | xx | 1236 |
| | | | precursor | | | | | | |
| | | | lumican precursor | 38747 | 6.16 | 7 | x | | 130 |
| | | | hemopexin | 52385 | 6.55 | 4 | | x | 73 |
| | | | precursor | | | | | | |
| | 8.9 | 31.0-36.5 | apolipoprotein D | 21547 | 5.06 | 28 | xx | x | 229 |
| | | | precursor | | | | | | |
| | 10.11 | 10-14.4 | complement C4 A | 194247 | 6.65 | 0 | x | | 49 |
| | | | precursor | | | | | | |
| F2 (pH | 12.13 | 116.3-200.00 | α2-macroglobulin | 164600 | 6.00 | 1 | x | | 63 |
| 4.6-5.4) | | | AMBP protein | 39886 | 5.95 | 3 | x | | 54 |
| | | | precursor | | | | | | |
| | 14.15 | 97.4-116.3 | Serum albumin | 71317 | 5.92 | 12 | x | | 396 |
| | | | precursor | | | | | | |
| | | | ceruloplasmin | 122983 | 5.44 | 12 | x | xx | 529 |
| | | | precursor | | | | | | |
| | | | apolipoprotein A-I | 30759 | 5.56 | 12 | | x | 112 |
| | | | precursor | | | | | | |
| | | | complement C4 A | 194247 | 6.65 | 1 | | x | 78 |
| | | | precursor | | | | | | |
| | | | plasma protease C1 | 55347 | 6.09 | 4 | | x | 65 |
| | | | inhibitor precursor | | | | | | |
| | | | hemopexin | 52385 | 6.55 | 2 | | x | 49 |
| | | | precursor | | | | | | |
| | | | apolipoprotein B- | 516666 | 6.61 | 0 | | x | 45 |
| | | | 100 | | | | | | |
| | 16.17 | 66.3-97.4 | prothrombin | 71475 | 5.64 | 42 | x | xx | 1187 |
| | | | precursor | | | | | | |
| | | | lumican precursor | 38747 | 6.16 | 14 | xx | x | 225 |
| | | | insulin-like growth | 66735 | 6.33 | 9 | x | xx | 299 |
| | | | factor-binding | | | | | | |
| | | | protein complex | | | | | | |
| | | | acid labile chain | | | | | | |
| | | | precursor | | | | | | |
| | | | α1B-glycoprotein | 54809 | 5.58 | 11 | xx | x | 187 |
| | | | precursor | | | | | | |
| | | | apolipoprotein A-I | 30759 | 5.56 | 12 | xx | x | 154 |
| | | | precursor | | | | | | |
| | | | afamin precursor | 70963 | 5.64 | 10 | xx | x | 154 |
| | | | | | | | | 9% | |
| | | | | | | | | cov. | |
| | | | | | | | | scor | |
| | | | | | | | | 219 | |
| | | | haptoglobin-related | 39496 | 6.42 | 6 | x | | 99 |
| | | | protein precursor | | | | | | |
| | | | vitamin K- | 77127 | 5.48 | 5 | x | xx | 184 |
| | | | dependent protein S | | | | | | |
| | | | precursor | | | | | | |
| | | | complement C4 A | 194247 | 6.65 | 0 | x | | 67 |
| | | | precursor | | | | | | |
| | | | apolipoprotein A-IV | 45371 | 5.28 | 2 | x | | 49 |
| | | | precursor | | | | | | |
| | | | hemopexin | 52385 | 6.55 | 4 | x | x | 95 |
| | | | precursor | | | | | | |
| | | | vitronectin precursor | 55069 | 5.55 | 3 | | x | 92 |
| | 18.19 | 14.4-21.5 | apolipoprotein A-I | 30759 | 5.56 | 19 | xx | x | 213 |
| | | | precursor | | | | | | |
| | | | haptoglobin-related | 39496 | 6.42 | 3 | x | xx | 137 |
| | | | protein precursor | | | | | | |
| F3 | 20.21 | 31.0-36.5 | mannose binding | 26526 | 5.39 | 26 | x | xx | 292 |
| (pH 5.4-7.0) | | | protein C precursor | | | | | | |
| | | | complement C4 A | 194247 | 6.65 | 4 | x | x | 277 |
| | | | precursor | | | | | | |
| | | | prothrombin | 71475 | 5.64 | 5 | x | | 154 |
| | | | precursor | | | | | | |
| | | | haptoglobin-related | 39496 | 6.42 | 3 | x | | 62 |
| | | | protein precursor | | | | | | |
| | | | fibrinogen α chain | 95656 | 5.70 | 2 | | x | 73 |
| | | | precursor | | | | | | |
| | | | Serum albumin | 71317 | 5.92 | 23 | x | xx | 715 |
| | | | precursor | | | | | | |
| | | | complement C3 | 188569 | 6.02 | 0 | | x | 46 |
| | | | precursor | | | | | | |
| F4 | 24.25 | ~36.5 | complement C1q | 26670 | 8.83 | 19 | x | xx | 216 |
| (pH 7.0-9.1) | | | subcomponent | | | | | | |
| | | | subunit B precursor | | | | | | |
| | | | Ig gamma-1 chain C | 36596 | 8.46 | 32 | x | xx | 426 |
| | | | region | | | | | | |
| | | | Serum albumin | 71317 | 5.92 | 6 | x | | 206 |
| | | | precursor | | | | | | |
| | | | serotransferrin | 79280 | 6.81 | 4 | x | | 120 |
| | | | precursor | | | | | | |
| | | | complement C4 A | 194247 | 6.65 | 5 | x | xx | 319 |
| | | | precursor | | | | | | |
| | | | Ig gamma-2 chain C | 36489 | 7.66 | 11 | x | xx | 146 |
| | | | region | | | | | | |
| | | | Ig gamma-4 chain C | 36431 | 7.18 | 13 | | x | 94 |
| | | | region | | | | | | |
| | | | prothrombin | 71475 | 5.64 | 1 | x | | 90 |
| | | | precursor | | | | | | |
| | | | complement C3 | 188569 | 6.02 | 3 | x | xx | 268 |
| | | | precursor | | | | | | |
| | | | fibrinogen α chain | 95656 | 5.70 | 10 | | x | 314 |
| | | | precursor | | | | | | |
| | 26.27 | 14.4-21.5 | haptoglobin-related | 39496 | 6.42 | 3 | xx | x | 127 |
| | | | protein precursor | | | | | | |
| | | | Serum albumin | 71317 | 5.92 | 8 | xx | x | 228 |
| | | | precursor | | | | | | |
| | | | Hemoglobin subunit | 15305 | 8.72 | 25 | x | | 221 |
| | | | alpha | | | | | | |
| | | | Ig gamma-1 chain C | 36596 | 8.46 | 10 | x | xx | 136 |
| | | | region | | | | | | |
| | | | complement | 22435 | 8.49 | 5 | xx | x | 63 |
| | | | component C8 γ | | | | | | |
| | | | chain precursor | | | | | | |
| F5 | 28.29 | 116.3-200.00 | Ig gamma-1 chain C | 36596 | 8.46 | 3 | x | | 47 |
| (pH 9.1-10.0) | | | region | | | | | | |
| | 30.31 | ~116.3 | Ig gamma-1 chain C | 36596 | 8.46 | 8 | xx | x | 110 |
| | | | region | | | | | | |
| | | | Ig gamma-2 chain C | 36489 | 7.66 | 5 | x | | 64 |
| | | | region | | | | | | |
| | | | tRNA(Ile)-lysine | 50497 | 9.57 | 3 | | x | 46 |
| | | | synthase- | | | | | | |
| | | | Streptococcus | | | | | | |
| | | | mutans | | | | | | |
| | 32.33 | 55.4-66.3 | complement C3 | 188569 | 6.02 | 4 | x | | 266 |
| | | | precursor | | | | | | |
| | | | Ig gamma-1 chain C | 36596 | 8.46 | 19 | xx | x | 268 |
| | | | region | | | | | | |
| | | | Ig gamma-2 chain C | 36489 | 7.66 | 12 | x | | 167 |
| | | | region | | | | | | |
| | | | complement C4 A | 194247 | 6.65 | 1 | x | | 111 |
| | | | precursor | | | | | | |
| | 34.35 | 36.5-55.4 | Ig gamma-1 chain C | 36596 | 8.46 | 3 | x | | 59 |
| | | | region | | | | | | |

TABLE 3
Table 3. Predicted correlation between biomarkers discovered employing differential SDS-PAGE gel followed by
protein identification by matrix-assisted laser desorption/ionization mass spectroscopy and those employing SELDI
technology.
TABLE 3
Proposed Proteins

| | | Gel | | SELDI | | | |
|---|---|---|---|---|---|---|---|
| | | Sample found | | | M/Z Average | | |
| Protein | Mass | Ct | DFNV | Fraction | Ct1_2 | DF1_2 | DHF1_2 |
| [symbol] | 70963 | x | x | x | x | | |
| precursor | | | | | | | |
| AMBE protein | 39686 | x | x | F6ISH | 39686.176 ± 8.381 | 39887.686 ± 8.490 | 39891.827 ± 7.9 |
| precursor | | | | | | | |
| Apolipoprotein | [symbol] | x | x | x | x | | |
| Acl | | | | | | | |
| precursor | | | | | | | |
| Apolipoprotein | 46371 | x | x | F6ISH | [symbol] ± 19.421 | [symbol] ± 22.022 | 46363.08 ± 0.08049 |
| [symbol] | | | | F6CSH | 45682.198 ± 4.882 | 45581.119 ± 9.163 | 0.08639 ± 0.02933 |
| precursor | | | | | | | |
| Apolipoprotein | 616668 | x | x | | x | | |
| B-100 | | | | | | | |
| Apolipoprotein | 21547 | xx | x | F6CSL | 33613.916 ± 3.735 | 88614.675 ± 8.733 | 88863.863 ± 4.863 |
| D precursor | [symbol] ) | | | F6ISL | 33656.728 ± 0.81 | 33556.081 ± 1.271 | 33558.493 ± 0.836 |
| C4b-binding | 69042 | x | x | F1CSH | 89023.681 ± 7.865 | 89821.468 ± 9.752 | 89023.41 ± 10.408 |
| protein α | | | | F6CSL | 34624.834 ± 0.893 | 34623.590 ± 4.66 | 34821.235 ± 7.390 |
| chain | | | | | | | |
| precursor | | | | | | | |
| Carboxypeptidase | 61431 | x | xx | F6CSH | 61383.104 ± 3.227 | 61384.759 ± 5.492 | 61.383.023 ± 4.071 |
| N subunit | | | | | | | |
| 2 precursor | | | | | | | |
| [symbol] | 122963 | x | xx | F6CSH | 108960.906 ± 12.625 | 108981.316 ± 8.176 | 108960.41 ± 2.362 |
| precursor | | | | | | | |
| Complement | 26670 | x | xx | F6CSH | 25404.284 ± 6.504 | 25403.649 ± 4.573 | 26404.844 ± 4.573 |
| C1q | | | | | | | |
| subcomponent | | | | | | | |
| [symbol] & | | | | | | | |
| precursor | | | | | | | |
| Complement | 188669 | x | x | | x | | |
| C2 precursor | | | | | | | |
| Complement | 194247 | x | x | x | x | | |
| C4A | | | | | | | |
| precursor | | | | | | | |
| Complement | 62435 | xx | x | | x | | |
| component C3 | | | | | | | |
| [symbol] chain | | | | | | | |
| precursor | | | | | | | |
| Fibrinogen α | 95856 | x | x | x | x | | |
| chain | | | | | | | |
| precursor | | | | | | | |
| Haptoglobin- | 39496 | xx | x | x | x | | |
| related protein | | | | | | | |
| precursor | | | | | | | |
| Hemoglobin | 16385 | x | x | F1CSH | 16308.449 ± 0.683 | 16308.648 ± 0.829 | 16308.689 ± 1.241 |
| subunit alpha | | | | | | | |
| [symbol] | 62365 | x | xx | F6ISH | 52579.804 ± 14.203 | 62678.178 ± 13.289 | 52686.671 ± 7.276 |
| precursor | | | | | | | |
| Ic mu heavy | 43543 | x | x | x | x | | |
| chain disease | | | | | | | |
| protein | | | | | | | |
| Ic gamma-1 | 38686 | x | x | x | x | | |
| chain C region | | | | | | | |
| Ic gamma-2 | 38489 | x | x | x | x | | |
| chain C region | | | | | | | |
| Insulin-like | 66735 | x | xx | F8CSH | 66724.441 ± 0.384 | 66724.559 ± 0.338 | 68724.523 ± 8.481 |
| growth factor- | | | | F6ISH | 66626.217 ± 0.43 | 66626.311 ± 0.478 | 66626.319 ± 0.550 |
| binding, | | | | | | | |
| protein, | | | | | | | |
| complex acid | | | | | | | |
| labile chain, | | | | | | | |
| precursor | | | | | | | |
| Lumican | 387.57 | x | x | x | x | | |
| crocuses | | | | | | | |
| Mammas, | 25526 | x | xx | x | | | |
| binding, | | | | | | | |
| protein C, | | | | | | | |
| precursor | | | | | | | |
| Plasma | 55347 | x | x | F1ISH | 55237.604 ± 23.135 | 55287.389 ± 33.414 | 55305.740 ± 28.403 |
| protease C1 | | | | | | | |
| inhibitor | | | | | | | |
| precursor | | | | | | | |
| Prothrombin | 71470 | x | xx | x | x | | |
| precursor | | | | | | | |
| Serotransferrin | 79280 | x | x | F0CSH | 79341.09 ± 4.442 | 79342.512 ± 4.3002 | 79341.031 ± 2.621 |
| precursor | | | | | | | |
| Serum, | 71317 | x | x | x | x | | |
| Albumin, | | | | | | | |
| precursor | | | | | | | |
| IRNA [symbol] ab | 50497 | x | x | x | x | | |
| lysine | | | | | | | |
| sy [symbol] base, | | | | | | | |
| Stropto [symbol] | | | | | | | |
| matura | | | | | | | |
| Vitamin K1 | 77127 | x | xx | F5CSH | 75141.288 ± 2.803 | 75142.892 ± 2.12 | 75141.517 ± 1.906 |
| da [symbol] dent, | (75123) | | | | | | |
| protein S, | | | | | | | |
| precursor | | | | | | | |
| V [symbol] actin, | 55669 | x | x | F6CSH | 53489.215 ± 11.828 | 53486.247 ± 3.714 | 63466.088 ± 8.800 |
| precursor | | | | | | | |
| α1-acid, | 23725 | x | x | x | x | | |
| phy [symbol] protein 1, | | | | | | | |
| precursor | | | | | | | |
| α18- | 54809 | xx | x | F1ISH | 51693.736 ± 27.672 | 54594.675 ± 29.019 | 54692 ± 956 ± 26.418 |
| pty [symbol] protein | | | | | | | |
| precursor | | | | | | | |
| α [symbol] - | 184600 | x | x | x | x | | |
| macroglobulin | | | | | | | |

| | SELDI | | | | | |
|---|---|---|---|---|---|---|
| | | | | | DHF1_DHF2 vs | |
| | Intensity average | | | Average | DF1_DF2 | |
| Protein | Ct1_2 | DF1_2 | DHF1_2 | M/Z | p value | roc |
| [symbol] | | | | x | x | x |
| precursor | | | | | | |
| AMBE protein | 0.02268 ± 0.00357 | 0.03806 ± 0.02312 | 0.04406 ± 0.01453 | 39668.6485 | [symbol] | 0.3478861 |
| precursor | | | | | | |
| Apolipoprotein | | | | x | x | x |
| Acl | | | | | | |
| precursor | | | | | | |
| Apolipoprotein | 0.17419 ± 0.05317 | 0.23873 ± 0.09065 | 45368.9521 | 45368.9621 | 0.02369367 | 0.31481481 |
| [symbol] | 0.06414 ± [symbol] | 0.06085 ± 0.01474 | 45581.8839 | 45581.6839 | 1.0000000 | 0.5142045 |
| precursor | | | | | | |
| Apolipoprotein | | | | x | x | x |
| B-100 | | | | | | |
| Apolipoprotein | 0.40412 ± 0.15261 | 0.23087 ± 0.1193 | 0.30821 ± 0.14392 | 33614.1787 | 0.35620899 | 0.80897438 |
| D precursor | 0.58585 ± 0.0234 | 0.41558 ± 0.0689 | 0.50485 ± 0.11008 | 33665.3982 | 0.2779327 | 0.8028431 |
| C4b-binding | 0.13767 ± 0.14204 | 0.09796 ± 0.04115 | 0.12274 ± 0.11603 | 60023.5419 | 0.9024018 | 0.4717282 |
| protein α | 0.41100 ± 0.16538 | 0.07664 ± 0.1223 | 0.32514 ± 0.15886 | 34523.5514 | 0.37303721 | 0.59466128 |
| chain | | | | | | |
| precursor | | | | | | |
| Carboxypeptidase | 0.10387 ± 0.03894 | 0.0672 ± 0.2669 | 0.10007 ± 0.02767 | 61383.3748 | 0.0021063 | 0.2073864 |
| N subunit | | | | | | |
| 2 precursor | | | | | | |
| [symbol] | 0.0628 ± 0.01843 | 0.07162 ± 0.02190 | 0.08466 ± 0.02747 | 108961.408 | 0.0614734 | 0.3258999 |
| precursor | | | | | | |
| Complement | 0.07889 ± 0.08123 | 0.13804 ± 0.05838 | 0.13699 ± 0.04529 | 25404.0365 | 0.6574155 | 0.4886384 |
| C1q | | | | | | |
| subcomponent | | | | | | |
| [symbol] & | | | | | | |
| precursor | | | | | | |
| Complement | | | | x | x | x |
| C2 precursor | | | | | | |
| Complement | | | | x | x | x |
| C4A | | | | | | |
| precursor | | | | | | |
| Complement | | | | x | x | x |
| component C3 | | | | | | |
| [symbol] chain | | | | | | |
| precursor | | | | | | |
| Fibrinogen α | | | | x | x | x |
| chain | | | | | | |
| precursor | | | | | | |
| Haptoglobin- | | | | x | x | x |
| related protein | | | | | | |
| precursor | | | | | | |
| Hemoglobin | 0.72968 ± 0.42315 | 0.68053 ± 0.42315 | 0.41601 ± 0.22400 | 15308.5557 | 0.0805598 | 0.6517857 |
| subunit alpha | | | | | | |
| [symbol] | 0.02969 ± 0.01659 | 0.038181 ± 0.01165 | 0.05034 ± 0.01527 | 89680.3719 | 0.0108684 | 0.2376812 |
| precursor | | | | | | |
| Ic mu heavy | | | | x | x | x |
| chain disease | | | | | | |
| protein | | | | | | |
| Ic gamma-1 | | | | x | x | x |
| chain C region | | | | | | |
| Ic gamma-2 | | | | x | x | x |
| chain C region | | | | | | |
| Insulin-like | 2.80614 ± 0.68767 | 1.62726 ± 0.45856 | 1.70258 ± 0.41922 | 66794.6829 | 0.676938.7 | 0.4546466 |
| growth factor- | 1.91881 ± 0.43148 | 1.45151 ± 0.36184 | 1.79486 ± 0.42081 | 66626.9708 | 0.0190614 | 0.2724638 |
| binding, | | | | | | |
| protein, | | | | | | |
| complex acid | | | | | | |
| labile chain, | | | | | | |
| precursor | | | | | | |
| Lumican | | | | x | x | x |
| crocuses | | | | | | |
| Mammas, | | | | x | x | x |
| binding, | | | | | | |
| protein C, | | | | | | |
| precursor | | | | | | |
| Plasma | 0.15307 ± 0.07217 | 0.03087 ± 0.03844 | 0.09185 ± 0.03664 | 55291.4169 | 0.53341644 | 0.43333333 |
| protease C1 | | | | | | |
| inhibitor | | | | | | |
| precursor | | | | | | |
| Prothrombin | | | | x | x | x |
| precursor | | | | | | |
| Serotransferrin | 0.05046 ± 0.02730 | 0.06479 ± 0.03165 | 0.09141 ± 0.04297 | 29341.4807 | 0.0363029 | 0.3465908 |
| precursor | | | | | | |
| Serum, | | | | x | x | x |
| Albumin, | | | | | | |
| precursor | | | | | | |
| IRNA [symbol] ab | | | | x | x | x |
| lysine | | | | | | |
| sy [symbol] base, | | | | | | |
| Stropto [symbol] | | | | | | |
| matura | | | | | | |
| Vitamin K1 | 0.87103 ± 0.23150 | 0.86353 ± 0.29625 | 0.76484 ± 0.22220 | 75141.8895 | 0.1684346 | 0.6175595 |
| da [symbol] dent, | | | | | | |
| protein S, | | | | | | |
| precursor | | | | | | |
| V [symbol] actin, | 0.0265 ± 0.01133 | 0.0339 ± 0.01367 | 0.04679 ± 0.01447 | 63487.7965 | 0.00860 [symbol] 40 | 0.227 [symbol] 722 |
| precursor | | | | | | |
| α1-acid, | | | | x | x | x |
| phy [symbol] protein 1, | | | | | | |
| precursor | | | | | | |
| α18- | 0.21745 ± 0.09393 | 0.11499 ± 0.95443 | 0.12176 ± 0.04866 | 54593.1773 | 0.6909161 | 0.45833333 |
| pty [symbol] protein | | | | | | |
| precursor | | | | | | |
| α [symbol] - | | | | x | x | x |
| macroglobulin | | | | | | |

| | SELDI | | | |
|---|---|---|---|---|
| | Ct1_2 vs DHF1_2 | | Ct1_2 vs DF1_2 | |
| Protein | p value | roc | p value | roc |
| [symbol] | x | x | x | x |
| precursor | | | | |
| AMBE protein | 0.0000889 | 0.8400000 | 0.0019049 | 0.7946377 |
| precursor | | | | |
| Apolipoprotein | x | x | x | x |
| Acl | | | | |
| precursor | | | | |
| Apolipoprotein | 0.2187700 | 0.8 [symbol] | 0.272779 | 0.4194444 |
| [symbol] | 0.0000113 | 0.0892857 | 0.0000728 | 0.1916584 |
| precursor | | | | |
| Apolipoprotein | x | x | x | x |
| B-100 | | | | |
| Apolipoprotein | 0.0317443 | 0.3078451 | 0.0042800 | 0.2208333 |
| D precursor | 0.0163588 | 0.2819473 | 0.0331955 | 0.3333333 |
| C4b-binding | 0.6084078 | 0.4419848 | 0.3632777 | 0.4396269 |
| protein α | 0.1211833 | 0.3960784 | 0.0014444 | 0.2347222 |
| chain | | | | |
| precursor | | | | |
| Carboxypeptidase | 0.6962703 | 0.4732143 | 0.0081005 | 0.1916684 |
| N subunit | | | | |
| 2 precursor | | | | |
| [symbol] | 0.0129359 | 0.7112500 | 0.0722886 | 0.6809624 |
| precursor | | | | |
| Complement | 0.0001048 | 0.8268928 | 0.0000222 | 0.8409091 |
| C1q | | | | |
| subcomponent | | | | |
| [symbol] & | | | | |
| precursor | | | | |
| Complement | x | x | x | x |
| C2 precursor | | | | |
| Complement | x | x | x | x |
| C4A | | | | |
| precursor | | | | |
| Complement | x | x | x | x |
| component C3 | | | | |
| [symbol] chain | | | | |
| precursor | | | | |
| Fibrinogen α | x | x | x | x |
| chain | | | | |
| precursor | | | | |
| Haptoglobin- | x | x | x | x |
| related protein | | | | |
| precursor | | | | |
| Hemoglobin | 0.0097068 | 0.2867143 | 0.1824001 | 0.3937075 |
| subunit alpha | | | | |
| [symbol] | 0.0006866 | 0.8000000 | 0.0504608 | 0.8427538 |
| precursor | | | | |
| Ic mu heavy | x | x | x | x |
| chain disease | | | | |
| protein | | | | |
| Ic gamma-1 | x | x | x | x |
| chain C region | | | | |
| Ic gamma-2 | x | x | x | x |
| chain C region | | | | |
| Insulin-like | 0.0078297 | 0.2500000 | 0.0008317 | 0.2482687 |
| growth factor- | 0.4130817 | 0.4400000 | 0.0002888 | 0.2181359 |
| binding, | | | | |
| protein, | | | | |
| complex acid | | | | |
| labile chain, | | | | |
| precursor | | | | |
| Lumican | x | x | x | x |
| crocuses | | | | |
| Mammas, | x | x | x | x |
| binding, | | | | |
| protein C, | | | | |
| precursor | | | | |
| Plasma | 0.0088733 | 0.3314815 | 0.0004176 | 0.2129830 |
| protease C1 | | | | |
| inhibitor | | | | |
| precursor | | | | |
| Prothrombin | x | x | x | x |
| precursor | | | | |
| Serotransferrin | 0.0013931 | 0.7723214 | 0.1090298 | 0.6298701 |
| precursor | | | | |
| Serum, | x | x | x | x |
| Albumin, | | | | |
| precursor | | | | |
| IRNA [symbol] ab | x | x | x | x |
| lysine | | | | |
| sy [symbol] base, | | | | |
| Stropto [symbol] | | | | |
| matura | | | | |
| Vitamin K1 | 0.1727823 | 0.5300080 | 0.0073757 | 0.7295238 |
| da [symbol] dent, | | | | |
| protein S, | | | | |
| precursor | | | | |
| V [symbol] actin, | 0.0000609 | 0.8437500 | 0.1323513 | 0.62398701 |
| precursor | | | | |
| α1-acid, | x | x | x | x |
| phy [symbol] protein 1, | | | | |
| precursor | | | | |
| α18- | 0.0009107 | 0.2098788 | 0.0001174 | 0.1629830 |
| pty [symbol] protein | | | | |
| precursor | | | | |
| α [symbol] - | x | x | x | x |
| macroglobulin | | | | |

[symbol]

indicates data missing or illegible when filed

TABLE 4
Table 4. Summary list of most significant biomarkers discovered using SELDI technology.
TABLE 4

| | | DHF1_DHF2 vs | | | | | | |
|---|---|---|---|---|---|---|---|---|
| | | DF1_DF2 | | Ct1_2 vs DHF1_2 | | Ct1_2 vs DF1_2 | | Intensity average |
| | Index | p-value | roc | p-value | roc | p-value | roc | Ct1_2 |
| F1CSL | 20 | 0.0858999 | 0.3333333 | 0.0000002 | 0.9637815 | 0.0000000 | 0.9472789 | 0.13754 ± 0.09945 |
| | 40 | 0.4194702 | 0.5714286 | 0.0000000 | 0.9915966 | 0.0000000 | 0.9778912 | 0.20533 ± 0.13663 |
| F1CSH | 42 | 0.0868537 | 0.3258929 | 0.0000003 | 0.9842857 | 0.0000448 | 0.8248299 | 1.01776 ± 0.3444982 |
| F5CSL | 23 | 0.7526763 | 0.4915459 | 0.0000023 | 0.1071429 | 0.0000006 | 0.1894720 | 0.26052 ± 0.18249 |
| F5CSH | 68 | 0.6677795 | 0.4717262 | 0.0000002 | 0.0200000 | 0.0000000 | 0.0342867 | 1.25365 ± 0.73605 |
| F6CSL | 106 | 0.091766 | 0.3333333 | 0.0000019 | 0.0960734 | 0.0000000 | 0.0444444 | 0.33588 ± 0.23484 |
| F6ISL | 22 | 0.0006400 | 0.8149510 | 0.4876235 | 0.4361339 | 0.0025296 | 0.7205480 | 0.40871 ± 0.22847 |
| F6ISH | 67 | 0.5806161 | 0.6913043 | 0.0000002 | 0.0100000 | 0.0000000 | 0.0297101 | 0.75444 ± 0.52943 |

| | | Intensity average | | m/z, |
|---|---|---|---|---|
| | Index | DF1_2 | DHF1_2 | average |
| F1CSL | 20 | 0.79791 ± 0.40975 | 1.14175 ± 0.61843 | 3808.262 |
| | 40 | 4.5165 ± 3.52856 | 3.33219 ± 2.33453 | 4596.111 |
| F1CSH | 42 | 1.70585 ± 0.55744 | 2.07821 ± 0.42565 | 23260.27 |
| F5CSL | 23 | 0.04893 ± 0.02439 | 0.0486 ± 0.03122 | 12662.53 |
| F5CSH | 68 | 0.26347 ± 0.17809 | 0.2595 ± 0.13859 | 13295.38 |
| F6CSL | 106 | 0.04337 ± 0.02361 | 0.06224 ± 0.03383 | 12650.52 |
| F6ISL | 22 | 0.7085 ± 0.30474 | 0.39536 ± 0.32736 | 7605.507 |
| F6ISH | 67 | 0.12406 ± 0.0447 | 0.11899 ± 0.04494 | 13312.42 |

The biomarkers are characterized by their mass-to-charge ratio as determined by mass spectrometry. The mass-to-charge ratios were determined from mass spectra generated on a Ciphergen Biosystems, Inc. PBS IIc mass spectrometer. This instrument has a mass accuracy of about +/−0.15 percent. Additionally, the instrument has a mass resolution of about 400 to 1000 m/dm, where m is mass and dm is the mass spectral peak width at 0.5 peak height. The mass-to-charge ratio of the biomarkers was determined using Biomarker Wizard™ software (Ciphergen Biosystems, Inc.). Biomarker Wizard assigns a mass-to-charge ratio to a biomarker by clustering the mass-to-charge ratios of the same peaks from all the spectra analyzed, as determined by the PBSIIc, taking the maximum and minimum mass-to-charge-ratio in the cluster, and dividing by two. Accordingly, the masses provided reflect these specifications.

The identity of certain of the biomarkers of Tables 1-4 of this invention has been determined and is indicated in Tables 1-4 and/or Table 5. Table 5 shows the accession numbers for the biomarkers as determined on the NCBI web-site on Oct. 10, 2008. Thus, one of ordinary skill in the art could ascertain the nucleotide and amino acid sequences of the biomarkers based on this information without undue experimentation.

Tables 17-24 (below) show biomarkers of the invention. Table 17 shows the exemplary biomarkers for detecting primary DENV infection as detected by Biomarker Pattern Software (BPS). Tables B-D show all biomarkers detected by SELDI for primary DENV infection that had a p-value smaller than or equal to 0.05. Table 21 shows the exemplary biomarkers for detecting secondary DENV infection as detected by BPS. Tables F and G show the biomarkers for detecting secondary DENV infection. Table 24 shows the biomarkers that can be used to differentiate between primary and secondary DENV infection as detected by BPS.

For biomarkers whose identify has been determined, the presence of the biomarker can be determined by methods known in the art other than mass spectrometry.

TABLE 5
by alphabetical order
TABLE 5. Non-redundant list of the discovered biomarkers using
differential SDS-PAGE gel followed by protein identification by
matrix-assisted laser desorption/ionization mass spectroscopy.
Accession numbers as determined on the NCBI website on
Oct. 10^{th}, 2008.

| | | Predicted | | |
|---|---|---|---|---|
| | | molecular | Calculated | Accession |
| | ID | weight (Da) | pI value | number |
| 1 | afamin precursor | 70963 | 5.64 | NP_001124 |
| 2 | AMBP protein | 39886 | 5.95 | P02760 |
| | precursor | | | |
| 3 | apolipoprotein A-I | 30759 | 5.56 | NP_000030 |
| | precursor | | | |
| 4 | apolipoprotein A-IV | 45371 | 5.28 | NP_000473 |
| | precursor | | | |
| 5 | apolipoprotein B-100 | 516666 | 6.61 | P04114 |
| 6 | apolipoprotein D | 21547 | 5.06 | P05090 |
| | precursor | | | |
| 7 | C4b-binding protein α | 69042 | | P04003 |
| | chain precursor | | | |
| 8 | carboxypeptidase N | 61431 | 5.63 | P22792 |
| | subunit 2 precursor | | | |
| 9 | ceruloplasmin | 122983 | 5.44 | NP_000087 |
| | precursor | | | |
| 10 | complement C1q | 26670 | 8.83 | P02746 |
| | subcomponent subunit | | | |
| | B precursor | | | |
| 11 | complement C3 | 188569 | 6.02 | P01024 |
| | precursor | | | |
| 12 | complement C4 A | 194247 | 6.65 | P0C0L4 |
| | precursor | | | |
| 13 | complement | 22435 | 8.49 | P07360 |
| | component C8 γ chain | | | |
| | precursor | | | |
| 14 | fibrinogen α chain | 95656 | 5.70 | P02671 |
| | precursor | | | |
| 15 | haptoglobin-related | 39496 | 6.42 | Q28801 |
| | protein precursor | | | |
| 16 | Hemoglobin subunit | 15305 | 8.72 | P69905 |
| | alpha | | | |
| 17 | hemopexin precursor | 52385 | 6.55 | AAA52704 |
| 18 | Ig mu heavy chain | 43543 | 5.13 | P04220 |
| | disease protein | | | |
| 19 | Ig gamma-1 chain C | 36596 | 8.46 | P20759 |
| | region | | | |
| 20 | Ig gamma-2 chain C | 36489 | 7.66 | P01859 |
| | region | | | |
| 21 | insulin-like growth | 66735 | 6.33 | P35858 |
| | factor-binding protein | | | |
| | complex acid labile | | | |
| | chain precursor | | | |
| 22 | lumican precursor | 38747 | 6.16 | NP_002336 |
| 23 | mannose binding | 26526 | 5.39 | P08661 |
| | protein C precursor | | | |
| 24 | plasma protease C1 | 55347 | 6.09 | AAB59387 |
| | inhibitor precursor | | | |
| 25 | prothrombin precursor | 71475 | 5.64 | P00734 |
| 26 | serotransferrin | 79280 | 6.81 | P02787 |
| | precursor | | | |
| 27 | Serum albumin | 71317 | 5.92 | P02768 |
| | precursor | | | |
| 28 | tRNA(Ile)-lysine | 50497 | 9.57 | Q8DWM9 |
| | synthase- | | | |
| | Streptococcus mutans | | | |
| 29 | vitamin K-dependent | 77127 | 5.48 | P07225 |
| | protein S precursor | | | |
| 30 | vitronectin precursor | 55069 | 5.55 | NP_000629 |
| 31 | α1-acid glycoprotein 1 | 23725 | 4.93 | AAA40699 |
| | precursor | | | |
| 32 | α1B-glycoprotein | 54809 | 5.58 | Q9EPH1 |
| | precursor | | | |
| 33 | α2-macroglobulin | 164600 | 6.00 | CAA48670 |
| 34 | LEAP-2 precursor | | | NP_443203 |
| 35 | LEAP-2 | | | CAC51515 |

The biomarkers of this invention can be further characterized by the shape of their spectral peak in time-of-flight mass spectrometry.

The biomarkers of this invention can be further characterized by their binding properties on chromatographic surfaces.

Because the biomarkers are characterized by mass-to-charge ratio and binding properties, they can be detected by mass spectrometry without knowing their specific identity. The identity of certain of the biomarkers of Tables 1-4, and 17-24 is known and, if known, is shown in Tables 1-4 and/or Table 5. If desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

The preferred biological source for detection of the biomarkers is serum. However, in other aspects, the biomarkers are detected in urine and other biological samples.

The biomarkers of this invention are biomolecules. Accordingly, this invention provides these biomolecules in isolated form. The biomarkers can be isolated from biological fluids, such as serum. They can be isolated by any method known in the art, based on both their mass and their binding characteristics. For example, a sample comprising the biomolecules can be subject to chromatographic fractionation, as described herein, and subject to further separation by, e.g., acrylamide gel electrophoresis. Knowledge of the identity of the biomarker also allows their isolation by immunoaffinity chromatography.

Biomarkers and Modified Forms of a Protein

Proteins frequently exist in a sample in a plurality of different forms. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cysteinylation, sulphonation and acetylation. When detecting or measuring a protein in a sample, the ability to differentiate between different forms of a protein depends upon the nature of the difference and the method used to detect or measure. For example, immunological methods of detection typically cannot distinguish between different forms of a protein that contain the same epitope or epitopes to which the antibody or antibodies are directed. In diagnostic assays, the inability to distinguish different forms of a protein has little impact when the forms detected by the particular method used are equally good biomarkers as any particular form. However, when a particular form (or a subset of particular forms) of a protein is a better biomarker than the collection of modified forms detected together by a particular method, the power of the assay can suffer. In this case, it is useful to employ an assay method that distinguishes between forms of a protein and that specifically detects and measures a desired modified form or forms of the protein. Distinguishing different forms of an analyte or specifically detecting a particular form of an analyte is referred to as “resolving” the analyte.

The collection of analytes detected in an assay and the ability to resolve modified forms of a protein of course depends on the methodology used. For example, an immunoassay using a monoclonal antibody will detect all forms of a protein containing the eptiope and will not distinguish between them. However, a sandwich immunoassay that uses two antibodies directed against different epitopes on a protein will detect all forms of the protein that contain both epitope and will not detect those forms that contain only one of the epitopes. Accordingly this method can be useful when the modified forms differ in a terminal amino acid and one of the antibodies is directed to the terminus of one of these forms.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

Mass spectrometry is a particularly powerful resolving methodology because different forms of a protein typically have different masses and can be differentiated by mass spectrometry. One useful methodology combines mass spectrometry with immunoassay. First, a biospecific capture reagent (e.g., an antibody, aptamer or Affibody that recognizes the biomarker and modified forms of it) is used to capture the biomarker of interest. Preferably, the biospecific capture reagent is bound to a solid phase, such as a bead, a plate, a membrane or a chip. After unbound materials are washed away, the captured analytes are detected and/or measured by mass spectrometry. (This method also will also result in the capture of protein interactors that are bound to the proteins or that are otherwise recognized by antibodies and that, themselves, can be biomarkers.) Then, the captured proteins can be detected by SELDI mass spectrometry or by eluting the proteins from the capture reagent and detecting the eluted proteins by traditional MALDI, SELDI or any other ionization method for mass spectrometry (e.g., electrospray).

Thus, when reference is made herein to detecting a particular protein or to measuring the amount of a particular protein, it means detecting and measuring the protein with or without resolving modified forms of protein. For example, the step of “measuring Apolipoprotein A-IV precursor” includes measuring Apolipoprotein A-IV precursor by means that do not differentiate between various forms of the protein (e.g., certain immunoassays) as well as by means that differentiate some forms from other forms or that measure a specific form of the protein. In contrast, when it is desired to measure a particular form or forms of a protein, the particular form (or forms) is specified. For example, “measuring M7.065159” or a biomarker of 7.065159 kDa means measuring it in a way that distinguishes it from forms of the protein that do not have the characteristic properties identified in Tables 1-5.

Detection of Biomarkers for Dengue

The biomarkers of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one aspect, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. No. 6,225,047 (Hutchens & Yip); U.S. Pat. No. 6,537,749 (Kuimelis and Wagner); U.S. Pat. No. 6,329,209 (Wagner et al.); PCT International Publication No. WO 00/56934 (Englert et al.); PCT International Publication No. WO 03/048768 (Boutell et al.); and U.S. Pat. No. 5,242,828 (Bergstrom et al.).

Detection by Mass Spectrometry

In a preferred aspect, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

SELDI

A preferred mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain aspects the probes have the capture reagent already attached to the surface. In other aspects, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and HSO (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” Can 24, 2005); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Um et al., “Hydrophobic Surface Chip,” Can 15, 2003); U.S. Patent Application No. US 2003/0218130 A1 (Boschetti et al., “Biochips With Surfaces Coated With Polysaccharide-Based Hydrogels,” Apr. 14, 2003) and U.S. Patent Application No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow the biomarker or biomarkers that can be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain aspects, the energy absorbing molecule is incorporated into a linear or cross-linked polymer, e.g., a polymethacrylate. For example, the composition can be a co-polymer of a-cyano-4-methacryloyloxycinnamic acid and acrylate. In another aspect, the composition is a co-polymer of a-cyano-4-methacryloyloxycinnamic acid, acrylate and 3-(tri-ethoxy)silyl propyl methacrylate. In another aspect, the composition is a co-polymer of a-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

Other Mass Spectrometry Methods

In another mass spectrometry method, the biomarkers are first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI. In yet another method, one could isolate the biomarkers using gel elecrophoresis and detect the biomarkers by MALDI OR SELDI.

Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data can be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

General Protocol for SELDI Detection of Biomarkers for Dengue

A preferred protocol for the detection of the biomarkers of this invention is as follows. The biological sample to be tested, e.g., serum, preferably is subject to pre-fractionation before SELDI analysis. This simplifies the sample and improves sensitivity. A preferred method of pre-fractionation involves contacting the sample with an anion exchange chromatographic material, such as Q HyperD (BioSepra, SA). The bound materials are then subject to stepwise pH elution using buffers at pH 9, pH 7, pH 5 and pH 4. (The fractions in which the biomarkers are eluted also are indicated in Tables 1-2, and 4) Various fractions containing the biomarker are collected.

The sample to be tested (preferably pre-fractionated) is then contacted with an affinity capture probe comprising an cation exchange adsorbent (preferably a WCX ProteinChip array (Ciphergen Biosystems, Inc.)) or an IMAC adsorbent (preferably an IMAC3 ProteinChip array (Ciphergen Biosystems, Inc.)). The probe is washed with a buffer that will retain the biomarker while washing away unbound molecules. The biomarkers are detected by laser desorption/ionization mass spectrometry.

Alternatively, if antibodies that recognize the biomarker are available, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

Detection by Immunoassay

In another aspect of the invention, the biomarkers of the invention are measured by a method other than mass spectrometry or other than methods that rely on a measurement of the mass of the biomarker. In one such aspect that does not rely on mass, the biomarkers of this invention are measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. Nephelometry is an assay done in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

Determination of Subject Dengue Status

Single Markers

The biomarkers of the invention can be used in diagnostic tests to assess dengue status in a subject, e.g., to diagnose Dengue. The phrase “Dengue status” includes any distinguishable manifestation of the disease, including non-disease. For example, disease status includes, without limitation, the presence or absence of disease (e.g., dengue v. non dengue or Dengue v. other disease (e.g., OFIs), the risk of developing disease, the stage of the disease, the progress of disease (e.g., progress of disease or remission of disease over time) and the effectiveness or response to treatment of disease. The status of the subject can inform the practitioner about what status set is being distinguished. For example, a subject that presents with signs of a disease could be classed into Dengue v. non-Dengue disease, while a person exposed to a situation in which Dengue infection is possible and who is presenting with signs of Dengue infection could be classified into Dengue v. non-Dengue. Based on this status, further procedures can be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

The biomarkers of this invention show a statistical difference in different dengue statuses of at least p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at least 98% and about 100%.

Each biomarker listed in Tables 1-5 and 17-24 is differentially present in dengue, and, therefore, each is individually useful in aiding in the determination of dengue status. The method involves, first, measuring the selected biomarker in a subject sample using the methods described herein, e.g., capture on a SELDI biochip followed by detection by mass spectrometry and, second, comparing the measurement with a diagnostic amount or cut-off that distinguishes a positive dengue status from a negative dengue status. The diagnostic amount represents a measured amount of a biomarker above which or below which a subject is classified as having a particular dengue status, e.g. DF, DHF, DSS. For example, if the biomarker is up-regulated compared to normal during dengue, then a measured amount above the diagnostic cutoff provides a diagnosis of dengue status. Alternatively, if the biomarker is down-regulated during dengue, then a measured amount below the diagnostic cutoff provides a diagnosis of dengue status. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different dengue statuses, as was done here, and drawing the cut-off to suit the diagnostician's desired levels of specificity and sensitivity.

Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. A combination of at least two biomarkers is sometimes referred to as a “biomarker profile” or “biomarker fingerprint.”

Presence of Dengue

In one aspect, this invention provides methods for determining the presence or absence of dengue in a subject (status: dengue v. non-dengue). The presence or absence of dengue is determined by measuring the relevant biomarker or biomarkers and then either submitting them to a classification algorithm or comparing them with a reference amount and/or pattern of biomarkers that is associated with the particular risk level.

Determining Risk of Developing Disease

In one aspect, this invention provides methods for determining the risk of developing disease in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level.

Determining Stage of Disease

In one aspect, this invention provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

Determining Course (Progression/Remission) of Disease

In one aspect, this invention provides methods for determining the course of disease in a subject. Disease course refers to changes in disease status over time, including disease progression (worsening) and disease regression (improvement). Over time, the amounts or relative amounts (e.g., the pattern) of the biomarkers changes. Therefore, the trend of these markers, either increased or decreased over time toward diseased or non-diseased indicates the course of the disease. Accordingly, this method involves measuring one or more biomarkers in a subject at least two different time points, e.g., a first time and a second time, and comparing the change in amounts, if any. The course of disease is determined based on these comparisons.

Subject Management

In certain aspects of the methods of qualifying dengue status, the methods further comprise managing subject treatment based on the status. Such management includes the actions of the physician or clinician subsequent to determining dengue status. For example, if a physician makes a diagnosis of dengue, then a certain regime of treatment, such as prescription or administration of paracetamol, antipyretics or a combination thereof, might follow. Alternatively, a diagnosis of non-dengue might be followed with further testing to determine a specific disease that might the patient might be suffering from. Also, if the diagnostic test gives an inconclusive result on dengue status, further tests can be called for.

The methods described herein can be used in combination with and other tests and/or methods that are used to qualify dengue status in a subject. For example, in certain aspects, the methods described herein are used to determine whether or not a subject has an increased likelihood of having dengue. These methods can be used in combination with other tests that are useful for either diagnosing dengue in a subject or ruling out other diagnoses.

Additional aspects of the invention relate to the communication of assay results or diagnoses or both to technicians, physicians or patients, for example. In certain aspects, computers will be used to communicate assay results or diagnoses or both to interested parties, e.g., physicians and their patients. In some aspects, the assays will be performed or the assay results analyzed in a country or jurisdiction which differs from the country or jurisdiction to which the results or diagnoses are communicated.

In a preferred aspect of the invention, a diagnosis based on the presence or absence in a test subject of any the biomarkers of Table 1-5, and 17-24 is communicated to the subject as soon as possible after the diagnosis is obtained. The diagnosis can be communicated to the subject by the subject's treating physician. Alternatively, the diagnosis can be sent to a test subject by email or communicated to the subject by phone. A computer can be used to communicate the diagnosis by email or phone. In certain aspects, the message containing results of a diagnostic test can be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain aspects of the methods of the invention, all or some of the method steps, including the assaying of samples, diagnosing of diseases, and communicating of assay results or diagnoses, can be carried out in diverse (e.g., foreign) jurisdictions.

Determining Therapeutic Efficacy of Pharmaceutical Drug

In another aspect, this invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen can involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of the biomarkers of this invention changes toward a non-disease profile. One can follow the course of the amounts of these biomarkers in the subject during the course of treatment. Accordingly, this method involves measuring one or more biomarkers in a subject receiving drug therapy, and correlating the amounts of the biomarkers with the disease status of the subject. One aspect of this method involves determining the levels of the biomarkers at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications. If a treatment is effective, then the biomarkers will trend toward normal, while if treatment is ineffective, the biomarkers will trend toward disease indications.

Generation of Classification Algorithms for Qualifying Dengue Status

In some aspects, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model can comprise raw data or pre-processed data. In some aspects, raw data can be obtained directly from time-of-flight spectra or mass spectra, and then can be optionally “pre-processed” as described above.

Classification models can be formed using any suitable statistical classification (or “learning”) method that attempts to segregate bodies of data into classes based on objective parameters present in the data. Classification methods can be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data can then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other aspects, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used can be physically separate from the mass spectrometer that is used to create the spectra of interest, or it can be coupled to the mass spectrometer.

The training data set and the classification models according to aspects of the invention can be embodied by computer code that is executed or used by a digital computer. The computer code can be stored on any suitable computer readable media including optical or magnetic disks, sticks, tapes, and the like, and can be written in any suitable computer programming language including C, C++, visual basic, and the like

The learning algorithms described above are useful both for developing classification algorithms for the biomarkers already discovered, or for finding new biomarkers for dengue. The classification algorithms, in turn, form the base for diagnostic tests by providing diagnostic values (e.g., cut-off points) for biomarkers used singly or in combination.

Compositions of Matter

In another aspect, this invention provides compositions of matter based on the biomarkers of this invention.

In one aspect, this invention provides biomarkers of this invention in purified form. Purified biomarkers have utility as antigens to raise antibodies. Purified biomarkers also have utility as standards in assay procedures. As used herein, a “purified biomarker” is a biomarker that has been isolated from other proteins and peptdies, and/or other material from the biological sample in which the biomarker is found. Biomarkers can be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods can be performed at any appropriate scale, for example, in a chromatography column, or on a biochip.

In another aspect, this invention provides a biospecific capture reagent, optionally in purified form, that specifically binds a biomarker of this invention. In one aspect, the biospecific capture reagent is an antibody. Such compositions are useful for detecting the biomarker in a detection assay, e.g., for diagnostics.

In another aspect, this invention provides an article comprising a biospecific capture reagent that binds a biomarker of this invention, wherein the reagent is bound to a solid phase. For example, this invention contemplates a device comprising bead, chip, membrane, monolith or microtiter plate derivatized with the biospecific capture reagent. Such articles are useful in biomarker detection assays.

In another aspect this invention provides a composition comprising a biospecific capture reagent, such as an antibody, bound to a biomarker of this invention, the composition optionally being in purified form. Such compositions are useful for purifying the biomarker or in assays for detecting the biomarker.

In another aspect, this invention provides an article comprising a solid substrate to which is attached an adsorbent, e.g., a chromatographic adsorbent or a biospecific capture reagent, to which is further bound a biomarker of this invention. In one aspect, the article is a biochip or a probe for mass spectrometry, e.g., a SELDI probe. Such articles are useful for purifying the biomarker or detecting the biomarker.

Kits for Detection of Biomarkers for Dengue

In another aspect, the present invention provides kits for qualifying dengue status, which kits are used to detect biomarkers according to the invention. In one aspect, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip® arrays. In the case of biospecfic capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit can include more than type of adsorbent, each present on a different solid support.

In a further aspect, such a kit can comprise instructions for suitable operational parameters in the form of a label or separate insert. For example, the instructions can inform a consumer about how to collect the sample, how to wash the probe or the particular biomarkers to be detected.

In yet another aspect, the kit can comprise one or more containers with biomarker samples, to be used as standard(s) for calibration.

Use of Biomarkers for Dengue in Screening Assays and Methods of Treating Dengue

The methods of the present invention have other applications as well. For example, the biomarkers can be used to screen for compounds that modulate the expression of the biomarkers in vitro or in vivo, which compounds in turn can be useful in treating or preventing dengue in patients. In another example, the biomarkers can be used to monitor the response to treatments for dengue. In yet another example, the biomarkers can be used in heredity studies to determine if the subject is at risk for developing dengue.

Thus, for example, the kits of this invention could include a solid substrate having a hydrophobic function, such as a protein biochip (e.g., a Ciphergen H50 ProteinChip array, e.g., ProteinChip array) and a sodium acetate buffer for washing the substrate, as well as instructions providing a protocol to measure the biomarkers of this invention on the chip and to use these measurements to diagnose dengue.

Compounds suitable for therapeutic testing can be screened initially by identifying compounds which interact with one or more biomarkers listed in Table 1, 2, 3, 4, 5, 17, 18, 19, 20, 21, 22, 23, or 24. By way of example, screening might include recombinantly expressing a biomarker listed in Table 1, 2, 3, 4, 5, 17, 18, 19, 20, 21, 22, 23, or 24, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins can recognize and cleave one or more biomarkers of Table 1, 2, 3, 4, 5, 17, 18, 19, 20, 21, 22, 23, or 24, in which case the proteins can be detected by monitoring the digestion of one or more biomarkers in a standard assay, e.g., by gel electrophoresis of the proteins.

In a related aspect, the ability of a test compound to inhibit the activity of one or more of the biomarkers of Table 1, 2, 3, 4, 5, 17, 18, 19, 20, 21, 22, 23, or 24, can be measured. One of skill in the art will recognize that the techniques used to measure the activity of a particular biomarker will vary depending on the function and properties of the biomarker. For example, an enzymatic activity of a biomarker can be assayed provided that an appropriate substrate is available and provided that the concentration of the substrate or the appearance of the reaction product is readily measurable. The ability of potentially therapeutic test compounds to inhibit or enhance the activity of a given biomarker can be determined by measuring the rates of catalysis in the presence or absence of the test compounds. The ability of a test compound to interfere with a non-enzymatic (e.g., structural) function or activity of one of the biomarkers of Table 1, 2, 3, 4, 5, 17, 18, 19, 20, 21, 22, 23, or 24, can also be measured. For example, the self-assembly of a multi-protein complex which includes one of the biomarkers of Table 1, 2, 3, 4, 5, 17, 18, 19, 20, 21, 22, 23, or 24, can be monitored by spectroscopy in the presence or absence of a test compound. Alternatively, if the biomarker is a non-enzymatic enhancer of transcription, test compounds which interfere with the ability of the biomarker to enhance transcription can be identified by measuring the levels of biomarker-dependent transcription in vivo or in vitro in the presence and absence of the test compound.

Test compounds capable of modulating the activity of any of the biomarkers of Table 1, 2, 3, 4, 5, 17, 18, 19, 20, 21, 22, 23, or 24, can be administered to patients who are suffering from or are at risk of developing dengue. For example, the administration of a test compound which increases the activity of a particular biomarker can decrease the risk of dengue in a patient if the activity of the particular biomarker in vivo prevents the accumulation of proteins for dengue. Conversely, the administration of a test compound which decreases the activity of a particular biomarker can decrease the risk of dengue in a patient if the increased activity of the biomarker is responsible, at least in part, for the onset of dengue.

In an additional aspect, the invention provides a method for identifying compounds useful for the treatment of disorders such as dengue which are associated with increased levels of modified forms of the biomarkers in Table 1, 2, 3, 4, 5, 17, 18, 19, 20, 21, 22, 23, or 24. For example, in one aspect, cell extracts or expression libraries can be screened for compounds which catalyze the cleavage of a full-length biomarker to form truncated forms of the biomarker. In one aspect of such a screening assay, cleavage of the biomarker can be detected by attaching a fluorophore to the biomarker which remains quenched when the biomarker is uncleaved but which fluoresces when the protein is cleaved. Alternatively, a version of full-length biomarker modified so as to render the amide bond between amino acids x and y uncleavable can be used to selectively bind or “trap” the cellular protesase which cleaves full-length biomarker at that site in vivo. Methods for screening and identifying proteases and their targets are well-documented in the scientific literature, e.g., in Lopez-Ottin et al. (Nature Reviews, 2002, 3:509-519).

In yet another aspect, the invention provides a method for treating or reducing the progression or likelihood of a disease, e.g., dengue, which is associated with the increased levels of a truncated biomarker. For example, after one or more proteins have been identified which cleave the full-length biomarker, combinatorial libraries can be screened for compounds which inhibit the cleavage activity of the identified proteins. Methods of screening chemical libraries for such compounds are well-known in art. See, e.g., Lopez-Otin et al. (2002). Alternatively, inhibitory compounds can be intelligently designed based on the structure of the biomarker.

At the clinical level, screening a test compound includes obtaining samples from test subjects before and after the subjects have been exposed to a test compound. The levels in the samples of one or more of the biomarkers listed in Table 1, 2, 3, 4, 5, 17, 18, 19, 20, 21, 22, 23, or 24, can be measured and analyzed to determine whether the levels of the biomarkers change after exposure to a test compound. The samples can be analyzed by mass spectrometry, as described herein, or the samples can be analyzed by any appropriate means known to one of skill in the art. For example, the levels of one or more of the biomarkers listed in Table 1, 2, 3, 4, 5, 17, 18, 19, 20, 21, 22, 23, or 24, can be measured directly by Western blot using radio- or fluorescently-labeled antibodies which specifically bind to the biomarkers. Alternatively, changes in the levels of mRNA encoding the one or more biomarkers can be measured and correlated with the administration of a given test compound to a subject. In a further aspect, the changes in the level of expression of one or more of the biomarkers can be measured using in vitro methods and materials. For example, human tissue cultured cells which express, or are capable of expressing; one or more of the biomarkers of Table 1, 2, 3, 4, 5, 17, 18, 19, 20, 21, 22, 23, or 24, can be contacted with test compounds. Subjects who have been treated with test compounds will be routinely examined for any physiological effects which can result from the treatment. In particular, the test compounds will be evaluated for their ability to decrease disease likelihood in a subject. Alternatively, if the test compounds are administered to subjects who have previously been diagnosed with dengue, test compounds will be screened for their ability to slow or stop the progression of the disease.

The invention will be further described with reference to the following exemplary aspects; however, it is to be understood that the invention is not limited to such exemplary aspects.

### Exemplary Aspects

Below are examples of specific aspects for carrying out the present invention. The examples are offered for illustrative purposes only, and are not intended to limit the scope of the present invention in any way. Efforts have been made to ensure accuracy with respect to numbers used (e.g., amounts, temperatures, and the like), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1

### Discovery of Dengue Biomarkers

Two complimentary approaches to identifying potential biomarkers for the diagnosis or prognosis of dengue have been taken: 1) SELDI-based and 2) gel-based. Based on estimated molecular weight, there is an overlap of biomarkers identified by both approaches (Tables 1-5). Similar methods for the discovery of biomarkers for babesia were used in U.S. provisional application Ser. No. 60/749,449 filed on Dec. 12, 2005 and U.S. provisional application Ser. No. 60/752,285 filed on Dec. 20, 2005, both of which are herein incorporated by reference for all purposes. The discovered biomarkers are shown in Tables 1-5, and Tables 17-24.

Sample Collection

Sample collection was previously peformed by Takol.

Plasma samples from pediatric That patients were obtained. For each dengue infected patient, 3 blood samples were taken at 3 different time points: t1 (1^{st} day of admission), t2 (fever decreased to normal), t3 (convalescence stage 30 days after admission). Each probable dengue diagnosis was confirmed and the serotype as well as the type (primary or secondary) of the infection recorded. Samples of patients with other febrile illnesses (OFIs) were also collected to be used as controls. The samples were stored at −80° C. (Table 6). Table 6 shows a list of specimens collected in Thailand from pediatric patients. The list of 15 controls is not included.

TABLE 6
Classification of DENV infected pediatric patients.

TABLE 6

| | | |
|---|---|---|
| DENV | Primary Infection | Secondary Infection |

| Serotype | DF | DHF | DF | DHF |
|---|---|---|---|---|
| 1 | 3 | 1 | 7 | 6 |
| 2 | 3 | 6 | 7 | 10 |
| 3 | 5 | 0 | 2 | 4 |
| 4 | 1 | 2 | 8 | 7 |
| Total | 12 | 9 | 24 | 27 |

Preparation and Fractionation of Serum Samples

Preparation and fractionation of serum samples was previously performed by Takol.

Fractionation of serum samples was performed with the use of the Biomek 2000 Laboratory Automation Workstation (Beckman Coulter, USA) using software protocols provided by Ciphergen (Ciphergen Biosystems, Fremont, Calif., USA). An Expression Difference Mapping Kit (Ciphergen Biosystems, Fremont, Calif., USA) was also used according to the manufacturer's instructions. Six fractions obtained through Fractionation of serum samples was performed with the use of the Biomek 2000 Laboratory Automation Workstation (Beckman Coulter, USA) using software protocols provided by Ciphergen (Ciphergen Biosystems, Fremont, Calif., USA). An Expression Difference Mapping Kit (Ciphergen Biosystems, Fremont, Calif., USA) was also used according to the manufacturer's instructions. Six fractions obtained through isoelectric point separation were obtained and collected using different buffers: F1 (pH 9), F2 (pH 7), F3 (pH5), F4 (pH 4), F5 (pH 3), F6 (organic solvent). The fractions were stored at −80° C.

SELDI Analysis

Protein Binding Using ProteinChip Arrays

Protein binding using ProteinChip Arrays was previously performed by Takol.

The following chip binding protocol was followed and the samples were processed using an IMAC-3 ProteinChip Array according to the protocol below:

Chip Binding Protocol

Weak Cation Exchange (WCX2) ProteinChip Array

Materials:

Bioprocessor

WCX-2 chip

Vortex

CM low stringency buffer

Deionized water

EAM solution
1. Assemble the WCX-2 protein chip in the bioprocessor.
2. Add 150 ul of CM low stringency buffer to each well.
3. Vortex for 5 minutes (speed 100 rpm) at room temperature.
4. Remove the buffer from the wells.
5. Repeat steps 2 to 3 for a total of 2 washes.
6. Add 90 ul of CM low stringency buffer to each well.
7. Add 10 ul of sample (fractions) to each well.
8. Vortex for 30 minutes (speed 100 rpm) at room temperature.
9. Remove the samples from the wells.
10. Wash each well with 150 ul CM low stringency buffer.
11. Vortex for 5 minutes (100 rpm).
12. Repeat twice for a total of three buffer washes.
13. Remove the washing buffer from the wells and rinse each well with deionized water.
14. Drain the wells and remove the chip from the bioprocessor.
15. Allow the chip to air dry.
16. Apply 0.5-1 ul of EAM solution per spot twice.
17. Allow to air dry after each application.
18. Analyze the chip.

Processing Samples Using an IMAC-3 ProteinChip Array

Material:

Bioprocessors

IMAC Chips

Pap Pen

Votex (VWR VX-2500 Multitube Vortexer)

IMAC3 Chip Buffer:

A) Binding Buffer: 100 mM Sodium Phosphate+0.5M NaCl pH 7.0+0.1% Triton X

B) Charging Buffer (Copper): 100 mM CuSO₄+0.1% Triton X 20

C) Neutralizing Buffer:100 mM NaAcetate pH 4.0+0.1% Triton X 20
1. Place Chip in bioprocessor
2. Load IMAC chips with copper: Apply 50 μl/well of 100 mM CuSO₄
3. Vortex 5 min (speed 100 rpm) at room temperature
4. Remove CuSO₄
5. Wash with water 120 μl/well
6. Vortex 5 min (speed 100 rpm)
7. Neutralize chips: Add 50 μl/well of 100 mM NaAcetate pH 4.0
8. Remove solution
9. Wash with water 120 μl/well
10. Vortex 5 min (speed 100 rpm)
11. Repeat steps 9 & 10 a further two times
12. Equilibrate Chips: Add 120 μl Binding Buffer (PBS/0.5 M NaCl, pH 7.5)
13. Vortex 5 min (100 rpm)
14. Bind fractions to chips: Discard waste and add 80 μl Binding Buffer and 20 μl of fractions (containing samples)
15. Vortex 45-60 min (100 rpm)
16. Discard and wash (PBS/0.5M NaCl, 150 μl/well)
17. Vortex 5 min (100 rpm)
18. Repeat steps 16 & 17 a further two times
19. Rinse chip with dH₂O (150 μl/well)
20. Add Matrix: Remove bioproceesor top and gasket
21. Rinse the Chips quickly with dH₂O
22. Dry chips
23. Circle spots with PAP pen
24. Add 0.5 μl SPA to Chips two times (air dry the spots between addition) Ciphergen normally supplies EAM as 5 mg of dried powder in a tube. Add 100 μl of 100% Acetonitrile (final concentration 50% ACN)+50 μl 2% Trifluoroacetic acid (final conc. 0.5% TFA)+50 μl dH₂O.
   Vortex 1 min (high speed) and leave it in the bunch for 5 min
   Spin 2 min at high speed to pellet any particulates
25. Dry
26. Read within 1 hour

Protein binding to ProteinChip Arrays was performed using the Biomek 2000 Laboratory Automation Workstation (Beckman Coulter) and protein binding software protocols provided by Ciphergen Biosystems. Immobilized affinity capture (IMAC3), weak cation-exchange (CM10) and hydrophobic (H50) ProteinChip Array types (eight spot format) were used (Ciphergen Biosystems). ProteinChip arrays were analyzed in the ProteinChip Biology System reader (model PBS IIc, Ciphergen Biosystems).

### Reading and Analysis of ProteinChip Arrays

To initially compare data between different diseases tested, arrays were read at low (intensity=175, sensitivity=7, optimization range=2000-20,000 Da, high range=50,000 Da) and high (intensity=175, sensitivity=8, optimization range=20,000-50,000 Da, high range=150,000 Da) laser settings. The data was analyzed using ProteinChip software (version 3.2.1) and Ciphergen Express Data Manager (version 2.1) (Ciphergen Biosystems).

All data were imported into Ciphergen Express (CE) and grouped according to each condition (e.g., DHF fraction 1 bound to a WCX2 array, read at low laser intensity). Each data set was calibrated using an equation generated from a spectrum of protein standards, which were collected at the same laser intensity as the collected sample data.

The Baseline for all data was set at 15, and Noise set at 2000 Da (for arrays read at low laser energy) or 10,000 Da (high laser energy). Sample spectra for each group were normalized using a specific set of conditions. Arrays read at low laser intensity were normalized between 2000-100,000 Da, and 10,000-200,000 Da for high laser intensity. An external normalization coefficient of 0.2 was applied for both conditions. As a quality control measure for the comparison of spectra processed on different days, the average normalization factor was first calculated for all spectra within the condition. Any spectra that did not fall within twice the overall average normalization factor were discarded from the analysis.

Peak and Cluster detection (EDM) was then performed for both low and high laser intensities for each sample condition. A distinct set of variables were set for each of the samples collected depending on if they were obtained using low or high laser intensity.

The first set of comparisons was carried between control1 and 1DF1 and 1DHF1, control2 and 1DF2 and 1DHF2, 1DF1 and 1DHF1, 1DF2 and 1DHF2, 1DF3 and 1DHF3 plasma samples. After the first-pass analysis, all clusters found to have a p-value ≦0.05 were visually inspected for peak quality. High quality protein peaks were manually relabelled. A second-pass analysis was carried out; the EDM was run again using only user-detected peaks. Using Biomarker Pattern Software (BPS), a decision analysis software, combination of these candidate biomarkers was determined as well as their specificity and sensitivity using pooled data from 1DF1, 1DF2, 1DHF1 and 1DHF2 versus pooled data from control 1 and 2. These candidate biomarkers represent potential diagnostic biomarkers.

A second set of comparisons was carried out between secondary 2DF1 and 2DF1, 2DF2 and 2DHF2, 2DF3 and 2DHF3. The same first- and second-pass analysis protocol was followed with the same p-value limit.

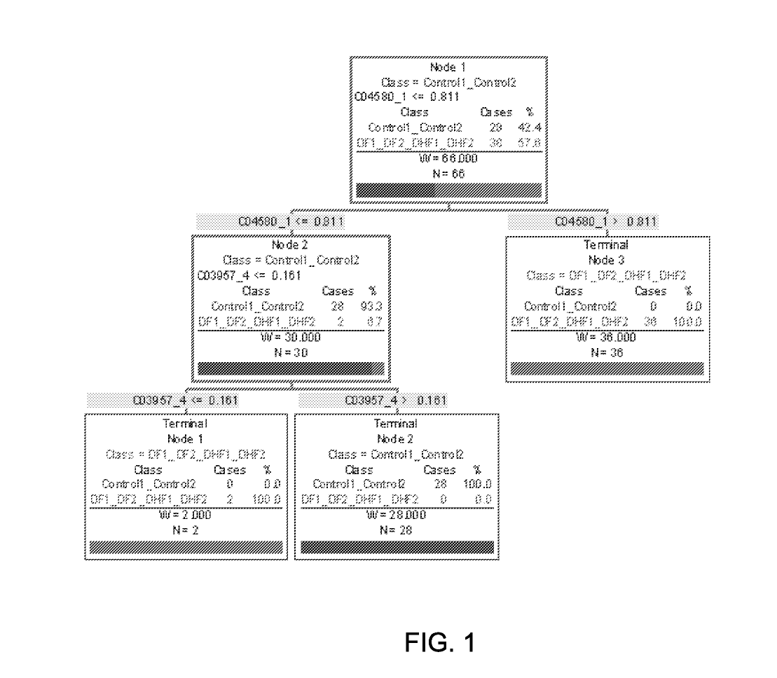

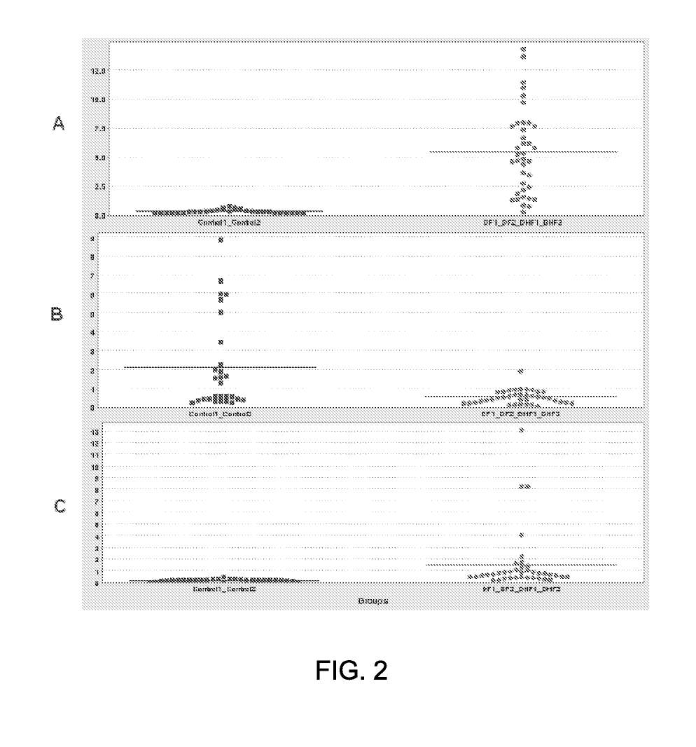

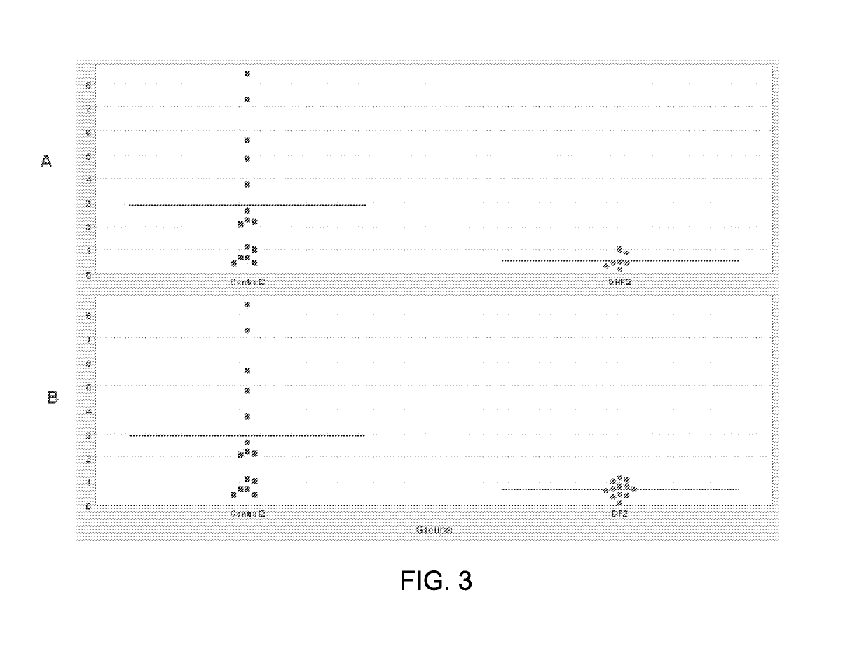

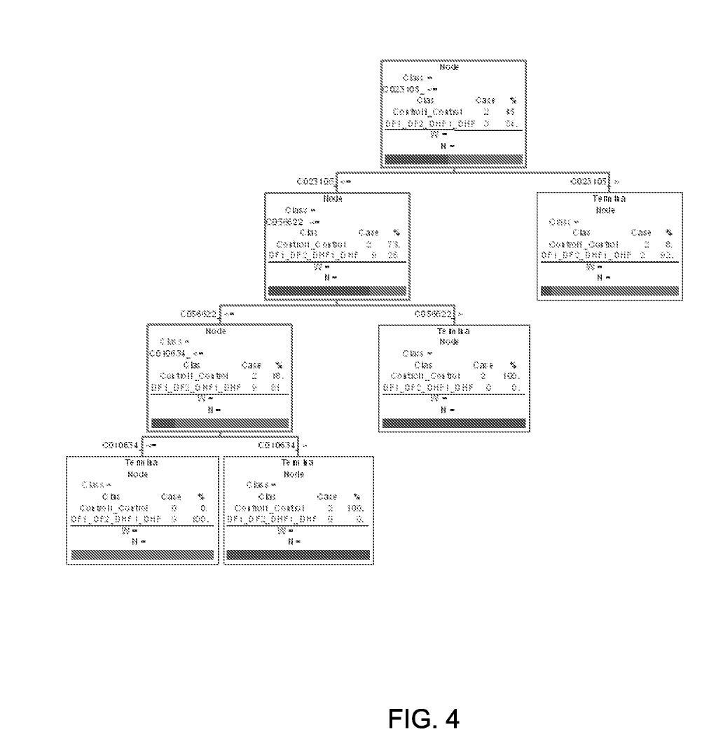

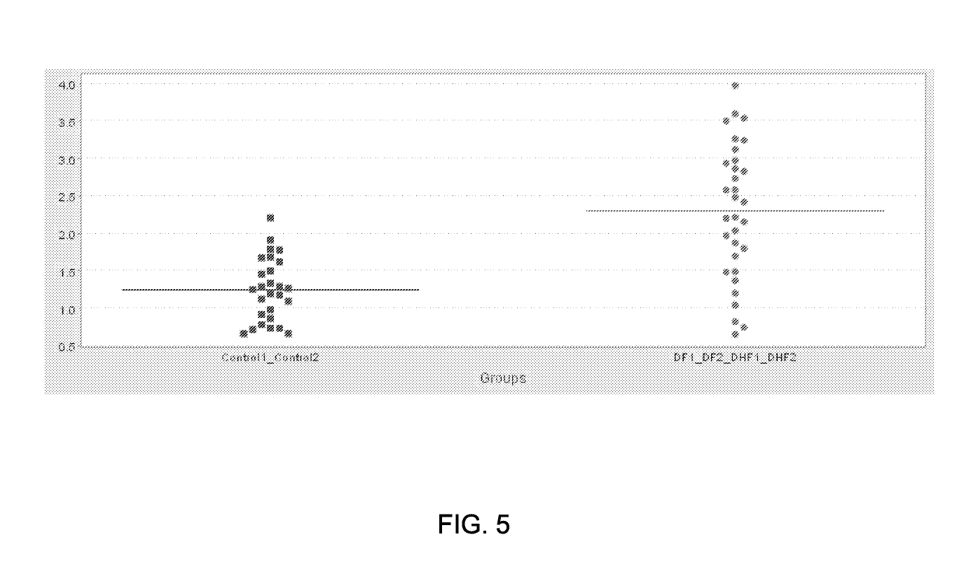

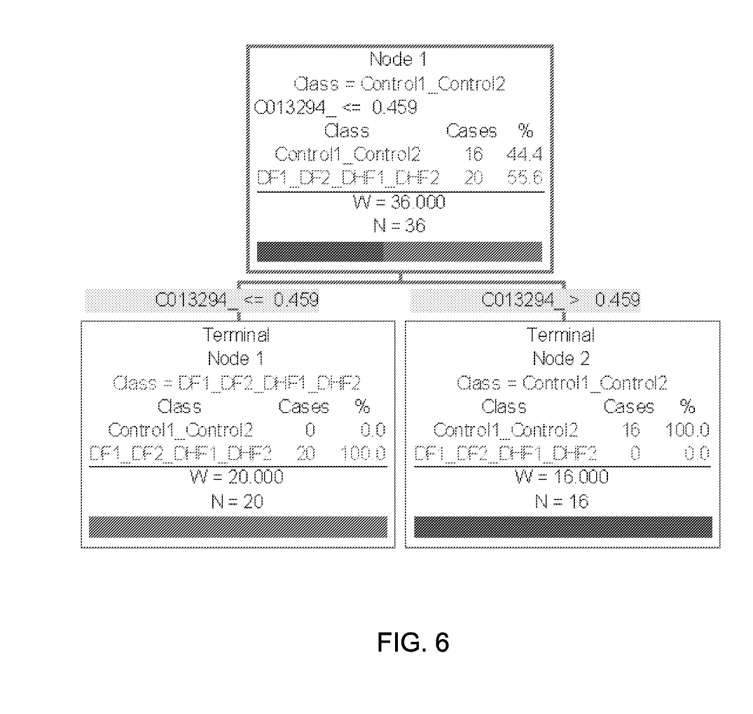

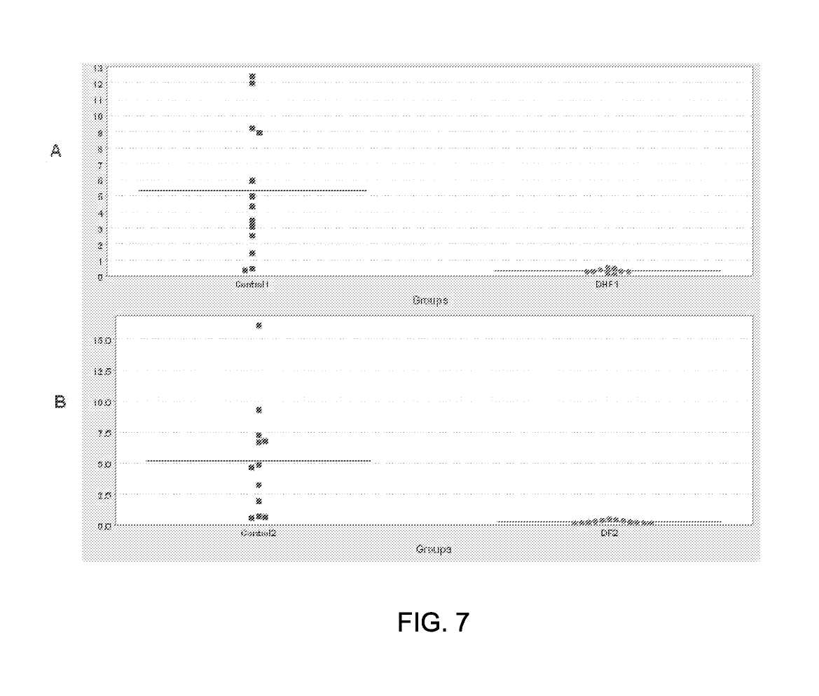

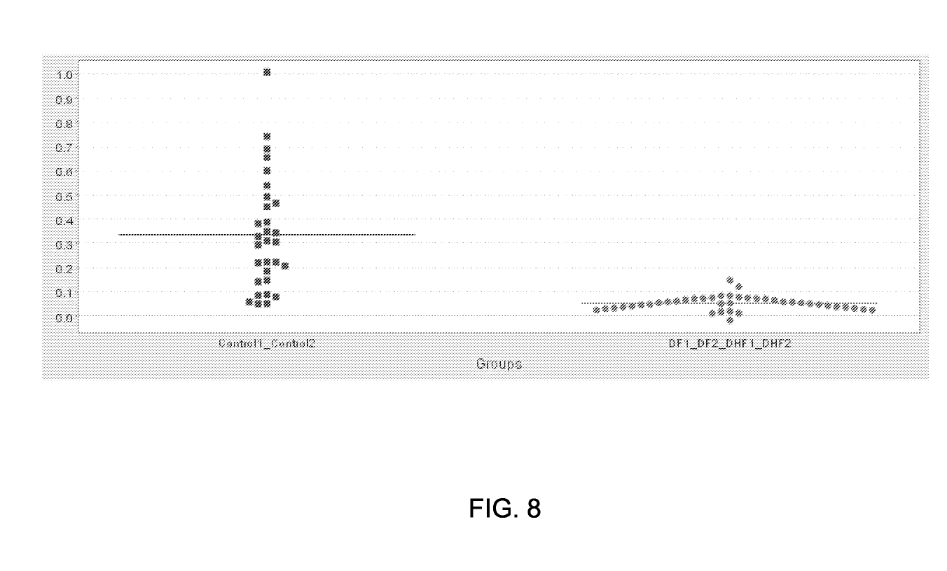

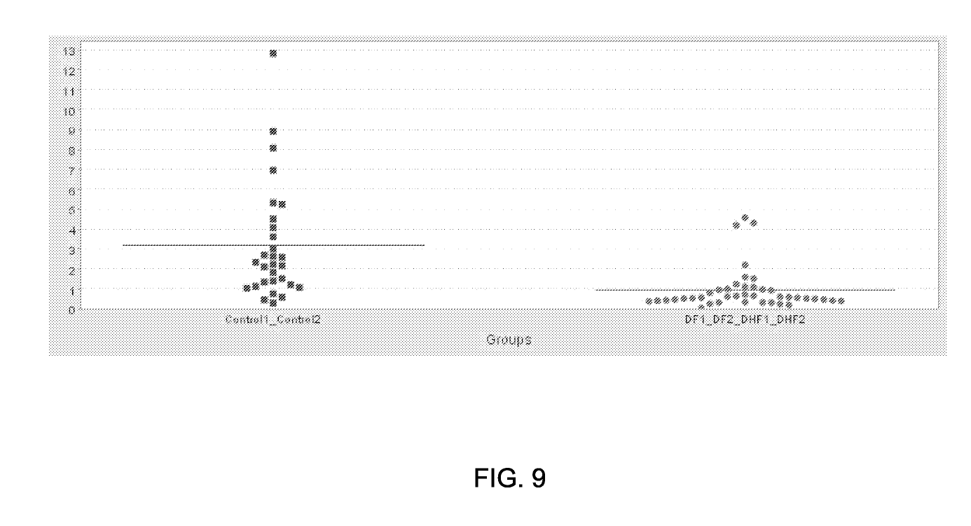

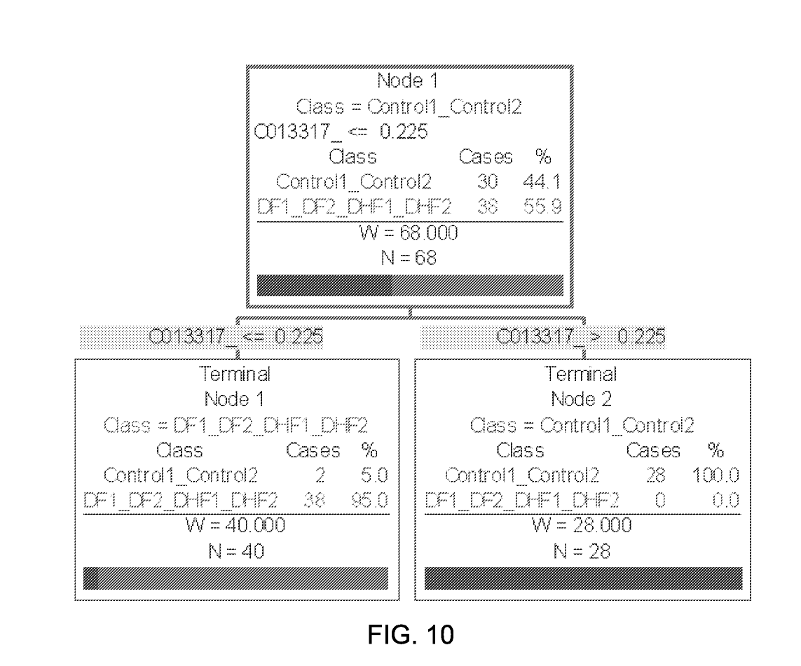

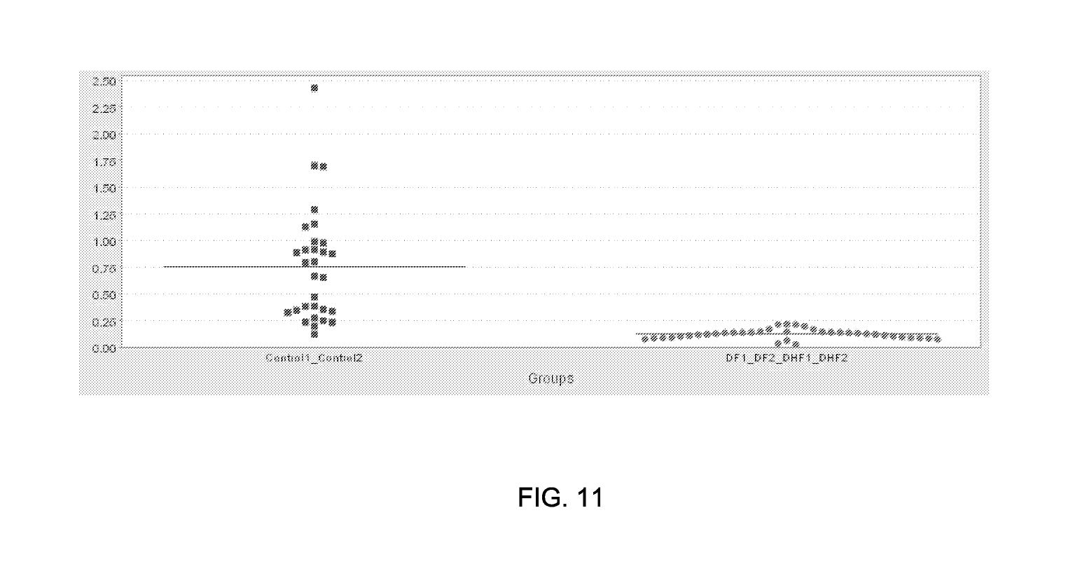

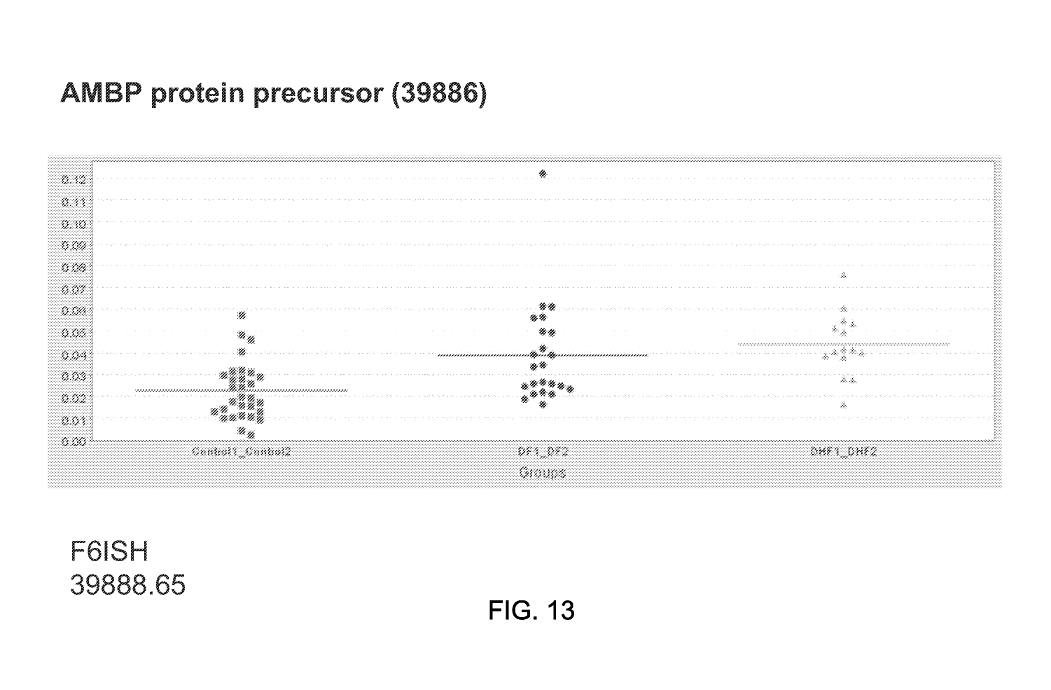

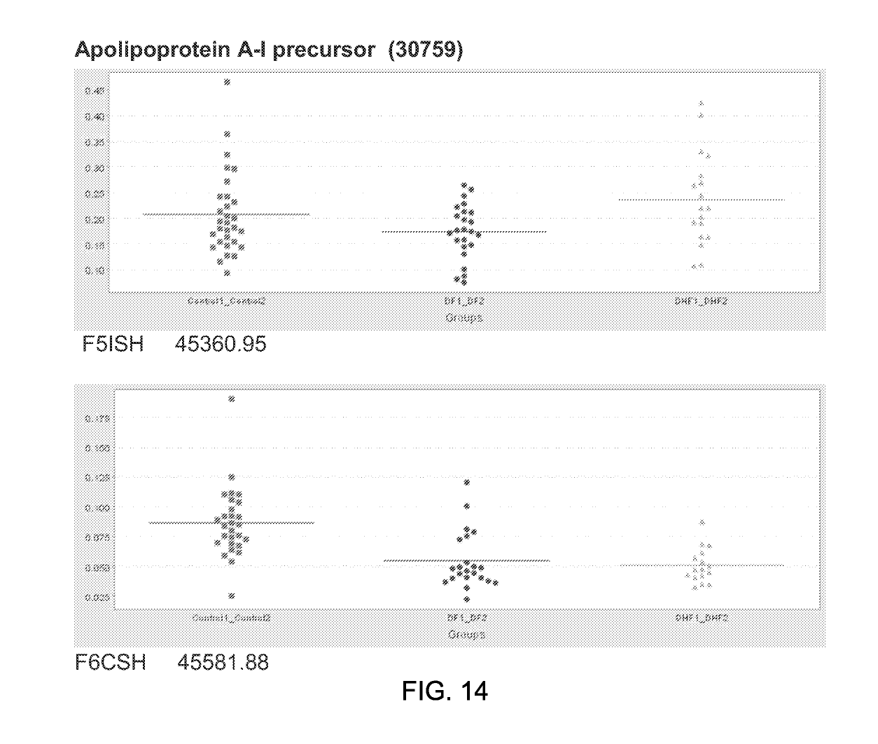

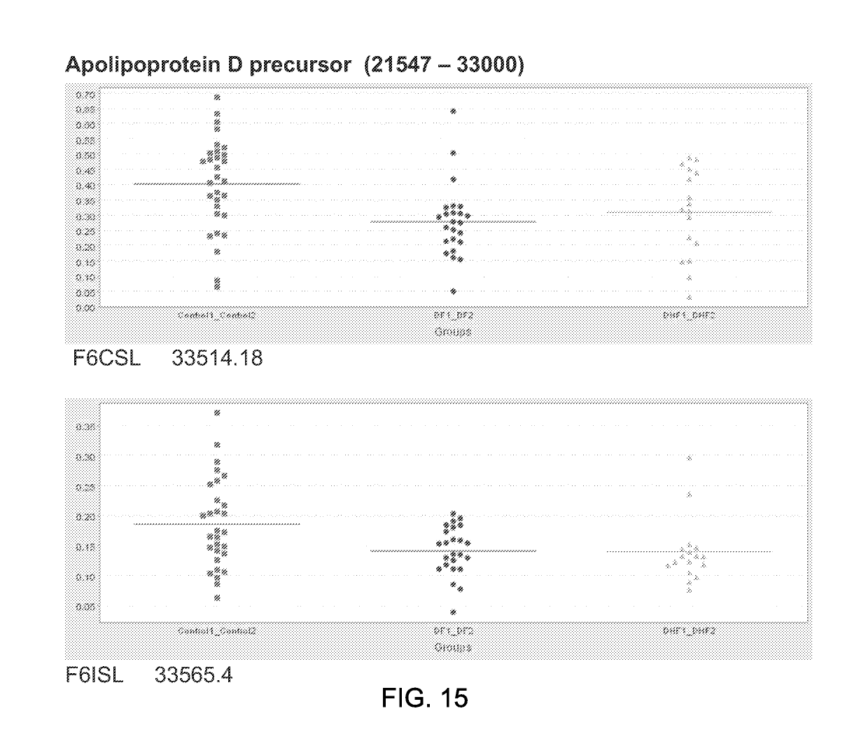

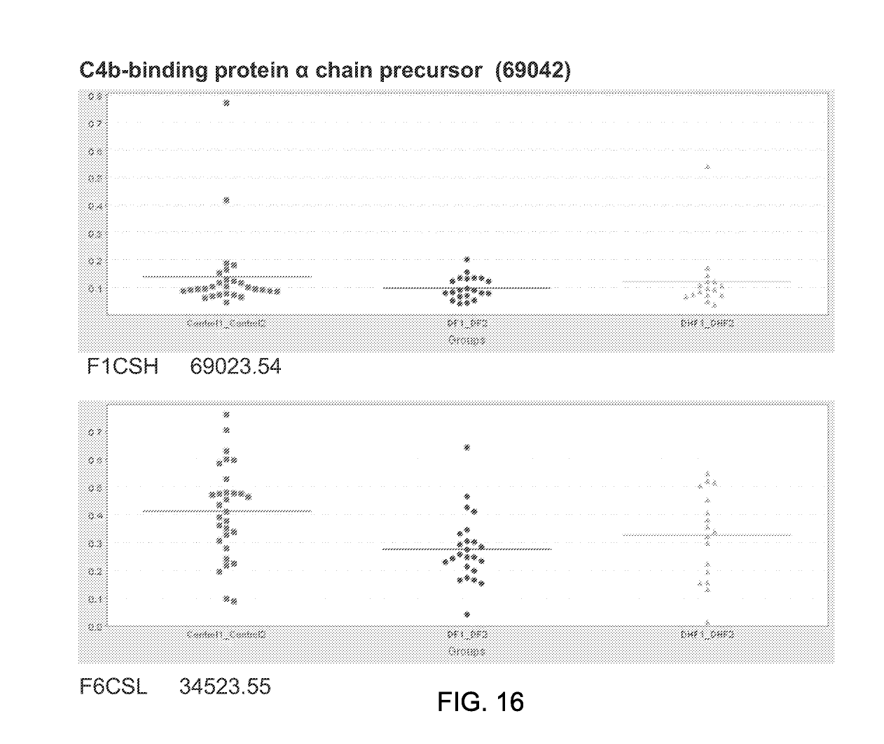

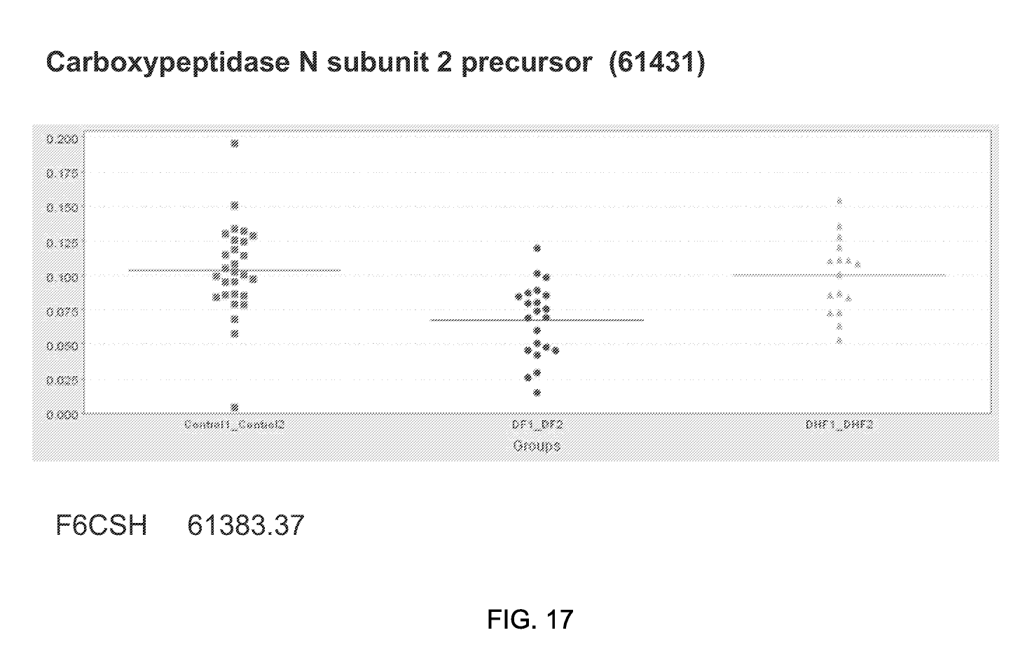

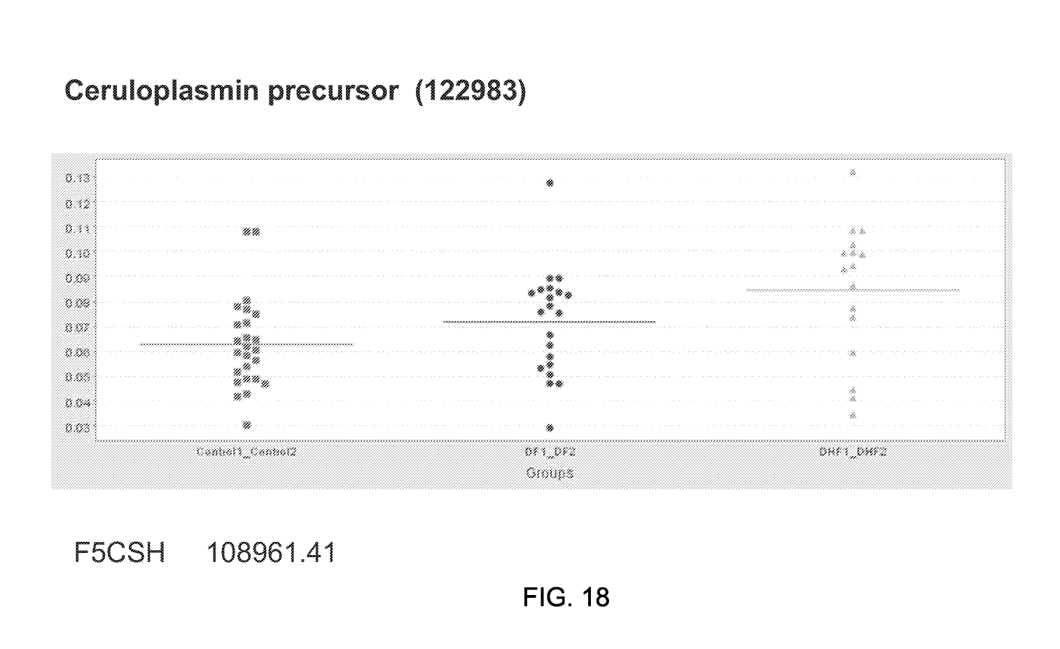

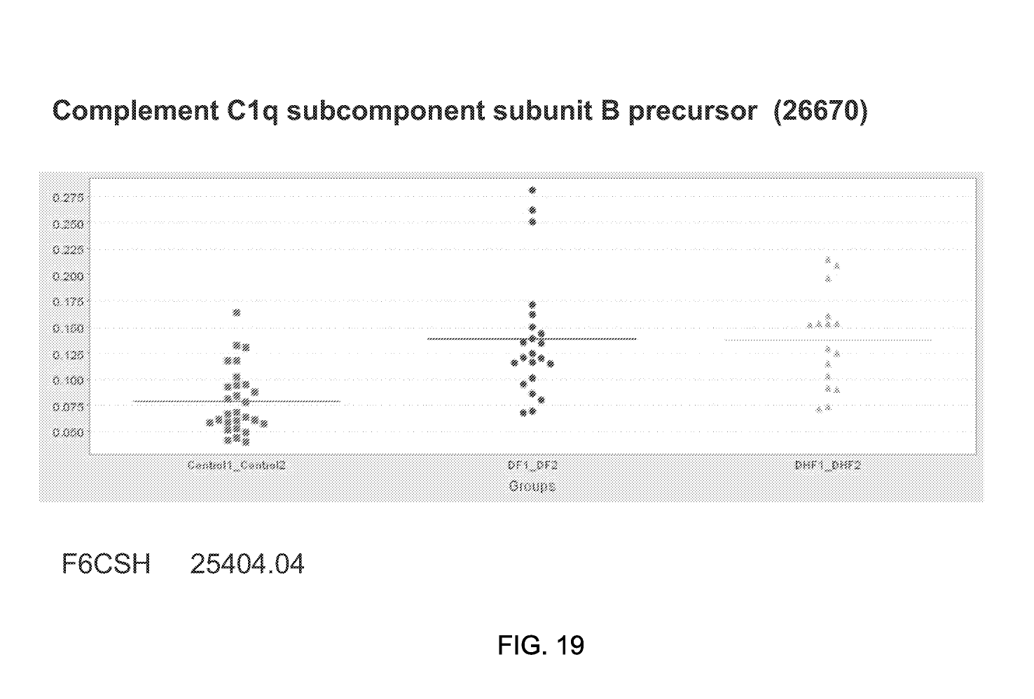

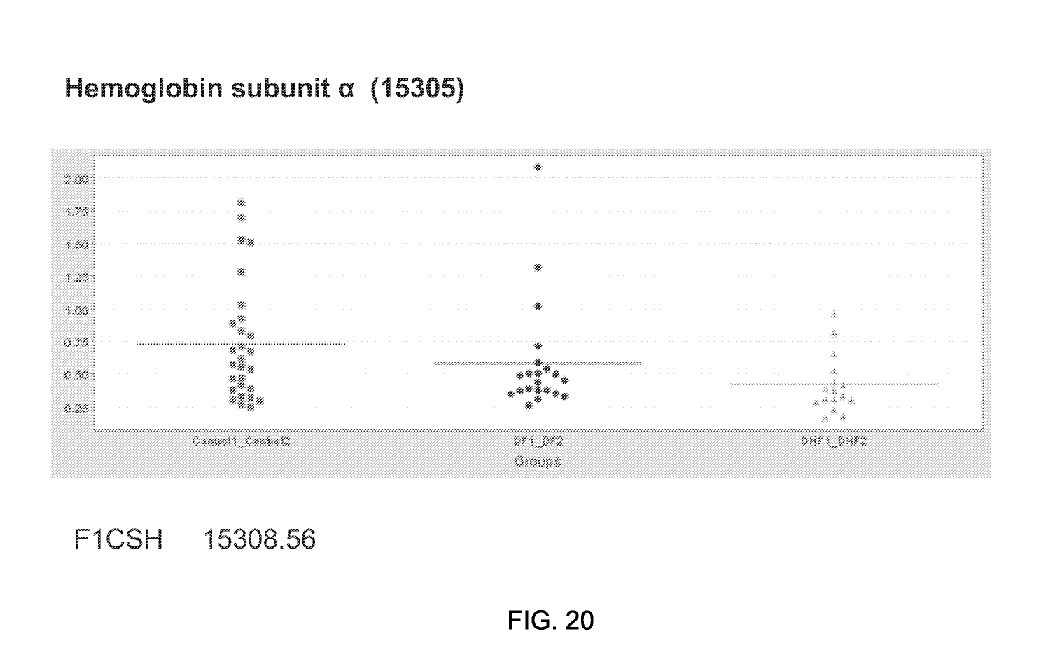

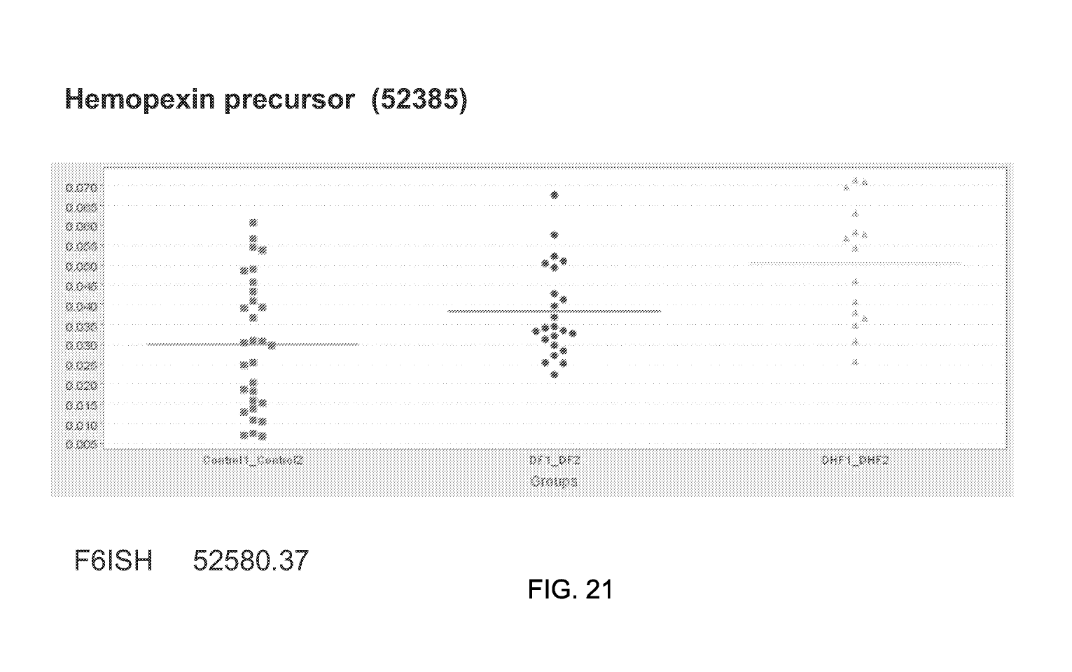

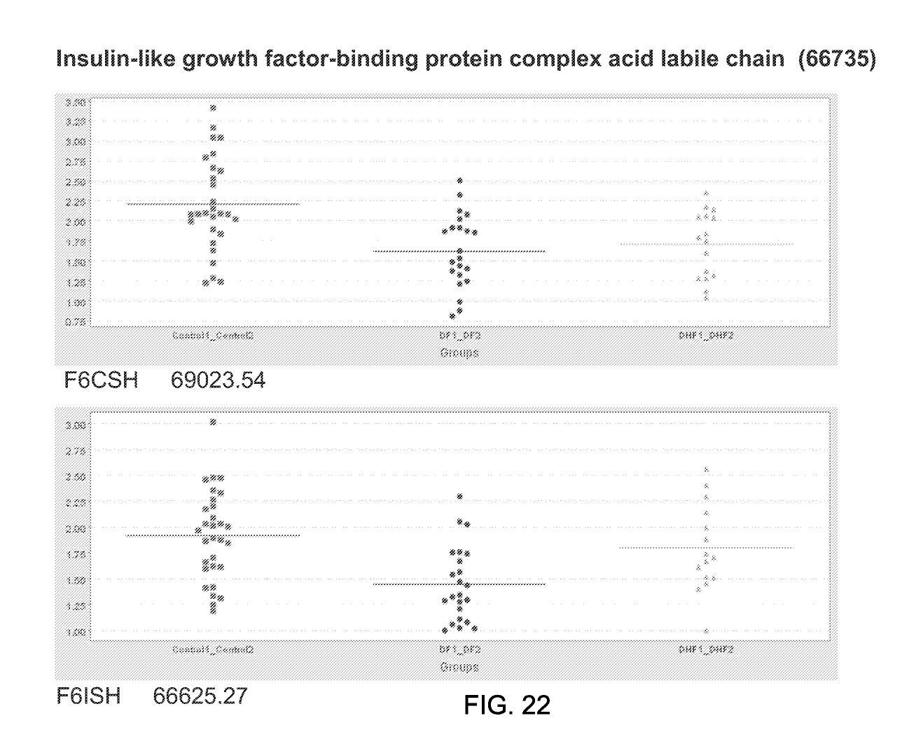

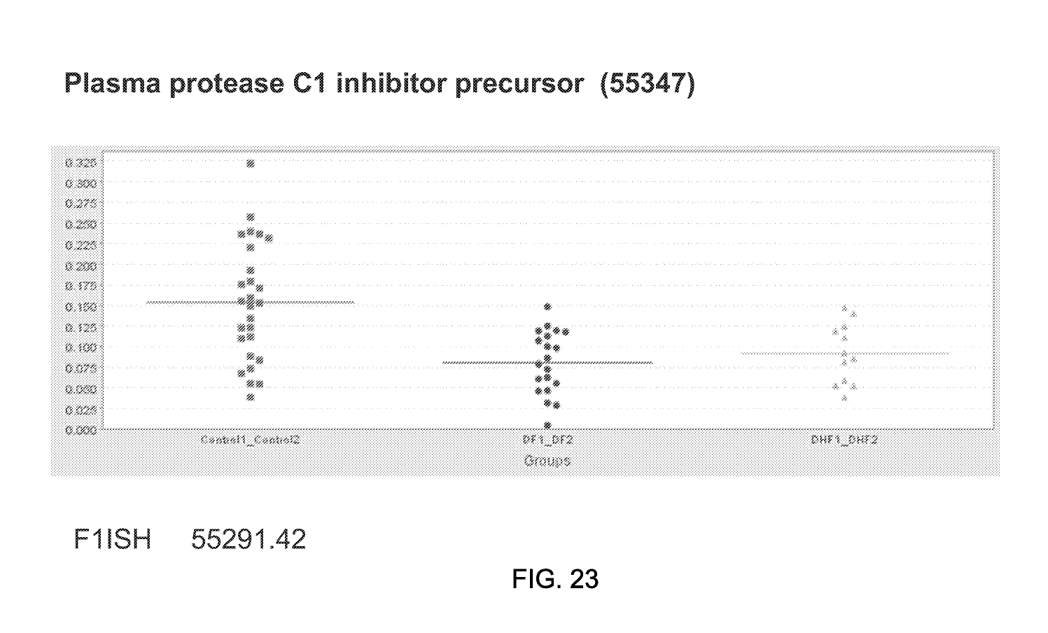

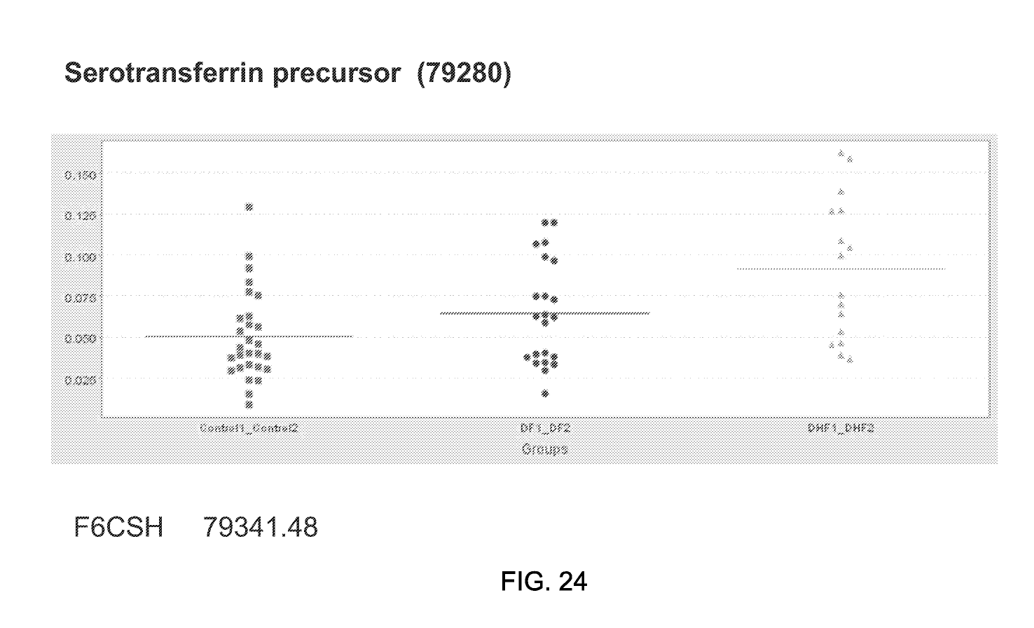

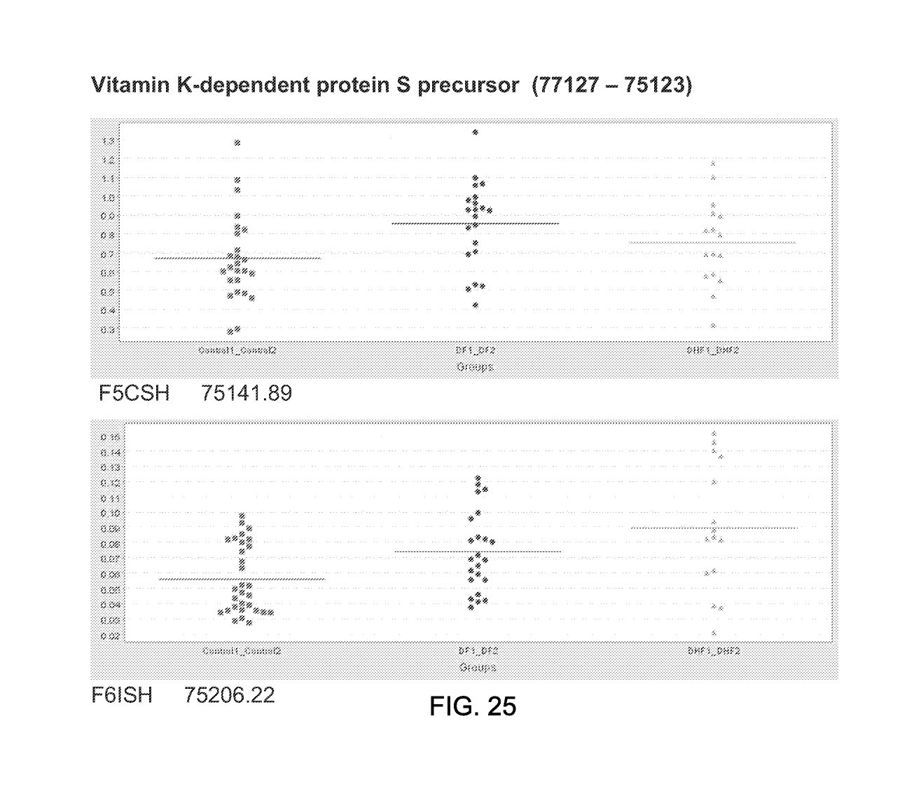

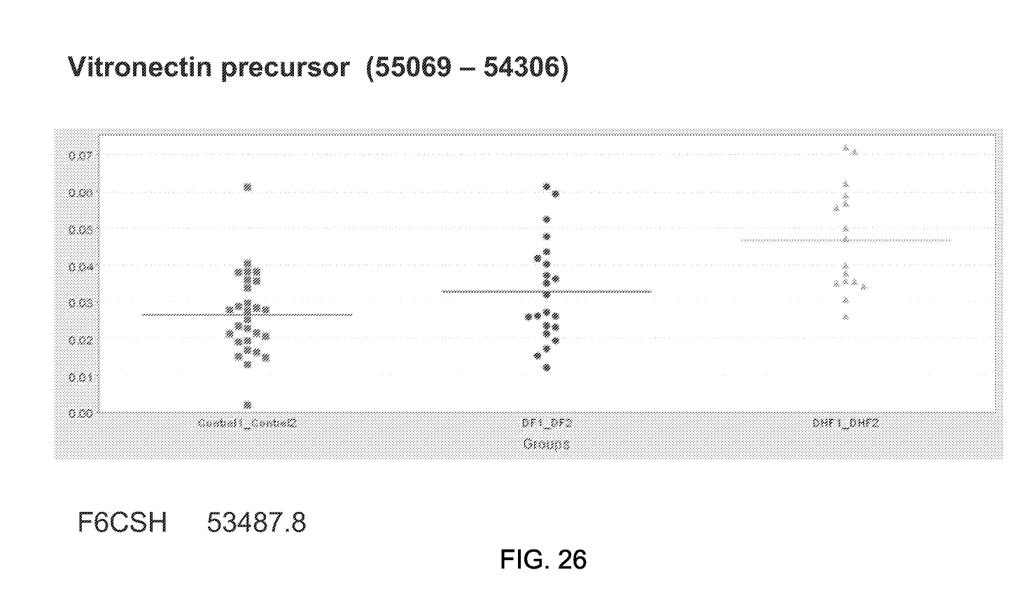

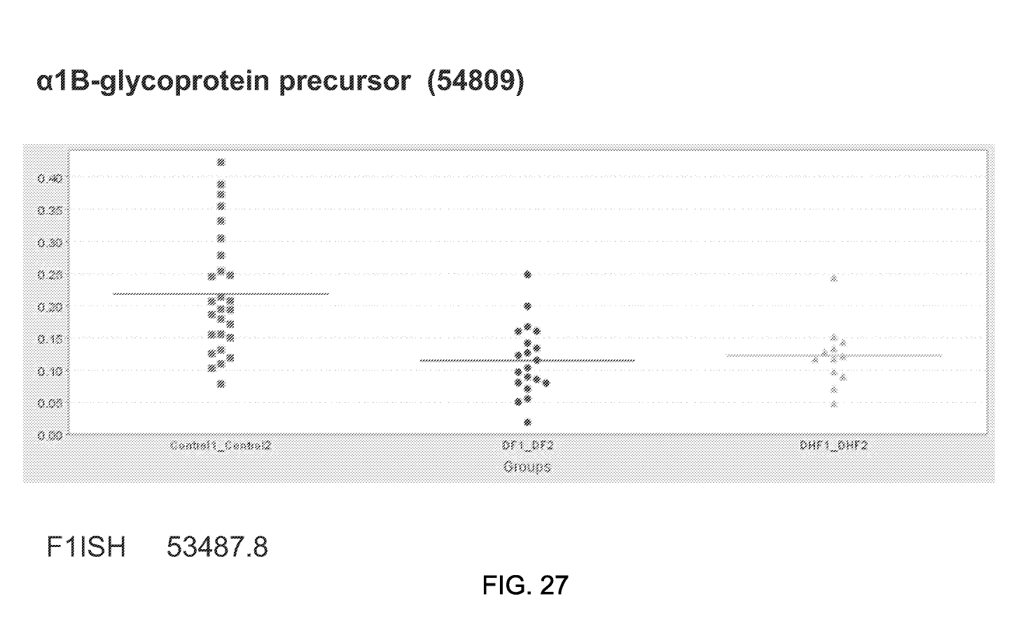

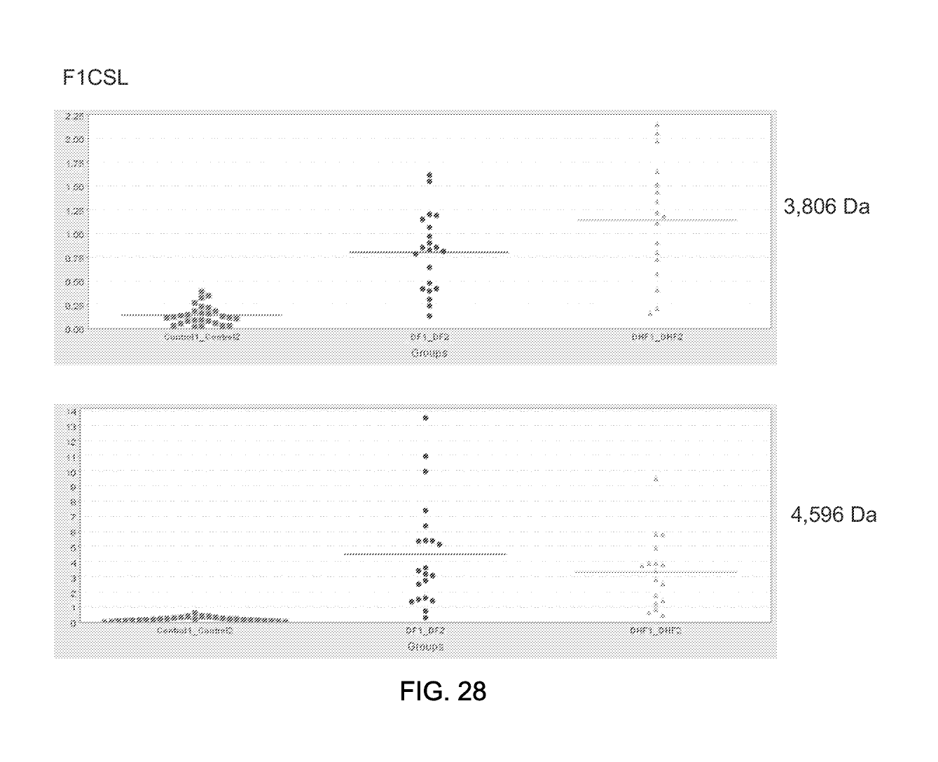

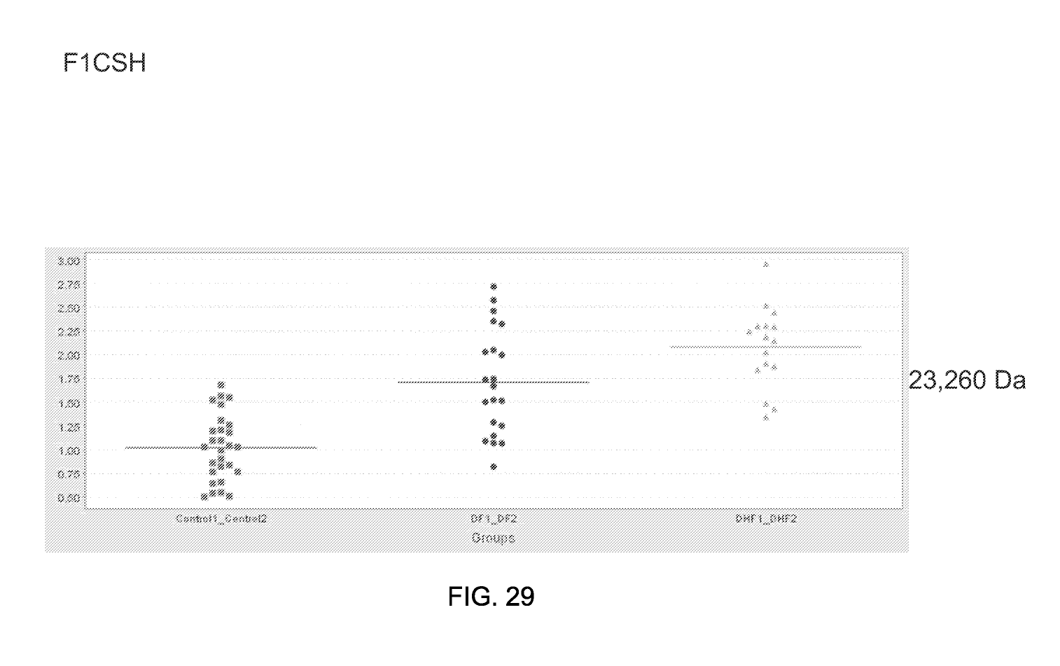

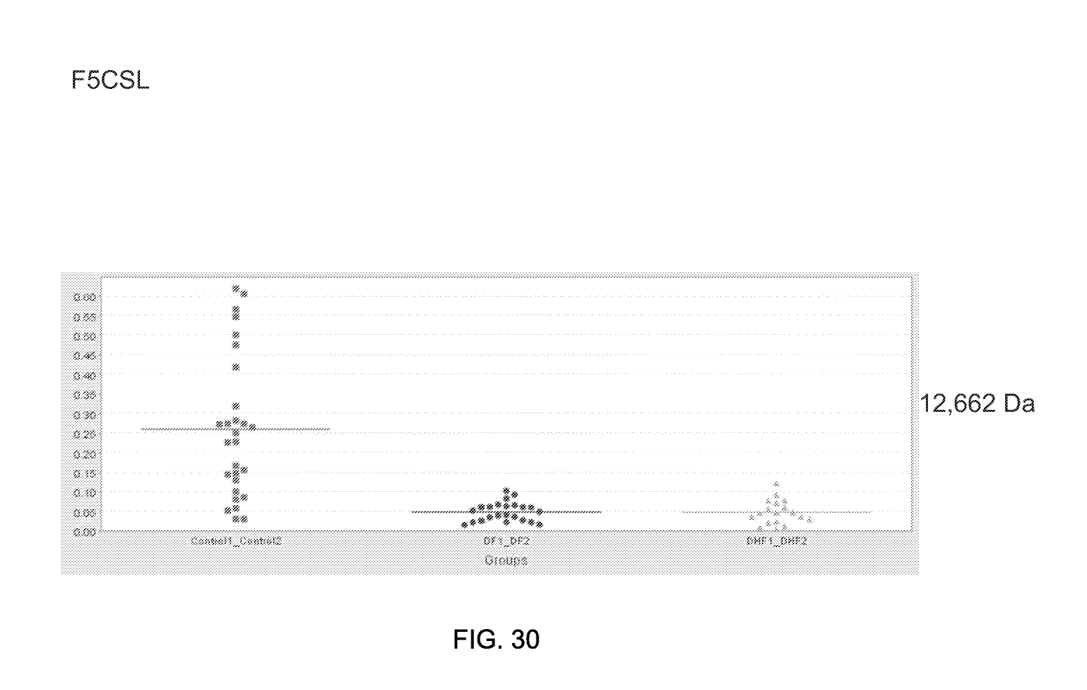

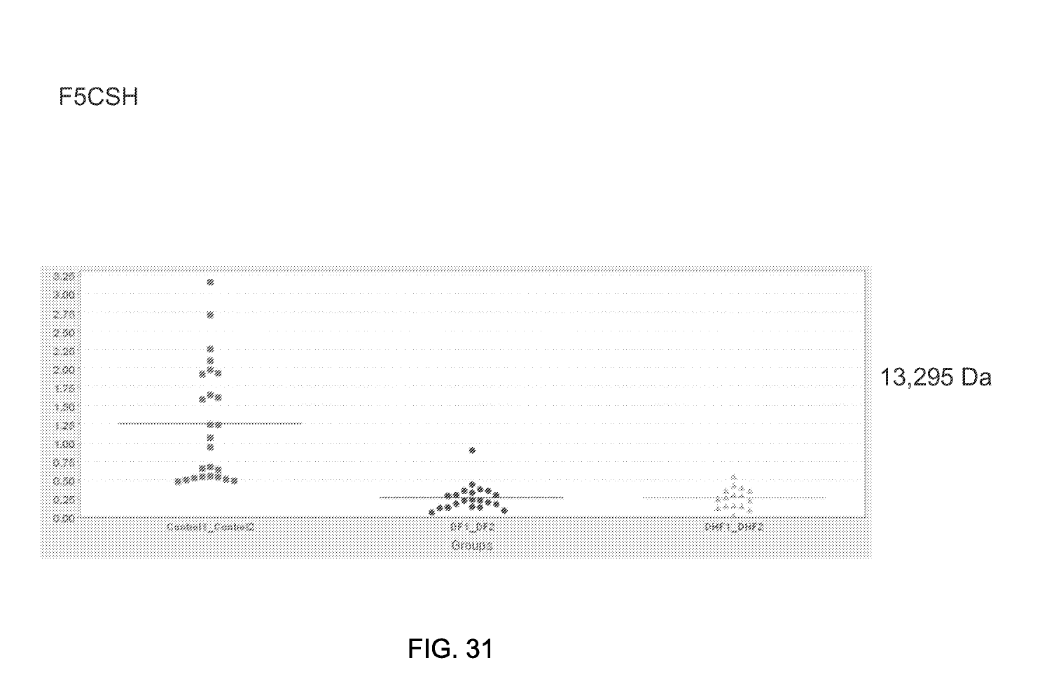

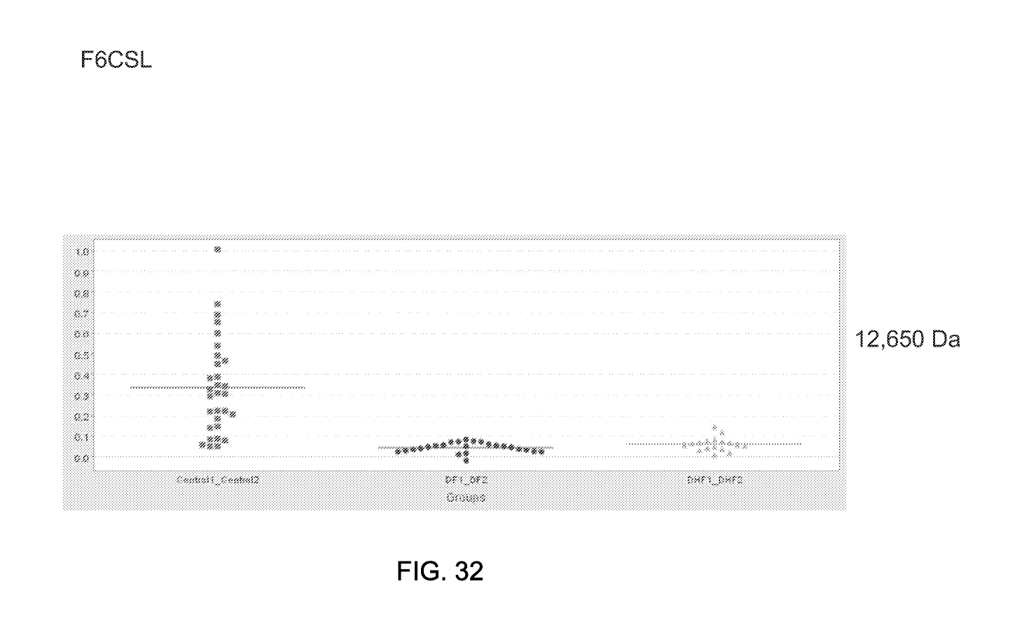

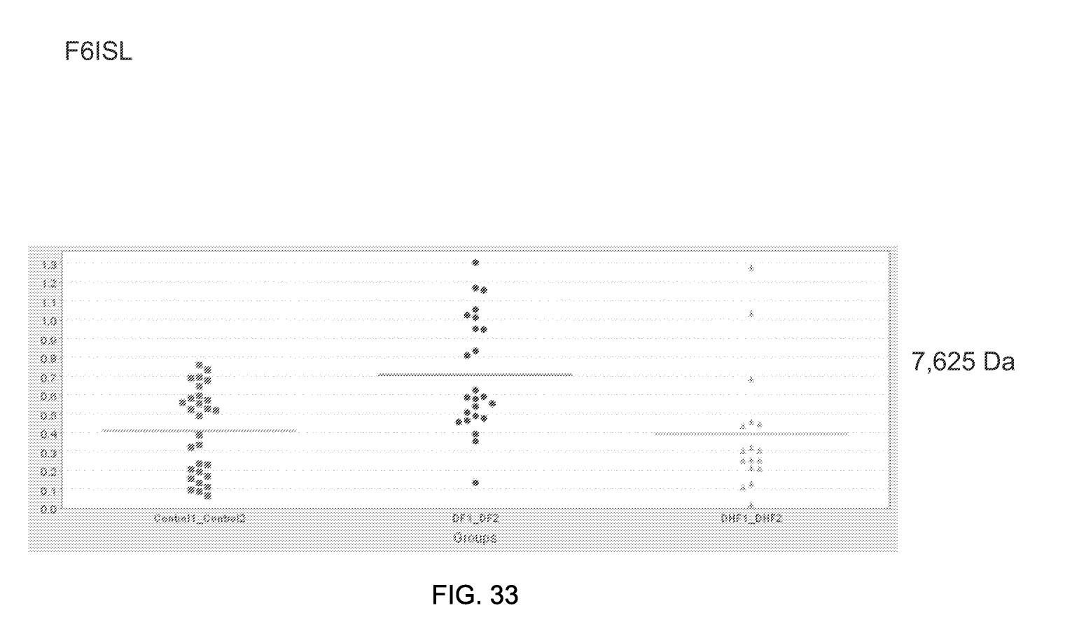

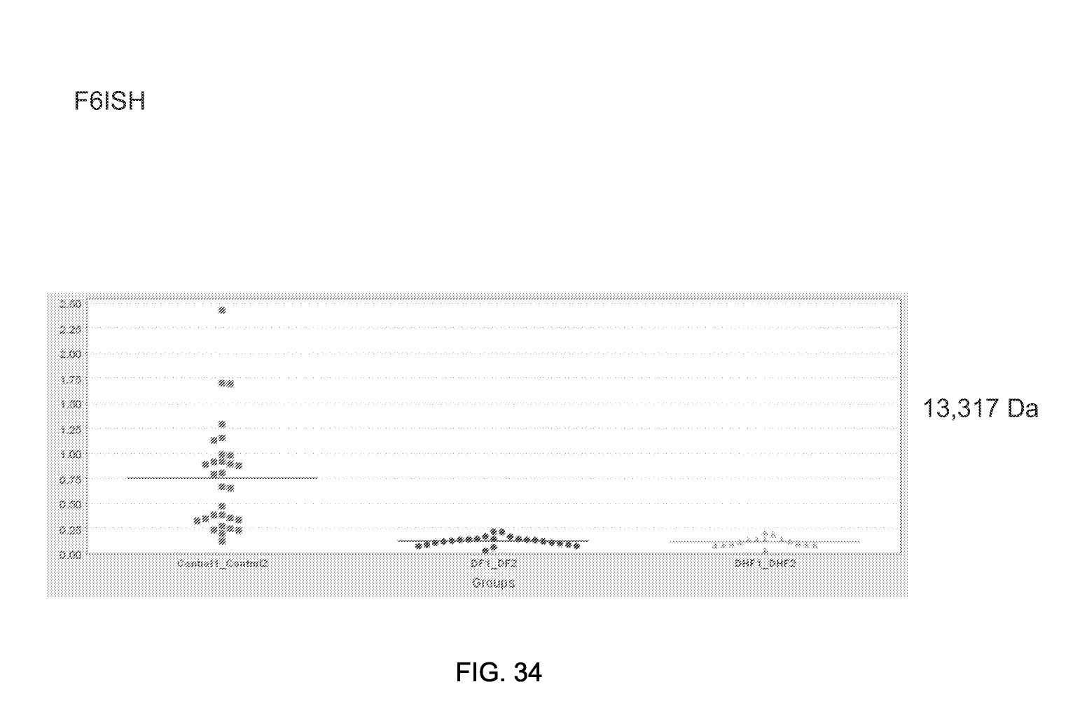

Since the samples from primary and secondary infections were carried on 2 separate bioprocessors on 2 different days, the quality method described above was applied before the following analyses were carried out. A third set of comparisons was carried out between primary and secondary DF at each 3 time point as well as between primary and secondary DHF at each 3 time points. A comparison between control1 and 2DF1 and 2DHF1 as well as between control2 and 2DF2 and 2DHF2 was also carried. The same first- and second-pass analysis protocol was followed but only clusters found to have a p-value ≦0.005 were kept. BPS analysis was also carried using the same comparisons above. FIGS. 1-11, 13-34, and Tables 3-4, 7-16, 17-24 show the results of a SELDI-based biomarker discovery study. The biomarkers presented in these tables and figures can be used in all aspects of the present invention. F1CSL and F1CSH refers to Fraction 1, WCX2, SPA, Low or High intensity; F1ISL and F1ISH refer to Fraction 1, IMAC, SPA, Low or High intensity; F3CSL and F3CSH refer to Fraction 3, WCX2, SPA, Low or High intensity; F5CSL or F5CSH refer to Fraction 5, WCX2, SPA, Low or High intensity; F51SL and F51SH refer to Fraction 5, IMAC, SPA, Low or High intensity; F6CSL and F6CSH refer to Fraction 6, WCX2, SPA, Low or High intensity; and F61SL and F6ISH refer to Fraction 6, IMAC, SPA, Low or High intensity.

ZOOM Fractionation and SDS PAGE

Control 1 and 2 samples were pooled together and 1DF1,2 samples were pooled with 1DHF1,2. The plasma samples were prepared following Invitrogen's recommendations. 650 μl of the prepared samples were dispensed in 5 of the ZOOM® IEF Fractionator chambers. The ZOOM was run under standard conditions (100V for 20 min, 200V for 80 min, and 600V for 80 min). Once completed, the fractions from each chamber were kept at −20° C.

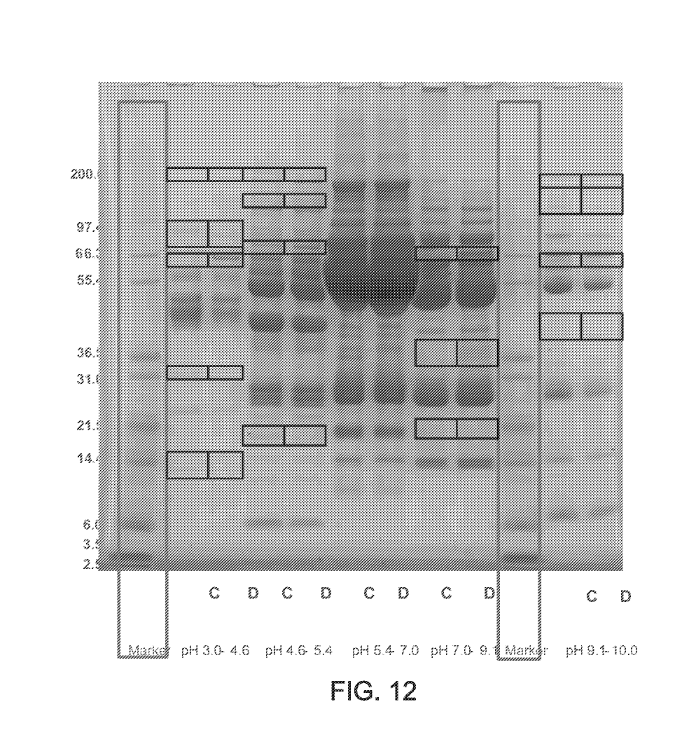

40 μl of for each fraction was desalted. Each aliquot was run on a Denaturing 4-12% Bis-Tris NuPAGE Gel Electrophoresis using Mark12 MW Marker 1× (Invitrogen) as the molecular weight ladder. The gel was run at 200V for 45 min with an expected current of 100-125 mA at the beginning and 60-80 mA towards the end. The gel was stained using a Coomassie stain for 2 days. It was destained with MiliQ water until band visualization was satisfying. The gel was kept in acetic acid. The candidate biomarkers were cut and kept in 2% acetic acid tubes and were sent for sequencing using mass spectrometry. Tables 1-2 and FIG. 12 show the results of a biomarker gel-based discovery study. The biomarkers presented in these tables and figures can be used in all aspects of the present invention.

While the invention has been particularly shown and described with reference to a preferred aspect and various alternate aspects, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 7

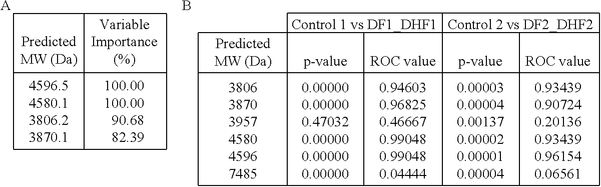

(A) Variable importance of other potential splitter as predicted by BPS in the F1CSL fraction to discriminate between dengue and OFI at t1 (day of admission) and t2 (fever decreases to normal).
(B) p-value and ROC value for all candidate biomarkers found in F1CSL either using BPS or CE. The values highlighted in gray indicate their irrelevance as a potential biomarker at t1 of DENV infection.

TABLE 8
Table 8. (A) Variable importance of other potential splitter as predicted by
BPS in the F1CSH (fraction 1 using CM10 at high laser intensity) fraction
to discriminate between dengue and OFI at t1 (day of admission) and t2
(fever decreases to normal). (B) p-value and ROC value for all candidate
biomarkers found in F1CSH either using BPS or CE.
A

| | Variable |
|---|---|
| Predicted | Importance |
| MW (Da) | (%) |
| 238240 | 100.00% |
| 23260 | 78.50% |

B

| Predicted | Control 1 vs DF1_DHF1 | | Control 2 vs DF2_DHF2 | |
|---|---|---|---|---|
| MW (Da) | p-value | ROC value | p-value | ROC value |
| 11203 | 0.00005 | 0.12667 | 0.00695 | 0.22851 |
| 11605 | 0.00010 | 0.12667 | 0.00473 | 0.22851 |
| 23260 | 0.00032 | 0.87333 | 0.00006 | 0.90724 |
| 23824 | 0.00022 | 0.85000 | 0.00015 | 0.90724 |

TABLE 9

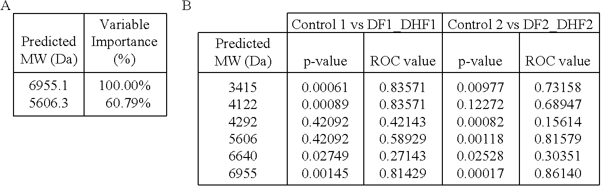

(A) Variable importance of other potential splitter as predicted by BPS in the F1ISL (fraction 1 using IMAC at low laser intensity) fraction to discriminate between dengue and OFI at t1 (day of admission) and t2 (fever decresases to normal).
(B) p-value and ROC value for all candidate biomarkers found in F1ISL either using BPS or CE. The values highlighted in gray indicate their irrelevance as a potential biomarker at t1 of DENV infection.

TABLE 10
Table 10. (A) Variable importance of other potential splitter as predicted
by BPS in the F1ISH (fraction 1 using IMAC at high laser intensity)
fraction to discriminate between dengue and OFI at t1 (day of admission)
and t2 (fever decreases to normal). (B) p-value and ROC value for all
candidate biomarkers found in F1ISH either using BPS or CE.
A

| | Variable |
|---|---|
| Predicted | Importance |
| MW (Da) | (%) |
| 23105 | 100.00 |
| 23638 | 76.33 |
| 56622 | 72.35 |

B

| Predicted | Control 1 vs DF1_DHF1 | | Control 2 vs DF2_DHF2 | |
|---|---|---|---|---|
| MW (Da) | p-value | ROC value | p-value | ROC value |
| 10614 | 0.02586 | 0.23958 | 0.00007 | 0.09167 |
| 10634 | 0.00255 | 0.16667 | 0.00090 | 0.16667 |
| 10649 | 0.04109 | 0.27083 | 0.00157 | 0.19167 |
| 23105 | 0.00059 | 0.90625 | 0.00501 | 0.78125 |
| 23638 | 0.00098 | 0.85417 | 0.01019 | 0.75625 |
| 56622 | 0.00159 | 0.14063 | 0.00719 | 0.19167 |

TABLE 11

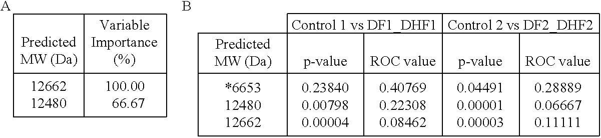

(A) Variable importance of other potential splitter as predicted by BPS in the F5CSL (fraction 5 using CM10 at low laser intensity) fraction to discrimiated between dengue and OFI at t1 (day of admission) and t2 (fever decreases to normal).
(B) p-value and ROC value for all candidate biomarkers found in F5CSL either using BPS or CE. The values highlighted in gray indicate their irrelevance as a potential biomarker at t1 of DENV infection.
*Splitter used in BPS analysis.

TABLE 12
Table 12. (A) Variable importance of other potential splitter as predicted
by BPS in the F5CSH (fraction 5 using CM10 at high laser intensity)
fraction to discriminate between dengue and OFI at t1 (day of admission)
and t2 (fever decreases to normal). (B) p-value and ROC value for all
candidate biomarkers found in F5CSH either using BPS or CE.
A

| | Variable |
|---|---|
| Predicted | Importance |
| MW (Da) | (%) |
| 13294 | 100.00 |
| 13092 | 78.78 |
| 13325 | 68.42 |

B

| Predicted | Control 1 vs DF1_DHF1 | | Control 2 vs DF2_DHF2 | |
|---|---|---|---|---|
| MW (Da) | p-value | ROC value | p-value | ROC value |
| 12919 | 0.00031 | 0.12821 | 0.00042 | 0.14254 |
| 13092 | 0.00006 | 0.07692 | 0.00001 | 0.03728 |
| 13294 | 0.00001 | 0.02564 | 0.00001 | 0.01096 |
| 13325 | 0.00003 | 0.05128 | 0.00002 | 0.03728 |

TABLE 13

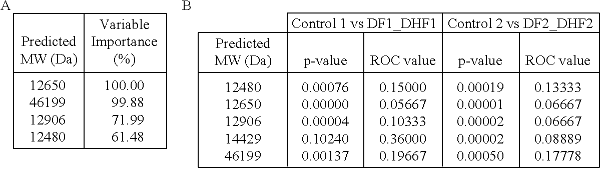

(A) Variable importance of other potential splitter as predicted by BPS in the F6CSL (fraction 6 using CM10 at low laser intensity) fraction to discriminate between dengue and OFI at t1 (day of admission) and t2 (fever decreases to normal).
(B) p-value and ROC value for all candidate biomarkers found in F6CSL either using BPS or CE. The values highlighted in gray indicate their irrelevance as a potential biomarker at t1 of DENV infection.

TABLE 14
Table 14. (A) Variable importance of other potential splitter as predicted
by BPS in the F6CSH (fraction 6 using CM10 at high laser intensity)
fraction to discriminate between dengue and OFI at t1 (day of admission)
and t2 (fever decreases to normal). (B) p-value and ROC value for all
candidate biomarkers found in F6CSH either using BPS or CE.
A

| | Variable |
|---|---|
| Predicted | Importance |
| MW (Da) | (%) |
| 44705 | 100.00 |
| 46584 | 88.26 |
| 13359 | 76.16 |

B

| Predicted | Control 1 vs DF1_DHF1 | | Control 2 vs DF2_DHF2 | |
|---|---|---|---|---|
| MW (Da) | p-value | ROC value | p-value | ROC value |
| 25402 | 0.00119 | 0.82885 | 0.00011 | 0.87222 |
| 44705 | 0.00002 | 0.06154 | 0.00040 | 0.15556 |
| 45584 | 0.00008 | 0.07885 | 0.00188 | 0.17778 |
| 117245 | 0.00715 | 0.21731 | 0.00537 | 0.23889 |
| 133359 | 0.00061 | 0.17692 | 0.00002 | 0.06667 |

TABLE 15

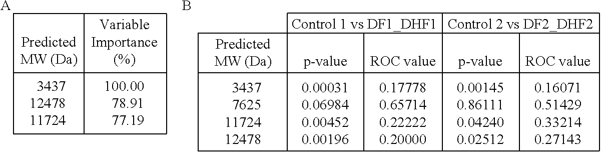

(A) Variable importance of other potential splitter as predicted by BPS in the F6ISL (fraction 6 using IMAC at low laser intensity) fraction to discriminate between dengue and OFI at t1 (day of admission) and t2 (fever decreases to normal).
(B) p-value and ROC value for all candidate biomarkers found in F6ISL either using BPS or CE. The values highlighted in gray indicate their irrelevance as a potential biomarker at t1 of DENV infection, however, BPS analysis defined this peptide as a potential splitter.

TABLE 16
Table 16. (A) Variable importance of other potential splitter as predicted
by BPS in the F6ISH (fraction 6 using IMAC at high laser intensity)
fraction to discriminate between dengue and OFI at t1 (day of
admission) and t2 (fever decreases to normal). (B) p-value and ROC
value for all candidate biomarkers found in F6ISH either using BPS or CE.
A

| | Variable |
|---|---|
| Predicted | Importance |
| MW (Da) | (%) |
| 13317 | 100.00 |
| 13181 | 60.63 |

B

| Predicted | Control 1 vs DF1_DHF1 | | Control 2 vs DF2_DHF2 | |
|---|---|---|---|---|
| MW (Da) | p-value | ROC value | p-value | ROC value |
| 11502 | 0.01192 | 0.75614 | 0.00049 | 0.83684 |
| 13181 | 0.00043 | 0.15614 | 0.00003 | 0.09298 |
| 13317 | 0.00000 | 0.00877 | 0.00001 | 0.05088 |
| 13400 | 0.00001 | 0.05088 | 0.00015 | 0.11404 |
| 133676 | 0.00049 | 0.17368 | 0.00003 | 0.07193 |

TABLE 17
Most significant biomarkers identified by SELDI technology and Biomarker Pattern Software (BPS)
for detecting primary DENV infection at different stages of the disease.
Primary Infection
Biomarkers detected by SELDI and BPS (Ct vs DENV)

| | | | Control 2 vs | | | | | | |
|---|---|---|---|---|---|---|---|---|---|
| | Control 1 vs DHF1 | | DHF2 | | Control 1 vs DF1 | | Control 2 vs DF2 | | |
| | p value | roc | p value | roc | p value | roc | p value | roc | m/z average |
| F1CSL | 0.00667 | 0.84074 | 0.19238 | 0.63942 | 0.00004 | 0.94444 | 0.01014 | 0.85043 | 3187.92612 |
| | 0.00061 | 0.92963 | 0.02981 | 0.79327 | 0.00006 | 0.94444 | 0.00238 | 0.88462 | 3431.45742 |
| | 0.00039 | 0.92963 | 0.01685 | 0.79327 | 0.00001 | 1.00000 | 0.00120 | 0.91880 | 3522.24286 |
| | 0.00015 | 0.95926 | 0.00051 | 0.94712 | 0.00003 | 0.94444 | 0.00058 | 0.91880 | 3806.26212 |
| | 0.00012 | 0.98889 | 0.00017 | 0.98558 | 0.00002 | 0.97222 | 0.00238 | 0.88462 | 3870.26222 |
| | 0.00116 | 0.90000 | 0.00112 | 0.90865 | 0.00017 | 0.91667 | 0.00296 | 0.85043 | 3933.13794 |
| | 0.61227 | 0.42593 | 0.00474 | 0.15385 | 0.49452 | 0.46111 | 0.01227 | 0.21795 | 3957.45555 |
| | 0.57107 | 0.48519 | 0.01685 | 0.23077 | 1.00000 | 0.46111 | 0.00367 | 0.14957 | 3976.20723 |
| | 0.00049 | 0.90000 | 0.00235 | 0.87019 | 0.00004 | 0.94444 | 0.00367 | 0.81624 | 4441.16417 |
| | 0.00025 | 0.95926 | 0.02048 | 0.75481 | 0.00006 | 0.94444 | 0.00558 | 0.85043 | 4459.77765 |
| | 0.00006 | 0.98889 | 0.00022 | 0.98558 | 0.00001 | 1.00000 | 0.00075 | 0.91880 | 4579.92629 |
| | 0.00006 | 0.98889 | 0.00022 | 0.98558 | 0.00001 | 1.00000 | 0.00021 | 0.95299 | 4596.11099 |
| | 0.00012 | 0.98889 | 0.00112 | 0.90865 | 0.20456 | 0.61111 | 0.05702 | 0.71368 | 4990.19603 |
| | 0.00012 | 0.98889 | 0.00144 | 0.92308 | 0.00025 | 0.88889 | 0.04884 | 0.72222 | 6941.41838 |
| | 0.00009 | 0.01111 | 0.00144 | 0.11538 | 0.00003 | 0.04444 | 0.00035 | 0.04701 | 7485.6467 |
| F1CSH | 0.02819 | 0.75833 | 0.34646 | 0.60096 | 0.00728 | 0.77778 | 0.06630 | 0.74786 | 10757.7961 |
| | 0.00813 | 0.16667 | 0.02048 | 0.19231 | 0.02811 | 0.26667 | 0.05702 | 0.25214 | 11076.6199 |
| | 0.00241 | 0.13333 | 0.16882 | 0.34615 | 0.01680 | 0.23889 | 0.01014 | 0.14957 | 13292.3331 |
| | 0.00099 | 0.92083 | 0.00017 | 0.98558 | 0.00541 | 0.83333 | 0.00367 | 0.85043 | 23260.272 |
| | 0.00156 | 0.88750 | 0.00022 | 0.98558 | 0.00248 | 0.86111 | 0.00835 | 0.81624 | 23823.3829 |
| | 0.47768 | 0.55833 | 0.08219 | 0.69231 | 0.49452 | 0.42500 | 0.48320 | 0.44872 | 125373.713 |
| F1ISL | 0.00950 | 0.80357 | 0.12963 | 0.72857 | 0.00203 | 0.83929 | 0.00971 | 0.77778 | 3415.12898 |
| | 0.02896 | 0.76786 | 0.30673 | 0.65238 | 0.00203 | 0.83929 | 0.09711 | 0.66667 | 3457.99405 |
| | 0.04778 | 0.73214 | 0.08416 | 0.71905 | 0.05064 | 0.72024 | 0.00292 | 0.83333 | 3920.28814 |
| | 0.00213 | 0.91071 | 0.97188 | 0.50000 | 0.01012 | 0.77976 | 0.03179 | 0.75000 | 4122.87062 |
| | 0.63282 | 0.42857 | 0.00431 | 0.12857 | 0.57154 | 0.46429 | 0.01117 | 0.21111 | 4276.3415 |
| | 0.49491 | 0.42857 | 0.00431 | 0.12857 | 0.50372 | 0.40476 | 0.00629 | 0.23889 | 4292.76446 |
| | 0.00950 | 0.83929 | 0.19221 | 0.61429 | 0.00397 | 0.80952 | 0.00076 | 0.86111 | 4432.61693 |
| | 0.00415 | 0.83929 | 0.50307 | 0.61429 | 0.00170 | 0.86905 | 0.00044 | 0.86111 | 4449.12593 |
| | 0.04778 | 0.76786 | 0.00081 | 0.94762 | 0.75762 | 0.57143 | 0.40681 | 0.61111 | 4994.29386 |
| | 0.05600 | 0.25000 | 0.04454 | 0.24286 | 0.07183 | 0.28571 | 0.07898 | 0.32222 | 6640.40179 |
| | 0.01401 | 0.80357 | 0.00105 | 0.91905 | 0.00467 | 0.80952 | 0.00248 | 0.84167 | 6955.23285 |
| F1ISH | 0.01921 | 0.16667 | 0.00644 | 0.11111 | 0.00835 | 0.15000 | 0.00657 | 0.16667 | 10634.4592 |
| | 0.03123 | 0.16667 | 0.00185 | 0.06667 | 0.46826 | 0.38333 | 0.31806 | 0.60000 | 12534.592 |
| | 0.00145 | 0.95833 | 0.00046 | 0.98333 | 0.00835 | 0.80000 | 0.14924 | 0.66667 | 23104.0813 |
| | 0.00200 | 0.95833 | 0.00108 | 0.98333 | 0.01222 | 0.80000 | 0.20202 | 0.60000 | 23638.655 |
| | 0.03123 | 0.16667 | 0.01952 | 0.15556 | 0.00301 | 0.11667 | 0.03504 | 0.23333 | 56616.0019 |
| F5CSL | 0.11658 | 0.32051 | 0.05263 | 0.27778 | 0.62239 | 0.43007 | 0.14323 | 0.35000 | 6653.05308 |
| | 0.02528 | 0.79060 | 0.22156 | 0.66296 | 0.00344 | 0.84965 | 0.00025 | 0.91667 | 8961.93991 |
| | 0.01776 | 0.18376 | 0.00260 | 0.15926 | 0.03446 | 0.26224 | 0.00002 | 0.01667 | 12480.7656 |
| | 0.00058 | 0.04701 | 0.00116 | 0.10000 | 0.00046 | 0.06643 | 0.00025 | 0.07222 | 12662.5319 |
| | 0.02999 | 0.25214 | 0.42083 | 0.42593 | 0.00344 | 0.17832 | 0.30551 | 0.37778 | 44676.2104 |
| F5CSH | 0.04168 | 0.78205 | 0.10832 | 0.71429 | 0.00058 | 0.91880 | 0.41892 | 0.61111 | 10211.8483 |
| | 0.02999 | 0.78205 | 0.03461 | 0.78571 | 0.00095 | 0.91880 | 0.56370 | 0.58333 | 10313.1139 |
| | 0.48320 | 0.42308 | 0.61209 | 0.44048 | 0.04884 | 0.75641 | 0.72903 | 0.47222 | 10913.8933 |
| | 0.01227 | 0.18376 | 0.79985 | 0.54762 | 0.05702 | 0.25214 | 0.90807 | 0.50000 | 12195.5553 |
| | 0.00035 | 0.04701 | 0.00235 | 0.08333 | 0.00238 | 0.11538 | 0.00043 | 0.08333 | 12979.039 |
| | 0.00035 | 0.04701 | 0.00177 | 0.08333 | 0.00190 | 0.11538 | 0.00003 | 0.00000 | 13092.8135 |
| | 0.00009 | 0.01282 | 0.00072 | 0.01190 | 0.00035 | 0.04701 | 0.00003 | 0.00000 | 13295.3566 |
| | 0.00016 | 0.01282 | 0.00177 | 0.08333 | 0.00151 | 0.11538 | 0.00007 | 0.02778 | 13325.8983 |
| | 0.11658 | 0.68803 | 0.12819 | 0.26190 | 0.00684 | 0.85043 | 0.08326 | 0.72222 | 14029.4062 |
| | 0.57030 | 0.42308 | 0.05191 | 0.22619 | 0.19286 | 0.66239 | 0.38648 | 0.63889 | 28370.513 |
| | 0.86741 | 0.51709 | 0.00039 | 1.00000 | 0.92021 | 0.48291 | 0.00558 | 0.80556 | 108961.408 |
| F5ISH | 0.07857 | 0.30741 | 0.22156 | 0.36667 | 0.02811 | 0.72222 | 0.02480 | 0.26667 | 10226.9872 |
| F6CSL | 0.97622 | 0.48519 | 0.00062 | 0.92083 | 0.97930 | 0.49697 | 0.05591 | 0.71795 | 5289.43252 |
| | 0.78845 | 0.57407 | 0.00062 | 0.95417 | 0.97930 | 0.52727 | 0.13436 | 0.67179 | 5474.34274 |
| | 0.01127 | 0.18889 | 0.00671 | 0.16667 | 0.00240 | 0.13333 | 0.00060 | 0.11795 | 12481.1861 |
| | 0.00049 | 0.10000 | 0.00125 | 0.10000 | 0.00005 | 0.04242 | 0.00008 | 0.09231 | 12650.5231 |
| | 0.00039 | 0.07037 | 0.00099 | 0.06667 | 0.00098 | 0.13333 | 0.00012 | 0.06667 | 12906.1983 |
| | 0.14404 | 0.33704 | 0.00049 | 0.06667 | 0.20353 | 0.37576 | 0.00030 | 0.11795 | 14429.2788 |
| | 0.0112701 | 0.218518 | 0.00099 | 0.06667 | 0.00021 | 0.10303 | 0.00344 | 0.16923 | 45465.06 |
| | 0.0295224 | 0.2481481 | 0.00368 | 0.13333 | 0.0020148 | 0.1636364 | 0.00344 | 0.19487 | 46196.85 |
| F6CSH | 0.15108 | 0.65385 | 0.01008 | 0.83333 | 0.54297 | 0.43007 | 0.81533 | 0.46667 | 10031.5232 |
| | 0.11658 | 0.72222 | 0.00344 | 0.90952 | 0.83931 | 0.51399 | 0.93795 | 0.46667 | 10128.7882 |
| | 0.00684 | 0.85043 | 0.00217 | 0.94762 | 0.00592 | 0.82168 | 0.00098 | 0.84848 | 25404.0365 |
| | 0.00021 | 0.01282 | 0.01502 | 0.20476 | 0.00037 | 0.09441 | 0.00098 | 0.13333 | 44706.8965 |
| | 0.00035 | 0.08120 | 0.00665 | 0.12857 | 0.00194 | 0.12238 | 0.01369 | 0.22424 | 45581.8839 |
| | 0.05702 | 0.24359 | 0.91579 | 0.46190 | 0.01173 | 0.23427 | 0.01820 | 0.22424 | 46366.8165 |
| | 0.97336 | 0.50855 | 0.86012 | 0.50000 | 0.00019 | 0.06643 | 0.00336 | 0.16364 | 59365.1146 |
| | 0.44252 | 0.41453 | 0.75109 | 0.47143 | 0.00037 | 0.09441 | 0.00017 | 0.07273 | 117244.849 |
| | 0.00190 | 0.11538 | 0.00081 | 0.05238 | 0.00706 | 0.17832 | 0.00025 | 0.07273 | 133359.809 |
| | 0.02123 | 0.18376 | 0.00431 | 0.12857 | 0.07722 | 0.29021 | 0.00068 | 0.13333 | 198260.477 |
| F6ISL | 0.00556 | 0.15926 | 0.01401 | 0.20536 | 0.00128 | 0.12778 | 0.00467 | 0.22619 | 3437.48403 |
| | 0.88150 | 0.51481 | 0.17224 | 0.27679 | 0.00842 | 0.80556 | 0.19849 | 0.63095 | 7625.50723 |
| | 0.00316 | 0.15926 | 0.05600 | 0.28571 | 0.05704 | 0.29444 | 0.12282 | 0.34524 | 11724.8512 |
| | 0.01127 | 0.15926 | 0.07597 | 0.28571 | 0.00842 | 0.21111 | 0.05064 | 0.28571 | 12478.098 |
| | 0.01574 | 0.21852 | 0.00777 | 0.21429 | 0.02480 | 0.23889 | 0.00641 | 0.16667 | 34219.2306 |
| F6ISH | 0.00671 | 0.13333 | 0.00105 | 0.05238 | 0.00201 | 0.13333 | 0.00037 | 0.10000 | 13181.7725 |
| | 0.00011 | 0.00000 | 0.00048 | 0.05238 | 0.00002 | 0.01212 | 0.00006 | 0.07222 | 13317.4205 |
| | 0.00062 | 0.03333 | 0.00536 | 0.12857 | 0.00009 | 0.04242 | 0.00064 | 0.12778 | 13400.7181 |
| | 0.27249 | 0.59583 | 0.80513 | 0.47143 | 0.00336 | 0.19394 | 0.00064 | 0.10000 | 59524.38 |
| | 0.008132 | 0.1666667 | 0.00665 | 0.12857 | 0.00201 | 0.16364 | 0.00005 | 0.07222 | 133676.05 |

TABLE 18
Biomarkers identified by SELDI technology with a p-value smaller
or equal to 0.05 that can discriminate primary DHF infection from
OFI. Grouped according to fraction it was detected in.
Diagnostic

Ct1_2 vs DHF1_2

| | p value | roc | m/z averaqe |
|---|---|---|---|
| F1CSL | 0.0000000 | 0.9915966 | 4579.926292 |
| | 0.0000000 | 0.9915966 | 4596.110987 |
| | 0.0000001 | 0.9726891 | 5583.561565 |
| | 0.0000001 | 0.9726891 | 3870.26222 |
| | 0.0000002 | 0.9537815 | 3806.262116 |
| | 0.0000003 | 0.9348739 | 4990.19603 |
| | 0.0000004 | 0.9537815 | 6941.418376 |
| | 0.0000006 | 0.0651261 | 7485.646702 |
| | 0.0000025 | 0.8970588 | 3933.137941 |
| | 0.0000025 | 0.8970588 | 4441.164166 |
| | 0.0000078 | 0.8781513 | 4459.777653 |
| | 0.0000078 | 0.8781513 | 4800.693027 |
| | 0.0000097 | 0.8592437 | 3061.572119 |
| | 0.0000120 | 0.8592437 | 3522.242859 |
| | 0.0000133 | 0.8592437 | 4020.472527 |
| | 0.0000165 | 0.8781513 | 6140.608328 |
| | 0.0000183 | 0.8781513 | 6138.119764 |
| | 0.0000204 | 0.8403361 | 4423.747371 |
| | 0.0000278 | 0.8592437 | 4654.384151 |
| | 0.0000342 | 0.8403361 | 5266.529592 |
| | 0.0000925 | 0.8592437 | 3431.457425 |
| | 0.0000925 | 0.8403361 | 5183.584315 |
| | 0.0001234 | 0.1596639 | 7658.7026 |
| | 0.0001975 | 0.8403361 | 4488.791713 |
| | 0.0004078 | 0.8025210 | 2517.703609 |
| | 0.0005789 | 0.8025210 | 2886.289725 |
| | 0.0005789 | 0.8025210 | 23588.47849 |
| | 0.0006875 | 0.8025210 | 3821.714797 |
| | 0.0007487 | 0.8025210 | 3248.144023 |
| | 0.0018483 | 0.2542017 | 2752.206092 |
| | 0.0020004 | 0.2163866 | 2980.456404 |
| | 0.0029479 | 0.2542017 | 10556.99254 |
| | 0.0031808 | 0.7647059 | 3187.926121 |
| | 0.0039834 | 0.7647059 | 6456.367141 |
| | 0.0046163 | 0.2731092 | 7940.572626 |
| | 0.0053390 | 0.7457983 | 9107.540827 |
| | 0.0061624 | 0.7268908 | 8780.719118 |
| | 0.0076127 | 0.7457983 | 38593.25709 |
| | 0.0081601 | 0.7079832 | 5912.839452 |
| | 0.0107190 | 0.7268908 | 4471.501882 |
| | 0.0122483 | 0.2920168 | 3957.455551 |
| | 0.0149050 | 0.7079832 | 4527.180907 |
| | 0.0204696 | 0.6701681 | 2683.722459 |
| | 0.0204696 | 0.7268908 | 3224.576562 |
| | 0.0204696 | 0.6890756 | 37462.00218 |
| | 0.0246148 | 0.6890756 | 6487.177441 |
| | 0.0261495 | 0.3109244 | 4189.213856 |
| | 0.0312595 | 0.3109244 | 3321.159048 |
| | 0.0351234 | 0.3109244 | 3976.207226 |
| | 0.0466040 | 0.3109244 | 7195.954395 |
| F1CSH | 0.0011751 | 0.8080357 | 10064.31699 |
| | 0.0003352 | 0.8303571 | 10143.66604 |
| | 0.0078297 | 0.7321429 | 10267.57388 |
| | 0.0137340 | 0.6964286 | 10299.90259 |
| | 0.0016480 | 0.7946429 | 10527.69159 |
| | 0.0036925 | 0.7410714 | 10655.02359 |
| | 0.0168046 | 0.7187500 | 10757.79609 |
| | 0.0005816 | 0.8125000 | 10802.97632 |
| | 0.0003680 | 0.1785714 | 11076.61987 |
| | 0.0062852 | 0.2678571 | 11157.80715 |
| | 0.0000302 | 0.1250000 | 11203.02954 |
| | 0.0046532 | 0.7589286 | 11324.62766 |
| | 0.0005313 | 0.1607143 | 11396.01715 |
| | 0.0119729 | 0.7053571 | 11451.56989 |
| | 0.0002777 | 0.1785714 | 11605.52007 |
| | 0.0481324 | 0.3214286 | 12494.15007 |
| | 0.0299026 | 0.3214286 | 12562.70923 |
| | 0.0062852 | 0.2500000 | 12955.299 |
| | 0.0005313 | 0.1607143 | 13292.33306 |
| | 0.0072807 | 0.2678571 | 13419.85168 |
| | 0.0004851 | 0.1785714 | 13474.4264 |
| | 0.0005816 | 0.1785714 | 13841.62893 |
| | 0.0157200 | 0.2678571 | 14022.97966 |
| | 0.0001558 | 0.1785714 | 15094.27149 |
| | 0.0097059 | 0.2857143 | 15308.55574 |
| | 0.0000003 | 0.9642857 | 23260.27196 |
| | 0.0000007 | 0.9464286 | 23823.38294 |
| | 0.0004851 | 0.8214286 | 25774.83879 |
| | 0.0067665 | 0.7589286 | 29110.79546 |
| | 0.0017909 | 0.7589286 | 30257.11683 |
| | 0.0146974 | 0.7232143 | 38507.26592 |
| | 0.0157200 | 0.2857143 | 53621.70016 |
| | 0.0072807 | 0.2678571 | 54009.77568 |
| | 0.0050205 | 0.7366071 | 173467.5879 |
| F1ISL | 0.0111540 | 0.7114943 | 2716.553062 |
| | 0.0490254 | 0.6517241 | 2862.774426 |
| | 0.0014647 | 0.7804598 | 2923.355944 |
| | 0.0057683 | 0.2287356 | 3277.946842 |
| | 0.0017358 | 0.7804598 | 3415.128978 |
| | 0.0220073 | 0.6931034 | 3457.994054 |
| | 0.0462472 | 0.6517241 | 3501.960593 |
| | 0.0004593 | 0.7988506 | 3793.065138 |
| | 0.0077763 | 0.7252874 | 3920.288144 |
| | 0.0206128 | 0.7114943 | 4122.870618 |
| | 0.0168814 | 0.2839080 | 4276.341498 |
| | 0.0103893 | 0.2471264 | 4292.764456 |
| | 0.0364235 | 0.6517241 | 4414.952812 |
| | 0.0042403 | 0.7482759 | 4432.616925 |
| | 0.0137566 | 0.7114943 | 4449.125935 |
| | 0.0000574 | 0.8724138 | 4994.293858 |
| | 0.0010358 | 0.7850575 | 5272.160739 |
| | 0.0077763 | 0.7344828 | 5606.87859 |
| | 0.0490254 | 0.6655172 | 5908.422969 |
| | 0.0049512 | 0.7574713 | 6127.388003 |
| | 0.0250440 | 0.6977011 | 6455.01391 |
| | 0.0111540 | 0.7344828 | 6488.439228 |
| | 0.0284370 | 0.7022989 | 6508.732475 |
| | 0.0284370 | 0.6931034 | 6588.428983 |
| | 0.0053457 | 0.2471264 | 6640.401792 |
| | 0.0000271 | 0.8586207 | 6955.232848 |
| | 0.0234831 | 0.7114943 | 23565.20853 |
| F1ISH | 0.0385298 | 0.7145062 | 10294.4998 |
| | 0.0243382 | 0.7191358 | 10308.54818 |
| | 0.0176221 | 0.2700617 | 10495.53183 |
| | 0.0357638 | 0.2962963 | 10587.75825 |
| | 0.0005226 | 0.1620370 | 10614.34283 |
| | 0.0003706 | 0.1450617 | 10634.45923 |
| | 0.0034870 | 0.2098765 | 10649.30096 |
| | 0.0009107 | 0.1666667 | 10687.34192 |
| | 0.0331690 | 0.2746914 | 10715.49742 |
| | 0.0067653 | 0.7577160 | 10832.80337 |
| | 0.0074117 | 0.7361111 | 10965.73793 |
| | 0.0115498 | 0.2314815 | 12051.19993 |
| | 0.0034870 | 0.2314815 | 12135.8102 |
| | 0.0002054 | 0.1234568 | 12534.59195 |
| | 0.0067653 | 0.2314815 | 12582.46907 |
| | 0.0162215 | 0.2314815 | 13073.75509 |
| | 0.0385298 | 0.2962963 | 13927.30735 |
| | 0.0000013 | 0.9691358 | 23104.0813 |
| | 0.0000050 | 0.9475309 | 23638.655 |
| | 0.0105871 | 0.7793210 | 46473.02215 |
| | 0.0162215 | 0.7361111 | 47298.31195 |
| | 0.0191276 | 0.2746914 | 50851.35303 |
| | 0.0331690 | 0.2962963 | 51514.98459 |
| | 0.0042320 | 0.2098765 | 53216.07485 |
| | 0.0038431 | 0.2098765 | 53984.24055 |
| | 0.0009107 | 0.2098765 | 54593.1773 |
| | 0.0088733 | 0.2314815 | 55291.41693 |
| | 0.0010150 | 0.1666667 | 56616.00188 |
| | 0.0115498 | 0.2530864 | 69203.03801 |
| | 0.0284597 | 0.2916667 | 75424.41325 |
| | 0.0385298 | 0.2916667 | 125490.8119 |
| | 0.0331690 | 0.2700617 | 135956.4468 |
| F5CSL | 0.0073978 | 0.2678571 | 2580.061762 |
| | 0.0079102 | 0.7242063 | 2714.357253 |
| | 0.0022059 | 0.2321429 | 3432.717673 |
| | 0.0324998 | 0.3035714 | 4788.767558 |
| | 0.0383881 | 0.3035714 | 4818.40977 |
| | 0.0031934 | 0.2678571 | 5006.990761 |
| | 0.0109798 | 0.2678571 | 6653.053079 |
| | 0.0005280 | 0.1964286 | 7785.325256 |
| | 0.0244017 | 0.6706349 | 8961.939907 |
| | 0.0029685 | 0.2500000 | 9320.83479 |
| | 0.0363319 | 0.6964286 | 9763.784856 |
| | 0.0001187 | 0.2142857 | 12480.76558 |
| | 0.0000023 | 0.1071429 | 12662.53194 |
| | 0.0011908 | 0.7420635 | 41655.94365 |
| | 0.0258646 | 0.3214286 | 45581.34627 |
| | 0.0307173 | 0.3035714 | 46027.40418 |
| | 0.0324998 | 0.3392857 | 47028.57811 |
| F5CSH | 0.0095299 | 0.7512500 | 10018.90389 |
| | 0.0161569 | 0.7112500 | 10086.0305 |
| | 0.0075263 | 0.7312500 | 10143.05116 |
| | 0.0081477 | 0.7512500 | 10153.05258 |
| | 0.0046118 | 0.7512500 | 10166.44834 |
| | 0.0111176 | 0.6912500 | 10183.30263 |
| | 0.0119962 | 0.7112500 | 10201.4022 |
| | 0.0075263 | 0.7312500 | 10211.84828 |
| | 0.0042405 | 0.7312500 | 10223.78504 |
| | 0.0021156 | 0.7712500 | 10235.16667 |
| | 0.0111176 | 0.7312500 | 10278.21222 |
| | 0.0035781 | 0.7512500 | 10296.22207 |
| | 0.0019337 | 0.7512500 | 10313.11386 |
| | 0.0011118 | 0.7912500 | 10356.48068 |
| | 0.0013406 | 0.7912500 | 10373.5117 |
| | 0.0017663 | 0.7912500 | 10388.54465 |
| | 0.0004633 | 0.8112500 | 10403.84952 |
| | 0.0005654 | 0.8112500 | 10419.84632 |
| | 0.0003419 | 0.8112500 | 10436.95581 |
| | 0.0004190 | 0.8112500 | 10463.24082 |
| | 0.0017663 | 0.7712500 | 10483.65617 |
| | 0.0017663 | 0.7712500 | 10496.39967 |
| | 0.0012213 | 0.7712500 | 10504.37612 |
| | 0.0006882 | 0.7912500 | 10509.50161 |
| | 0.0000854 | 0.8512500 | 10518.98678 |
| | 0.0000190 | 0.8912500 | 10534.02712 |
| | 0.0284126 | 0.6912500 | 10614.76076 |
| | 0.0150125 | 0.7512500 | 10627.7903 |
| | 0.0075263 | 0.7312500 | 10643.0144 |
| | 0.0042405 | 0.7712500 | 10663.86727 |
| | 0.0054440 | 0.7312500 | 10689.51826 |
| | 0.0247685 | 0.6712500 | 10713.27982 |
| | 0.0150125 | 0.7112500 | 10750.54704 |
| | 0.0088147 | 0.2712500 | 11044.03097 |
| | 0.0027595 | 0.2200000 | 11064.19634 |
| | 0.0347411 | 0.3000000 | 11957.03925 |
| | 0.0450209 | 0.3000000 | 12241.63856 |
| | 0.0161569 | 0.2800000 | 12320.35659 |
| | 0.0325094 | 0.3000000 | 12450.7298 |
| | 0.0000386 | 0.1200000 | 12919.66629 |
| | 0.0000033 | 0.0600000 | 12979.03902 |
| | 0.0004633 | 0.1800000 | 13032.98071 |
| | 0.0000010 | 0.0400000 | 13092.81349 |
| | 0.0000117 | 0.0800000 | 13195.95886 |
| | 0.0000002 | 0.0200000 | 13295.35656 |
| | 0.0000008 | 0.0600000 | 13325.89827 |
| | 0.0000055 | 0.0800000 | 13355.13127 |
| | 0.0000764 | 0.1400000 | 13405.34327 |
| | 0.0009196 | 0.2200000 | 13486.03676 |
| | 0.0265365 | 0.2912500 | 14217.37567 |
| | 0.0247685 | 0.3112500 | 14521.99131 |
| | 0.0161569 | 0.2800000 | 14618.82347 |
| | 0.0139401 | 0.2600000 | 14800.15747 |
| | 0.0304018 | 0.7000000 | 17985.77938 |
| | 0.0027595 | 0.7912500 | 18369.45605 |
| | 0.0347411 | 0.7112500 | 33613.09559 |
| | 0.0095299 | 0.7512500 | 33970.50674 |
| | 0.0038965 | 0.7512500 | 34365.74762 |
| | 0.0042405 | 0.7512500 | 34643.275 |
| | 0.0231034 | 0.2800000 | 42755.36144 |
| | 0.0325094 | 0.2800000 | 42989.41225 |
| | 0.0247685 | 0.2600000 | 43408.65433 |
| | 0.0032835 | 0.2400000 | 44690.90268 |
| | 0.0027595 | 0.2600000 | 45327.80096 |
| | 0.0075263 | 0.2400000 | 47174.35563 |
| | 0.0119962 | 0.7312500 | 52881.10915 |
| | 0.0075263 | 0.7712500 | 53767.53507 |
| | 0.0265365 | 0.3200000 | 89686.37727 |
| | 0.0347411 | 0.2800000 | 101489.63 |
| | 0.0129359 | 0.7112500 | 108961.4085 |
| | 0.0001643 | 0.1600000 | 118140.2057 |
| | 0.0000854 | 0.1400000 | 134053.0066 |
| | 0.0325094 | 0.2912500 | 151649.6484 |
| | 0.0173773 | 0.3000000 | 168760.9312 |
| | 0.0002508 | 0.1600000 | 199067.5944 |
| F5ISH | 0.0267757 | 0.3166667 | 10194.75241 |
| | 0.0214507 | 0.3166667 | 10265.10592 |
| | 0.0298421 | 0.3166667 | 10268.93612 |
| | 0.0368884 | 0.3166667 | 10370.69947 |
| | 0.0151936 | 0.3000000 | 10413.75186 |
| | 0.0052747 | 0.2833333 | 10416.27302 |
| | 0.0368884 | 0.3333333 | 10419.51807 |
| | 0.0388567 | 0.3333333 | 10424.409 |
| | 0.0099719 | 0.2666667 | 10454.03617 |
| | 0.0476454 | 0.3277778 | 10753.95946 |
| | 0.0010397 | 0.7833333 | 11814.91128 |
| | 0.0191526 | 0.7166667 | 11926.05207 |
| | 0.0127163 | 0.7166667 | 12162.99607 |
| | 0.0368884 | 0.6888889 | 12771.26536 |
| | 0.0388567 | 0.6888889 | 63202.19281 |
| | 0.0282731 | 0.3111111 | 134528.9143 |
| F6CSL | 0.0394890 | 0.3078431 | 3357.303486 |
| | 0.0013257 | 0.2019608 | 3428.881722 |
| | 0.0253401 | 0.3078431 | 4126.549336 |
| | 0.0064640 | 0.7598039 | 4409.265978 |
| | 0.0056480 | 0.7245098 | 4868.735212 |
| | 0.0108921 | 0.7245098 | 4966.887753 |
| | 0.0416586 | 0.6892157 | 5026.966286 |
| | 0.0239258 | 0.6960784 | 5070.710423 |
| | 0.0283870 | 0.6784314 | 5098.522928 |
| | 0.0108921 | 0.7313725 | 5260.926275 |
| | 0.0300253 | 0.7068627 | 5289.432519 |
| | 0.0253401 | 0.7068627 | 5366.242108 |
| | 0.0189314 | 0.6960784 | 5390.905048 |
| | 0.0178353 | 0.6784314 | 5474.342743 |
| | 0.0108921 | 0.6784314 | 5502.673564 |
| | 0.0178353 | 0.7068627 | 5557.327902 |
| | 0.0178353 | 0.6960784 | 5594.085902 |
| | 0.0148737 | 0.7137255 | 5612.225059 |
| | 0.0189314 | 0.6892157 | 5656.80656 |
| | 0.0213014 | 0.6960784 | 5713.615364 |
| | 0.0189314 | 0.6960784 | 5733.192122 |
| | 0.0439284 | 0.6892157 | 5779.276452 |
| | 0.0283870 | 0.6784314 | 5831.045095 |
| | 0.0200859 | 0.6960784 | 5913.554395 |
| | 0.0463016 | 0.6539216 | 5946.280315 |
| | 0.0213014 | 0.6715686 | 5967.465293 |
| | 0.0374161 | 0.6892157 | 6005.550627 |
| | 0.0268262 | 0.6715686 | 6032.27113 |
| | 0.0283870 | 0.6715686 | 6049.453271 |
| | 0.0189314 | 0.6892157 | 6097.371545 |
| | 0.0213014 | 0.6715686 | 6117.220263 |
| | 0.0374161 | 0.6892157 | 6138.640057 |
| | 0.0102204 | 0.7598039 | 6186.818987 |
| | 0.0089866 | 0.7137255 | 6208.87014 |
| | 0.0374161 | 0.6892157 | 6269.981914 |
| | 0.0317443 | 0.6715686 | 6291.763152 |
| | 0.0158088 | 0.7068627 | 6368.819184 |
| | 0.0283870 | 0.6784314 | 6405.544153 |
| | 0.0022478 | 0.8019608 | 6489.894258 |
| | 0.0102204 | 0.7313725 | 6505.638023 |
| | 0.0335469 | 0.6715686 | 6545.783991 |
| | 0.0463016 | 0.6892157 | 6777.027523 |
| | 0.0148737 | 0.2480392 | 7784.588778 |
| | 0.0002376 | 0.1882353 | 8237.401638 |
| | 0.0253401 | 0.7176471 | 9047.708867 |
| | 0.0416586 | 0.7000000 | 9960.034625 |
| | 0.0084211 | 0.2725490 | 11573.82995 |
| | 0.0123542 | 0.2725490 | 11751.31327 |
| | 0.0000811 | 0.2019608 | 12481.18612 |
| | 0.0000019 | 0.0960784 | 12650.52312 |
| | 0.0000019 | 0.0960784 | 12906.19826 |
| | 0.0416586 | 0.3078431 | 14103.42005 |
| | 0.0003075 | 0.1843137 | 14429.27882 |
| | 0.0463016 | 0.3186275 | 15184.98539 |
| | 0.0064640 | 0.2549020 | 17435.05944 |
| | 0.0189314 | 0.2901961 | 28157.11019 |
| | 0.0317443 | 0.3078431 | 33514.17865 |
| | 0.0084211 | 0.7352941 | 41482.3358 |
| | 0.0024195 | 0.2549020 | 43439.78321 |
| | 0.0014315 | 0.2019608 | 44236.21224 |
| | 0.0003348 | 0.1843137 | 44629.01998 |
| | 0.0000347 | 0.1490196 | 44938.74105 |
| | 0.0000213 | 0.1490196 | 45465.05824 |
| | 0.0002590 | 0.2019608 | 46196.8513 |
| F6CSH | 0.0046532 | 0.7500000 | 10031.5232 |
| | 0.0029156 | 0.7723214 | 10128.78825 |
| | 0.0218270 | 0.6875000 | 10197.87061 |
| | 0.0168046 | 0.7053571 | 10244.13547 |
| | 0.0015156 | 0.7767857 | 10283.30231 |
| | 0.0358888 | 0.6875000 | 10376.97318 |
| | 0.0072807 | 0.7410714 | 10461.31639 |
| | 0.0380990 | 0.6875000 | 10503.04585 |
| | 0.0179544 | 0.6875000 | 10764.84395 |
| | 0.0024839 | 0.7723214 | 10769.30644 |
| | 0.0404240 | 0.7053571 | 10804.20204 |
| | 0.0157200 | 0.7053571 | 10851.39035 |
| | 0.0358888 | 0.6696429 | 10909.85409 |
| | 0.0264071 | 0.6830357 | 10978.99413 |
| | 0.0026919 | 0.7544643 | 11049.12454 |
| | 0.0002526 | 0.1785714 | 12717.12602 |
| | 0.0006957 | 0.1785714 | 12888.24561 |
| | 0.0247957 | 0.3035714 | 13164.31745 |
| | 0.0004037 | 0.8035714 | 23169.52525 |
| | 0.0017909 | 0.7946429 | 23418.55947 |
| | 0.0015156 | 0.8035714 | 23683.93102 |
| | 0.0001048 | 0.8258929 | 25404.03647 |
| | 0.0004037 | 0.8258929 | 25984.38257 |
| | 0.0058349 | 0.7232143 | 39174.72596 |
| | 0.0005313 | 0.8080357 | 39939.38857 |
| | 0.0000126 | 0.1250000 | 44706.89653 |
| | 0.0000113 | 0.0892857 | 45581.88392 |
| | 0.0000271 | 0.8571429 | 51329.3608 |
| | 0.0000699 | 0.8437500 | 53487.79549 |
| | 0.0078297 | 0.2500000 | 66724.50295 |
| | 0.0013931 | 0.7723214 | 79341.48074 |
| | 0.0004037 | 0.1964286 | 100935.9107 |
| | 0.0000032 | 0.0714286 | 133359.809 |
| | 0.0001279 | 0.1785714 | 198260.4773 |
| F6ISL | 0.0297570 | 0.6937120 | 2640.69294 |
| | 0.0464532 | 0.6653144 | 2689.783828 |
| | 0.0280872 | 0.6754564 | 2995.703282 |
| | 0.0394441 | 0.6572008 | 3030.319866 |
| | 0.0046075 | 0.7484787 | 3056.069215 |
| | 0.0315115 | 0.6572008 | 3258.38603 |
| | 0.0297570 | 0.3002028 | 3365.113073 |
| | 0.0002376 | 0.1906694 | 3437.484027 |
| | 0.0046075 | 0.2454361 | 7781.264988 |
| | 0.0163556 | 0.7119675 | 8842.911218 |
| | 0.0053075 | 0.2819473 | 11569.16318 |
| | 0.0006149 | 0.2271805 | 11724.85116 |
| | 0.0022072 | 0.2271805 | 12478.09797 |
| | 0.0144293 | 0.3002028 | 12902.9216 |
| | 0.0163556 | 0.2819473 | 33565.39821 |
| | 0.0003097 | 0.1724138 | 34219.23059 |
| F6ISH | 0.0321237 | 0.6800000 | 10518.14292 |
| | 0.0044957 | 0.7400000 | 10601.92366 |
| | 0.0140543 | 0.7000000 | 10692.28071 |
| | 0.0208103 | 0.7200000 | 10772.27076 |
| | 0.0060548 | 0.7200000 | 10787.02725 |
| | 0.0024156 | 0.7600000 | 10815.66745 |
| | 0.0075274 | 0.7200000 | 10847.40644 |
| | 0.0236200 | 0.7200000 | 10866.3702 |
| | 0.0048470 | 0.7400000 | 10883.24318 |
| | 0.0483445 | 0.6800000 | 10898.15904 |
| | 0.0195181 | 0.7200000 | 10915.09457 |
| | 0.0284502 | 0.6800000 | 10922.99947 |
| | 0.0122789 | 0.7000000 | 10936.0038 |
| | 0.0251444 | 0.6800000 | 10954.03205 |
| | 0.0140543 | 0.7000000 | 10975.52556 |
| | 0.0182965 | 0.7000000 | 10994.92546 |
| | 0.0361964 | 0.6800000 | 11025.95351 |
| | 0.0321237 | 0.7000000 | 11041.94186 |
| | 0.0361964 | 0.6800000 | 11461.63754 |
| | 0.0030615 | 0.7400000 | 11480.30167 |
| | 0.0005261 | 0.8200000 | 11502.5506 |
| | 0.0030615 | 0.7600000 | 11552.19581 |
| | 0.0048470 | 0.7600000 | 11568.9046 |
| | 0.0028305 | 0.7800000 | 11588.80348 |
| | 0.0041677 | 0.7400000 | 11651.54813 |
| | 0.0026155 | 0.7600000 | 11900.72691 |
| | 0.0383928 | 0.7000000 | 11987.52615 |
| | 0.0208103 | 0.3000000 | 12191.72667 |
| | 0.0341081 | 0.2800000 | 12227.2304 |
| | 0.0456728 | 0.3200000 | 12252.12747 |
| | 0.0341081 | 0.3000000 | 12311.09499 |
| | 0.0080853 | 0.2400000 | 12335.34416 |
| | 0.0070042 | 0.2600000 | 12372.53121 |
| | 0.0131401 | 0.2600000 | 12426.65893 |
| | 0.0035759 | 0.2000000 | 12566.3271 |
| | 0.0075274 | 0.2400000 | 12630.52717 |
| | 0.0302390 | 0.3000000 | 12703.38429 |
| | 0.0080853 | 0.2600000 | 12763.65458 |
| | 0.0431265 | 0.3000000 | 12851.09203 |
| | 0.0000203 | 0.1200000 | 13181.77247 |
| | 0.0004393 | 0.2000000 | 13231.38136 |
| | 0.0000002 | 0.0400000 | 13317.42046 |
| | 0.0000118 | 0.1000000 | 13400.71809 |
| | 0.0018967 | 0.2200000 | 13464.97455 |
| | 0.0022297 | 0.2200000 | 13586.74139 |
| | 0.0013635 | 0.1800000 | 13711.01396 |
| | 0.0011524 | 0.2000000 | 13796.51085 |
| | 0.0383928 | 0.6800000 | 23411.55 |
| | 0.0052229 | 0.7400000 | 23915.54212 |
| | 0.0004011 | 0.8400000 | 39529.93455 |
| | 0.0000869 | 0.8400000 | 39888.64853 |
| | 0.0140543 | 0.7400000 | 40571.4705 |
| | 0.0052229 | 0.2200000 | 41956.29129 |
| | 0.0016099 | 0.2600000 | 44526.33609 |
| | 0.0002298 | 0.1800000 | 45322.88979 |
| | 0.0361964 | 0.3400000 | 46419.54842 |
| | 0.0208103 | 0.2800000 | 47393.37799 |
| | 0.0002090 | 0.8200000 | 51260.79681 |
| | 0.0002525 | 0.8200000 | 51799.54682 |
| | 0.0006866 | 0.8000000 | 52580.37193 |
| | 0.0080853 | 0.7400000 | 75206.21809 |
| | 0.0010585 | 0.7800000 | 79541.53333 |
| | 0.0000251 | 0.1200000 | 100624.8699 |
| | 0.0001290 | 0.1600000 | 133676.0475 |

TABLE 19
Biomarkers identified by SELDI technology with a p-value smaller
or equal to 0.05 that can discriminate primary DF infection from
OFI. Grouped according to fraction it was detected in.
Diagnostic

Ct1_2 vs DF1_2

| | p value | roc | m/z averaqe |
|---|---|---|---|
| F1CSL | 0.0000000 | 0.9625850 | 4579.926292 |
| | 0.0000000 | 0.9778912 | 4596.110987 |
| | 0.0000004 | 0.9013605 | 5583.561565 |
| | 0.0000001 | 0.9319728 | 3870.26222 |
| | 0.0000000 | 0.9472789 | 3806.262116 |
| | 0.0236516 | 0.6564626 | 4990.19603 |
| | 0.0000448 | 0.8290816 | 6941.418376 |
| | 0.0000001 | 0.0459184 | 7485.646702 |
| | 0.0000011 | 0.9013605 | 3933.137941 |
| | 0.0000004 | 0.9013605 | 4441.164166 |
| | 0.0000008 | 0.9013605 | 4459.777653 |
| | 0.0000140 | 0.8401361 | 4800.693027 |
| | 0.0000005 | 0.9166667 | 3061.572119 |
| | 0.0000000 | 0.9472789 | 3522.242859 |
| | 0.0000128 | 0.8639456 | 6140.608328 |
| | 0.0000411 | 0.8375850 | 6138.119764 |
| | 0.0000097 | 0.8750000 | 4423.747371 |
| | 0.0000184 | 0.8554422 | 4654.384151 |
| | 0.0000007 | 0.9013605 | 3431.457425 |
| | 0.0153351 | 0.6913265 | 5183.584315 |
| | 0.0000014 | 0.1071429 | 7658.7026 |
| | 0.0000154 | 0.8554422 | 4488.791713 |
| | 0.0053031 | 0.7372449 | 2517.703609 |
| | 0.0067852 | 0.7414966 | 2886.289725 |
| | 0.0630724 | 0.6564626 | 23588.47849 |
| | 0.0027893 | 0.7414966 | 3821.714797 |
| | 0.0072096 | 0.7261905 | 3248.144023 |
| | 0.0002988 | 0.1989796 | 2752.206092 |
| | 0.0000106 | 0.1377551 | 2980.456404 |
| | 0.0000011 | 0.9013605 | 3187.926121 |
| | 0.0009909 | 0.2295918 | 7940.572626 |
| | 0.0000128 | 0.8401361 | 9107.540827 |
| | 0.0000965 | 0.8401361 | 8780.719118 |
| | 0.0005509 | 0.7763605 | 5912.839452 |
| | 0.0016234 | 0.7568027 | 4471.501882 |
| | 0.0137100 | 0.2908163 | 3957.455551 |
| | 0.0412997 | 0.3324830 | 4189.213856 |
| | 0.0097100 | 0.2755102 | 3321.159048 |
| | 0.0322319 | 0.3061224 | 3976.207226 |
| | 0.0072096 | 0.2755102 | 7195.954395 |
| | 0.0000184 | 0.8401361 | 3448.844444 |
| | 0.0009220 | 0.2295918 | 2672.066808 |
| | 0.0433518 | 0.3367347 | 8142.685699 |
| | 0.0026103 | 0.7636054 | 4150.652833 |
| | 0.0053031 | 0.2823129 | 3141.691497 |
| | 0.0477149 | 0.3367347 | 4304.939937 |
| | 0.0097100 | 0.7108844 | 3893.413384 |
| | 0.0000489 | 0.8248299 | 4115.711469 |
| | 0.0393306 | 0.3214286 | 3087.325152 |
| | 0.0097100 | 0.3018707 | 2902.12753 |
| | 0.0004070 | 0.7789116 | 10092.33514 |
| F1CSH | 0.0322319 | 0.3435374 | 10483.4761 |
| | 0.0013168 | 0.7636054 | 10757.79609 |
| | 0.0031814 | 0.2602041 | 11076.61987 |
| | 0.0003769 | 0.2142857 | 11157.80715 |
| | 0.0001140 | 0.1989796 | 11203.02954 |
| | 0.0137100 | 0.3129252 | 11350.40235 |
| | 0.0002988 | 0.1836735 | 11396.01715 |
| | 0.0000168 | 0.1683673 | 11498.19671 |
| | 0.0007411 | 0.2142857 | 11528.91453 |
| | 0.0000097 | 0.1530612 | 11605.52007 |
| | 0.0374418 | 0.3061224 | 11657.66336 |
| | 0.0276559 | 0.3061224 | 12494.15007 |
| | 0.0007974 | 0.2295918 | 13292.33306 |
| | 0.0477149 | 0.3520408 | 13419.85168 |
| | 0.0006394 | 0.2295918 | 13841.62893 |
| | 0.0000448 | 0.8248299 | 23260.27196 |
| | 0.0000533 | 0.8248299 | 23823.38294 |
| | 0.0338949 | 0.6564626 | 25774.83879 |
| | 0.0454892 | 0.6802721 | 29110.79546 |
| | 0.0129560 | 0.6955782 | 30257.11683 |
| | 0.0076577 | 0.7108844 | 38507.26592 |
| | 0.0249266 | 0.6955782 | 46535.55302 |
| | 0.0356307 | 0.3324830 | 53621.70016 |
| | 0.0122390 | 0.3171769 | 54009.77568 |
| | 0.0374418 | 0.3282313 | 57090.27251 |
| | 0.0171275 | 0.3324830 | 63488.45467 |
| | 0.0433518 | 0.3214286 | 149368.5056 |
| F1ISL | 0.0019931 | 0.7399425 | 2716.553062 |
| | 0.0006862 | 0.7923851 | 2862.774426 |
| | 0.0011457 | 0.7492816 | 2923.355944 |
| | 0.0026829 | 0.7492816 | 3166.902131 |
| | 0.0000396 | 0.8211207 | 3415.128978 |
| | 0.0006428 | 0.7636494 | 3457.994054 |
| | 0.0001752 | 0.7780172 | 3501.960593 |
| | 0.0010097 | 0.7636494 | 3793.065138 |
| | 0.0002672 | 0.7923851 | 3920.288144 |
| | 0.0059277 | 0.7061782 | 4103.744956 |
| | 0.0010757 | 0.7780172 | 4122.870618 |
| | 0.0166483 | 0.7061782 | 4136.963402 |
| | 0.0243579 | 0.3333333 | 4276.341498 |
| | 0.0130015 | 0.2902299 | 4292.764456 |
| | 0.0026829 | 0.7349138 | 4414.952812 |
| | 0.0000073 | 0.8354885 | 4432.616925 |
| | 0.0000028 | 0.8498563 | 4449.125935 |
| | 0.0100801 | 0.7112069 | 4470.030305 |
| | 0.0136685 | 0.7061782 | 5606.87859 |
| | 0.0008335 | 0.7500000 | 5908.422969 |
| | 0.0136685 | 0.6925287 | 5993.694087 |
| | 0.0255115 | 0.6637931 | 6127.388003 |
| | 0.0130015 | 0.6918103 | 6588.428983 |
| | 0.0095715 | 0.3045977 | 6640.401792 |
| | 0.0040169 | 0.3045977 | 6654.995846 |
| | 0.0000102 | 0.8498563 | 6955.232848 |
| | 0.0221857 | 0.3189655 | 8151.799057 |
| | 0.0493518 | 0.3333333 | 41115.90512 |
| F1ISH | 0.0477569 | 0.3296296 | 10064.08045 |
| | 0.0252391 | 0.2962963 | 10415.77202 |
| | 0.0225628 | 0.3129630 | 10428.31602 |
| | 0.0453879 | 0.3462963 | 10442.19383 |
| | 0.0238684 | 0.2962963 | 10587.75825 |
| | 0.0001075 | 0.1796296 | 10614.34283 |
| | 0.0001525 | 0.1962963 | 10634.45923 |
| | 0.0013461 | 0.2296296 | 10649.30096 |
| | 0.0266773 | 0.3296296 | 10662.8102 |
| | 0.0000477 | 0.1462963 | 10687.34192 |
| | 0.0032007 | 0.2796296 | 10715.49742 |
| | 0.0058850 | 0.2629630 | 10750.05013 |
| | 0.0201368 | 0.2898148 | 10777.41795 |
| | 0.0409462 | 0.3231481 | 10884.94807 |
| | 0.0453879 | 0.3296296 | 11025.00949 |
| | 0.0150417 | 0.3129630 | 11091.8077 |
| | 0.0169247 | 0.2962963 | 11117.15405 |
| | 0.0314243 | 0.6666667 | 12231.72039 |
| | 0.0039363 | 0.7000000 | 23104.0813 |
| | 0.0104530 | 0.7000000 | 23638.655 |
| | 0.0213198 | 0.3129630 | 49665.31317 |
| | 0.0169247 | 0.2962963 | 50851.35303 |
| | 0.0067063 | 0.2796296 | 51514.98459 |
| | 0.0003546 | 0.1962963 | 52248.13667 |
| | 0.0012488 | 0.2129630 | 53216.07485 |
| | 0.0003849 | 0.2129630 | 53984.24055 |
| | 0.0001174 | 0.1629630 | 54593.1773 |
| | 0.0004176 | 0.2129630 | 55291.41693 |
| | 0.0002149 | 0.1962963 | 56616.00188 |
| | 0.0331600 | 0.3296296 | 67189.1931 |
| | 0.0081320 | 0.2962963 | 69203.03801 |
| | 0.0297672 | 0.3296296 | 70488.37194 |
| F5CSL | 0.0447967 | 0.3268634 | 3360.320143 |
| | 0.0009885 | 0.2336957 | 3432.717673 |
| | 0.0255017 | 0.3268634 | 4788.767558 |
| | 0.0011302 | 0.2336957 | 5006.990761 |
| | 0.0447967 | 0.6459627 | 5165.803217 |
| | 0.0080451 | 0.2802795 | 7785.325256 |
| | 0.0076054 | 0.7150621 | 8726.282926 |
| | 0.0000824 | 0.8167702 | 8846.247547 |
| | 0.0000094 | 0.8633540 | 8961.939907 |
| | 0.0002587 | 0.8012422 | 9192.684623 |
| | 0.0003725 | 0.7701863 | 9468.993468 |
| | 0.0004957 | 0.7701863 | 9679.241772 |
| | 0.0000055 | 0.1560559 | 12480.76558 |
| | 0.0000006 | 0.1094720 | 12662.53194 |
| | 0.0294860 | 0.3423913 | 44310.14724 |
| | 0.0080451 | 0.2647516 | 44676.21036 |
| | 0.0067902 | 0.2958075 | 45036.99926 |
| | 0.0131455 | 0.2958075 | 45285.08473 |
| | 0.0085073 | 0.2802795 | 45581.34627 |
| | 0.0111937 | 0.2958075 | 46027.40418 |
| | 0.0100399 | 0.2958075 | 47028.57811 |
| F5CSH | 0.0001302 | 0.8152381 | 10018.90389 |
| | 0.0026108 | 0.7466667 | 10086.0305 |
| | 0.0037324 | 0.7466667 | 10143.05116 |
| | 0.0028068 | 0.7638095 | 10153.05258 |
| | 0.0034781 | 0.7638095 | 10166.44834 |
| | 0.0052762 | 0.7466667 | 10183.30263 |
| | 0.0056468 | 0.7295238 | 10201.4022 |
| | 0.0020955 | 0.7466667 | 10211.84828 |
| | 0.0011428 | 0.7638095 | 10223.78504 |
| | 0.0012348 | 0.7638095 | 10235.16667 |
| | 0.0188434 | 0.6952381 | 10278.21222 |
| | 0.0131040 | 0.6952381 | 10296.22207 |
| | 0.0060407 | 0.7295238 | 10313.11386 |
| | 0.0052762 | 0.7123810 | 10356.48068 |
| | 0.0108626 | 0.6952381 | 10373.5117 |
| | 0.0095644 | 0.7123810 | 10388.54465 |
| | 0.0016751 | 0.7809524 | 10403.84952 |
| | 0.0042925 | 0.7123810 | 10419.84632 |
| | 0.0056468 | 0.7295238 | 10436.95581 |
| | 0.0167240 | 0.6780952 | 10463.24082 |
| | 0.0131040 | 0.7123810 | 10483.65617 |
| | 0.0095644 | 0.6952381 | 10496.39967 |
| | 0.0123151 | 0.6952381 | 10504.37612 |
| | 0.0049277 | 0.7123810 | 10509.50161 |
| | 0.0018057 | 0.7638095 | 10518.98678 |
| | 0.0069036 | 0.7123810 | 10534.02712 |
| | 0.0593614 | 0.6438095 | 10614.76076 |
| | 0.0095644 | 0.6952381 | 10627.7903 |
| | 0.0131040 | 0.6609524 | 10643.0144 |
| | 0.0237962 | 0.6780952 | 10663.86727 |
| | 0.0224624 | 0.3085714 | 12130.19557 |
| | 0.0157451 | 0.2742857 | 12810.96828 |
| | 0.0000088 | 0.1200000 | 12919.66629 |
| | 0.0000028 | 0.1028571 | 12979.03902 |
| | 0.0000072 | 0.1542857 | 13032.98071 |
| | 0.0000001 | 0.0514286 | 13092.81349 |
| | 0.0000006 | 0.1028571 | 13195.95886 |
| | 0.0000000 | 0.0342857 | 13295.35656 |
| | 0.0000001 | 0.0514286 | 13325.89827 |
| | 0.0000133 | 0.1371429 | 13355.13127 |
| | 0.0000038 | 0.1200000 | 13405.34327 |
| | 0.0000147 | 0.1200000 | 13486.03676 |
| | 0.0037324 | 0.2571429 | 13633.86949 |
| | 0.0009777 | 0.7809524 | 14029.40621 |
| | 0.0019456 | 0.7638095 | 14996.39253 |
| | 0.0484138 | 0.6857143 | 17665.44985 |
| | 0.0089687 | 0.7295238 | 17790.31008 |
| | 0.0004734 | 0.8152381 | 17985.77938 |
| | 0.0000754 | 0.8323810 | 18369.45605 |
| | 0.0459589 | 0.6514286 | 29222.15188 |
| | 0.0046002 | 0.2571429 | 42755.36144 |
| | 0.0064592 | 0.2742857 | 42989.41225 |
| | 0.0030161 | 0.2571429 | 43408.65433 |
| | 0.0010573 | 0.2228571 | 44690.90268 |
| | 0.0022559 | 0.2228571 | 45327.80096 |
| | 0.0089687 | 0.2914286 | 47174.35563 |
| | 0.0056468 | 0.7123810 | 51440.05877 |
| | 0.0069036 | 0.7200000 | 51889.38929 |
| | 0.0049277 | 0.7295238 | 52881.10915 |
| | 0.0078757 | 0.7295238 | 75141.88947 |
| | 0.0237962 | 0.7123810 | 79071.92294 |
| | 0.0157451 | 0.7123810 | 82425.00046 |
| | 0.0237962 | 0.6780952 | 84062.87518 |
| | 0.0028068 | 0.2400000 | 101489.63 |
| | 0.0008348 | 0.2228571 | 118140.2057 |
| | 0.0030161 | 0.2742857 | 134053.0066 |
| | 0.0037324 | 0.2571429 | 168760.9312 |
| | 0.0089687 | 0.3085714 | 199067.5944 |
| F5ISL | 0.0351749 | 0.3500000 | 43525.88014 |
| F5ISH | 0.0367139 | 0.3416667 | 11796.34403 |
| | 0.0094934 | 0.3222222 | 13078.15809 |
| | 0.0351749 | 0.3458333 | 13157.26855 |
| | 0.0282801 | 0.3222222 | 13222.277 |
| | 0.0148061 | 0.3222222 | 13254.13984 |
| | 0.0050685 | 0.2944444 | 13274.84863 |
| | 0.0170854 | 0.3138889 | 13538.51377 |
| F6CSL | 0.0032619 | 0.2527778 | 3357.303486 |
| | 0.0000898 | 0.1930556 | 3428.881722 |
| | 0.0104991 | 0.3041667 | 3473.520711 |
| | 0.0141083 | 0.3180556 | 4126.549336 |
| | 0.0077358 | 0.6944444 | 4178.147403 |
| | 0.0007803 | 0.7583333 | 4409.265978 |
| | 0.0134396 | 0.3041667 | 4788.788756 |
| | 0.0270506 | 0.6833333 | 7636.624747 |
| | 0.0270506 | 0.6666667 | 7661.987216 |
| | 0.0062757 | 0.2944444 | 7784.588778 |
| | 0.0023163 | 0.2527778 | 8237.401638 |
| | 0.0032619 | 0.7166667 | 8296.864065 |
| | 0.0472018 | 0.6708333 | 8722.157337 |
| | 0.0247295 | 0.6986111 | 8746.621393 |
| | 0.0008846 | 0.7638889 | 9047.708867 |
| | 0.0491743 | 0.6569444 | 9463.07485 |
| | 0.0322615 | 0.6847222 | 10472.88205 |
| | 0.0029134 | 0.2805556 | 11573.82995 |
| | 0.0015338 | 0.2527778 | 11751.31327 |
| | 0.0000060 | 0.1277778 | 12481.18612 |
| | 0.0000000 | 0.0444444 | 12650.52312 |
| | 0.0000002 | 0.0861111 | 12906.19826 |
| | 0.0003358 | 0.2111111 | 14429.27882 |
| | 0.0434560 | 0.3083333 | 23566.3546 |
| | 0.0434560 | 0.6652778 | 25601.84995 |
| | 0.0012800 | 0.2208333 | 33514.17865 |
| | 0.0014444 | 0.2625000 | 34088.06629 |
| | 0.0014444 | 0.2347222 | 34523.55143 |
| | 0.0036480 | 0.2805556 | 43439.78321 |
| | 0.0010016 | 0.2527778 | 44236.21224 |
| | 0.0000538 | 0.1555556 | 44629.01998 |
| | 0.0000234 | 0.1694444 | 44938.74105 |
| | 0.0000343 | 0.1833333 | 45465.05824 |
| | 0.0000115 | 0.1694444 | 46196.8513 |
| F6CSH | 0.0162203 | 0.3214286 | 11147.59114 |
| | 0.0286014 | 0.3214286 | 11670.74819 |
| | 0.0069950 | 0.2889610 | 11750.95799 |
| | 0.0021519 | 0.2240260 | 11780.11833 |
| | 0.0017657 | 0.2402597 | 11832.56962 |
| | 0.0035905 | 0.2564935 | 11908.94273 |
| | 0.0078607 | 0.2889610 | 11993.02686 |
| | 0.0420946 | 0.3214286 | 12057.41098 |
| | 0.0008922 | 0.2727273 | 12717.12602 |
| | 0.0005822 | 0.2402597 | 12888.24561 |
| | 0.0110621 | 0.7110390 | 13612.48842 |
| | 0.0051931 | 0.7272727 | 13846.52517 |
| | 0.0002574 | 0.7759740 | 14825.26488 |
| | 0.0002778 | 0.7759740 | 15055.63774 |
| | 0.0078607 | 0.7110390 | 15411.25871 |
| | 0.0029712 | 0.7272727 | 15613.40893 |
| | 0.0009567 | 0.7435065 | 15812.1091 |
| | 0.0015448 | 0.7272727 | 16257.83812 |
| | 0.0029712 | 0.7272727 | 16334.75585 |
| | 0.0026143 | 0.7435065 | 16782.20178 |
| | 0.0022970 | 0.7272727 | 17134.49865 |
| | 0.0145653 | 0.3051948 | 21786.35664 |
| | 0.0000222 | 0.8409091 | 25404.03647 |
| | 0.0002996 | 0.7759740 | 25984.38257 |
| | 0.0331465 | 0.6948052 | 30482.78007 |
| | 0.0162203 | 0.3051948 | 33560.43936 |
| | 0.0016518 | 0.7597403 | 39939.38857 |
| | 0.0074165 | 0.2564935 | 43596.91071 |
| | 0.0000019 | 0.1103896 | 44706.89653 |
| | 0.0000726 | 0.1915584 | 45581.88392 |
| | 0.0005036 | 0.2240260 | 46366.8165 |
| | 0.0021519 | 0.7435065 | 51329.3608 |
| | 0.0258789 | 0.3214286 | 56681.92589 |
| | 0.0004040 | 0.2240260 | 57685.14725 |
| | 0.0000204 | 0.1590909 | 58847.42102 |
| | 0.0000030 | 0.1266234 | 59365.11459 |
| | 0.0000120 | 0.1590909 | 60097.13372 |
| | 0.0001005 | 0.1915584 | 61383.37478 |
| | 0.0008317 | 0.2402597 | 66724.50295 |
| | 0.0365087 | 0.6623377 | 75362.49021 |
| | 0.0401572 | 0.6298701 | 82507.09819 |
| | 0.0029712 | 0.2727273 | 89782.65711 |
| | 0.0001005 | 0.2077922 | 100935.9107 |
| | 0.0000001 | 0.0779221 | 117244.8486 |
| | 0.0000030 | 0.1428571 | 133359.809 |
| | 0.0233833 | 0.3214286 | 149435.3445 |
| | 0.0001751 | 0.2077922 | 198260.4773 |
| F6ISL | 0.0095715 | 0.2995690 | 2739.450599 |
| | 0.0123635 | 0.3045977 | 3145.966693 |
| | 0.0010757 | 0.2614943 | 3365.113073 |
| | 0.0000102 | 0.1609195 | 3437.484027 |
| | 0.0025296 | 0.7205460 | 7625.50723 |
| | 0.0095715 | 0.2902299 | 7781.264988 |
| | 0.0130015 | 0.6824713 | 8842.911218 |
| | 0.0143656 | 0.2902299 | 11724.85116 |
| | 0.0010097 | 0.2327586 | 12478.09797 |
| | 0.0381955 | 0.3189655 | 12902.9216 |
| | 0.0381955 | 0.6681034 | 15922.78821 |
| | 0.0211642 | 0.6537356 | 16108.41436 |
| | 0.0381955 | 0.3333333 | 33565.39821 |
| | 0.0002019 | 0.2040230 | 34219.23059 |
| F6ISH | 0.0298954 | 0.6862319 | 10518.14292 |
| | 0.0216133 | 0.6811594 | 10601.92366 |
| | 0.0312772 | 0.6666667 | 10692.28071 |
| | 0.0407710 | 0.6811594 | 10815.66745 |
| | 0.0619867 | 0.6666667 | 10866.3702 |
| | 0.0342060 | 0.6811594 | 10883.24318 |
| | 0.0312772 | 0.6717391 | 10898.15904 |
| | 0.0113934 | 0.6956522 | 10915.09457 |
| | 0.0067313 | 0.6956522 | 10922.99947 |
| | 0.0032489 | 0.7442029 | 10936.0038 |
| | 0.0132657 | 0.6956522 | 10954.03205 |
| | 0.0108237 | 0.7101449 | 10975.52556 |
| | 0.0097596 | 0.7101449 | 10994.92546 |
| | 0.0206106 | 0.6956522 | 11025.95351 |
| | 0.0169945 | 0.6956522 | 11041.94186 |
| | 0.0483741 | 0.6521739 | 11095.63804 |
| | 0.0373659 | 0.6521739 | 11111.495 |
| | 0.0012366 | 0.7536232 | 11461.63754 |
| | 0.0048394 | 0.7101449 | 11480.30167 |
| | 0.0003096 | 0.7826087 | 11502.5506 |
| | 0.0014909 | 0.7536232 | 11535.71087 |
| | 0.0030654 | 0.7536232 | 11552.19581 |
| | 0.0206106 | 0.6956522 | 11568.9046 |
| | 0.0206106 | 0.7101449 | 11588.80348 |
| | 0.0011612 | 0.7731884 | 11900.72691 |
| | 0.0357562 | 0.3340580 | 12191.72667 |
| | 0.0407710 | 0.3340580 | 12227.2304 |
| | 0.0260603 | 0.3050725 | 12252.12747 |
| | 0.0425699 | 0.3340580 | 12311.09499 |
| | 0.0119896 | 0.3195652 | 12335.34416 |
| | 0.0030654 | 0.2326087 | 12372.53121 |
| | 0.0154049 | 0.3340580 | 12426.65893 |
| | 0.0051168 | 0.2760870 | 12566.3271 |
| | 0.0067313 | 0.2760870 | 12630.52717 |
| | 0.0444355 | 0.3340580 | 12660.70452 |
| | 0.0327135 | 0.3340580 | 12703.38429 |
| | 0.0951206 | 0.3579710 | 12763.65458 |
| | 0.0169945 | 0.3195652 | 12816.16883 |
| | 0.0000028 | 0.1166667 | 13181.77247 |
| | 0.0000228 | 0.1601449 | 13231.38136 |
| | 0.0000000 | 0.0297101 | 13317.42046 |
| | 0.0000001 | 0.0876812 | 13400.71809 |
| | 0.0005330 | 0.2181159 | 13464.97455 |
| | 0.0028915 | 0.2471014 | 13586.74139 |
| | 0.0001228 | 0.2036232 | 13711.01396 |
| | 0.0001141 | 0.2181159 | 13796.51085 |
| | 0.0373659 | 0.3485507 | 14550.43665 |
| | 0.0216133 | 0.3340580 | 14599.05541 |
| | 0.0139476 | 0.3050725 | 15028.22953 |
| | 0.0048394 | 0.7297101 | 15772.51523 |
| | 0.0040870 | 0.7391304 | 15807.50552 |
| | 0.0051168 | 0.7152174 | 15961.34377 |
| | 0.0022821 | 0.7297101 | 16106.21548 |
| | 0.0000003 | 0.1021739 | 33558.37315 |
| | 0.0006502 | 0.7876812 | 39529.93455 |
| | 0.0019049 | 0.7246377 | 39888.64853 |
| | 0.0285663 | 0.6717391 | 40571.4705 |
| | 0.0021494 | 0.3050725 | 41956.29129 |
| | 0.0146603 | 0.3195652 | 43536.29481 |
| | 0.0000093 | 0.1456522 | 44526.33609 |
| | 0.0000009 | 0.1311594 | 45322.88979 |
| | 0.0015858 | 0.2615942 | 46419.54842 |
| | 0.0079070 | 0.2855072 | 47393.37799 |
| | 0.0057151 | 0.7152174 | 51260.79681 |
| | 0.0060373 | 0.7152174 | 51799.54682 |
| | 0.0272885 | 0.3340580 | 57741.52729 |
| | 0.0000179 | 0.1746377 | 58960.28998 |
| | 0.0000056 | 0.1456522 | 59524.3758 |
| | 0.0002033 | 0.2181159 | 60636.58339 |
| | 0.0002888 | 0.2181159 | 66625.27095 |
| | 0.0113934 | 0.6956522 | 75206.21809 |
| | 0.0000006 | 0.1166667 | 100624.8699 |
| | 0.0000011 | 0.1311594 | 117726.354 |
| | 0.0000003 | 0.1166667 | 133676.0475 |
| | 0.0097596 | 0.2905797 | 149351.6364 |

TABLE 20
Biomarkers identified by SELDI technology with a p-value smaller
or equal to 0.05 that can discriminate primary DF from primary
DHF infection. Grouped according to fraction it was detected in.
Prognostic

DHF1_DHF2 vs DF1_DF2

| | p value | roc | m/z averaqe |
|---|---|---|---|
| F1CSL | 0.0038311 | 0.2436975 | 4990.19603 |
| | 0.0014459 | 0.1904762 | 4020.472527 |
| | 0.0211895 | 0.2829132 | 6138.119764 |
| | 0.0031730 | 0.2436975 | 5266.529592 |
| | 0.0413134 | 0.6890756 | 3431.457425 |
| | 0.0443241 | 0.3081232 | 5183.584315 |
| | 0.0413134 | 0.3081232 | 2752.206092 |
| | 0.0142310 | 0.7282913 | 9107.540827 |
| | 0.0007753 | 0.1904762 | 38593.25709 |
| | 0.0266561 | 0.3025210 | 2683.722459 |
| | 0.0211895 | 0.3025210 | 3224.576562 |
| | 0.0072261 | 0.2633053 | 37462.00218 |
| | 0.0167265 | 0.2689076 | 35401.0858 |
| | 0.0384778 | 0.7086835 | 3448.844444 |
| | 0.0102048 | 0.7478992 | 3685.41489 |
| | 0.0111026 | 0.7282913 | 10287.01359 |
| | 0.0131111 | 0.2829132 | 2902.12753 |
| | 0.0004510 | 0.8263305 | 10092.33514 |
| F1CSH | 0.0039547 | 0.2425595 | 10527.69159 |
| | 0.0344018 | 0.3050595 | 10802.97632 |
| | 0.0017659 | 0.2008929 | 11324.62766 |
| | 0.0399741 | 0.3258929 | 11350.40235 |
| | 0.0024050 | 0.2217262 | 11451.56989 |
| | 0.0043569 | 0.2500000 | 11498.19671 |
| | 0.0014310 | 0.1800595 | 11528.91453 |
| | 0.0462940 | 0.3258929 | 12013.23362 |
| | 0.0214895 | 0.7351190 | 14022.97966 |
| | 0.0295071 | 0.6726190 | 14346.75421 |
| | 0.0252238 | 0.7142857 | 15094.27149 |
| | 0.0232917 | 0.7008929 | 40033.21376 |
| | 0.0430361 | 0.3467262 | 74862.91741 |
| | 0.0232917 | 0.7008929 | 79154.5458 |
| | 0.0318740 | 0.6875000 | 89630.65823 |
| | 0.0318740 | 0.2916667 | 125373.7131 |
| | 0.0252238 | 0.2842262 | 149368.5056 |
| F1ISL | 0.03038282 | 0.68055556 | 4432.616925 |
| | 0.01657493 | 0.73888889 | 4449.125935 |
| | 0.00937477 | 0.26111111 | 4994.293858 |
| | 0.01937332 | 0.28055556 | 5272.160739 |
| | 0.04040412 | 0.31944444 | 6455.01391 |
| | 0.03038282 | 0.3 | 7167.316325 |
| | 0.01657493 | 0.28472222 | 35469.71663 |
| F1ISH | 0.0292725 | 0.2583333 | 10129.14503 |
| | 0.0114023 | 0.2583333 | 10147.96592 |
| | 0.0114023 | 0.2500000 | 10777.41795 |
| | 0.0030935 | 0.1833333 | 10832.80337 |
| | 0.0141957 | 0.2333333 | 10965.73793 |
| | 0.0158068 | 0.2333333 | 11025.00949 |
| | 0.0216432 | 0.2833333 | 11057.71207 |
| | 0.0018457 | 0.8000000 | 12135.8102 |
| | 0.0064351 | 0.7750000 | 12231.72039 |
| | 0.0001164 | 0.9000000 | 12534.59195 |
| | 0.0030935 | 0.8250000 | 12582.46907 |
| | 0.0355579 | 0.7250000 | 13927.30735 |
| | 0.0039711 | 0.2083333 | 23104.0813 |
| | 0.0391105 | 0.2833333 | 23638.655 |
| | 0.0391105 | 0.7083333 | 79113.00591 |
| F5CSL | 0.0379534 | 0.3176329 | 2714.357253 |
| | 0.0116705 | 0.7342995 | 5165.803217 |
| | 0.0013507 | 0.7729469 | 8726.282926 |
| | 0.0009327 | 0.7729469 | 8846.247547 |
| | 0.0073716 | 0.7342995 | 9192.684623 |
| | 0.0100387 | 0.7342995 | 9320.83479 |
| | 0.0116705 | 0.7149758 | 9468.993468 |
| | 0.0156545 | 0.7536232 | 9679.241772 |
| | 0.0238679 | 0.7149758 | 15227.01519 |
| | 0.0062929 | 0.7536232 | 28203.24796 |
| | 0.0049399 | 0.7342995 | 28984.77362 |
| | 0.0049399 | 0.2596618 | 41655.94365 |
| | 0.0333455 | 0.3176329 | 43694.32147 |
| F5CSH | 0.0198099 | 0.6934524 | 10913.89329 |
| | 0.0017659 | 0.7976190 | 14029.40621 |
| | 0.0370989 | 0.6592262 | 14521.99131 |
| | 0.0063629 | 0.7633929 | 14996.39253 |
| | 0.0076496 | 0.7767857 | 15572.71556 |
| | 0.0091649 | 0.7559524 | 15774.3005 |
| | 0.0344018 | 0.7142857 | 17665.44985 |
| | 0.0009298 | 0.8184524 | 28370.51303 |
| | 0.0014310 | 0.8184524 | 29222.15188 |
| | 0.0119414 | 0.2633929 | 53767.53507 |
| | 0.0462940 | 0.6800595 | 84062.87518 |
| F5ISH | 0.04465415 | 0.65277778 | 10194.75241 |
| | 0.03714679 | 0.6712963 | 10255.15564 |
| | 0.04202078 | 0.68981481 | 10265.10592 |
| | 0.03489597 | 0.68981481 | 10275.98212 |
| | 0.03489597 | 0.68981481 | 10389.26482 |
| | 0.0157542 | 0.72685185 | 10413.75186 |
| | 0.0180921 | 0.72685185 | 10416.27302 |
| | 0.04742539 | 0.6712963 | 10419.51807 |
| | 0.030742 | 0.32638889 | 11124.07478 |
| | 0.02369357 | 0.27083333 | 11796.34403 |
| | 0.0180921 | 0.27777778 | 11926.05207 |
| | 0.030742 | 0.31481481 | 12771.26536 |
| | 0.003468 | 0.25925926 | 13423.48434 |
| | 0.02701997 | 0.31481481 | 13888.42124 |
| | 0.02369357 | 0.31481481 | 45360.95214 |
| | 0.030742 | 0.2962963 | 47280.55103 |
| F6CSL | 0.0046335 | 0.81089744 | 4178.147403 |
| | 0.00308916 | 0.25801282 | 4966.887753 |
| | 0.04503826 | 0.30288462 | 6005.550627 |
| | 0.04503826 | 0.34775641 | 6368.819184 |
| | 0.03864703 | 0.30288462 | 6405.544153 |
| | 0.0219851 | 0.28044872 | 6730.940725 |
| | 0.0259475 | 0.28044872 | 6777.027523 |
| | 0.03304353 | 0.30288462 | 6814.090876 |
| | 0.00996754 | 0.22115385 | 6852.503653 |
| | 0.00827638 | 0.25801282 | 6868.685109 |
| | 0.04503826 | 0.32532051 | 6892.31088 |
| | 0.03864703 | 0.28044872 | 6962.971613 |
| | 0.04503826 | 0.32532051 | 7002.421072 |
| | 0.04855452 | 0.32532051 | 7049.864742 |
| | 0.04855452 | 0.32532051 | 7065.348357 |
| | 0.01561114 | 0.30288462 | 7089.314476 |
| | 0.01561114 | 0.75160256 | 7636.624747 |
| | 0.02020929 | 0.72115385 | 7661.987216 |
| | 0.03051287 | 0.70673077 | 8296.864065 |
| | 0.00753117 | 0.72916667 | 15184.98539 |
| | 0.00996754 | 0.72916667 | 15365.82604 |
| | 0.02815052 | 0.28846154 | 23566.3546 |
| F6CSH | 0.0054498 | 0.2613636 | 10031.5232 |
| | 0.0054498 | 0.2414773 | 10128.78825 |
| | 0.0141298 | 0.2812500 | 10197.87061 |
| | 0.0071351 | 0.2613636 | 10244.13547 |
| | 0.0015587 | 0.2017045 | 10283.30231 |
| | 0.0413476 | 0.3210227 | 10327.90565 |
| | 0.0130094 | 0.3011364 | 10461.31639 |
| | 0.0110017 | 0.2613636 | 10503.04585 |
| | 0.0332771 | 0.2897727 | 10769.30644 |
| | 0.0009285 | 0.1676136 | 10851.39035 |
| | 0.0049737 | 0.2414773 | 10909.85409 |
| | 0.0025639 | 0.2073864 | 11049.12454 |
| | 0.0031109 | 0.2073864 | 11147.59114 |
| | 0.0265943 | 0.3011364 | 11185.14578 |
| | 0.0228130 | 0.2812500 | 11246.64624 |
| | 0.0153345 | 0.2954545 | 11750.95799 |
| | 0.0332771 | 0.3352273 | 11832.56962 |
| | 0.0476029 | 0.3551136 | 11908.94273 |
| | 0.0246409 | 0.2954545 | 11993.02686 |
| | 0.0476029 | 0.3409091 | 12057.41098 |
| | 0.0153345 | 0.2414773 | 12176.73044 |
| | 0.0059668 | 0.7528409 | 13612.48842 |
| | 0.0211040 | 0.7471591 | 13846.52517 |
| | 0.0413476 | 0.6875000 | 14825.26488 |
| | 0.0023248 | 0.7869318 | 15055.63774 |
| | 0.0025639 | 0.7528409 | 15411.25871 |
| | 0.0054498 | 0.7727273 | 15613.40893 |
| | 0.0085049 | 0.7528409 | 15812.1091 |
| | 0.0005418 | 0.1875000 | 23169.52525 |
| | 0.0211040 | 0.3011364 | 23418.55947 |
| | 0.0246409 | 0.3068182 | 23683.93102 |
| | 0.0332771 | 0.3153409 | 43596.91071 |
| | 0.0358029 | 0.3068182 | 51329.3608 |
| | 0.0085049 | 0.2272727 | 53487.79549 |
| | 0.0045355 | 0.2414773 | 57685.14725 |
| | 0.0010315 | 0.2073864 | 58847.42102 |
| | 0.0014076 | 0.2073864 | 59365.11459 |
| | 0.0007504 | 0.1676136 | 60097.13372 |
| | 0.0021063 | 0.2073864 | 61383.37478 |
| | 0.0130094 | 0.2812500 | 91892.51895 |
| | 0.0008350 | 0.1875000 | 117244.8486 |
| F6ISL | 0.0280624 | 0.3051471 | 2592.104434 |
| | 0.0198735 | 0.2855392 | 2649.485504 |
| | 0.0128643 | 0.2745098 | 2995.703282 |
| | 0.0342578 | 0.3137255 | 3030.319866 |
| | 0.0280624 | 0.2745098 | 3056.069215 |
| | 0.0390083 | 0.2745098 | 4019.571278 |
| | 0.0006409 | 0.8149510 | 7625.50723 |
| | 0.0280624 | 0.7058824 | 15922.78821 |
| | 0.0198735 | 0.7169118 | 16108.41436 |
| F6ISH | 0.0029637 | 0.2318841 | 14550.43665 |
| | 0.0137479 | 0.2724638 | 14599.05541 |
| | 0.0149363 | 0.7130435 | 15772.51523 |
| | 0.0149363 | 0.7333333 | 15807.50552 |
| | 0.0162143 | 0.7130435 | 15961.34377 |
| | 0.0327382 | 0.6927536 | 16106.21548 |
| | 0.0010750 | 0.1710145 | 23411.55 |
| | 0.0006275 | 0.1768116 | 23915.54212 |
| | 0.0106684 | 0.2782609 | 33558.37315 |
| | 0.0116189 | 0.2782609 | 51260.79681 |
| | 0.0260898 | 0.2724638 | 51799.54682 |
| | 0.0106684 | 0.2376812 | 52580.37193 |
| | 0.0470378 | 0.2927536 | 57741.52729 |
| | 0.0002258 | 0.1768116 | 58960.28998 |
| | 0.0000238 | 0.1159420 | 59524.3758 |
| | 0.0001100 | 0.1565217 | 60636.58339 |
| | 0.0190614 | 0.2724638 | 66625.27095 |
| | 0.0149363 | 0.2579710 | 79541.53333 |
| | 0.0019953 | 0.1768116 | 117726.354 |

TABLE 21
Most significant biomarkers identified by SELDI and BPS that can discriminate
between secondary DF and secondary DHF infection at different stages of the
disease. Grouped according to fraction it was found in.
Secondary Infection
p-value top 15% or < or = 0.05 AND
ROC value < or = 0.25 or > or = 0.75

| | | 2DHF1 vs | | 2DHF2 vs | | 2DHF3 vs | | |
|---|---|---|---|---|---|---|---|---|
| | | 2DF1 | | 2DF2 | | 2DF3 | | m/z |
| | Index | p value | roc | p value | roc | p value | roc | average |
| F1CSL | 5 | 0.00671 | 0.23469 | 0.00113 | 0.18000 | 0.59628 | 0.55385 | 5268.88284 |
| | 6 | 0.03867 | 0.26531 | 0.27169 | 0.36667 | 0.36904 | 0.57949 | 5513.34534 |
| | 7 | 0.01310 | 0.21429 | 0.00953 | 0.23333 | 0.42016 | 0.60000 | 5559.47729 |
| | 10 | 0.01688 | 0.23980 | 0.16468 | 0.36667 | 0.94491 | 0.47692 | 5675.03009 |
| | 11 | 0.01688 | 0.23469 | 0.07118 | 0.34000 | 0.69539 | 0.57949 | 5688.54253 |
| | 16 | 0.52005 | 0.56633 | 0.03620 | 0.68667 | 0.06882 | 0.68205 | 6887.48718 |
| | 22 | 0.31209 | 0.38776 | 0.91741 | 0.52667 | 0.02000 | 0.75385 | 7434.41892 |
| | 23 | 0.11824 | 0.64286 | 0.22110 | 0.63333 | 0.00461 | 0.17436 | 9314.81895 |
| | 25 | 0.38266 | 0.61224 | 0.54755 | 0.55333 | 0.00344 | 0.20000 | 9526.36673 |
| | 26 | 0.31209 | 0.63776 | 0.75574 | 0.55333 | 0.00532 | 0.20000 | 9550.87281 |
| | 32 | 0.64589 | 0.46429 | 0.04006 | 0.28667 | 0.56474 | 0.43077 | 55807.6598 |
| F1CSH | 10 | 0.38266 | 0.40816 | 0.10134 | 0.31333 | 0.00397 | 0.19762 | 13462.0865 |
| | 15 | 0.31209 | 0.38776 | 0.01074 | 0.23333 | 0.51269 | 0.43333 | 63122.4241 |
| F1ISL | 1 | 0.17765 | 0.66000 | 0.91741 | 0.50000 | 0.04881 | 0.74000 | 2565.21994 |
| | 2 | 0.00844 | 0.23333 | 0.69355 | 0.58000 | 0.52028 | 0.60667 | 2614.7931 |
| | 3 | 0.81955 | 0.55333 | 0.57551 | 0.58000 | 0.06493 | 0.71333 | 2656.55417 |
| | 4 | 0.20584 | 0.63333 | 0.01708 | 0.74000 | 0.49373 | 0.44667 | 2842.30118 |
| | 12 | 0.25402 | 0.60667 | 0.01910 | 0.71333 | 0.32969 | 0.39333 | 3485.24531 |
| | 19 | 0.02379 | 0.76667 | 0.06493 | 0.71333 | 0.14089 | 0.36667 | 9298.45491 |
| | 22 | 0.01209 | 0.76667 | 0.78746 | 0.47333 | 0.75574 | 0.47333 | 44956.201 |
| F1ISH | 1 | 0.10930 | 0.65385 | 0.27014 | 0.61735 | 0.79999 | 0.47692 | 10323.6689 |
| | 2 | 0.03275 | 0.74451 | 0.35812 | 0.61224 | 0.11200 | 0.68205 | 10911.3918 |
| | 3 | 1.00000 | 0.52473 | 0.04818 | 0.71429 | 0.53402 | 0.55385 | 10967.399 |
| | 5 | 0.92268 | 0.50549 | 0.02742 | 0.71429 | 0.24013 | 0.63077 | 11308.5233 |
| | 6 | 0.26438 | 0.38736 | 0.00671 | 0.81122 | 0.79999 | 0.48205 | 11468.1974 |
| | 9 | 0.80829 | 0.55220 | 0.29060 | 0.63776 | 0.00461 | 0.83077 | 13486.9672 |
| | 13 | 0.33179 | 0.59890 | 0.33459 | 0.61224 | 0.27901 | 0.58462 | 39896.2782 |
| | 15 | 0.05225 | 0.70879 | 0.29060 | 0.61224 | 0.53402 | 0.58462 | 43960.757 |
| | 14 | 0.01985 | 0.76374 | 0.35812 | 0.59184 | 0.79999 | 0.53333 | 42259.3628 |
| | 16 | 0.04664 | 0.70879 | 0.49069 | 0.56122 | 0.94491 | 0.53333 | 44577.8155 |
| | 21 | 0.92268 | 0.49725 | 0.01688 | 0.73980 | 0.39410 | 0.58462 | 88302.0484 |
| | 24 | 0.46668 | 0.57143 | 0.49069 | 0.56122 | 0.56474 | 0.54872 | 111262.512 |
| F5CSL | 1 | 0.78279 | 0.51531 | 0.57551 | 0.52667 | 0.09870 | 0.68182 | 5077.61334 |
| | 2 | 0.81830 | 0.46429 | 0.02944 | 0.68667 | 0.31064 | 0.40210 | 6658.89951 |
| | 4 | 0.52005 | 0.44388 | 0.00953 | 0.74000 | 0.33910 | 0.37413 | 6688.97411 |
| | 5 | 0.64589 | 0.43878 | 0.17765 | 0.63333 | 0.02981 | 0.76573 | 6826.12668 |
| | 9 | 0.71319 | 0.54082 | 0.22110 | 0.63333 | 0.01380 | 0.20629 | 8845.3908 |
| | 10 | 0.43474 | 0.61224 | 0.06493 | 0.66000 | 0.01380 | 0.17832 | 8964.59038 |
| | 11 | 0.46224 | 0.58673 | 0.07793 | 0.71333 | 0.00839 | 0.17832 | 8982.52189 |
| | 13 | 0.55029 | 0.56122 | 0.17765 | 0.66000 | 0.00496 | 0.17832 | 9316.3678 |
| | 14 | 0.71319 | 0.56122 | 0.37251 | 0.58000 | 0.00706 | 0.20629 | 9469.87316 |
| | 17 | 0.06608 | 0.70918 | 0.07793 | 0.71333 | 0.66391 | 0.43007 | 11760.7821 |
| | 21 | 0.00380 | 0.81122 | 0.88457 | 0.44667 | 0.66391 | 0.45804 | 25640.9662 |
| | 24 | 0.25068 | 0.60714 | 0.30953 | 0.58000 | 0.02981 | 0.23427 | 53647.346 |
| | 25 | 0.64589 | 0.53571 | 0.88457 | 0.52667 | 0.05227 | 0.26224 | 67060.0819 |
| | 26 | 0.96335 | 0.46429 | 0.11029 | 0.66000 | 0.00285 | 0.15035 | 75331.1654 |
| F5CSH | 4 | 0.16586 | 0.63889 | 0.52815 | 0.52473 | 0.00526 | 0.82231 | 12378.5059 |
| | 6 | 0.03266 | 0.75000 | 0.22507 | 0.59890 | 0.62237 | 0.53306 | 12606.1744 |
| F6CSL | 1 | 0.51269 | 0.58810 | 0.04024 | 0.31429 | 0.96519 | 0.49524 | 4198.06271 |
| | 2 | 0.11614 | 0.33333 | 0.07355 | 0.29048 | 0.79343 | 0.49524 | 5162.57462 |
| | 3 | 0.03247 | 0.70714 | 0.51269 | 0.41190 | 0.31547 | 0.61190 | 6952.81298 |
| | 4 | 0.04469 | 0.72857 | 0.07355 | 0.66905 | 0.51269 | 0.56429 | 11719.8723 |
| | 5 | 0.01638 | 0.75238 | 0.60047 | 0.55238 | 0.04469 | 0.70476 | 11917.5926 |
| | 6 | 0.04469 | 0.73095 | 0.07355 | 0.71667 | 0.08874 | 0.66190 | 12913.932 |
| | 7 | 0.13784 | 0.63810 | 0.03618 | 0.71429 | 0.22170 | 0.61190 | 46601.9329 |
| | 8 | 0.31547 | 0.61190 | 0.45812 | 0.57619 | 0.79343 | 0.47143 | 54417.1306 |
| F6CSH | 2 | 0.82726 | 0.51905 | 0.80829 | 0.51648 | 0.23865 | 0.37857 | 11783.4664 |
| | 4 | 0.02324 | 0.72857 | 0.14545 | 0.66209 | 0.69447 | 0.44524 | 12436.087 |
| | 5 | 0.03618 | 0.72857 | 0.10930 | 0.63462 | 0.96519 | 0.47143 | 12569.9265 |
| | 8 | 0.03618 | 0.28571 | 0.12046 | 0.28571 | 0.75998 | 0.56429 | 32267.4992 |
| | 10 | 0.31547 | 0.63571 | 0.26438 | 0.66209 | 0.57047 | 0.42857 | 39867.5323 |
| | 12 | 0.79343 | 0.54524 | 0.01985 | 0.76374 | 0.17607 | 0.61429 | 46697.2707 |
| | 14 | 0.00048 | 0.11905 | 0.88425 | 0.46978 | 0.93044 | 0.49524 | 150108.44 |
| F6ISH | 3 | 0.00040 | 0.86224 | 0.00394 | 0.79333 | 0.72442 | 0.47333 | 13533.1707 |
| | 4 | 0.00282 | 0.81122 | 0.17765 | 0.66000 | 0.54755 | 0.55333 | 13816.9437 |
| | 13 | 0.00770 | 0.21429 | 0.98345 | 0.52667 | 0.60413 | 0.47333 | 49749.6447 |

*No data for fractions F5ISL, F5ISH and F6ISL

TABLE 22
Biomarkers detected by SELDI technology with a p-value smaller or equal to 0.05 that can
discriminate secondary DENV infection from OFI. Grouped according to fraction it was found in.

| | Ct1_2 vs 2DF1_2 | | | | Ct1_2 vs 2DHF1_2 | | |
|---|---|---|---|---|---|---|---|
| | p-value | roc value | m/z average | | p-value | roc value | m/z average |
| F1CSL | 0.0000000 | 0.0151515 | 2625.181487 | F1CSL | 0.0000000 | 0.0454545 | 2625.181487 |
| | 0.0000000 | 0.0303030 | 2667.845783 | | 0.0000000 | 0.0454545 | 2667.845783 |
| | 0.0000000 | 0.0151515 | 2741.725854 | | 0.0000000 | 0.0303030 | 2741.725854 |
| | 0.0000000 | 0.0606061 | 2856.727191 | | 0.0000001 | 0.0757576 | 2856.727191 |
| | 0.0000000 | 0.0151515 | 2872.154934 | | 0.0000000 | 0.0303030 | 2872.154934 |
| | 0.0001261 | 0.1969697 | 2897.527715 | | 0.0001169 | 0.1969697 | 2897.527715 |
| | 0.0000000 | 0.0454545 | 2921.681011 | | 0.0000004 | 0.0909091 | 2921.681011 |
| | 0.0000334 | 0.1666667 | 2938.078868 | | 0.0000461 | 0.1818182 | 2938.078868 |
| | 0.0000000 | 0.0454545 | 2990.831829 | | 0.0000000 | 0.0303030 | 2990.831829 |
| | 0.0000000 | 0.0303030 | 3043.041845 | | 0.0000002 | 0.0757576 | 3043.041845 |
| | 0.0000001 | 0.0606061 | 3071.048466 | | 0.0000008 | 0.1303030 | 3071.048466 |
| | 0.0000000 | 0.0303030 | 3145.264181 | | 0.0000004 | 0.1060606 | 3145.264181 |
| | 0.0000000 | 0.0151515 | 3175.340726 | | 0.0000000 | 0.0757576 | 3175.340726 |
| | 0.0000000 | 0.0303030 | 3209.081505 | | 0.0000000 | 0.0757576 | 3209.081505 |
| | 0.0000000 | 0.0151515 | 3262.112346 | | 0.0000000 | 0.0606061 | 3262.112346 |
| | 0.0000000 | 0.0000000 | 3280.760876 | | 0.0000000 | 0.0151515 | 3280.760876 |
| | 0.0000000 | 0.0606061 | 3307.659966 | | 0.0000001 | 0.0757576 | 3307.659966 |
| | 0.0000000 | 0.0303030 | 3358.521584 | | 0.0000052 | 0.1303030 | 3358.521584 |
| | 0.0000005 | 0.0848485 | 3420.037403 | | 0.0002280 | 0.1848485 | 3420.037403 |
| | 0.0000000 | 0.0151515 | 3437.42944 | | 0.0000009 | 0.1363636 | 3437.42944 |
| | 0.0000001 | 0.0757576 | 3511.538012 | | 0.0000012 | 0.1212121 | 3511.538012 |
| | 0.0000000 | 0.0151515 | 3589.060244 | | 0.0000000 | 0.0454545 | 3589.060244 |
| | 0.0000001 | 0.0757576 | 3631.061336 | | 0.0001084 | 0.2000000 | 3631.061336 |
| | 0.0000000 | 0.0151515 | 3680.115892 | | 0.0000000 | 0.0303030 | 3680.115892 |
| | 0.0001970 | 0.1969697 | 3799.639473 | | 0.0250180 | 0.2969697 | 3799.639473 |
| | 0.0000000 | 0.0151515 | 3814.303638 | | 0.0000000 | 0.0757576 | 3814.303638 |
| | 0.0000003 | 0.0909091 | 3863.107595 | | 0.0018605 | 0.2363636 | 3863.107595 |
| | 0.0000040 | 0.1363636 | 3884.602302 | | 0.0000461 | 0.1606061 | 3884.602302 |
| | 0.0064753 | 0.2818182 | 3923.916704 | | 0.0000008 | 0.1060606 | 3949.327603 |
| | 0.0000000 | 0.0606061 | 3949.327603 | | 0.0000023 | 0.1363636 | 3965.762902 |
| | 0.0000001 | 0.0606061 | 3965.762902 | | 0.0000000 | 0.0303030 | 4063.241745 |
| | 0.0000000 | 0.0151515 | 4063.241745 | | 0.0051615 | 0.2757576 | 4103.551305 |
| | 0.0000683 | 0.1848485 | 4103.551305 | | 0.0117705 | 0.2818182 | 4128.656395 |
| | 0.0001169 | 0.2060606 | 4128.656395 | | 0.0003042 | 0.2272727 | 4143.916106 |
| | 0.0000052 | 0.1363636 | 4143.916106 | | 0.0000001 | 0.0757576 | 4182.768879 |
| | 0.0000000 | 0.0454545 | 4182.768879 | | 0.0000016 | 0.1212121 | 4279.033423 |
| | 0.0000000 | 0.0454545 | 4279.033423 | | 0.0000003 | 0.0909091 | 4299.724384 |
| | 0.0000000 | 0.0606061 | 4299.724384 | | 0.0001578 | 0.1818182 | 4468.900331 |
| | 0.0454632 | 0.3515152 | 4450.645147 | | 0.0250180 | 0.3272727 | 4492.301874 |
| | 0.0000114 | 0.1666667 | 4468.900331 | | 0.0000009 | 0.1060606 | 4524.063737 |
| | 0.0007491 | 0.2515152 | 4492.301874 | | 0.0000062 | 0.8666667 | 4573.192933 |
| | 0.0000000 | 0.0303030 | 4524.063737 | | 0.0000019 | 0.8818182 | 4588.379469 |
| | 0.0001084 | 0.8060606 | 4573.192933 | | 0.0117705 | 0.3121212 | 4646.271132 |
| | 0.0000334 | 0.8363636 | 4588.379469 | | 0.0003266 | 0.7909091 | 4972.46769 |
| | 0.0017471 | 0.2363636 | 4646.271132 | | 0.0000008 | 0.8969697 | 4985.052133 |
| | 0.0160481 | 0.7000000 | 4972.46769 | | 0.0000062 | 0.8666667 | 4995.082985 |
| | 0.0001700 | 0.8212121 | 4985.052133 | | 0.0000930 | 0.8060606 | 5487.254589 |
| | 0.0007491 | 0.7606061 | 4995.082985 | | 0.0000000 | 0.9727273 | 5559.119903 |
| | 0.0006544 | 0.7606061 | 5487.254589 | | 0.0000000 | 0.9727273 | 5574.710692 |
| | 0.0000000 | 0.9575758 | 5559.119903 | | 0.0000003 | 0.8969697 | 5590.063516 |
| | 0.0000000 | 0.9424242 | 5574.710692 | | 0.0000000 | 0.9878788 | 5675.016336 |
| | 0.0000048 | 0.8727273 | 5590.063516 | | 0.0000000 | 0.9878788 | 5689.765089 |
| | 0.0000000 | 0.9727273 | 5675.016336 | | 0.0474992 | 0.6454545 | 5767.809439 |
| | 0.0000000 | 0.9727273 | 5689.765089 | | 0.0001700 | 0.8030303 | 5912.163434 |
| | 0.0000584 | 0.8181818 | 5912.163434 | | 0.0004642 | 0.7606061 | 5996.460394 |
| | 0.0000135 | 0.8424242 | 5996.460394 | | 0.0057849 | 0.7303030 | 6013.779325 |
| | 0.0025358 | 0.7151515 | 6013.779325 | | 0.0000030 | 0.8666667 | 6127.503716 |
| | 0.0000004 | 0.8969697 | 6127.503716 | | 0.0000000 | 0.9424242 | 6143.838117 |
| | 0.0000000 | 0.9575758 | 6143.838117 | | 0.0038600 | 0.7454545 | 6454.996063 |
| | 0.0000088 | 0.8363636 | 6454.996063 | | 0.0000028 | 0.8666667 | 6487.509564 |
| | 0.0000000 | 0.9424242 | 6487.509564 | | 0.0000004 | 0.9121212 | 6591.475901 |
| | 0.0000000 | 0.9575758 | 6591.475901 | | 0.0454632 | 0.6454545 | 6642.439305 |
| | 0.0005710 | 0.7606061 | 6642.439305 | | 0.0117705 | 0.6848485 | 6652.107044 |
| | 0.0000204 | 0.8212121 | 6652.107044 | | 0.0000241 | 0.8363636 | 6683.82645 |
| | 0.0000002 | 0.8969697 | 6683.82645 | | 0.0000930 | 0.8060606 | 6805.698908 |
| | 0.0000002 | 0.9121212 | 6805.698908 | | 0.0007003 | 0.7757576 | 6860.453871 |
| | 0.0000011 | 0.8969697 | 6860.453871 | | 0.0000000 | 0.9575758 | 6944.325104 |
| | 0.0000000 | 0.9878788 | 6944.325104 | | 0.0002831 | 0.7757576 | 7160.085957 |
| | 0.0000683 | 0.8212121 | 7160.085957 | | 0.0001084 | 0.8060606 | 7331.96616 |
| | 0.0000096 | 0.8363636 | 7331.96616 | | 0.0111656 | 0.7151515 | 7350.027102 |
| | 0.0160481 | 0.7000000 | 7350.027102 | | 0.0000135 | 0.8515152 | 7415.948421 |
| | 0.0000114 | 0.8666667 | 7415.948421 | | 0.0011899 | 0.7454545 | 7589.702883 |
| | 0.0006544 | 0.7757576 | 7589.702883 | | 0.0380394 | 0.6606061 | 7661.734977 |
| | 0.0000006 | 0.1212121 | 8150.546744 | | 0.0000007 | 0.1060606 | 8150.546744 |
| | 0.0036386 | 0.2727273 | 8314.622635 | | 0.0137632 | 0.3121212 | 8314.622635 |
| | 0.0000135 | 0.1666667 | 8356.79503 | | 0.0000683 | 0.1969697 | 8356.79503 |
| | 0.0000005 | 0.1060606 | 8459.519265 | | 0.0000036 | 0.1363636 | 8459.519265 |
| | 0.0004330 | 0.2121212 | 8625.552866 | | 0.0168806 | 0.2969697 | 8625.552866 |
| | 0.0015391 | 0.2363636 | 8954.752999 | | 0.0013541 | 0.2363636 | 8954.752999 |
| | 0.0022425 | 0.2454545 | 9170.910437 | | 0.0015391 | 0.2303030 | 9170.910437 |
| | 0.0000004 | 0.9121212 | 10101.46975 | | 0.0000146 | 0.8515152 | 10101.46975 |
| | 0.0012696 | 0.7666667 | 10289.18681 | | 0.0022425 | 0.7363636 | 10289.18681 |
| | 0.0000135 | 0.8363636 | 11135.15931 | | 0.0000425 | 0.8060606 | 11135.15931 |
| | 0.0000159 | 0.8515152 | 11491.70855 | | 0.0000540 | 0.8060606 | 11491.70855 |
| | 0.0000284 | 0.8363636 | 11677.83181 | | 0.0004037 | 0.7969697 | 11677.83181 |
| | 0.0000008 | 0.8818182 | 11743.17361 | | 0.0000044 | 0.8666667 | 11743.17361 |
| | 0.0000002 | 0.9272727 | 11902.07203 | | 0.0000001 | 0.9272727 | 11902.07203 |
| | 0.0000006 | 0.9121212 | 12083.60453 | | 0.0000001 | 0.9121212 | 12083.60453 |
| | 0.0000632 | 0.8363636 | 12590.76335 | | 0.0032301 | 0.7303030 | 12590.76335 |
| | 0.0028638 | 0.7606061 | 12896.80544 | | 0.0009152 | 0.7606061 | 13115.34777 |
| | 0.0000021 | 0.8818182 | 13115.34777 | | 0.0000000 | 0.9727273 | 13375.41938 |
| | 0.0000000 | 0.9727273 | 13375.41938 | | 0.0000003 | 0.9272727 | 14757.7248 |
| | 0.0000096 | 0.8666667 | 14757.7248 | | 0.0000006 | 0.9121212 | 15198.38517 |
| | 0.0000002 | 0.9272727 | 15198.38517 | | 0.0000334 | 0.8060606 | 15906.99196 |
| | 0.0000146 | 0.8515152 | 15906.99196 | | 0.0006114 | 0.7757576 | 16080.83999 |
| | 0.0001578 | 0.7909091 | 16080.83999 | | 0.0000000 | 0.9424242 | 22937.26034 |
| | 0.0000000 | 0.9424242 | 22937.26034 | | 0.0000000 | 0.9424242 | 23544.76704 |
| | 0.0000000 | 0.9575758 | 23544.76704 | | 0.0000011 | 0.8969697 | 25497.91473 |
| | 0.0000013 | 0.8818182 | 25497.91473 | | 0.0000461 | 0.1606061 | 35405.0346 |
| | 0.0000104 | 0.1454545 | 35405.0346 | | 0.0000081 | 0.8666667 | 37705.72097 |
| | 0.0000284 | 0.8212121 | 37705.72097 | | 0.0000001 | 0.9121212 | 44810.68881 |
| | 0.0000000 | 0.9575758 | 44810.68881 | | 0.0000003 | 0.9121212 | 45184.10294 |
| | 0.0000000 | 0.9575758 | 45184.10294 | | 0.0000006 | 0.8969697 | 45616.26741 |
| | 0.0000000 | 0.9575758 | 45616.26741 | | 0.0000001 | 0.9272727 | 46357.92645 |
| | 0.0000000 | 0.9575758 | 46357.92645 | F1CSH | 0.0005411 | 0.7533333 | 10025.87122 |
| F1CSH | 0.0194944 | 0.6866667 | 10025.87122 | | 0.0000004 | 0.8866667 | 10130.28134 |
| | 0.0000061 | 0.8333333 | 10130.28134 | | 0.0000002 | 0.8866667 | 10149.11798 |
| | 0.0000037 | 0.8466667 | 10149.11798 | | 0.0000006 | 0.8866667 | 10171.11128 |
| | 0.0000113 | 0.8066667 | 10171.11128 | | 0.0000057 | 0.8466667 | 10196.44234 |
| | 0.0000743 | 0.7933333 | 10196.44234 | | 0.0000070 | 0.8466667 | 10215.38716 |
| | 0.0001286 | 0.7933333 | 10215.38716 | | 0.0000022 | 0.8600000 | 10224.52891 |
| | 0.0000286 | 0.8200000 | 10224.52891 | | 0.0000003 | 0.8733333 | 10241.85866 |
| | 0.0000046 | 0.8333333 | 10241.85866 | | 0.0000001 | 0.8733333 | 10297.3244 |
| | 0.0000019 | 0.8466667 | 10297.3244 | | 0.0000002 | 0.8866667 | 10351.51024 |
| | 0.0000011 | 0.8466667 | 10351.51024 | | 0.0000001 | 0.8733333 | 10369.2482 |
| | 0.0000009 | 0.8600000 | 10369.2482 | | 0.0000001 | 0.9000000 | 10413.34138 |
| | 0.0000019 | 0.8466667 | 10413.34138 | | 0.0000001 | 0.8866667 | 10444.68912 |
| | 0.0000040 | 0.8466667 | 10444.68912 | | 0.0000001 | 0.9000000 | 10461.79685 |
| | 0.0000026 | 0.8466667 | 10461.79685 | | 0.0000002 | 0.8733333 | 10481.71491 |
| | 0.0000037 | 0.8333333 | 10481.71491 | | 0.0000001 | 0.9000000 | 10490.47668 |
| | 0.0000005 | 0.8466667 | 10490.47668 | | 0.0000001 | 0.9000000 | 10496.60495 |
| | 0.0000018 | 0.8600000 | 10496.60495 | | 0.0000001 | 0.8866667 | 10507.40716 |
| | 0.0000007 | 0.8600000 | 10507.40716 | | 0.0000001 | 0.9000000 | 10514.61735 |
| | 0.0000008 | 0.8600000 | 10514.61735 | | 0.0000004 | 0.1133333 | 10755.48072 |
| | 0.0000001 | 0.1266667 | 10755.48072 | | 0.0000017 | 0.1533333 | 10783.37546 |
| | 0.0000005 | 0.1533333 | 10783.37546 | | 0.0000006 | 0.1266667 | 10808.95097 |
| | 0.0000000 | 0.1000000 | 10808.95097 | | 0.0000001 | 0.1133333 | 10835.54836 |
| | 0.0000000 | 0.0733333 | 10835.54836 | | 0.0000000 | 0.0733333 | 10882.15141 |
| | 0.0000000 | 0.0466667 | 10882.15141 | | 0.0000000 | 0.0466667 | 10908.0378 |
| | 0.0000000 | 0.0333333 | 10908.0378 | | 0.0000000 | 0.0333333 | 10926.28045 |
| | 0.0000000 | 0.0200000 | 10926.28045 | | 0.0000000 | 0.0200000 | 10951.30804 |
| | 0.0000000 | 0.0200000 | 10951.30804 | | 0.0000000 | 0.0866667 | 11040.90474 |
| | 0.0000000 | 0.0333333 | 11040.90474 | | 0.0000000 | 0.0466667 | 11078.55501 |
| | 0.0000000 | 0.0333333 | 11078.55501 | | 0.0000000 | 0.0600000 | 11154.55919 |
| | 0.0000000 | 0.0466667 | 11154.55919 | | 0.0000000 | 0.0733333 | 11197.83206 |
| | 0.0000000 | 0.0466667 | 11197.83206 | | 0.0129995 | 0.6733333 | 11324.57488 |
| | 0.0357827 | 0.6866667 | 11383.78669 | | 0.0059613 | 0.7000000 | 11383.78669 |
| | 0.0000698 | 0.8066667 | 11420.34027 | | 0.0001365 | 0.7800000 | 11420.34027 |
| | 0.0006373 | 0.7400000 | 11450.04285 | | 0.0003465 | 0.7400000 | 11450.04285 |
| | 0.0371053 | 0.3400000 | 11526.18986 | | 0.0153233 | 0.3133333 | 11679.13472 |
| | 0.0071288 | 0.3000000 | 11679.13472 | | 0.0000326 | 0.2066667 | 11725.21956 |
| | 0.0000049 | 0.1800000 | 11725.21956 | | 0.0000138 | 0.1933333 | 11790.8581 |
| | 0.0000043 | 0.1533333 | 11790.8581 | | 0.0000024 | 0.1533333 | 11823.52687 |
| | 0.0000021 | 0.1400000 | 11823.52687 | | 0.0000002 | 0.1133333 | 11853.93279 |
| | 0.0000001 | 0.1000000 | 11853.93279 | | 0.0000000 | 0.0200000 | 12009.62084 |
| | 0.0000000 | 0.0333333 | 12009.62084 | | 0.0000000 | 0.0333333 | 12080.71023 |
| | 0.0000000 | 0.0200000 | 12080.71023 | | 0.0000000 | 0.0466667 | 12112.68049 |
| | 0.0000000 | 0.0200000 | 12112.68049 | | 0.0000000 | 0.0600000 | 12132.64107 |
| | 0.0000000 | 0.0466667 | 12132.64107 | | 0.0000000 | 0.0866667 | 12163.0594 |
| | 0.0000000 | 0.0466667 | 12163.0594 | | 0.0000000 | 0.0600000 | 12268.93847 |
| | 0.0000000 | 0.0466667 | 12268.93847 | | 0.0000000 | 0.0333333 | 12358.50727 |
| | 0.0000000 | 0.0333333 | 12358.50727 | | 0.0000000 | 0.0466667 | 12493.48295 |
| | 0.0000000 | 0.0466667 | 12493.48295 | | 0.0000003 | 0.1266667 | 12562.71997 |
| | 0.0000001 | 0.1133333 | 12562.71997 | | 0.0074511 | 0.6866667 | 12954.81113 |
| | 0.0088747 | 0.6866667 | 12954.81113 | | 0.0345005 | 0.6733333 | 13063.59409 |
| | 0.0014059 | 0.7266667 | 13086.15535 | | 0.0005411 | 0.7666667 | 13086.15535 |
| | 0.0018115 | 0.2866667 | 13289.07095 | | 0.0059613 | 0.3000000 | 13289.07095 |
| | 0.0001449 | 0.2333333 | 13468.91732 | | 0.0020019 | 0.2600000 | 13468.91732 |
| | 0.0000001 | 0.1000000 | 13655.28043 | | 0.0000002 | 0.1133333 | 13655.28043 |
| | 0.0194944 | 0.3400000 | 13733.51807 | | 0.0047454 | 0.2733333 | 13733.51807 |
| | 0.0000000 | 0.0866667 | 13839.31124 | | 0.0000000 | 0.0733333 | 13839.31124 |
| | 0.0000000 | 0.0466667 | 13930.38141 | | 0.0000000 | 0.0200000 | 13930.38141 |
| | 0.0000003 | 0.1266667 | 14140.38344 | | 0.0000000 | 0.0866667 | 14140.38344 |
| | 0.0000286 | 0.7933333 | 14449.85539 | | 0.0000113 | 0.8200000 | 14449.85539 |
| | 0.0049689 | 0.7000000 | 14639.78045 | | 0.0056977 | 0.7000000 | 14639.78045 |
| | 0.0475786 | 0.6466667 | 14934.23152 | | 0.0219295 | 0.6600000 | 14934.23152 |
| | 0.0000000 | 0.0866667 | 15200.55252 | | 0.0000000 | 0.0866667 | 15200.55252 |
| | 0.0001009 | 0.1933333 | 15603.49216 | | 0.0014059 | 0.2733333 | 15603.49216 |
| | 0.0000046 | 0.8333333 | 16111.96967 | | 0.0000840 | 0.8066667 | 16111.96967 |
| | 0.0000268 | 0.8200000 | 16791.53773 | | 0.0028223 | 0.7266667 | 16791.53773 |
| | 0.0000268 | 0.8066667 | 16986.38906 | | 0.0035851 | 0.7133333 | 16986.38906 |
| | 0.0022106 | 0.2866667 | 18269.77418 | | 0.0001286 | 0.2200000 | 18269.77418 |
| | 0.0428195 | 0.6466667 | 23397.5494 | | 0.0021039 | 0.7400000 | 23397.5494 |
| | 0.0002459 | 0.2200000 | 28456.83502 | | 0.0159586 | 0.3266667 | 28456.83502 |
| | 0.0002759 | 0.2333333 | 29189.74355 | | 0.0088747 | 0.3133333 | 33656.05696 |
| | 0.0371053 | 0.3666667 | 33656.05696 | | 0.0000510 | 0.2066667 | 34696.86044 |
| | 0.0000021 | 0.1533333 | 34696.86044 | | 0.0000479 | 0.2200000 | 35998.90348 |
| | 0.0000061 | 0.1800000 | 35998.90348 | | 0.0013356 | 0.2466667 | 50105.04286 |
| | 0.0018115 | 0.7133333 | 38857.74158 | | 0.0000007 | 0.1400000 | 51139.33489 |
| | 0.0023222 | 0.7000000 | 39959.10698 | | 0.0000008 | 0.1666667 | 53826.57027 |
| | 0.0000024 | 0.8333333 | 44257.94633 | | 0.0005715 | 0.2466667 | 57323.39913 |
| | 0.0000790 | 0.7933333 | 44763.72492 | | 0.0000098 | 0.1666667 | 62969.82218 |
| | 0.0000698 | 0.1933333 | 50105.04286 | | 0.0000950 | 0.1933333 | 63794.78358 |
| | 0.0000001 | 0.1000000 | 51139.33489 | | 0.0026888 | 0.3133333 | 68276.74582 |
| | 0.0000001 | 0.1000000 | 53826.57027 | | 0.0012685 | 0.2600000 | 68908.94541 |
| | 0.0021039 | 0.2866667 | 57323.39913 | | 0.0071288 | 0.3133333 | 74844.60315 |
| | 0.0000000 | 0.0866667 | 62969.82218 | | 0.0000004 | 0.1133333 | 100409.516 |
| | 0.0000011 | 0.1400000 | 63794.78358 | | 0.0000004 | 0.1266667 | 101137.8334 |
| | 0.0297570 | 0.3533333 | 68276.74582 | | 0.0000005 | 0.1266667 | 102198.3507 |
| | 0.0014796 | 0.2733333 | 68908.94541 | | 0.0000000 | 0.0866667 | 107068.7099 |
| | 0.0000543 | 0.2333333 | 74844.60315 | | 0.0000001 | 0.1000000 | 113333.4714 |
| | 0.0000001 | 0.1133333 | 100409.516 | | 0.0009273 | 0.2866667 | 118685.9279 |
| | 0.0000001 | 0.1133333 | 101137.8334 | | 0.0000235 | 0.2200000 | 125659.3312 |
| | 0.0000001 | 0.1000000 | 102198.3507 | | 0.0332578 | 0.3533333 | 149568.5425 |
| | 0.0000000 | 0.0866667 | 107068.7099 | | 0.0001365 | 0.2200000 | 166914.0871 |
| | 0.0000002 | 0.1133333 | 113333.4714 | | 0.0000053 | 0.1666667 | 174369.2245 |
| | 0.0003093 | 0.2466667 | 118685.9279 | | 0.0000000 | 0.0200000 | 192541.7062 |
| | 0.0000129 | 0.1933333 | 125659.3312 | F1ISL | 0.0000000 | 0.9690476 | 2503.154582 |
| | 0.0003465 | 0.2600000 | 149568.5425 | | 0.0000000 | 0.9559524 | 2513.073032 |
| | 0.0006036 | 0.2600000 | 166914.0871 | | 0.0000001 | 0.9035714 | 2517.656451 |
| | 0.0000011 | 0.1533333 | 174369.2245 | | 0.0002404 | 0.7857143 | 2521.649116 |
| | 0.0000000 | 0.0733333 | 192541.7062 | | 0.0000001 | 0.9035714 | 2522.859861 |
| F1ISL | 0.0000000 | 0.9821429 | 2503.154582 | | 0.0000000 | 0.9297619 | 2523.669165 |
| | 0.0000000 | 0.9297619 | 2513.073032 | | 0.0000000 | 0.9428571 | 2524.520651 |
| | 0.0000000 | 0.9166667 | 2517.656451 | | 0.0000012 | 0.8511905 | 2525.251223 |
| | 0.0000000 | 0.9166667 | 2521.649116 | | 0.0000006 | 0.8904762 | 2526.594055 |
| | 0.0000002 | 0.9035714 | 2522.859861 | | 0.0000940 | 0.8250000 | 2527.751996 |
| | 0.0000051 | 0.8511905 | 2523.669165 | | 0.0004370 | 0.7857143 | 2534.62918 |
| | 0.0000030 | 0.8511905 | 2524.520651 | | 0.0003250 | 0.7595238 | 2540.500802 |
| | 0.0000092 | 0.8380952 | 2525.251223 | | 0.0000000 | 0.9166667 | 2549.242272 |
| | 0.0000048 | 0.8511905 | 2526.594055 | | 0.0000048 | 0.8380952 | 2556.57727 |
| | 0.0000152 | 0.8119048 | 2527.751996 | | 0.0000000 | 0.9297619 | 2565.111611 |
| | 0.0071030 | 0.7333333 | 2530.612478 | | 0.0000000 | 0.9166667 | 2570.087202 |
| | 0.0000016 | 0.8380952 | 2534.62918 | | 0.0000003 | 0.9035714 | 2578.662526 |
| | 0.0000163 | 0.8119048 | 2540.500802 | | 0.0000007 | 0.8773810 | 2590.690565 |
| | 0.0000086 | 0.8380952 | 2549.242272 | | 0.0000000 | 0.9297619 | 2595.854473 |
| | 0.0000304 | 0.7988095 | 2556.57727 | | 0.0000001 | 0.9035714 | 2601.511801 |
| | 0.0000000 | 0.9559524 | 2565.111611 | | 0.0010261 | 0.7726190 | 2603.929365 |
| | 0.0000000 | 0.9166667 | 2570.087202 | | 0.0000265 | 0.8119048 | 2605.06365 |
| | 0.0000086 | 0.8380952 | 2578.662526 | | 0.0000009 | 0.8642857 | 2608.030336 |
| | 0.0000002 | 0.8904762 | 2590.690565 | | 0.0000881 | 0.7988095 | 2611.242105 |
| | 0.0000000 | 0.9297619 | 2595.854473 | | 0.0000000 | 0.9690476 | 2614.032211 |
| | 0.0000000 | 0.9166667 | 2601.511801 | | 0.0000000 | 0.9428571 | 2617.583867 |
| | 0.0044053 | 0.7202381 | 2603.929365 | | 0.0000000 | 0.9166667 | 2621.696749 |
| | 0.0212810 | 0.6678571 | 2608.030336 | | 0.0000123 | 0.8380952 | 2629.00519 |
| | 0.0001138 | 0.8119048 | 2611.242105 | | 0.0000000 | 0.9166667 | 2631.998607 |
| | 0.0000001 | 0.9035714 | 2614.032211 | | 0.0000000 | 0.9297619 | 2634.20847 |
| | 0.0000000 | 0.9428571 | 2617.583867 | | 0.0000000 | 0.9559524 | 2635.700585 |
| | 0.0000003 | 0.8904762 | 2621.696749 | | 0.0000000 | 0.9559524 | 2637.169955 |
| | 0.0002883 | 0.7857143 | 2629.00519 | | 0.0000000 | 0.9559524 | 2637.618569 |
| | 0.0021736 | 0.7202381 | 2631.998607 | | 0.0000000 | 0.9428571 | 2638.060903 |
| | 0.0000021 | 0.8380952 | 2634.20847 | | 0.0000000 | 0.9559524 | 2638.54484 |
| | 0.0000001 | 0.9035714 | 2635.700585 | | 0.0000000 | 0.9690476 | 2639.173428 |
| | 0.0000000 | 0.9690476 | 2637.169955 | | 0.0000000 | 0.9821429 | 2639.795493 |
| | 0.0000000 | 0.9559524 | 2637.618569 | | 0.0000000 | 0.9821429 | 2640.467576 |
| | 0.0000000 | 0.9559524 | 2638.060903 | | 0.0000000 | 0.9821429 | 2641.164374 |
| | 0.0000000 | 0.9559524 | 2638.54484 | | 0.0000000 | 0.9821429 | 2641.926515 |
| | 0.0000000 | 0.9559524 | 2639.173428 | | 0.0000000 | 0.9821429 | 2642.885878 |
| | 0.0000001 | 0.9035714 | 2639.795493 | | 0.0000000 | 0.9297619 | 2643.949918 |
| | 0.0000003 | 0.9035714 | 2640.467576 | | 0.0000000 | 0.9428571 | 2646.227988 |
| | 0.0000000 | 0.9428571 | 2641.164374 | | 0.0000099 | 0.8380952 | 2648.600691 |
| | 0.0000000 | 0.9821429 | 2641.926515 | | 0.0000106 | 0.8250000 | 2657.390501 |
| | 0.0000000 | 0.9821429 | 2642.885878 | | 0.0000060 | 0.8380952 | 2658.351659 |
| | 0.0000000 | 0.9559524 | 2643.949918 | | 0.0000022 | 0.8642857 | 2658.967508 |
| | 0.0000000 | 0.9428571 | 2646.227988 | | 0.0000001 | 0.9035714 | 2659.814331 |
| | 0.0002404 | 0.7595238 | 2648.600691 | | 0.0000000 | 0.9297619 | 2661.194232 |
| | 0.0000636 | 0.7988095 | 2657.390501 | | 0.0000000 | 0.9559524 | 2661.84737 |
| | 0.0000114 | 0.8380952 | 2658.351659 | | 0.0000000 | 0.9428571 | 2662.858635 |
| | 0.0000349 | 0.8250000 | 2658.967508 | | 0.0000000 | 0.9166667 | 2666.570765 |
| | 0.0000002 | 0.9035714 | 2659.814331 | | 0.0000940 | 0.7988095 | 2674.971433 |
| | 0.0000000 | 0.9297619 | 2661.194232 | | 0.0000006 | 0.8773810 | 2677.075716 |
| | 0.0000000 | 0.9559524 | 2661.84737 | | 0.0000000 | 0.9297619 | 2679.146726 |
| | 0.0000000 | 0.9821429 | 2662.858635 | | 0.0000000 | 0.9821429 | 2681.308781 |
| | 0.0000000 | 0.9166667 | 2666.570765 | | 0.0000000 | 0.9428571 | 2684.692433 |
| | 0.0000000 | 0.9166667 | 2674.971433 | | 0.0000000 | 0.9428571 | 2686.45453 |
| | 0.0000033 | 0.8511905 | 2677.075716 | | 0.0000000 | 0.9559524 | 2704.270371 |
| | 0.0000000 | 0.9297619 | 2679.146726 | | 0.0000000 | 0.9428571 | 2709.71622 |
| | 0.0000000 | 0.9821429 | 2681.308781 | | 0.0001661 | 0.7857143 | 2722.447549 |
| | 0.0000000 | 0.9821429 | 2684.692433 | | 0.0000010 | 0.8773810 | 2724.372029 |
| | 0.0000000 | 0.9821429 | 2686.45453 | | 0.0000000 | 0.9166667 | 2726.112884 |
| | 0.0000000 | 0.9821429 | 2704.270371 | | 0.0000010 | 0.8642857 | 2727.211009 |
| | 0.0000000 | 0.9821429 | 2709.71622 | | 0.0000000 | 0.9559524 | 2738.579429 |
| | 0.0031115 | 0.7202381 | 2721.081778 | | 0.0000000 | 0.9821429 | 2749.363653 |
| | 0.0000048 | 0.8511905 | 2722.447549 | | 0.0000000 | 0.9428571 | 2752.680443 |
| | 0.0000006 | 0.8642857 | 2724.372029 | | 0.0000001 | 0.9035714 | 2753.648132 |
| | 0.0000001 | 0.8904762 | 2726.112884 | | 0.0000002 | 0.9035714 | 2753.857675 |
| | 0.0000326 | 0.8250000 | 2727.211009 | | 0.0000002 | 0.9035714 | 2753.946851 |
| | 0.0000000 | 0.9166667 | 2738.579429 | | 0.0000000 | 0.9559524 | 2755.192724 |
| | 0.0000000 | 0.9428571 | 2749.363653 | | 0.0017620 | 0.7428571 | 2767.933706 |
| | 0.0000000 | 0.9821429 | 2752.680443 | | 0.0001377 | 0.7642857 | 2770.080973 |
| | 0.0000000 | 0.9821429 | 2753.648132 | | 0.0000679 | 0.7988095 | 2771.748664 |
| | 0.0000000 | 0.9821429 | 2753.857675 | | 0.0000000 | 0.9166667 | 2772.763638 |
| | 0.0000000 | 0.9821429 | 2753.946851 | | 0.0000010 | 0.8904762 | 2775.410216 |
| | 0.0000000 | 0.9690476 | 2755.192724 | | 0.0000000 | 0.9690476 | 2788.581536 |
| | 0.0048556 | 0.7166667 | 2767.933706 | | 0.0000000 | 0.9690476 | 2791.185756 |
| | 0.0000349 | 0.8166667 | 2770.080973 | | 0.0000000 | 0.9559524 | 2796.034541 |
| | 0.0000012 | 0.8642857 | 2771.748664 | | 0.0000000 | 0.9559524 | 2803.236487 |
| | 0.0000000 | 0.9297619 | 2772.763638 | | 0.0000000 | 0.9690476 | 2811.436125 |
| | 0.0000000 | 0.9428571 | 2775.410216 | | 0.0000000 | 0.9559524 | 2817.188037 |
| | 0.0000000 | 0.9690476 | 2788.581536 | | 0.0000000 | 0.9821429 | 2819.017882 |
| | 0.0000000 | 0.9297619 | 2791.185756 | | 0.0000008 | 0.8904762 | 2820.608441 |
| | 0.0000000 | 0.9428571 | 2796.034541 | | 0.0000000 | 0.9297619 | 2824.989455 |
| | 0.0000000 | 0.9297619 | 2803.236487 | | 0.0005205 | 0.7690476 | 2840.429828 |
| | 0.0000000 | 0.9821429 | 2811.436125 | | 0.0000000 | 0.9821429 | 2859.505809 |
| | 0.0000000 | 0.9428571 | 2817.188037 | | 0.0000000 | 0.9821429 | 2866.751136 |
| | 0.0000064 | 0.8642857 | 2819.017882 | | 0.0000000 | 0.9297619 | 2879.703192 |
| | 0.0000005 | 0.8773810 | 2820.608441 | | 0.0000000 | 0.9821429 | 2883.890105 |
| | 0.0000000 | 0.9428571 | 2824.989455 | | 0.0000000 | 0.9428571 | 2887.453423 |
| | 0.0000488 | 0.8166667 | 2840.429828 | | 0.0000000 | 0.9166667 | 2894.062647 |
| | 0.0000000 | 0.9559524 | 2859.505809 | | 0.0000000 | 0.9297619 | 2904.031336 |
| | 0.0000000 | 0.9690476 | 2866.751136 | | 0.0000000 | 0.9428571 | 2906.91955 |
| | 0.0000000 | 0.9821429 | 2879.703192 | | 0.0000000 | 0.9428571 | 2908.899563 |
| | 0.0000000 | 0.9821429 | 2883.890105 | | 0.0000003 | 0.9035714 | 2910.510035 |
| | 0.0000000 | 0.9821429 | 2887.453423 | | 0.0000000 | 0.9428571 | 2911.83993 |
| | 0.0000000 | 0.9559524 | 2894.062647 | | 0.0000000 | 0.9297619 | 2915.763212 |
| | 0.0000000 | 0.9428571 | 2904.031336 | | 0.0000000 | 0.9166667 | 2927.060302 |
| | 0.0000000 | 0.9690476 | 2906.91955 | | 0.0000001 | 0.9035714 | 2930.543416 |
| | 0.0000000 | 0.9428571 | 2908.899563 | | 0.0000000 | 0.9821429 | 2933.649099 |
| | 0.0000000 | 0.9690476 | 2910.510035 | | 0.0000002 | 0.8904762 | 2938.720507 |
| | 0.0000000 | 0.9690476 | 2911.83993 | | 0.0000000 | 0.9690476 | 2948.917414 |
| | 0.0000000 | 0.9166667 | 2915.763212 | | 0.0000000 | 0.9297619 | 2951.608271 |
| | 0.0000000 | 0.9297619 | 2927.060302 | | 0.0000001 | 0.9083333 | 2953.403838 |
| | 0.0000000 | 0.9559524 | 2930.543416 | | 0.0000099 | 0.8297619 | 2956.963771 |
| | 0.0000000 | 0.9690476 | 2933.649099 | | 0.0000000 | 0.9035714 | 2976.912269 |
| | 0.0000002 | 0.9035714 | 2938.720507 | | 0.0000000 | 0.9559524 | 2993.494244 |
| | 0.0000000 | 0.9428571 | 2948.917414 | | 0.0000000 | 0.9559524 | 2997.743006 |
| | 0.0000000 | 0.9297619 | 2951.608271 | | 0.0000022 | 0.8511905 | 3000.298656 |
| | 0.0000000 | 0.9476190 | 2953.403838 | | 0.0000826 | 0.7988095 | 3001.47028 |
| | 0.0000000 | 0.9214286 | 2956.963771 | | 0.0002555 | 0.7857143 | 3002.078041 |
| | 0.0000000 | 0.9428571 | 2976.912269 | | 0.0001138 | 0.7988095 | 3004.640515 |
| | 0.0000000 | 0.9559524 | 2993.494244 | | 0.0000123 | 0.8250000 | 3015.428516 |
| | 0.0000000 | 0.9428571 | 2997.743006 | | 0.0000044 | 0.8380952 | 3020.653111 |
| | 0.0000636 | 0.7988095 | 3000.298656 | | 0.0330237 | 0.6547619 | 3026.595384 |
| | 0.0000106 | 0.8380952 | 3001.47028 | | 0.0006550 | 0.7380952 | 3035.982566 |
| | 0.0000163 | 0.8119048 | 3002.078041 | | 0.0025382 | 0.7202381 | 3039.961971 |
| | 0.0053472 | 0.7071429 | 3004.640515 | | 0.0000006 | 0.8642857 | 3042.639369 |
| | 0.0000000 | 0.9297619 | 3015.428516 | | 0.0000152 | 0.8380952 | 3048.708995 |
| | 0.0000001 | 0.9035714 | 3020.653111 | | 0.0000005 | 0.8642857 | 3066.155713 |
| | 0.0077943 | 0.7071429 | 3026.595384 | | 0.0000001 | 0.9166667 | 3083.362119 |
| | 0.0000557 | 0.7988095 | 3035.982566 | | 0.0000000 | 0.9559524 | 3092.389989 |
| | 0.0028115 | 0.7333333 | 3039.961971 | | 0.0000000 | 0.9297619 | 3101.60504 |
| | 0.0000001 | 0.9035714 | 3042.639369 | | 0.0000002 | 0.8773810 | 3113.412549 |
| | 0.0000004 | 0.8904762 | 3048.708995 | | 0.0000001 | 0.8904762 | 3129.459825 |
| | 0.0000000 | 0.9166667 | 3066.155713 | | 0.0000028 | 0.8380952 | 3135.980164 |
| | 0.0000000 | 0.9297619 | 3083.362119 | | 0.0117085 | 0.6809524 | 3140.915312 |
| | 0.0000000 | 0.9559524 | 3092.389989 | | 0.0000002 | 0.9035714 | 3149.522222 |
| | 0.0000033 | 0.8380952 | 3101.60504 | | 0.0000000 | 0.9559524 | 3158.254319 |
| | 0.0000000 | 0.9428571 | 3113.412549 | | 0.0000456 | 0.8119048 | 3163.025153 |
| | 0.0000002 | 0.8904762 | 3129.459825 | | 0.0000000 | 0.9035714 | 3166.387243 |
| | 0.0000010 | 0.8642857 | 3135.980164 | | 0.0006933 | 0.7464286 | 3169.365587 |
| | 0.0000373 | 0.8119048 | 3140.915312 | | 0.0000003 | 0.8904762 | 3172.450888 |
| | 0.0000000 | 0.9035714 | 3149.522222 | | 0.0000003 | 0.8904762 | 3177.822062 |
| | 0.0000000 | 0.9559524 | 3158.254319 | | 0.0000006 | 0.8773810 | 3183.425475 |
| | 0.0016709 | 0.7464286 | 3163.025153 | | 0.0000001 | 0.9035714 | 3197.777988 |
| | 0.0001002 | 0.7988095 | 3166.387243 | | 0.0000002 | 0.8904762 | 3213.068347 |
| | 0.0034403 | 0.7071429 | 3169.365587 | | 0.0000000 | 0.9035714 | 3221.413524 |
| | 0.0000000 | 0.9297619 | 3172.450888 | | 0.0000092 | 0.8166667 | 3235.296597 |
| | 0.0000000 | 0.9821429 | 3177.822062 | | 0.0000216 | 0.8119048 | 3245.638764 |
| | 0.0000035 | 0.8511905 | 3183.425475 | | 0.0038005 | 0.7035714 | 3276.379562 |
| | 0.0000051 | 0.8250000 | 3197.777988 | | 0.0000064 | 0.8380952 | 3285.606735 |
| | 0.0000002 | 0.9035714 | 3213.068347 | | 0.0000055 | 0.8380952 | 3290.515874 |
| | 0.0000000 | 0.9559524 | 3221.413524 | | 0.0002262 | 0.7726190 | 3300.370909 |
| | 0.0002714 | 0.7773810 | 3235.296597 | | 0.0000041 | 0.8642857 | 3314.079782 |
| | 0.0000326 | 0.7988095 | 3245.638764 | | 0.0000006 | 0.8642857 | 3315.115414 |
| | 0.0000069 | 0.8380952 | 3285.606735 | | 0.0000004 | 0.8904762 | 3315.909084 |
| | 0.0000051 | 0.8380952 | 3290.515874 | | 0.0000003 | 0.8773810 | 3316.53443 |
| | 0.0000001 | 0.9166667 | 3314.079782 | | 0.0000000 | 0.9035714 | 3318.297755 |
| | 0.0000064 | 0.8380952 | 3315.115414 | | 0.0012773 | 0.7464286 | 3321.260411 |
| | 0.0000826 | 0.8119048 | 3315.909084 | | 0.0000152 | 0.8250000 | 3324.2993 |
| | 0.0000024 | 0.8773810 | 3316.53443 | | 0.0000064 | 0.8380952 | 3336.16938 |
| | 0.0000000 | 0.9166667 | 3318.297755 | | 0.0003450 | 0.7595238 | 3343.441682 |
| | 0.0009184 | 0.7464286 | 3321.260411 | | 0.0000086 | 0.8380952 | 3353.246902 |
| | 0.0001561 | 0.8119048 | 3322.335337 | | 0.0000000 | 0.9166667 | 3369.373189 |
| | 0.0000175 | 0.8250000 | 3324.2993 | | 0.0000005 | 0.8642857 | 3377.906174 |
| | 0.0000142 | 0.8250000 | 3336.16938 | | 0.0000000 | 0.9428571 | 3397.259967 |
| | 0.0000349 | 0.7857143 | 3343.441682 | | 0.0000000 | 0.9297619 | 3402.125169 |
| | 0.0001377 | 0.7857143 | 3349.175124 | | 0.0000002 | 0.9035714 | 3417.509394 |
| | 0.0000051 | 0.8511905 | 3353.246902 | | 0.0000000 | 0.9559524 | 3430.642457 |
| | 0.0000003 | 0.8642857 | 3369.373189 | | 0.0000000 | 0.9821429 | 3445.720567 |
| | 0.0000001 | 0.9035714 | 3377.906174 | | 0.0000488 | 0.8119048 | 3456.301995 |
| | 0.0000000 | 0.9690476 | 3397.259967 | | 0.0000000 | 0.9690476 | 3468.46665 |
| | 0.0000000 | 0.9297619 | 3402.125169 | | 0.0000051 | 0.8380952 | 3478.572514 |
| | 0.0000000 | 0.9166667 | 3417.509394 | | 0.0000000 | 0.9428571 | 3488.857635 |
| | 0.0000000 | 0.9297619 | 3430.642457 | | 0.0000015 | 0.8642857 | 3499.370274 |
| | 0.0000000 | 0.9428571 | 3445.720567 | | 0.0000000 | 0.9821429 | 3515.357908 |
| | 0.0000000 | 0.9297619 | 3456.301995 | | 0.0000006 | 0.8904762 | 3528.345658 |
| | 0.0000000 | 0.9821429 | 3468.46665 | | 0.0000000 | 0.9166667 | 3536.763944 |
| | 0.0000000 | 0.9166667 | 3478.572514 | | 0.0000304 | 0.7988095 | 3573.694129 |
| | 0.0000000 | 0.9559524 | 3488.857635 | | 0.0000399 | 0.8214286 | 3588.453952 |
| | 0.0000326 | 0.7988095 | 3499.370274 | | 0.0000002 | 0.8773810 | 3604.347348 |
| | 0.0000000 | 0.9690476 | 3515.357908 | | 0.0000001 | 0.8773810 | 3612.0972 |
| | 0.0000000 | 0.9428571 | 3528.345658 | | 0.0005841 | 0.7595238 | 3623.497822 |
| | 0.0000000 | 0.9297619 | 3536.763944 | | 0.0000000 | 0.9166667 | 3644.486854 |
| | 0.0000069 | 0.8380952 | 3573.694129 | | 0.0000231 | 0.8380952 | 3670.116515 |
| | 0.0001002 | 0.7904762 | 3588.453952 | | 0.0000007 | 0.8511905 | 3678.462544 |
| | 0.0000022 | 0.8642857 | 3604.347348 | | 0.0000008 | 0.8642857 | 3687.780782 |
| | 0.0000002 | 0.8773810 | 3612.0972 | | 0.0000030 | 0.8642857 | 3699.672565 |
| | 0.0000051 | 0.8250000 | 3623.497822 | | 0.0000048 | 0.8380952 | 3739.751205 |
| | 0.0000000 | 0.9428571 | 3644.486854 | | 0.0000010 | 0.8511905 | 3772.107649 |
| | 0.0000026 | 0.8380952 | 3670.116515 | | 0.0000000 | 0.9166667 | 3790.742865 |
| | 0.0000060 | 0.8511905 | 3678.462544 | | 0.0000000 | 0.9297619 | 3807.332552 |
| | 0.0000000 | 0.9166667 | 3687.780782 | | 0.0000679 | 0.7988095 | 3817.046282 |
| | 0.0000001 | 0.8904762 | 3699.672565 | | 0.0000030 | 0.8511905 | 3827.712827 |
| | 0.0000010 | 0.8773810 | 3739.751205 | | 0.0000010 | 0.8773810 | 3891.091076 |
| | 0.0000000 | 0.9297619 | 3772.107649 | | 0.0000163 | 0.7988095 | 3922.074093 |
| | 0.0000000 | 0.9035714 | 3790.742865 | | 0.0000123 | 0.8380952 | 3929.390604 |
| | 0.0000001 | 0.9035714 | 3807.332552 | | 0.0481333 | 0.6547619 | 3955.346561 |
| | 0.0000201 | 0.8250000 | 3817.046282 | | 0.0015842 | 0.7380952 | 3972.21957 |
| | 0.0000000 | 0.9428571 | 3827.712827 | | 0.0282327 | 0.6642857 | 3983.675826 |
| | 0.0000000 | 0.9035714 | 3891.091076 | | 0.0032721 | 0.7297619 | 4005.668752 |
| | 0.0001561 | 0.7595238 | 3922.074093 | | 0.0025382 | 0.7166667 | 4029.284592 |
| | 0.0000016 | 0.8511905 | 3929.390604 | | 0.0028115 | 0.7333333 | 4110.556156 |
| | 0.0370590 | 0.6726190 | 3972.21957 | | 0.0000725 | 0.7904762 | 4119.607136 |
| | 0.0127855 | 0.6821429 | 3983.675826 | | 0.0000004 | 0.8773810 | 4127.77626 |
| | 0.0007338 | 0.7428571 | 4005.668752 | | 0.0000019 | 0.8511905 | 4137.751828 |
| | 0.0384942 | 0.6511905 | 4029.284592 | | 0.0000011 | 0.8511905 | 4144.116439 |
| | 0.0000099 | 0.8511905 | 4110.556156 | | 0.0004911 | 0.7726190 | 4182.996313 |
| | 0.0002714 | 0.7726190 | 4119.607136 | | 0.0000349 | 0.7857143 | 4191.218296 |
| | 0.0003661 | 0.7857143 | 4127.77626 | | 0.0000004 | 0.8904762 | 4203.02242 |
| | 0.0000005 | 0.8511905 | 4137.751828 | | 0.0067785 | 0.2761905 | 4275.126841 |
| | 0.0000175 | 0.8250000 | 4144.116439 | | 0.0016709 | 0.7511905 | 4332.585207 |
| | 0.0000725 | 0.8250000 | 4182.996313 | | 0.0003250 | 0.7726190 | 4392.121326 |
| | 0.0000022 | 0.8511905 | 4191.218296 | | 0.0000001 | 0.9166667 | 4418.467916 |
| | 0.0000595 | 0.7726190 | 4203.02242 | | 0.0000163 | 0.7988095 | 4434.401871 |
| | 0.0038005 | 0.2761905 | 4275.126841 | | 0.0001292 | 0.7857143 | 4451.561047 |
| | 0.0067785 | 0.6940476 | 4332.585207 | | 0.0017620 | 0.7595238 | 4472.538662 |
| | 0.0006550 | 0.7642857 | 4392.121326 | | 0.0000265 | 0.8250000 | 4491.879558 |
| | 0.0000679 | 0.7988095 | 4418.467916 | | 0.0010842 | 0.7464286 | 4644.394801 |
| | 0.0000060 | 0.8380952 | 4434.401871 | | 0.0012097 | 0.7333333 | 4761.564855 |
| | 0.0006550 | 0.7726190 | 4451.561047 | | 0.0041946 | 0.7119048 | 4820.900844 |
| | 0.0117085 | 0.6809524 | 4472.538662 | | 0.0187914 | 0.6857143 | 4919.007415 |
| | 0.0006186 | 0.7464286 | 4491.879558 | | 0.0010842 | 0.7202381 | 4994.422542 |
| | 0.0000265 | 0.7988095 | 4644.394801 | | 0.0004633 | 0.2238095 | 5765.752659 |
| | 0.0006550 | 0.7642857 | 4761.564855 | | 0.0010842 | 0.2500000 | 7167.554217 |
| | 0.0028115 | 0.7333333 | 4820.900844 | | 0.0056095 | 0.2892857 | 7665.827257 |
| | 0.0026717 | 0.7119048 | 4919.007415 | | 0.0000163 | 0.1845238 | 8149.336429 |
| | 0.0071030 | 0.6940476 | 4994.422542 | | 0.0102437 | 0.3285714 | 8313.570899 |
| | 0.0003061 | 0.2238095 | 5765.752659 | | 0.0015842 | 0.2500000 | 8472.644418 |
| | 0.0002404 | 0.2107143 | 7167.554217 | | 0.0039932 | 0.2761905 | 8598.621326 |
| | 0.0003061 | 0.2369048 | 7665.827257 | | 0.0000373 | 0.1845238 | 8953.916453 |
| | 0.0036163 | 0.2892857 | 7781.136159 | | 0.0000060 | 0.1845238 | 9309.466303 |
| | 0.0000026 | 0.1583333 | 8149.336429 | | 0.0384942 | 0.6416667 | 15170.38885 |
| | 0.0077943 | 0.3023810 | 8313.570899 | | 0.0003661 | 0.7857143 | 31172.33879 |
| | 0.0004370 | 0.2238095 | 8472.644418 | | 0.0399764 | 0.6464286 | 31844.2444 |
| | 0.0004633 | 0.2238095 | 8598.621326 | | 0.0293705 | 0.6416667 | 34078.02645 |
| | 0.0000123 | 0.1666667 | 8953.916453 | | 0.0133562 | 0.7071429 | 36236.07045 |
| | 0.0008686 | 0.2583333 | 9309.466303 | | 0.0152056 | 0.6726190 | 37339.38185 |
| | 0.0029580 | 0.7202381 | 15170.38885 | | 0.0093602 | 0.6940476 | 37681.0348 |
| | 0.0003450 | 0.7595238 | 31172.33879 | | 0.0012773 | 0.7511905 | 38035.46314 |
| | 0.0012773 | 0.7202381 | 36236.07045 | | 0.0021736 | 0.7380952 | 38449.75553 |
| | 0.0250443 | 0.6726190 | 37339.38185 | | 0.0038005 | 0.7119048 | 44944.74849 |
| | 0.0005514 | 0.7595238 | 37681.0348 | | 0.0221724 | 0.6809524 | 45471.54051 |
| | 0.0356696 | 0.6547619 | 38035.46314 | | 0.0293705 | 0.6678571 | 45847.89453 |
| | 0.0050960 | 0.7071429 | 38449.75553 | | 0.0009709 | 0.7726190 | 46550.98173 |
| | 0.0240531 | 0.6857143 | 39042.10283 | | 0.0003250 | 0.7726190 | 46955.84213 |
| | 0.0000248 | 0.8250000 | 44944.74849 | | 0.0000679 | 0.7857143 | 48592.66902 |
| | 0.0000326 | 0.7726190 | 45471.54051 | F1ISH | 0.0000000 | 0.9855967 | 10011.99352 |
| | 0.0003450 | 0.7595238 | 45847.89453 | | 0.0000000 | 0.9855967 | 10020.52117 |
| | 0.0000456 | 0.7988095 | 46550.98173 | | 0.0000000 | 0.9855967 | 10025.43594 |
| | 0.0003661 | 0.7857143 | 46955.84213 | | 0.0000000 | 0.9855967 | 10028.64467 |
| | 0.0000002 | 0.8904762 | 48592.66902 | | 0.0000000 | 0.9855967 | 10036.97216 |
| F1ISH | 0.0000000 | 0.9855967 | 10011.99352 | | 0.0000000 | 0.9855967 | 10051.63047 |
| | 0.0000000 | 0.9855967 | 10020.52117 | | 0.0000000 | 0.9855967 | 10061.98167 |
| | 0.0000000 | 0.9855967 | 10025.43594 | | 0.0000000 | 0.9855967 | 10072.44679 |
| | 0.0000000 | 0.9855967 | 10028.64467 | | 0.0000000 | 0.9855967 | 10081.58862 |
| | 0.0000000 | 0.9855967 | 10036.97216 | | 0.0000000 | 0.9855967 | 10090.2363 |
| | 0.0000000 | 0.9855967 | 10051.63047 | | 0.0000000 | 0.9855967 | 10098.45601 |
| | 0.0000000 | 0.9855967 | 10061.98167 | | 0.0000000 | 0.9855967 | 10105.92173 |
| | 0.0000000 | 0.9855967 | 10072.44679 | | 0.0000000 | 0.9855967 | 10114.78187 |
| | 0.0000000 | 0.9855967 | 10081.58862 | | 0.0000000 | 0.9855967 | 10135.6053 |
| | 0.0000000 | 0.9855967 | 10090.2363 | | 0.0000000 | 0.9855967 | 10147.36272 |
| | 0.0000000 | 0.9855967 | 10098.45601 | | 0.0000000 | 0.9855967 | 10162.00816 |
| | 0.0000000 | 0.9855967 | 10105.92173 | | 0.0000000 | 0.9718793 | 10175.58746 |
| | 0.0000000 | 0.9855967 | 10114.78187 | | 0.0000000 | 0.9581619 | 10187.91964 |
| | 0.0000000 | 0.9855967 | 10135.6053 | | 0.0000000 | 0.9307270 | 10198.46953 |
| | 0.0000000 | 0.9855967 | 10147.36272 | | 0.0000000 | 0.9581619 | 10208.17418 |
| | 0.0000000 | 0.9855967 | 10162.00816 | | 0.0000000 | 0.9444444 | 10217.54983 |
| | 0.0000000 | 0.9855967 | 10175.58746 | | 0.0000000 | 0.9170096 | 10230.57425 |
| | 0.0000000 | 0.9718793 | 10187.91964 | | 0.0000002 | 0.8895748 | 10239.94928 |
| | 0.0000000 | 0.9444444 | 10198.46953 | | 0.0000004 | 0.8895748 | 10247.57672 |
| | 0.0000000 | 0.9718793 | 10208.17418 | | 0.0000000 | 0.9307270 | 10262.82559 |
| | 0.0000000 | 0.9718793 | 10217.54983 | | 0.0000000 | 0.9444444 | 10294.56991 |
| | 0.0000000 | 0.9718793 | 10230.57425 | | 0.0000000 | 0.9581619 | 10306.64326 |
| | 0.0000000 | 0.9581619 | 10239.94928 | | 0.0000001 | 0.9170096 | 10324.32336 |
| | 0.0000000 | 0.9581619 | 10247.57672 | | 0.0000002 | 0.9170096 | 10346.11123 |
| | 0.0000000 | 0.9718793 | 10262.82559 | | 0.0000012 | 0.8758573 | 10363.09118 |
| | 0.0000000 | 0.9718793 | 10294.56991 | | 0.0000024 | 0.8758573 | 10383.28509 |
| | 0.0000000 | 0.9718793 | 10306.64326 | | 0.0013313 | 0.7386831 | 10402.18844 |
| | 0.0000000 | 0.9581619 | 10324.32336 | | 0.0054923 | 0.7112483 | 10408.85968 |
| | 0.0000000 | 0.9581619 | 10346.11123 | | 0.0017923 | 0.7386831 | 10414.59628 |
| | 0.0000000 | 0.9581619 | 10363.09118 | | 0.0033641 | 0.7112483 | 10425.2516 |
| | 0.0000000 | 0.9581619 | 10383.28509 | | 0.0150719 | 0.6975309 | 10434.47733 |
| | 0.0000000 | 0.9170096 | 10402.18844 | | 0.0199734 | 0.6700960 | 10456.2903 |
| | 0.0000017 | 0.8758573 | 10408.85968 | | 0.0387015 | 0.6426612 | 10472.20507 |
| | 0.0000019 | 0.8758573 | 10414.59628 | | 0.0041946 | 0.7249657 | 10488.20672 |
| | 0.0000099 | 0.8484225 | 10425.2516 | | 0.0006337 | 0.7524005 | 10500.0895 |
| | 0.0000957 | 0.7935528 | 10434.47733 | | 0.0000125 | 0.8347051 | 10509.73309 |
| | 0.0001801 | 0.7935528 | 10442.82927 | | 0.0000010 | 0.8895748 | 10521.88611 |
| | 0.0006337 | 0.7798354 | 10456.2903 | | 0.0000017 | 0.8758573 | 10546.67852 |
| | 0.0001929 | 0.7935528 | 10472.20507 | | 0.0262126 | 0.6700960 | 10562.0077 |
| | 0.0000234 | 0.8209877 | 10488.20672 | | 0.0476084 | 0.6426612 | 10567.23236 |
| | 0.0000107 | 0.8484225 | 10500.0895 | | 0.0158068 | 0.6838134 | 10574.35037 |
| | 0.0000066 | 0.8484225 | 10509.73309 | | 0.0173716 | 0.6783265 | 10877.21485 |
| | 0.0000024 | 0.8758573 | 10521.88611 | | 0.0190707 | 0.6783265 | 10890.43701 |
| | 0.0000044 | 0.8621399 | 10546.67852 | | 0.0438578 | 0.6604938 | 10909.77131 |
| | 0.0041946 | 0.7249657 | 10562.0077 | | 0.0209133 | 0.6604938 | 10928.38566 |
| | 0.0209133 | 0.6838134 | 10567.23236 | | 0.0037585 | 0.7249657 | 10944.27704 |
| | 0.0158068 | 0.6975309 | 10574.35037 | | 0.0020148 | 0.7524005 | 10962.30392 |
| | 0.0001681 | 0.7935528 | 10855.25459 | | 0.0000234 | 0.8484225 | 10978.46135 |
| | 0.0000429 | 0.8072702 | 10877.21485 | | 0.0001364 | 0.7976680 | 10994.43525 |
| | 0.0002533 | 0.7935528 | 10890.43701 | | 0.0075211 | 0.6879287 | 11014.94996 |
| | 0.0001463 | 0.7935528 | 10909.77131 | | 0.0009820 | 0.7386831 | 11035.719 |
| | 0.0005578 | 0.7798354 | 10928.38566 | | 0.0004307 | 0.7524005 | 11046.66769 |
| | 0.0000620 | 0.8072702 | 10944.27704 | | 0.0000829 | 0.7798354 | 11057.41916 |
| | 0.0000003 | 0.9032922 | 10962.30392 | | 0.0002066 | 0.8072702 | 11071.04109 |
| | 0.0000125 | 0.8484225 | 10978.46135 | | 0.0001104 | 0.8072702 | 11087.90879 |
| | 0.0000012 | 0.8621399 | 10994.43525 | | 0.0000077 | 0.8484225 | 11105.68287 |
| | 0.0000462 | 0.8072702 | 11014.94996 | | 0.0000497 | 0.7798354 | 11118.40053 |
| | 0.0000005 | 0.8895748 | 11035.719 | | 0.0009232 | 0.7524005 | 11123.52137 |
| | 0.0000006 | 0.8895748 | 11046.66769 | | 0.0001929 | 0.7839506 | 11130.42377 |
| | 0.0000005 | 0.9032922 | 11057.41916 | | 0.0041946 | 0.7386831 | 11147.3563 |
| | 0.0000002 | 0.8895748 | 11071.04109 | | 0.0001681 | 0.7702332 | 11163.91984 |
| | 0.0000002 | 0.8895748 | 11087.90879 | | 0.0011100 | 0.7290809 | 11178.66084 |
| | 0.0000009 | 0.8895748 | 11105.68287 | | 0.0229091 | 0.6646091 | 11191.97145 |
| | 0.0000003 | 0.8895748 | 11118.40053 | | 0.0001463 | 0.8072702 | 11212.36119 |
| | 0.0000136 | 0.8484225 | 11123.52137 | | 0.0025374 | 0.7386831 | 11226.91495 |
| | 0.0000077 | 0.8347051 | 11130.42377 | | 0.0274017 | 0.6742112 | 11244.4887 |
| | 0.0000077 | 0.8484225 | 11147.3563 | | 0.0000016 | 0.1104252 | 11488.09557 |
| | 0.0000029 | 0.8758573 | 11163.91984 | | 0.0000001 | 0.0829904 | 11508.64295 |
| | 0.0000066 | 0.8525377 | 11178.66084 | | 0.0000002 | 0.0967078 | 11533.00585 |
| | 0.0000342 | 0.8347051 | 11191.97145 | | 0.0000000 | 0.0281207 | 11553.43803 |
| | 0.0000022 | 0.8484225 | 11212.36119 | | 0.0000000 | 0.0555556 | 11576.23632 |
| | 0.0000019 | 0.8621399 | 11226.91495 | | 0.0000001 | 0.0692730 | 11596.70327 |
| | 0.0007659 | 0.7565158 | 11244.4887 | | 0.0000001 | 0.0967078 | 11607.15919 |
| | 0.0030077 | 0.2613169 | 11488.09557 | | 0.0000000 | 0.0692730 | 11623.79327 |
| | 0.0000077 | 0.1515775 | 11508.64295 | | 0.0000000 | 0.0555556 | 11642.6909 |
| | 0.0000001 | 0.0692730 | 11533.00585 | | 0.0000000 | 0.0281207 | 11662.86838 |
| | 0.0000000 | 0.0692730 | 11553.43803 | | 0.0000000 | 0.0555556 | 11682.71261 |
| | 0.0000003 | 0.1104252 | 11576.23632 | | 0.0000013 | 0.1241427 | 11706.67327 |
| | 0.0000000 | 0.0555556 | 11596.70327 | | 0.0000004 | 0.0967078 | 11737.66458 |
| | 0.0000000 | 0.0555556 | 11607.15919 | | 0.0000005 | 0.1104252 | 11758.16873 |
| | 0.0000000 | 0.0692730 | 11623.79327 | | 0.0000024 | 0.1378601 | 11778.74667 |
| | 0.0000000 | 0.0555556 | 11642.6909 | | 0.0000071 | 0.1652949 | 11785.26908 |
| | 0.0000000 | 0.0418381 | 11662.86838 | | 0.0000398 | 0.1790123 | 11789.43722 |
| | 0.0000000 | 0.0692730 | 11682.71261 | | 0.0003778 | 0.2475995 | 11794.63204 |
| | 0.0000007 | 0.1104252 | 11706.67327 | | 0.0000014 | 0.1241427 | 11810.11077 |
| | 0.0000002 | 0.0967078 | 11737.66458 | | 0.0000000 | 0.0692730 | 11849.00542 |
| | 0.0000001 | 0.0829904 | 11758.16873 | | 0.0000000 | 0.0692730 | 11881.99843 |
| | 0.0000071 | 0.1515775 | 11778.74667 | | 0.0000000 | 0.0418381 | 11909.00114 |
| | 0.0000159 | 0.1652949 | 11785.26908 | | 0.0000000 | 0.0555556 | 11918.54028 |
| | 0.0000620 | 0.2201646 | 11789.43722 | | 0.0000000 | 0.0692730 | 11925.28834 |
| | 0.0001801 | 0.2201646 | 11794.63204 | | 0.0000003 | 0.1104252 | 11930.35684 |
| | 0.0000234 | 0.1652949 | 11810.11077 | | 0.0000000 | 0.0692730 | 11946.65943 |
| | 0.0000013 | 0.1241427 | 11849.00542 | | 0.0000020 | 0.1241427 | 11977.13849 |
| | 0.0000001 | 0.0829904 | 11881.99843 | | 0.0000022 | 0.1241427 | 11992.80904 |
| | 0.0000001 | 0.0829904 | 11909.00114 | | 0.0000001 | 0.0967078 | 12009.23838 |
| | 0.0000002 | 0.0967078 | 11918.54028 | | 0.0000000 | 0.0555556 | 12043.45183 |
| | 0.0000001 | 0.0829904 | 11925.28834 | | 0.0000000 | 0.0144033 | 12063.7875 |
| | 0.0000002 | 0.0829904 | 11930.35684 | | 0.0000000 | 0.0555556 | 12080.17934 |
| | 0.0000006 | 0.1104252 | 11946.65943 | | 0.0000000 | 0.0692730 | 12095.12377 |
| | 0.0004307 | 0.2434842 | 11977.13849 | | 0.0000000 | 0.0692730 | 12101.64446 |
| | 0.0002533 | 0.2064472 | 11992.80904 | | 0.0000000 | 0.0555556 | 12119.25076 |
| | 0.0000020 | 0.1337449 | 12009.23838 | | 0.0000000 | 0.0418381 | 12141.1169 |
| | 0.0000000 | 0.0555556 | 12043.45183 | | 0.0000017 | 0.1241427 | 12160.76776 |
| | 0.0000000 | 0.0418381 | 12063.7875 | | 0.0035564 | 0.2750343 | 12181.97513 |
| | 0.0000000 | 0.0829904 | 12080.17934 | | 0.0476084 | 0.6467764 | 12343.70114 |
| | 0.0000000 | 0.0692730 | 12095.12377 | | 0.0312522 | 0.6604938 | 12359.32901 |
| | 0.0000001 | 0.0829904 | 12101.64446 | | 0.0030077 | 0.7427984 | 12404.11342 |
| | 0.0000000 | 0.0692730 | 12119.25076 | | 0.0030077 | 0.7427984 | 12428.61842 |
| | 0.0000029 | 0.1515775 | 12141.1169 | | 0.0229091 | 0.6838134 | 12526.96894 |
| | 0.0000040 | 0.1378601 | 12160.76776 | | 0.0000200 | 0.8209877 | 12586.1786 |
| | 0.0039711 | 0.2750343 | 12181.97513 | | 0.0000620 | 0.8072702 | 12616.77768 |
| | 0.0000429 | 0.8113855 | 12343.70114 | | 0.0158068 | 0.6783265 | 12657.85998 |
| | 0.0000829 | 0.7976680 | 12359.32901 | | 0.0000772 | 0.8072702 | 12725.27638 |
| | 0.0000342 | 0.8251029 | 12404.11342 | | 0.0061058 | 0.7153635 | 12755.4225 |
| | 0.0009232 | 0.7606310 | 12428.61842 | | 0.0015927 | 0.7290809 | 12792.9585 |
| | 0.0000620 | 0.8072702 | 12526.96894 | | 0.0083336 | 0.2887517 | 13272.14785 |
| | 0.0000034 | 0.8621399 | 12586.1786 | | 0.0002211 | 0.2475995 | 13425.04846 |
| | 0.0001364 | 0.7976680 | 12616.77768 | | 0.0000000 | 0.0281207 | 13517.3522 |
| | 0.0006752 | 0.7469136 | 12657.85998 | | 0.0000001 | 0.0829904 | 13572.89265 |
| | 0.0001463 | 0.7935528 | 12725.27638 | | 0.0000000 | 0.0555556 | 13664.61109 |
| | 0.0000234 | 0.8251029 | 12755.4225 | | 0.0000000 | 0.0555556 | 13743.15877 |
| | 0.0000462 | 0.8251029 | 12792.9585 | | 0.0006337 | 0.2338820 | 13857.21058 |
| | 0.0112625 | 0.3024691 | 13272.14785 | | 0.0000017 | 0.1337449 | 13933.3224 |
| | 0.0004307 | 0.2338820 | 13425.04846 | | 0.0000147 | 0.8484225 | 14144.36601 |
| | 0.0000000 | 0.0555556 | 13517.3522 | | 0.0000099 | 0.8484225 | 14330.61107 |
| | 0.0000001 | 0.0967078 | 13572.89265 | | 0.0000000 | 0.0281207 | 14868.45486 |
| | 0.0001185 | 0.2064472 | 13664.61109 | | 0.0000000 | 0.0418381 | 15277.38043 |
| | 0.0000000 | 0.0555556 | 13743.15877 | | 0.0000002 | 0.0967078 | 15477.52932 |
| | 0.0003537 | 0.2338820 | 13857.21058 | | 0.0002897 | 0.2338820 | 15627.65375 |
| | 0.0000020 | 0.1378601 | 13933.3224 | | 0.0218914 | 0.6700960 | 15817.19277 |
| | 0.0000037 | 0.8621399 | 14144.36601 | | 0.0000084 | 0.8347051 | 16525.3951 |
| | 0.0000185 | 0.8429355 | 14330.61107 | | 0.0031814 | 0.7112483 | 16735.85641 |
| | 0.0000000 | 0.0418381 | 14868.45486 | | 0.0165730 | 0.3079561 | 23638.15252 |
| | 0.0000000 | 0.0555556 | 15277.38043 | | 0.0209133 | 0.3299040 | 42740.48185 |
| | 0.0326348 | 0.3299040 | 15397.6027 | | 0.0000084 | 0.1515775 | 50962.01399 |
| | 0.0000000 | 0.0829904 | 15477.52932 | | 0.0000136 | 0.1652949 | 51505.99647 |
| | 0.0000317 | 0.1927298 | 15627.65375 | | 0.0000159 | 0.1748971 | 52094.18838 |
| | 0.0057917 | 0.7112483 | 15817.19277 | | 0.0000429 | 0.1844993 | 68311.65124 |
| | 0.0000147 | 0.8347051 | 16525.3951 | | 0.0021352 | 0.2530864 | 69531.81016 |
| | 0.0001028 | 0.7798354 | 16735.85641 | | 0.0007192 | 0.2393690 | 70239.10741 |
| | 0.0173716 | 0.3257888 | 23638.15252 | | 0.0001185 | 0.1886145 | 71096.60784 |
| | 0.0000014 | 0.1337449 | 50962.01399 | | 0.0000099 | 0.1611797 | 75269.22784 |
| | 0.0000006 | 0.1104252 | 51505.99647 | | 0.0021352 | 0.2572016 | 102406.355 |
| | 0.0000014 | 0.1241427 | 52094.18838 | | 0.0000536 | 0.2297668 | 124396.8269 |
| | 0.0000047 | 0.1652949 | 68311.65124 | | 0.0000667 | 0.2064472 | 135047.7781 |
| | 0.0003310 | 0.2201646 | 69531.81016 | | 0.0000185 | 0.1790123 | 150365.8563 |
| | 0.0010442 | 0.2572016 | 70239.10741 | F5CSL | 0.0000000 | 0.0287356 | 3439.012926 |
| | 0.0000217 | 0.1652949 | 71096.60784 | | 0.0377842 | 0.6574713 | 6466.54012 |
| | 0.0000000 | 0.0281207 | 75269.22784 | | 0.0001916 | 0.7839080 | 6490.498273 |
| | 0.0004904 | 0.2256516 | 102406.355 | | 0.0000286 | 0.8091954 | 6689.07332 |
| | 0.0000016 | 0.1378601 | 124396.8269 | | 0.0301465 | 0.6574713 | 6863.56499 |
| | 0.0000003 | 0.1104252 | 135047.7781 | | 0.0113399 | 0.3114943 | 7781.442162 |
| | 0.0000000 | 0.0281207 | 150365.8563 | | 0.0146436 | 0.6701149 | 8845.312346 |
| F5CSL | 0.0000000 | 0.0540230 | 3439.012926 | | 0.0055263 | 0.7080460 | 8966.56789 |
| | 0.0118397 | 0.3114943 | 4793.423523 | | 0.0091111 | 0.6827586 | 9174.061297 |
| | 0.0364072 | 0.6574713 | 6466.54012 | | 0.0016980 | 0.7333333 | 25619.17312 |
| | 0.0002738 | 0.7586207 | 6490.498273 | | 0.0005779 | 0.7459770 | 33593.85227 |
| | 0.0123590 | 0.6741379 | 6661.944 | | 0.0018823 | 0.7206897 | 34007.46796 |
| | 0.0000018 | 0.8597701 | 6689.07332 | | 0.0020848 | 0.7206897 | 34545.63707 |
| | 0.0015304 | 0.7333333 | 6863.56499 | | 0.0018823 | 0.7333333 | 34736.3154 |
| | 0.0060637 | 0.2862069 | 7781.442162 | | 0.0021934 | 0.7333333 | 34944.03116 |
| | 0.0016980 | 0.7333333 | 8845.312346 | | 0.0020848 | 0.7333333 | 35193.60994 |
| | 0.0001330 | 0.7839080 | 8966.56789 | | 0.0037803 | 0.7206897 | 35450.83994 |
| | 0.0000860 | 0.8091954 | 9174.061297 | | 0.0001504 | 0.2143678 | 47192.29627 |
| | 0.0187682 | 0.6574713 | 9319.311385 | F5CSH | 0.0200134 | 0.6771337 | 10008.14304 |
| | 0.0000135 | 0.8344828 | 25619.17312 | | 0.0171027 | 0.7093398 | 10016.99725 |
| | 0.0021934 | 0.7206897 | 33593.85227 | | 0.0024712 | 0.7576490 | 10028.78563 |
| | 0.0050322 | 0.7080460 | 34007.46796 | | 0.0000273 | 0.8381643 | 10047.62033 |
| | 0.0063497 | 0.7080460 | 34545.63707 | | 0.0000114 | 0.8542673 | 10060.25984 |
| | 0.0066478 | 0.6954023 | 34736.3154 | | 0.0000066 | 0.8703704 | 10073.33412 |
| | 0.0069584 | 0.7080460 | 34944.03116 | | 0.0000004 | 0.9186795 | 10120.52937 |
| | 0.0052740 | 0.7080460 | 35193.60994 | | 0.0000003 | 0.9186795 | 10146.3839 |
| | 0.0069584 | 0.6954023 | 35450.83994 | | 0.0000004 | 0.9186795 | 10162.43733 |
| | 0.0063497 | 0.2942529 | 47192.29627 | | 0.0000004 | 0.9186795 | 10178.08813 |
| F5CSH | 0.0046230 | 0.7254428 | 10008.14304 | | 0.0000006 | 0.9186795 | 10195.2301 |
| | 0.0016686 | 0.7415459 | 10016.99725 | | 0.0000010 | 0.9186795 | 10205.50105 |
| | 0.0001655 | 0.8220612 | 10028.78563 | | 0.0000011 | 0.9186795 | 10215.263 |
| | 0.0000079 | 0.8703704 | 10047.62033 | | 0.0000009 | 0.9186795 | 10227.99328 |
| | 0.0000031 | 0.8864734 | 10060.25984 | | 0.0000007 | 0.9186795 | 10237.23424 |
| | 0.0000015 | 0.8864734 | 10073.33412 | | 0.0000004 | 0.9186795 | 10254.36842 |
| | 0.0000004 | 0.9186795 | 10120.52937 | | 0.0000002 | 0.9186795 | 10281.47372 |
| | 0.0000003 | 0.9186795 | 10146.3839 | | 0.0000002 | 0.9186795 | 10296.18987 |
| | 0.0000004 | 0.9186795 | 10162.43733 | | 0.0000002 | 0.9186795 | 10308.69901 |
| | 0.0000003 | 0.9186795 | 10178.08813 | | 0.0000002 | 0.9186795 | 10341.43948 |
| | 0.0000003 | 0.9186795 | 10195.2301 | | 0.0000002 | 0.9186795 | 10359.22541 |
| | 0.0000002 | 0.9186795 | 10205.50105 | | 0.0000002 | 0.9186795 | 10373.50565 |
| | 0.0000005 | 0.9186795 | 10215.263 | | 0.0000001 | 0.9347826 | 10388.7689 |
| | 0.0000005 | 0.9186795 | 10227.99328 | | 0.0000001 | 0.9186795 | 10402.74887 |
| | 0.0000004 | 0.9186795 | 10237.23424 | | 0.0000001 | 0.9347826 | 10418.95812 |
| | 0.0000003 | 0.9186795 | 10254.36842 | | 0.0000004 | 0.9186795 | 10433.30772 |
| | 0.0000002 | 0.9186795 | 10281.47372 | | 0.0000002 | 0.9186795 | 10452.21013 |
| | 0.0000002 | 0.9186795 | 10296.18987 | | 0.0000000 | 0.9508857 | 10465.90587 |
| | 0.0000001 | 0.9186795 | 10308.69901 | | 0.0000001 | 0.9347826 | 10481.18415 |
| | 0.0000002 | 0.9186795 | 10341.43948 | | 0.0000002 | 0.9186795 | 10498.6017 |
| | 0.0000002 | 0.9186795 | 10359.22541 | | 0.0000006 | 0.9025765 | 10508.92986 |
| | 0.0000002 | 0.9186795 | 10373.50565 | | 0.0000004 | 0.9186795 | 10514.91432 |
| | 0.0000001 | 0.9186795 | 10388.7689 | | 0.0000004 | 0.9186795 | 10527.20915 |
| | 0.0000001 | 0.9186795 | 10402.74887 | | 0.0000006 | 0.9025765 | 10546.16486 |
| | 0.0000001 | 0.9347826 | 10418.95812 | | 0.0000007 | 0.8864734 | 10575.10057 |
| | 0.0000002 | 0.9186795 | 10433.30772 | | 0.0000002 | 0.9186795 | 10596.28169 |
| | 0.0000001 | 0.9186795 | 10452.21013 | | 0.0000007 | 0.9025765 | 10616.84987 |
| | 0.0000000 | 0.9508857 | 10465.90587 | | 0.0000007 | 0.9186795 | 10636.82577 |
| | 0.0000000 | 0.9508857 | 10481.18415 | | 0.0000002 | 0.9186795 | 10654.23706 |
| | 0.0000001 | 0.9347826 | 10498.6017 | | 0.0000002 | 0.9186795 | 10665.17962 |
| | 0.0000002 | 0.9186795 | 10508.92986 | | 0.0000055 | 0.8703704 | 10672.3618 |
| | 0.0000001 | 0.9347826 | 10514.91432 | | 0.0000013 | 0.8864734 | 10681.084 |
| | 0.0000001 | 0.9347826 | 10527.20915 | | 0.0000136 | 0.8542673 | 10694.26283 |
| | 0.0000002 | 0.9186795 | 10546.16486 | | 0.0000211 | 0.8220612 | 10711.81013 |
| | 0.0000001 | 0.9186795 | 10575.10057 | | 0.0000124 | 0.8542673 | 10725.60891 |
| | 0.0000000 | 0.9508857 | 10596.28169 | | 0.0001308 | 0.7898551 | 10736.52558 |
| | 0.0000001 | 0.9186795 | 10616.84987 | | 0.0000016 | 0.9025765 | 10751.55877 |
| | 0.0000000 | 0.9347826 | 10636.82577 | | 0.0000011 | 0.9025765 | 10773.15923 |
| | 0.0000000 | 0.9347826 | 10654.23706 | | 0.0000418 | 0.8220612 | 10790.91972 |
| | 0.0000002 | 0.9025765 | 10665.17962 | | 0.0074402 | 0.7254428 | 10816.55733 |
| | 0.0000021 | 0.8864734 | 10672.3618 | | 0.0001789 | 0.7898551 | 10874.11936 |
| | 0.0000012 | 0.8864734 | 10681.084 | | 0.0012752 | 0.7737520 | 10891.02957 |
| | 0.0000012 | 0.9025765 | 10694.26283 | | 0.0004110 | 0.7898551 | 10906.58841 |
| | 0.0000104 | 0.8381643 | 10711.81013 | | 0.0009691 | 0.7737520 | 10927.60682 |
| | 0.0002433 | 0.7898551 | 10725.60891 | | 0.0180288 | 0.7254428 | 10948.05849 |
| | 0.0000809 | 0.8059581 | 10736.52558 | | 0.0381678 | 0.6449275 | 11021.63296 |
| | 0.0003053 | 0.7898551 | 10751.55877 | | 0.0145703 | 0.7093398 | 11095.98577 |
| | 0.0000494 | 0.8381643 | 10773.15923 | | 0.0023167 | 0.7093398 | 11115.98954 |
| | 0.0004758 | 0.7737520 | 10790.91972 | | 0.0145703 | 0.7093398 | 11132.56083 |
| | 0.0001531 | 0.8059581 | 10874.11936 | | 0.0023167 | 0.2423510 | 11187.64311 |
| | 0.0020339 | 0.7415459 | 10891.02957 | | 0.0004758 | 0.1940419 | 11197.80359 |
| | 0.0009040 | 0.7737520 | 10906.58841 | | 0.0162187 | 0.3067633 | 11283.65755 |
| | 0.0110785 | 0.6932367 | 10927.60682 | | 0.0007858 | 0.2423510 | 11394.7004 |
| | 0.0162187 | 0.6932367 | 10948.05849 | | 0.0046230 | 0.2745572 | 11884.93051 |
| | 0.0078838 | 0.6932367 | 11021.63296 | | 0.0000298 | 0.1618357 | 11917.82491 |
| | 0.0233475 | 0.6771337 | 11076.12773 | | 0.0000809 | 0.1779388 | 11953.73243 |
| | 0.0003290 | 0.8059581 | 11095.98577 | | 0.0000384 | 0.1618357 | 11976.91805 |
| | 0.0017831 | 0.7737520 | 11115.98954 | | 0.0000012 | 0.1135266 | 11996.5121 |
| | 0.0439411 | 0.6610306 | 11132.56083 | | 0.0000004 | 0.0813205 | 12027.11641 |
| | 0.0110785 | 0.3067633 | 11187.64311 | | 0.0000003 | 0.0652174 | 12052.53166 |
| | 0.0285359 | 0.3550725 | 11197.80359 | | 0.0000494 | 0.1940419 | 12084.07622 |
| | 0.0346883 | 0.3389694 | 11210.19746 | | 0.0000079 | 0.1296296 | 12925.60447 |
| | 0.0271535 | 0.3389694 | 11283.65755 | | 0.0000013 | 0.1457327 | 12974.23339 |
| | 0.0000633 | 0.1618357 | 11394.7004 | | 0.0000004 | 0.0974235 | 13028.74639 |
| | 0.0000114 | 0.1457327 | 11884.93051 | | 0.0000000 | 0.0491143 | 13065.70027 |
| | 0.0000162 | 0.1457327 | 11917.82491 | | 0.0000000 | 0.0652174 | 13099.66919 |
| | 0.0000633 | 0.1779388 | 11953.73243 | | 0.0000066 | 0.1296296 | 13146.65925 |
| | 0.0000537 | 0.1618357 | 11976.91805 | | 0.0000003 | 0.0813205 | 13187.45517 |
| | 0.0000003 | 0.0974235 | 11996.5121 | | 0.0000009 | 0.0974235 | 13233.79106 |
| | 0.0000015 | 0.1296296 | 12027.11641 | | 0.0000002 | 0.0813205 | 13291.70681 |
| | 0.0000009 | 0.0974235 | 12052.53166 | | 0.0000002 | 0.0652174 | 13320.77403 |
| | 0.0000298 | 0.1779388 | 12084.07622 | | 0.0000001 | 0.0652174 | 13356.70436 |
| | 0.0138029 | 0.6932367 | 12493.00409 | | 0.0000230 | 0.1618357 | 13426.83767 |
| | 0.0088427 | 0.7093398 | 12511.09369 | | 0.0003817 | 0.2101449 | 13491.55847 |
| | 0.0000148 | 0.1457327 | 12925.60447 | | 0.0299785 | 0.3550725 | 13581.27869 |
| | 0.0000006 | 0.0974235 | 12974.23339 | | 0.0189985 | 0.3389694 | 13655.43547 |
| | 0.0000010 | 0.1135266 | 13028.74639 | | 0.0000087 | 0.1296296 | 14019.70586 |
| | 0.0000000 | 0.0330113 | 13065.70027 | | 0.0000000 | 0.0330113 | 14214.24986 |
| | 0.0000000 | 0.0330113 | 13099.66919 | | 0.0000000 | 0.0169082 | 14344.33114 |
| | 0.0000021 | 0.1135266 | 13146.65925 | | 0.0016686 | 0.7576490 | 14729.54802 |
| | 0.0000001 | 0.0652174 | 13187.45517 | | 0.0058812 | 0.7254428 | 14767.85965 |
| | 0.0000000 | 0.0169082 | 13233.79106 | | 0.0000005 | 0.1135266 | 15566.82603 |
| | 0.0000000 | 0.0330113 | 13291.70681 | | 0.0000353 | 0.1618357 | 15645.08235 |
| | 0.0000002 | 0.0813205 | 13320.77403 | | 0.0070191 | 0.2906602 | 16290.00891 |
| | 0.0000000 | 0.0491143 | 13356.70436 | | 0.0000000 | 0.0491143 | 17653.98849 |
| | 0.0000007 | 0.0974235 | 13426.83767 | | 0.0000015 | 0.1135266 | 17809.61765 |
| | 0.0001116 | 0.1940419 | 13491.55847 | | 0.0000011 | 0.8864734 | 17974.36692 |
| | 0.0043492 | 0.2906602 | 13581.27869 | | 0.0000746 | 0.8059581 | 29272.79419 |
| | 0.0000001 | 0.0813205 | 14019.70586 | | 0.0004758 | 0.7737520 | 33625.51714 |
| | 0.0000000 | 0.0169082 | 14214.24986 | | 0.0004423 | 0.7737520 | 33961.79795 |
| | 0.0000000 | 0.0169082 | 14344.33114 | | 0.0006821 | 0.2262480 | 42641.96081 |
| | 0.0000136 | 0.8703704 | 14729.54802 | | 0.0003290 | 0.2101449 | 42923.06964 |
| | 0.0000251 | 0.8220612 | 14767.85965 | | 0.0000951 | 0.2101449 | 43153.71795 |
| | 0.0093602 | 0.6932367 | 14855.80746 | | 0.0000537 | 0.1940419 | 43558.60561 |
| | 0.0000015 | 0.1296296 | 15566.82603 | | 0.0000072 | 0.1135266 | 44673.19334 |
| | 0.0000079 | 0.1457327 | 15645.08235 | | 0.0000001 | 0.0652174 | 45254.93201 |
| | 0.0033949 | 0.2906602 | 16290.00891 | | 0.0028089 | 0.7254428 | 60596.55945 |
| | 0.0000000 | 0.0491143 | 17653.98849 | | 0.0000011 | 0.9025765 | 61369.70702 |
| | 0.0000273 | 0.1618357 | 17809.61765 | | 0.0023167 | 0.2745572 | 66754.36307 |
| | 0.0000124 | 0.8703704 | 17974.36692 | | 0.0040902 | 0.2584541 | 75408.4976 |
| | 0.0020339 | 0.7576490 | 26021.04631 | | 0.0010385 | 0.2423510 | 79145.35138 |
| | 0.0000019 | 0.8864734 | 29272.79419 | | 0.0020339 | 0.2584541 | 91493.20762 |
| | 0.0110785 | 0.7093398 | 33625.51714 | | 0.0000324 | 0.1779388 | 133992.2232 |
| | 0.0145703 | 0.7093398 | 33961.79795 | | 0.0110785 | 0.2584541 | 168584.5002 |
| | 0.0001531 | 0.2101449 | 42641.96081 | | 0.0000124 | 0.1457327 | 198991.3517 |
| | 0.0001655 | 0.1940419 | 42923.06964 | F6CSL | 0.0000046 | 0.8403576 | 2990.018687 |
| | 0.0000211 | 0.1618357 | 43153.71795 | | 0.0000168 | 0.8122605 | 3360.866902 |
| | 0.0000273 | 0.1779388 | 43558.60561 | | 0.0000050 | 0.8263091 | 4179.401548 |
| | 0.0000005 | 0.1135266 | 44673.19334 | | 0.0000022 | 0.8544061 | 4198.411686 |
| | 0.0000001 | 0.0813205 | 45254.93201 | | 0.0000016 | 0.8684547 | 4252.48155 |
| | 0.0200134 | 0.6610306 | 60596.55945 | | 0.0000260 | 0.8122605 | 4354.864569 |
| | 0.0000177 | 0.8381643 | 61369.70702 | | 0.0000007 | 0.8544061 | 4410.72305 |
| | 0.0099045 | 0.2584541 | 66754.36307 | | 0.0000750 | 0.7982120 | 4466.484268 |
| | 0.0271535 | 0.3067633 | 75408.4976 | | 0.0000860 | 0.7841635 | 4536.675037 |
| | 0.0002831 | 0.2101449 | 79145.35138 | | 0.0002040 | 0.7560664 | 4626.037492 |
| | 0.0005117 | 0.2423510 | 91493.20762 | | 0.0030793 | 0.7279693 | 4818.821494 |
| | 0.0007322 | 0.1940419 | 133992.2232 | | 0.0231454 | 0.3346105 | 5882.598843 |
| | 0.0016686 | 0.2262480 | 168584.5002 | | 0.0000022 | 0.8544061 | 6487.935491 |
| | 0.0000136 | 0.1618357 | 198991.3517 | | 0.0027670 | 0.7279693 | 6544.667343 |
| F6CSL | 0.0000001 | 0.8965517 | 2990.018687 | | 0.0000019 | 0.8684547 | 6689.164478 |
| | 0.0000016 | 0.8544061 | 3360.866902 | | 0.0351130 | 0.6436782 | 6855.504433 |
| | 0.0001678 | 0.7701149 | 4179.401548 | | 0.0123987 | 0.6858238 | 6953.588087 |
| | 0.0001376 | 0.7701149 | 4198.411686 | | 0.0129846 | 0.6998723 | 7588.918733 |
| | 0.0000019 | 0.8825032 | 4252.48155 | | 0.0252060 | 0.6717752 | 8719.481654 |
| | 0.0018881 | 0.7139208 | 4354.864569 | | 0.0000099 | 0.8263091 | 8842.966359 |
| | 0.0000002 | 0.8965517 | 4410.72305 | | 0.0000039 | 0.8544061 | 9046.727666 |
| | 0.0000347 | 0.8263091 | 4466.484268 | | 0.0029193 | 0.7139208 | 9165.408341 |
| | 0.0002639 | 0.7841635 | 4536.675037 | | 0.0021086 | 0.7420179 | 9668.960682 |
| | 0.0011345 | 0.7560664 | 4626.037492 | | 0.0000115 | 0.1660281 | 10294.7238 |
| | 0.0098066 | 0.6922095 | 4818.821494 | | 0.0030793 | 0.2784163 | 10463.66285 |
| | 0.0186220 | 0.3128991 | 5014.908804 | | 0.0001288 | 0.2081737 | 10733.07419 |
| | 0.0252060 | 0.3269476 | 5017.339281 | | 0.0000001 | 0.0881226 | 12480.3404 |
| | 0.0023525 | 0.2848020 | 5882.598843 | | 0.0000000 | 0.0600255 | 12651.6673 |
| | 0.0000000 | 0.8965517 | 6487.935491 | | 0.0463412 | 0.6781609 | 13920.46348 |
| | 0.0004932 | 0.7560664 | 6544.667343 | | 0.0262945 | 0.6922095 | 14102.40615 |
| | 0.0118364 | 0.6858238 | 6645.390397 | | 0.0027670 | 0.7279693 | 15178.11257 |
| | 0.0000000 | 0.9386973 | 6689.164478 | | 0.0007091 | 0.7560664 | 15361.4929 |
| | 0.0026219 | 0.7279693 | 6855.504433 | | 0.0001912 | 0.7841635 | 15915.33173 |
| | 0.0014263 | 0.7420179 | 6871.100138 | | 0.0001471 | 0.7841635 | 16093.36476 |
| | 0.0030793 | 0.7139208 | 6953.588087 | | 0.0019956 | 0.7420179 | 17426.66866 |
| | 0.0274235 | 0.3128991 | 7781.319356 | | 0.0000429 | 0.7982120 | 18048.90648 |
| | 0.0012017 | 0.7279693 | 8606.881674 | | 0.0005243 | 0.2286079 | 23037.50948 |
| | 0.0001571 | 0.7701149 | 8719.481654 | | 0.0162966 | 0.3269476 | 23524.56381 |
| | 0.0000000 | 0.9386973 | 8842.966359 | | 0.0118364 | 0.7279693 | 28178.79099 |
| | 0.0000000 | 0.9527458 | 9046.727666 | | 0.0000429 | 0.8263091 | 29021.87789 |
| | 0.0000347 | 0.8122605 | 9165.408341 | | 0.0000429 | 0.8263091 | 30269.89108 |
| | 0.0000400 | 0.8122605 | 9316.869151 | | 0.0000530 | 0.7982120 | 31162.97802 |
| | 0.0000209 | 0.8122605 | 9668.960682 | | 0.0036083 | 0.2567050 | 42060.31032 |
| | 0.0013475 | 0.7420179 | 9771.571531 | | 0.0148918 | 0.3269476 | 44309.89849 |
| | 0.0003192 | 0.2081737 | 10294.7238 | | 0.0000985 | 0.1864623 | 44686.33634 |
| | 0.0054469 | 0.2924649 | 10463.66285 | | 0.0003192 | 0.2145594 | 45344.38107 |
| | 0.0000209 | 0.1724138 | 10733.07419 | | 0.0000323 | 0.1724138 | 46173.68592 |
| | 0.0000001 | 0.0740741 | 12480.3404 | | 0.0002996 | 0.2005109 | 47417.69242 |
| | 0.0000001 | 0.0881226 | 12651.6673 | F6CSH | 0.0000000 | 0.9801587 | 10034.41899 |
| | 0.0021086 | 0.7203065 | 13920.46348 | | 0.0000000 | 0.9801587 | 10134.16987 |
| | 0.0003619 | 0.7624521 | 14102.40615 | | 0.0000000 | 0.9801587 | 10164.38262 |
| | 0.0178165 | 0.6717752 | 15178.11257 | | 0.0000000 | 0.9801587 | 10179.25579 |
| | 0.0029193 | 0.7279693 | 15361.4929 | | 0.0000000 | 0.9801587 | 10199.786 |
| | 0.0001571 | 0.7841635 | 15915.33173 | | 0.0000000 | 0.9801587 | 10247.31042 |
| | 0.0000920 | 0.8122605 | 16093.36476 | | 0.0000000 | 0.9801587 | 10308.99693 |
| | 0.0010102 | 0.7279693 | 17426.66866 | | 0.0000000 | 0.9801587 | 10324.98028 |
| | 0.0000042 | 0.8544061 | 18048.90648 | | 0.0000000 | 0.9801587 | 10351.28255 |
| | 0.0186220 | 0.3128991 | 23037.50948 | | 0.0000000 | 0.9801587 | 10382.71287 |
| | 0.0000013 | 0.8684547 | 28178.79099 | | 0.0000000 | 0.9801587 | 10437.59021 |
| | 0.0000000 | 0.9527458 | 29021.87789 | | 0.0000000 | 0.9801587 | 10456.27805 |
| | 0.0000000 | 0.9246488 | 30269.89108 | | 0.0000000 | 0.9365079 | 10586.5933 |
| | 0.0000039 | 0.8403576 | 31162.97802 | | 0.0000000 | 0.9656085 | 10639.37916 |
| | 0.0186220 | 0.2988506 | 33490.36172 | | 0.0000000 | 0.9801587 | 10659.7841 |
| | 0.0089131 | 0.2988506 | 34479.07332 | | 0.0000000 | 0.9656085 | 10714.34361 |
| | 0.0001376 | 0.2362708 | 42060.31032 | | 0.0000000 | 0.9365079 | 10736.08599 |
| | 0.0036083 | 0.2848020 | 44686.33634 | | 0.0000002 | 0.8928571 | 10828.01891 |
| | 0.0066526 | 0.2848020 | 45344.38107 | | 0.0000001 | 0.9365079 | 10910.64978 |
| | 0.0005243 | 0.2286079 | 46173.68592 | | 0.0000004 | 0.8928571 | 10929.45696 |
| | 0.0040055 | 0.2848020 | 47417.69242 | | 0.0001155 | 0.7764550 | 10952.36983 |
| F6CSH | 0.0000000 | 0.9814815 | 10034.41899 | | 0.0078125 | 0.6746032 | 11084.38324 |
| | 0.0000000 | 0.9814815 | 10134.16987 | | 0.0018417 | 0.2526455 | 11471.46464 |
| | 0.0000000 | 0.9814815 | 10164.38262 | | 0.0001518 | 0.2089947 | 11571.8247 |
| | 0.0000000 | 0.9814815 | 10179.25579 | | 0.0000000 | 0.0925926 | 11623.61757 |
| | 0.0000000 | 0.9814815 | 10199.786 | | 0.0000001 | 0.0780423 | 11673.12834 |
| | 0.0000000 | 0.9814815 | 10247.31042 | | 0.0000000 | 0.0343915 | 11750.83857 |
| | 0.0000000 | 0.9814815 | 10308.99693 | | 0.0000000 | 0.0634921 | 11779.63819 |
| | 0.0000000 | 0.9814815 | 10324.98028 | | 0.0000000 | 0.0343915 | 11824.74215 |
| | 0.0000000 | 0.9814815 | 10351.28255 | | 0.0000000 | 0.0634921 | 11886.09971 |
| | 0.0000000 | 0.9814815 | 10382.71287 | | 0.0000000 | 0.0343915 | 11982.74557 |
| | 0.0000000 | 0.9814815 | 10437.59021 | | 0.0000000 | 0.0489418 | 12017.01197 |
| | 0.0000000 | 0.9814815 | 10456.27805 | | 0.0000000 | 0.0343915 | 12050.07302 |
| | 0.0000000 | 0.9543210 | 10586.5933 | | 0.0000000 | 0.0198413 | 12170.65112 |
| | 0.0000000 | 0.9679012 | 10639.37916 | | 0.0000000 | 0.0634921 | 12200.58095 |
| | 0.0000000 | 0.9814815 | 10659.7841 | | 0.0000007 | 0.1362434 | 12243.19154 |
| | 0.0000000 | 0.9679012 | 10714.34361 | | 0.0110152 | 0.6600529 | 12577.78835 |
| | 0.0000000 | 0.9679012 | 10736.08599 | | 0.0000021 | 0.1216931 | 12747.38612 |
| | 0.0000066 | 0.8456790 | 10828.01891 | | 0.0000000 | 0.0634921 | 12889.83948 |
| | 0.0000000 | 0.9271605 | 10910.64978 | | 0.0000000 | 0.0198413 | 13157.2016 |
| | 0.0000002 | 0.9000000 | 10929.45696 | | 0.0000000 | 0.0780423 | 13373.91926 |
| | 0.0000646 | 0.7777778 | 10952.36983 | | 0.0001624 | 0.2089947 | 13429.21615 |
| | 0.0000429 | 0.8049383 | 11084.38324 | | 0.0001624 | 0.2235450 | 13859.59685 |
| | 0.0096227 | 0.6827160 | 11141.2016 | | 0.0001738 | 0.2235450 | 13899.93271 |
| | 0.0213677 | 0.6827160 | 11175.10112 | | 0.0000000 | 0.0198413 | 14029.68761 |
| | 0.0000348 | 0.1913580 | 11471.46464 | | 0.0000000 | 0.0198413 | 14160.13964 |
| | 0.0004124 | 0.2537037 | 11571.8247 | | 0.0000000 | 0.0198413 | 14336.01183 |
| | 0.0000001 | 0.0907407 | 11623.61757 | | 0.0000000 | 0.0489418 | 14434.26849 |
| | 0.0000009 | 0.1179012 | 11673.12834 | | 0.0001078 | 0.8055556 | 14665.79315 |
| | 0.0000000 | 0.0419753 | 11750.83857 | | 0.0000221 | 0.8201058 | 14829.46698 |
| | 0.0000000 | 0.0419753 | 11779.63819 | | 0.0042085 | 0.2817460 | 15063.55589 |
| | 0.0000000 | 0.0283951 | 11824.74215 | | 0.0006316 | 0.2671958 | 15244.10632 |
| | 0.0000000 | 0.0419753 | 11886.09971 | | 0.0000000 | 0.0198413 | 15420.52874 |
| | 0.0000000 | 0.0283951 | 11982.74557 | | 0.0000055 | 0.1362434 | 15617.44859 |
| | 0.0000000 | 0.0827160 | 12017.01197 | | 0.0000002 | 0.8928571 | 15840.99957 |
| | 0.0000000 | 0.0283951 | 12050.07302 | | 0.0325041 | 0.6309524 | 16095.07951 |
| | 0.0000000 | 0.0283951 | 12170.65112 | | 0.0000002 | 0.9074074 | 16555.54127 |
| | 0.0000000 | 0.0555556 | 12200.58095 | | 0.0000001 | 0.9074074 | 16719.92602 |
| | 0.0000033 | 0.1506173 | 12243.19154 | | 0.0000000 | 0.0634921 | 17530.97848 |
| | 0.0000245 | 0.8049383 | 12577.78835 | | 0.0000000 | 0.0343915 | 17656.32521 |
| | 0.0000373 | 0.1641975 | 12747.38612 | | 0.0000000 | 0.9801587 | 18041.10658 |
| | 0.0000001 | 0.1098765 | 12889.83948 | | 0.0000000 | 0.9365079 | 18241.15243 |
| | 0.0000000 | 0.0148148 | 13157.2016 | | 0.0000939 | 0.7764550 | 18621.70523 |
| | 0.0000000 | 0.0691358 | 13373.91926 | | 0.0000238 | 0.1798942 | 23184.82846 |
| | 0.0013163 | 0.2592593 | 13429.21615 | | 0.0262606 | 0.3253968 | 23404.4619 |
| | 0.0006636 | 0.2592593 | 13859.59685 | | 0.0000004 | 0.0925926 | 33564.57869 |
| | 0.0013163 | 0.2592593 | 13899.93271 | | 0.0000037 | 0.1362434 | 33807.33449 |
| | 0.0000000 | 0.0283951 | 14029.68761 | | 0.0000010 | 0.1507937 | 39178.65241 |
| | 0.0000000 | 0.0148148 | 14160.13964 | | 0.0005578 | 0.2380952 | 39783.20564 |
| | 0.0000000 | 0.0148148 | 14336.01183 | | 0.0000276 | 0.1798942 | 43603.62674 |
| | 0.0000000 | 0.0283951 | 14434.26849 | | 0.0000002 | 0.1071429 | 44709.3435 |
| | 0.0000400 | 0.8185185 | 14665.79315 | | 0.0000176 | 0.1507937 | 45499.42435 |
| | 0.0000004 | 0.8728395 | 14829.46698 | | 0.0000040 | 0.1362434 | 46648.55176 |
| | 0.0040176 | 0.3080247 | 15063.55589 | | 0.0416359 | 0.3544974 | 47902.96874 |
| | 0.0000844 | 0.1913580 | 15244.10632 | | | | |
| | 0.0000000 | 0.0148148 | 15420.52874 | | | | |
| | 0.0000006 | 0.1506173 | 15617.44859 | | | | |
| | 0.0000373 | 0.8049383 | 15840.99957 | | | | |
| | 0.0000003 | 0.9000000 | 16555.54127 | | | | |
| | 0.0000002 | 0.9000000 | 16719.92602 | | | | |
| | 0.0000000 | 0.0555556 | 17530.97848 | | | | |
| | 0.0000000 | 0.0148148 | 17656.32521 | | | | |
| | 0.0000000 | 0.9814815 | 18041.10658 | | | | |
| | 0.0000000 | 0.9135802 | 18241.15243 | | | | |
| | 0.0000148 | 0.8185185 | 18621.70523 | | | | |
| | 0.0001099 | 0.2049383 | 23184.82846 | | | | |
| | 0.0000000 | 0.0555556 | 33564.57869 | | | | |
| | 0.0000000 | 0.0962963 | 33807.33449 | | | | |
| | 0.0000171 | 0.1913580 | 39178.65241 | | | | |
| | 0.0004651 | 0.2129630 | 39783.20564 | | | | |
| | 0.0000103 | 0.1777778 | 43603.62674 | | | | |
| | 0.0000000 | 0.0827160 | 44709.3435 | | | | |
| | 0.0000111 | 0.1506173 | 45499.42435 | | | | |
| | 0.0001252 | 0.2185185 | 46648.55176 | | | | |

TABLE 23
Biomarkers identified by SELDI technology with a p-value smaller or equal to 0.05 that can
discriminate secondary DENV infection from OFI. Grouped according to fraction it was found in.

| | Ct1_2 vs 2DF1_2 | | | | Ct1_2 vs 2DHF1_2 | | |
|---|---|---|---|---|---|---|---|
| | p-value | roc value | m/z average | | p-value | roc value | m/z average |
| F1CSL | 0.0000000 | 0.0151515 | 2625.181487 | F1CSL | 0.0000000 | 0.0454545 | 2625.181487 |
| | 0.0000000 | 0.0303030 | 2667.845783 | | 0.0000000 | 0.0454545 | 2667.845783 |
| | 0.0000000 | 0.0151515 | 2741.725854 | | 0.0000000 | 0.0303030 | 2741.725854 |
| | 0.0000000 | 0.0606061 | 2856.727191 | | 0.0000001 | 0.0757576 | 2856.727191 |
| | 0.0000000 | 0.0151515 | 2872.154934 | | 0.0000000 | 0.0303030 | 2872.154934 |
| | 0.0000000 | 0.0454545 | 2921.681011 | | 0.0000004 | 0.0909091 | 2921.681011 |
| | 0.0000000 | 0.0454545 | 2990.831829 | | 0.0000000 | 0.0303030 | 2990.831829 |
| | 0.0000000 | 0.0303030 | 3043.041845 | | 0.0000002 | 0.0757576 | 3043.041845 |
| | 0.0000001 | 0.0606061 | 3071.048466 | | 0.0000000 | 0.0757576 | 3175.340726 |
| | 0.0000000 | 0.0303030 | 3145.264181 | | 0.0000000 | 0.0757576 | 3209.081505 |
| | 0.0000000 | 0.0151515 | 3175.340726 | | 0.0000000 | 0.0606061 | 3262.112346 |
| | 0.0000000 | 0.0303030 | 3209.081505 | | 0.0000000 | 0.0151515 | 3280.760876 |
| | 0.0000000 | 0.0151515 | 3262.112346 | | 0.0000001 | 0.0757576 | 3307.659966 |
| | 0.0000000 | 0.0000000 | 3280.760876 | | 0.0000052 | 0.1303030 | 3358.521584 |
| | 0.0000000 | 0.0606061 | 3307.659966 | | 0.0000000 | 0.0454545 | 3589.060244 |
| | 0.0000000 | 0.0303030 | 3358.521584 | | 0.0000000 | 0.0303030 | 3680.115892 |
| | 0.0000005 | 0.0848485 | 3420.037403 | | 0.0000000 | 0.0757576 | 3814.303638 |
| | 0.0000000 | 0.0151515 | 3437.42944 | | 0.0000000 | 0.0303030 | 4063.241745 |
| | 0.0000001 | 0.0757576 | 3511.538012 | | 0.0000001 | 0.0757576 | 4182.768879 |
| | 0.0000000 | 0.0151515 | 3589.060244 | | 0.0000003 | 0.0909091 | 4299.724384 |
| | 0.0000001 | 0.0757576 | 3631.061336 | | 0.0000000 | 0.9727273 | 5559.119903 |
| | 0.0000000 | 0.0151515 | 3680.115892 | | 0.0000000 | 0.9727273 | 5574.710692 |
| | 0.0000000 | 0.0151515 | 3814.303638 | | 0.0000000 | 0.9878788 | 5675.016336 |
| | 0.0000003 | 0.0909091 | 3863.107595 | | 0.0000000 | 0.9878788 | 5689.765089 |
| | 0.0000000 | 0.0606061 | 3949.327603 | | 0.0000000 | 0.9424242 | 6143.838117 |
| | 0.0000001 | 0.0606061 | 3965.762902 | | 0.0000028 | 0.8666667 | 6487.509564 |
| | 0.0000000 | 0.0151515 | 4063.241745 | | 0.0000004 | 0.9121212 | 6591.475901 |
| | 0.0000000 | 0.0454545 | 4182.768879 | | 0.0000000 | 0.9575758 | 6944.325104 |
| | 0.0000000 | 0.0454545 | 4279.033423 | | 0.0015391 | 0.2303030 | 9170.910437 |
| | 0.0000000 | 0.0606061 | 4299.724384 | | 0.0000001 | 0.9272727 | 11902.07203 |
| | 0.0000000 | 0.0303030 | 4524.063737 | | 0.0000001 | 0.9121212 | 12083.60453 |
| | 0.0000000 | 0.9575758 | 5559.119903 | | 0.0000000 | 0.9727273 | 13375.41938 |
| | 0.0000000 | 0.9424242 | 5574.710692 | | 0.0000003 | 0.9272727 | 14757.7248 |
| | 0.0000000 | 0.9727273 | 5675.016336 | | 0.0000006 | 0.9121212 | 15198.38517 |
| | 0.0000000 | 0.9727273 | 5689.765089 | | 0.0000000 | 0.9424242 | 22937.26034 |
| | 0.0000000 | 0.9575758 | 6143.838117 | | 0.0000000 | 0.9424242 | 23544.76704 |
| | 0.0000000 | 0.9424242 | 6487.509564 | | 0.0000001 | 0.9121212 | 44810.68881 |
| | 0.0000000 | 0.9575758 | 6591.475901 | | 0.0000003 | 0.9121212 | 45184.10294 |
| | 0.0000002 | 0.8969697 | 6683.82645 | | 0.0000001 | 0.9272727 | 46357.92645 |
| | 0.0000002 | 0.9121212 | 6805.698908 | F1CSH | 0.0005411 | 0.7533333 | 10025.87122 |
| | 0.0000000 | 0.9878788 | 6944.325104 | | 0.0000004 | 0.8866667 | 10130.28134 |
| | 0.0000004 | 0.9121212 | 10101.46975 | | 0.0000057 | 0.8466667 | 10196.44234 |
| | 0.0000002 | 0.9272727 | 11902.07203 | | 0.0000001 | 0.9000000 | 10413.34138 |
| | 0.0000006 | 0.9121212 | 12083.60453 | | 0.0000001 | 0.8866667 | 10444.68912 |
| | 0.0000000 | 0.9727273 | 13375.41938 | | 0.0000001 | 0.9000000 | 10461.79685 |
| | 0.0000002 | 0.9272727 | 15198.38517 | | 0.0000001 | 0.9000000 | 10490.47668 |
| | 0.0000000 | 0.9424242 | 22937.26034 | | 0.0000001 | 0.9000000 | 10496.60495 |
| | 0.0000000 | 0.9575758 | 23544.76704 | | 0.0000001 | 0.9000000 | 10514.61735 |
| | 0.0000000 | 0.9575758 | 44810.68881 | | 0.0000000 | 0.0733333 | 10882.15141 |
| | 0.0000000 | 0.9575758 | 45184.10294 | | 0.0000000 | 0.0466667 | 10908.0378 |
| | 0.0000000 | 0.9575758 | 45616.26741 | | 0.0000000 | 0.0333333 | 10926.28045 |
| | 0.0000000 | 0.9575758 | 46357.92645 | | 0.0000000 | 0.0200000 | 10951.30804 |
| F1CSH | 0.0000061 | 0.8333333 | 10130.28134 | | 0.0000000 | 0.0866667 | 11040.90474 |
| | 0.0000743 | 0.7933333 | 10196.44234 | | 0.0000000 | 0.0466667 | 11078.55501 |
| | 0.0000046 | 0.8333333 | 10241.85866 | | 0.0000000 | 0.0600000 | 11154.55919 |
| | 0.0000040 | 0.8466667 | 10444.68912 | | 0.0000000 | 0.0733333 | 11197.83206 |
| | 0.0000026 | 0.8466667 | 10461.79685 | | 0.0000000 | 0.0200000 | 12009.62084 |
| | 0.0000000 | 0.1000000 | 10808.95097 | | 0.0000000 | 0.0333333 | 12080.71023 |
| | 0.0000000 | 0.0733333 | 10835.54836 | | 0.0000000 | 0.0466667 | 12112.68049 |
| | 0.0000000 | 0.0466667 | 10882.15141 | | 0.0000000 | 0.0600000 | 12132.64107 |
| | 0.0000000 | 0.0333333 | 10908.0378 | | 0.0000000 | 0.0866667 | 12163.0594 |
| | 0.0000000 | 0.0200000 | 10926.28045 | | 0.0000000 | 0.0600000 | 12268.93847 |
| | 0.0000000 | 0.0200000 | 10951.30804 | | 0.0000000 | 0.0333333 | 12358.50727 |
| | 0.0000000 | 0.0333333 | 11040.90474 | | 0.0000000 | 0.0466667 | 12493.48295 |
| | 0.0000000 | 0.0333333 | 11078.55501 | | 0.0000000 | 0.0733333 | 13839.31124 |
| | 0.0000000 | 0.0466667 | 11154.55919 | | 0.0000000 | 0.0200000 | 13930.38141 |
| | 0.0000000 | 0.0466667 | 11197.83206 | | 0.0000000 | 0.0866667 | 14140.38344 |
| | 0.0000001 | 0.1000000 | 11853.93279 | | 0.0000000 | 0.0866667 | 15200.55252 |
| | 0.0000000 | 0.0333333 | 12009.62084 | | 0.0000000 | 0.0866667 | 107068.7099 |
| | 0.0000000 | 0.0200000 | 12080.71023 | | 0.0000001 | 0.1000000 | 113333.4714 |
| | 0.0000000 | 0.0200000 | 12112.68049 | | 0.0000000 | 0.0200000 | 192541.7062 |
| | 0.0000000 | 0.0466667 | 12132.64107 | F1ISL | 0.0000000 | 0.9690476 | 2503.154582 |
| | 0.0000000 | 0.0466667 | 12163.0594 | | 0.0000000 | 0.9559524 | 2513.073032 |
| | 0.0000000 | 0.0466667 | 12268.93847 | | 0.0000001 | 0.9035714 | 2517.656451 |
| | 0.0000000 | 0.0333333 | 12358.50727 | | 0.0000001 | 0.9035714 | 2522.859861 |
| | 0.0000000 | 0.0466667 | 12493.48295 | | 0.0000000 | 0.9297619 | 2523.669165 |
| | 0.0000001 | 0.1000000 | 13655.28043 | | 0.0000000 | 0.9428571 | 2524.520651 |
| | 0.0000000 | 0.0866667 | 13839.31124 | | 0.0000000 | 0.9166667 | 2549.242272 |
| | 0.0000000 | 0.0466667 | 13930.38141 | | 0.0000000 | 0.9297619 | 2565.111611 |
| | 0.0000000 | 0.0866667 | 15200.55252 | | 0.0000000 | 0.9166667 | 2570.087202 |
| | 0.0000001 | 0.1000000 | 51139.33489 | | 0.0000003 | 0.9035714 | 2578.662526 |
| | 0.0000001 | 0.1000000 | 53826.57027 | | 0.0000000 | 0.9297619 | 2595.854473 |
| | 0.0000000 | 0.0866667 | 62969.82218 | | 0.0000001 | 0.9035714 | 2601.511801 |
| | 0.0000001 | 0.1000000 | 102198.3507 | | 0.0000000 | 0.9690476 | 2614.032211 |
| | 0.0000000 | 0.0866667 | 107068.7099 | | 0.0000000 | 0.9428571 | 2617.583867 |
| | 0.0000000 | 0.0733333 | 192541.7062 | | 0.0000000 | 0.9166667 | 2621.696749 |
| F1ISL | 0.0000000 | 0.9821429 | 2503.154582 | | 0.0000000 | 0.9166667 | 2631.998607 |
| | 0.0000000 | 0.9297619 | 2513.073032 | | 0.0000000 | 0.9297619 | 2634.20847 |
| | 0.0000000 | 0.9166667 | 2517.656451 | | 0.0000000 | 0.9559524 | 2635.700585 |
| | 0.0000000 | 0.9166667 | 2521.649116 | | 0.0000000 | 0.9559524 | 2637.169955 |
| | 0.0000002 | 0.9035714 | 2522.859861 | | 0.0000000 | 0.9559524 | 2637.618569 |
| | 0.0000000 | 0.9559524 | 2565.111611 | | 0.0000000 | 0.9428571 | 2638.060903 |
| | 0.0000000 | 0.9166667 | 2570.087202 | | 0.0000000 | 0.9559524 | 2638.54484 |
| | 0.0000000 | 0.9297619 | 2595.854473 | | 0.0000000 | 0.9690476 | 2639.173428 |
| | 0.0000000 | 0.9166667 | 2601.511801 | | 0.0000000 | 0.9821429 | 2639.795493 |
| | 0.0000001 | 0.9035714 | 2614.032211 | | 0.0000000 | 0.9821429 | 2640.467576 |
| | 0.0000000 | 0.9428571 | 2617.583867 | | 0.0000000 | 0.9821429 | 2641.164374 |
| | 0.0000001 | 0.9035714 | 2635.700585 | | 0.0000000 | 0.9821429 | 2641.926515 |
| | 0.0000000 | 0.9690476 | 2637.169955 | | 0.0000000 | 0.9821429 | 2642.885878 |
| | 0.0000000 | 0.9559524 | 2637.618569 | | 0.0000000 | 0.9297619 | 2643.949918 |
| | 0.0000000 | 0.9559524 | 2638.060903 | | 0.0000000 | 0.9428571 | 2646.227988 |
| | 0.0000000 | 0.9559524 | 2638.54484 | | 0.0000001 | 0.9035714 | 2659.814331 |
| | 0.0000000 | 0.9559524 | 2639.173428 | | 0.0000000 | 0.9297619 | 2661.194232 |
| | 0.0000001 | 0.9035714 | 2639.795493 | | 0.0000000 | 0.9559524 | 2661.84737 |
| | 0.0000003 | 0.9035714 | 2640.467576 | | 0.0000000 | 0.9428571 | 2662.858635 |
| | 0.0000000 | 0.9428571 | 2641.164374 | | 0.0000000 | 0.9166667 | 2666.570765 |
| | 0.0000000 | 0.9821429 | 2641.926515 | | 0.0000000 | 0.9297619 | 2679.146726 |
| | 0.0000000 | 0.9821429 | 2642.885878 | | 0.0000000 | 0.9821429 | 2681.308781 |
| | 0.0000000 | 0.9559524 | 2643.949918 | | 0.0000000 | 0.9428571 | 2684.692433 |
| | 0.0000000 | 0.9428571 | 2646.227988 | | 0.0000000 | 0.9428571 | 2686.45453 |
| | 0.0000002 | 0.9035714 | 2659.814331 | | 0.0000000 | 0.9559524 | 2704.270371 |
| | 0.0000000 | 0.9297619 | 2661.194232 | | 0.0000000 | 0.9428571 | 2709.71622 |
| | 0.0000000 | 0.9559524 | 2661.84737 | | 0.0000000 | 0.9166667 | 2726.112884 |
| | 0.0000000 | 0.9821429 | 2662.858635 | | 0.0000000 | 0.9559524 | 2738.579429 |
| | 0.0000000 | 0.9166667 | 2666.570765 | | 0.0000000 | 0.9821429 | 2749.363653 |
| | 0.0000000 | 0.9166667 | 2674.971433 | | 0.0000000 | 0.9428571 | 2752.680443 |
| | 0.0000000 | 0.9297619 | 2679.146726 | | 0.0000001 | 0.9035714 | 2753.648132 |
| | 0.0000000 | 0.9821429 | 2681.308781 | | 0.0000002 | 0.9035714 | 2753.857675 |
| | 0.0000000 | 0.9821429 | 2684.692433 | | 0.0000002 | 0.9035714 | 2753.946851 |
| | 0.0000000 | 0.9821429 | 2686.45453 | | 0.0000000 | 0.9559524 | 2755.192724 |
| | 0.0000000 | 0.9821429 | 2704.270371 | | 0.0000000 | 0.9166667 | 2772.763638 |
| | 0.0000000 | 0.9821429 | 2709.71622 | | 0.0000000 | 0.9690476 | 2788.581536 |
| | 0.0000000 | 0.9166667 | 2738.579429 | | 0.0000000 | 0.9690476 | 2791.185756 |
| | 0.0000000 | 0.9428571 | 2749.363653 | | 0.0000000 | 0.9559524 | 2796.034541 |
| | 0.0000000 | 0.9821429 | 2752.680443 | | 0.0000000 | 0.9559524 | 2803.236487 |
| | 0.0000000 | 0.9821429 | 2753.648132 | | 0.0000000 | 0.9690476 | 2811.436125 |
| | 0.0000000 | 0.9821429 | 2753.857675 | | 0.0000000 | 0.9559524 | 2817.188037 |
| | 0.0000000 | 0.9821429 | 2753.946851 | | 0.0000000 | 0.9821429 | 2819.017882 |
| | 0.0000000 | 0.9690476 | 2755.192724 | | 0.0000000 | 0.9297619 | 2824.989455 |
| | 0.0000000 | 0.9297619 | 2772.763638 | | 0.0000000 | 0.9821429 | 2859.505809 |
| | 0.0000000 | 0.9428571 | 2775.410216 | | 0.0000000 | 0.9821429 | 2866.751136 |
| | 0.0000000 | 0.9690476 | 2788.581536 | | 0.0000000 | 0.9297619 | 2879.703192 |
| | 0.0000000 | 0.9297619 | 2791.185756 | | 0.0000000 | 0.9821429 | 2883.890105 |
| | 0.0000000 | 0.9428571 | 2796.034541 | | 0.0000000 | 0.9428571 | 2887.453423 |
| | 0.0000000 | 0.9297619 | 2803.236487 | | 0.0000000 | 0.9166667 | 2894.062647 |
| | 0.0000000 | 0.9821429 | 2811.436125 | | 0.0000000 | 0.9297619 | 2904.031336 |
| | 0.0000000 | 0.9428571 | 2817.188037 | | 0.0000000 | 0.9428571 | 2906.91955 |
| | 0.0000000 | 0.9428571 | 2824.989455 | | 0.0000000 | 0.9428571 | 2908.899563 |
| | 0.0000000 | 0.9559524 | 2859.505809 | | 0.0000003 | 0.9035714 | 2910.510035 |
| | 0.0000000 | 0.9690476 | 2866.751136 | | 0.0000000 | 0.9428571 | 2911.83993 |
| | 0.0000000 | 0.9821429 | 2879.703192 | | 0.0000000 | 0.9297619 | 2915.763212 |
| | 0.0000000 | 0.9821429 | 2883.890105 | | 0.0000000 | 0.9166667 | 2927.060302 |
| | 0.0000000 | 0.9821429 | 2887.453423 | | 0.0000001 | 0.9035714 | 2930.543416 |
| | 0.0000000 | 0.9559524 | 2894.062647 | | 0.0000000 | 0.9821429 | 2933.649099 |
| | 0.0000000 | 0.9428571 | 2904.031336 | | 0.0000000 | 0.9690476 | 2948.917414 |
| | 0.0000000 | 0.9690476 | 2906.91955 | | 0.0000000 | 0.9297619 | 2951.608271 |
| | 0.0000000 | 0.9428571 | 2908.899563 | | 0.0000001 | 0.9083333 | 2953.403838 |
| | 0.0000000 | 0.9690476 | 2910.510035 | | 0.0000000 | 0.9035714 | 2976.912269 |
| | 0.0000000 | 0.9690476 | 2911.83993 | | 0.0000000 | 0.9559524 | 2993.494244 |
| | 0.0000000 | 0.9166667 | 2915.763212 | | 0.0000000 | 0.9559524 | 2997.743006 |
| | 0.0000000 | 0.9297619 | 2927.060302 | | 0.0000001 | 0.9166667 | 3083.362119 |
| | 0.0000000 | 0.9559524 | 2930.543416 | | 0.0000000 | 0.9559524 | 3092.389989 |
| | 0.0000000 | 0.9690476 | 2933.649099 | | 0.0000000 | 0.9297619 | 3101.60504 |
| | 0.0000002 | 0.9035714 | 2938.720507 | | 0.0000002 | 0.9035714 | 3149.522222 |
| | 0.0000000 | 0.9428571 | 2948.917414 | | 0.0000000 | 0.9559524 | 3158.254319 |
| | 0.0000000 | 0.9297619 | 2951.608271 | | 0.0000000 | 0.9035714 | 3166.387243 |
| | 0.0000000 | 0.9476190 | 2953.403838 | | 0.0000001 | 0.9035714 | 3197.777988 |
| | 0.0000000 | 0.9214286 | 2956.963771 | | 0.0000000 | 0.9035714 | 3221.413524 |
| | 0.0000000 | 0.9428571 | 2976.912269 | | 0.0000000 | 0.9035714 | 3318.297755 |
| | 0.0000000 | 0.9559524 | 2993.494244 | | 0.0000000 | 0.9166667 | 3369.373189 |
| | 0.0000000 | 0.9428571 | 2997.743006 | | 0.0000000 | 0.9428571 | 3397.259967 |
| | 0.0000000 | 0.9297619 | 3015.428516 | | 0.0000000 | 0.9297619 | 3402.125169 |
| | 0.0000001 | 0.9035714 | 3020.653111 | | 0.0000002 | 0.9035714 | 3417.509394 |
| | 0.0000001 | 0.9035714 | 3042.639369 | | 0.0000000 | 0.9559524 | 3430.642457 |
| | 0.0000000 | 0.9166667 | 3066.155713 | | 0.0000000 | 0.9821429 | 3445.720567 |
| | 0.0000000 | 0.9297619 | 3083.362119 | | 0.0000000 | 0.9690476 | 3468.46665 |
| | 0.0000000 | 0.9559524 | 3092.389989 | | 0.0000000 | 0.9428571 | 3488.857635 |
| | 0.0000000 | 0.9428571 | 3113.412549 | | 0.0000000 | 0.9821429 | 3515.357908 |
| | 0.0000000 | 0.9035714 | 3149.522222 | | 0.0000000 | 0.9166667 | 3536.763944 |
| | 0.0000000 | 0.9559524 | 3158.254319 | | 0.0000000 | 0.9166667 | 3644.486854 |
| | 0.0000000 | 0.9297619 | 3172.450888 | | 0.0000000 | 0.9166667 | 3790.742865 |
| | 0.0000000 | 0.9821429 | 3177.822062 | | 0.0000000 | 0.9297619 | 3807.332552 |
| | 0.0000002 | 0.9035714 | 3213.068347 | | 0.0000001 | 0.9166667 | 4418.467916 |
| | 0.0000000 | 0.9559524 | 3221.413524 | F1ISH | 0.0000000 | 0.9855967 | 10011.99352 |
| | 0.0000001 | 0.9166667 | 3314.079782 | | 0.0000000 | 0.9855967 | 10020.52117 |
| | 0.0000000 | 0.9166667 | 3318.297755 | | 0.0000000 | 0.9855967 | 10025.43594 |
| | 0.0000001 | 0.9035714 | 3377.906174 | | 0.0000000 | 0.9855967 | 10028.64467 |
| | 0.0000000 | 0.9690476 | 3397.259967 | | 0.0000000 | 0.9855967 | 10036.97216 |
| | 0.0000000 | 0.9297619 | 3402.125169 | | 0.0000000 | 0.9855967 | 10051.63047 |
| | 0.0000000 | 0.9166667 | 3417.509394 | | 0.0000000 | 0.9855967 | 10061.98167 |
| | 0.0000000 | 0.9297619 | 3430.642457 | | 0.0000000 | 0.9855967 | 10072.44679 |
| | 0.0000000 | 0.9428571 | 3445.720567 | | 0.0000000 | 0.9855967 | 10081.58862 |
| | 0.0000000 | 0.9297619 | 3456.301995 | | 0.0000000 | 0.9855967 | 10090.2363 |
| | 0.0000000 | 0.9821429 | 3468.46665 | | 0.0000000 | 0.9855967 | 10098.45601 |
| | 0.0000000 | 0.9166667 | 3478.572514 | | 0.0000000 | 0.9855967 | 10105.92173 |
| | 0.0000000 | 0.9559524 | 3488.857635 | | 0.0000000 | 0.9855967 | 10114.78187 |
| | 0.0000000 | 0.9690476 | 3515.357908 | | 0.0000000 | 0.9855967 | 10135.6053 |
| | 0.0000000 | 0.9428571 | 3528.345658 | | 0.0000000 | 0.9855967 | 10147.36272 |
| | 0.0000000 | 0.9297619 | 3536.763944 | | 0.0000000 | 0.9855967 | 10162.00816 |
| | 0.0000000 | 0.9428571 | 3644.486854 | | 0.0000000 | 0.9718793 | 10175.58746 |
| | 0.0000000 | 0.9166667 | 3687.780782 | | 0.0000000 | 0.9581619 | 10187.91964 |
| | 0.0000000 | 0.9297619 | 3772.107649 | | 0.0000000 | 0.9307270 | 10198.46953 |
| | 0.0000000 | 0.9035714 | 3790.742865 | | 0.0000000 | 0.9581619 | 10208.17418 |
| | 0.0000001 | 0.9035714 | 3807.332552 | | 0.0000000 | 0.9444444 | 10217.54983 |
| | 0.0000000 | 0.9428571 | 3827.712827 | | 0.0000000 | 0.9170096 | 10230.57425 |
| | 0.0000000 | 0.9035714 | 3891.091076 | | 0.0000000 | 0.9307270 | 10262.82559 |
| F1ISH | 0.0000000 | 0.9855967 | 10011.99352 | | 0.0000000 | 0.9444444 | 10294.56991 |
| | 0.0000000 | 0.9855967 | 10020.52117 | | 0.0000000 | 0.9581619 | 10306.64326 |
| | 0.0000000 | 0.9855967 | 10025.43594 | | 0.0000001 | 0.9170096 | 10324.32336 |
| | 0.0000000 | 0.9855967 | 10028.64467 | | 0.0000002 | 0.9170096 | 10346.11123 |
| | 0.0000000 | 0.9855967 | 10036.97216 | | 0.0000001 | 0.0829904 | 11508.64295 |
| | 0.0000000 | 0.9855967 | 10051.63047 | | 0.0000002 | 0.0967078 | 11533.00585 |
| | 0.0000000 | 0.9855967 | 10061.98167 | | 0.0000000 | 0.0281207 | 11553.43803 |
| | 0.0000000 | 0.9855967 | 10072.44679 | | 0.0000000 | 0.0555556 | 11576.23632 |
| | 0.0000000 | 0.9855967 | 10081.58862 | | 0.0000001 | 0.0692730 | 11596.70327 |
| | 0.0000000 | 0.9855967 | 10090.2363 | | 0.0000001 | 0.0967078 | 11607.15919 |
| | 0.0000000 | 0.9855967 | 10098.45601 | | 0.0000000 | 0.0692730 | 11623.79327 |
| | 0.0000000 | 0.9855967 | 10105.92173 | | 0.0000000 | 0.0555556 | 11642.6909 |
| | 0.0000000 | 0.9855967 | 10114.78187 | | 0.0000000 | 0.0281207 | 11662.86838 |
| | 0.0000000 | 0.9855967 | 10135.6053 | | 0.0000000 | 0.0555556 | 11682.71261 |
| | 0.0000000 | 0.9855967 | 10147.36272 | | 0.0000004 | 0.0967078 | 11737.66458 |
| | 0.0000000 | 0.9855967 | 10162.00816 | | 0.0000000 | 0.0692730 | 11849.00542 |
| | 0.0000000 | 0.9855967 | 10175.58746 | | 0.0000000 | 0.0692730 | 11881.99843 |
| | 0.0000000 | 0.9718793 | 10187.91964 | | 0.0000000 | 0.0418381 | 11909.00114 |
| | 0.0000000 | 0.9444444 | 10198.46953 | | 0.0000000 | 0.0555556 | 11918.54028 |
| | 0.0000000 | 0.9718793 | 10208.17418 | | 0.0000000 | 0.0692730 | 11925.28834 |
| | 0.0000000 | 0.9718793 | 10217.54983 | | 0.0000000 | 0.0692730 | 11946.65943 |
| | 0.0000000 | 0.9718793 | 10230.57425 | | 0.0000001 | 0.0967078 | 12009.23838 |
| | 0.0000000 | 0.9581619 | 10239.94928 | | 0.0000000 | 0.0555556 | 12043.45183 |
| | 0.0000000 | 0.9581619 | 10247.57672 | | 0.0000000 | 0.0144033 | 12063.7875 |
| | 0.0000000 | 0.9718793 | 10262.82559 | | 0.0000000 | 0.0555556 | 12080.17934 |
| | 0.0000000 | 0.9718793 | 10294.56991 | | 0.0000000 | 0.0692730 | 12095.12377 |
| | 0.0000000 | 0.9718793 | 10306.64326 | | 0.0000000 | 0.0692730 | 12101.64446 |
| | 0.0000000 | 0.9581619 | 10324.32336 | | 0.0000000 | 0.0555556 | 12119.25076 |
| | 0.0000000 | 0.9581619 | 10346.11123 | | 0.0000000 | 0.0418381 | 12141.1169 |
| | 0.0000000 | 0.9581619 | 10363.09118 | | 0.0000000 | 0.0281207 | 13517.3522 |
| | 0.0000000 | 0.9581619 | 10383.28509 | | 0.0000001 | 0.0829904 | 13572.89265 |
| | 0.0000000 | 0.9170096 | 10402.18844 | | 0.0000000 | 0.0555556 | 13664.61109 |
| | 0.0000003 | 0.9032922 | 10962.30392 | | 0.0000000 | 0.0555556 | 13743.15877 |
| | 0.0000005 | 0.9032922 | 11057.41916 | | 0.0000000 | 0.0281207 | 14868.45486 |
| | 0.0000001 | 0.0692730 | 11533.00585 | | 0.0000000 | 0.0418381 | 15277.38043 |
| | 0.0000000 | 0.0692730 | 11553.43803 | | 0.0000002 | 0.0967078 | 15477.52932 |
| | 0.0000000 | 0.0555556 | 11596.70327 | F5CSL | 0.0000000 | 0.0287356 | 3439.012926 |
| | 0.0000000 | 0.0555556 | 11607.15919 | | 0.0377842 | 0.6574713 | 6466.54012 |
| | 0.0000000 | 0.0692730 | 11623.79327 | | 0.0001916 | 0.7839080 | 6490.498273 |
| | 0.0000000 | 0.0555556 | 11642.6909 | | 0.0146436 | 0.6701149 | 8845.312346 |
| | 0.0000000 | 0.0418381 | 11662.86838 | | 0.0055263 | 0.7080460 | 8966.56789 |
| | 0.0000000 | 0.0692730 | 11682.71261 | | 0.0091111 | 0.6827586 | 9174.061297 |
| | 0.0000002 | 0.0967078 | 11737.66458 | | 0.0016980 | 0.7333333 | 25619.17312 |
| | 0.0000001 | 0.0829904 | 11758.16873 | | 0.0005779 | 0.7459770 | 33593.85227 |
| | 0.0000001 | 0.0829904 | 11881.99843 | | 0.0018823 | 0.7206897 | 34007.46796 |
| | 0.0000001 | 0.0829904 | 11909.00114 | | 0.0020848 | 0.7206897 | 34545.63707 |
| | 0.0000002 | 0.0967078 | 11918.54028 | | 0.0018823 | 0.7333333 | 34736.3154 |
| | 0.0000001 | 0.0829904 | 11925.28834 | | 0.0021934 | 0.7333333 | 34944.03116 |
| | 0.0000002 | 0.0829904 | 11930.35684 | | 0.0037803 | 0.7206897 | 35450.83994 |
| | 0.0000000 | 0.0555556 | 12043.45183 | | 0.0001504 | 0.2143678 | 47192.29627 |
| | 0.0000000 | 0.0418381 | 12063.7875 | F5CSH | 0.0000004 | 0.9186795 | 10120.52937 |
| | 0.0000000 | 0.0829904 | 12080.17934 | | 0.0000003 | 0.9186795 | 10146.3839 |
| | 0.0000000 | 0.0692730 | 12095.12377 | | 0.0000004 | 0.9186795 | 10162.43733 |
| | 0.0000001 | 0.0829904 | 12101.64446 | | 0.0000004 | 0.9186795 | 10178.08813 |
| | 0.0000000 | 0.0692730 | 12119.25076 | | 0.0000006 | 0.9186795 | 10195.2301 |
| | 0.0000000 | 0.0555556 | 13517.3522 | | 0.0000010 | 0.9186795 | 10205.50105 |
| | 0.0000001 | 0.0967078 | 13572.89265 | | 0.0000011 | 0.9186795 | 10215.263 |
| | 0.0000000 | 0.0555556 | 13743.15877 | | 0.0000009 | 0.9186795 | 10227.99328 |
| | 0.0000000 | 0.0418381 | 14868.45486 | | 0.0000007 | 0.9186795 | 10237.23424 |
| | 0.0000000 | 0.0555556 | 15277.38043 | | 0.0000004 | 0.9186795 | 10254.36842 |
| | 0.0000000 | 0.0829904 | 15477.52932 | | 0.0000002 | 0.9186795 | 10281.47372 |
| | 0.0000000 | 0.0281207 | 75269.22784 | | 0.0000002 | 0.9186795 | 10296.18987 |
| | 0.0000000 | 0.0281207 | 150365.8563 | | 0.0000002 | 0.9186795 | 10308.69901 |
| F5CSL | 0.0000000 | 0.0540230 | 3439.012926 | | 0.0000002 | 0.9186795 | 10341.43948 |
| | 0.0000018 | 0.8597701 | 6689.07332 | | 0.0000002 | 0.9186795 | 10359.22541 |
| | 0.0016980 | 0.7333333 | 8845.312346 | | 0.0000002 | 0.9186795 | 10373.50565 |
| | 0.0001330 | 0.7839080 | 8966.56789 | | 0.0000001 | 0.9347826 | 10388.7689 |
| | 0.0000860 | 0.8091954 | 9174.061297 | | 0.0000001 | 0.9186795 | 10402.74887 |
| | 0.0187682 | 0.6574713 | 9319.311385 | | 0.0000001 | 0.9347826 | 10418.95812 |
| | 0.0000135 | 0.8344828 | 25619.17312 | | 0.0000004 | 0.9186795 | 10433.30772 |
| | 0.0069584 | 0.6954023 | 35450.83994 | | 0.0000002 | 0.9186795 | 10452.21013 |
| | 0.0000004 | 0.9186795 | 10120.52937 | | 0.0000000 | 0.9508857 | 10465.90587 |
| | 0.0000003 | 0.9186795 | 10146.3839 | | 0.0000001 | 0.9347826 | 10481.18415 |
| | 0.0000004 | 0.9186795 | 10162.43733 | | 0.0000002 | 0.9186795 | 10498.6017 |
| | 0.0000003 | 0.9186795 | 10178.08813 | | 0.0000006 | 0.9025765 | 10508.92986 |
| | 0.0000003 | 0.9186795 | 10195.2301 | | 0.0000004 | 0.9186795 | 10514.91432 |
| | 0.0000002 | 0.9186795 | 10205.50105 | | 0.0000004 | 0.9186795 | 10527.20915 |
| | 0.0000005 | 0.9186795 | 10215.263 | | 0.0000006 | 0.9025765 | 10546.16486 |
| | 0.0000005 | 0.9186795 | 10227.99328 | | 0.0000002 | 0.9186795 | 10596.28169 |
| | 0.0000004 | 0.9186795 | 10237.23424 | | 0.0000007 | 0.9025765 | 10616.84987 |
| | 0.0000003 | 0.9186795 | 10254.36842 | | 0.0000007 | 0.9186795 | 10636.82577 |
| | 0.0000002 | 0.9186795 | 10281.47372 | | 0.0000002 | 0.9186795 | 10654.23706 |
| | 0.0000002 | 0.9186795 | 10296.18987 | | 0.0000002 | 0.9186795 | 10665.17962 |
| | 0.0000001 | 0.9186795 | 10308.69901 | | 0.0000016 | 0.9025765 | 10751.55877 |
| | 0.0000002 | 0.9186795 | 10341.43948 | | 0.0000011 | 0.9025765 | 10773.15923 |
| | 0.0000002 | 0.9186795 | 10359.22541 | | 0.0000004 | 0.0813205 | 12027.11641 |
| | 0.0000002 | 0.9186795 | 10373.50565 | | 0.0000003 | 0.0652174 | 12052.53166 |
| | 0.0000001 | 0.9186795 | 10388.7689 | | 0.0000004 | 0.0974235 | 13028.74639 |
| | 0.0000001 | 0.9186795 | 10402.74887 | | 0.0000000 | 0.0491143 | 13065.70027 |
| | 0.0000001 | 0.9347826 | 10418.95812 | | 0.0000000 | 0.0652174 | 13099.66919 |
| | 0.0000002 | 0.9186795 | 10433.30772 | | 0.0000003 | 0.0813205 | 13187.45517 |
| | 0.0000001 | 0.9186795 | 10452.21013 | | 0.0000009 | 0.0974235 | 13233.79106 |
| | 0.0000000 | 0.9508857 | 10465.90587 | | 0.0000002 | 0.0813205 | 13291.70681 |
| | 0.0000000 | 0.9508857 | 10481.18415 | | 0.0000002 | 0.0652174 | 13320.77403 |
| | 0.0000001 | 0.9347826 | 10498.6017 | | 0.0000001 | 0.0652174 | 13356.70436 |
| | 0.0000002 | 0.9186795 | 10508.92986 | | 0.0000000 | 0.0330113 | 14214.24986 |
| | 0.0000001 | 0.9347826 | 10514.91432 | | 0.0000000 | 0.0169082 | 14344.33114 |
| | 0.0000001 | 0.9347826 | 10527.20915 | | 0.0000000 | 0.0491143 | 17653.98849 |
| | 0.0000002 | 0.9186795 | 10546.16486 | | 0.0000001 | 0.0652174 | 45254.93201 |
| | 0.0000001 | 0.9186795 | 10575.10057 | | 0.0000011 | 0.9025765 | 61369.70702 |
| | 0.0000000 | 0.9508857 | 10596.28169 | F6CSL | 0.0000046 | 0.8403576 | 2990.018687 |
| | 0.0000001 | 0.9186795 | 10616.84987 | | 0.0000168 | 0.8122605 | 3360.866902 |
| | 0.0000000 | 0.9347826 | 10636.82577 | | 0.0000050 | 0.8263091 | 4179.401548 |
| | 0.0000000 | 0.9347826 | 10654.23706 | | 0.0000022 | 0.8544061 | 4198.411686 |
| | 0.0000002 | 0.9025765 | 10665.17962 | | 0.0000016 | 0.8684547 | 4252.48155 |
| | 0.0000012 | 0.9025765 | 10694.26283 | | 0.0000260 | 0.8122605 | 4354.864569 |
| | 0.0000003 | 0.0974235 | 11996.5121 | | 0.0000007 | 0.8544061 | 4410.72305 |
| | 0.0000009 | 0.0974235 | 12052.53166 | | 0.0000022 | 0.8544061 | 6487.935491 |
| | 0.0000006 | 0.0974235 | 12974.23339 | | 0.0000019 | 0.8684547 | 6689.164478 |
| | 0.0000000 | 0.0330113 | 13065.70027 | | 0.0000099 | 0.8263091 | 8842.966359 |
| | 0.0000000 | 0.0330113 | 13099.66919 | | 0.0000039 | 0.8544061 | 9046.727666 |
| | 0.0000001 | 0.0652174 | 13187.45517 | | 0.0029193 | 0.7139208 | 9165.408341 |
| | 0.0000000 | 0.0169082 | 13233.79106 | | 0.0001288 | 0.2081737 | 10733.07419 |
| | 0.0000000 | 0.0330113 | 13291.70681 | | 0.0000001 | 0.0881226 | 12480.3404 |
| | 0.0000002 | 0.0813205 | 13320.77403 | | 0.0000000 | 0.0600255 | 12651.6673 |
| | 0.0000000 | 0.0491143 | 13356.70436 | F6CSH | 0.0000000 | 0.9801587 | 10034.41899 |
| | 0.0000007 | 0.0974235 | 13426.83767 | | 0.0000000 | 0.9801587 | 10134.16987 |
| | 0.0000001 | 0.0813205 | 14019.70586 | | 0.0000000 | 0.9801587 | 10164.38262 |
| | 0.0000000 | 0.0169082 | 14214.24986 | | 0.0000000 | 0.9801587 | 10179.25579 |
| | 0.0000000 | 0.0169082 | 14344.33114 | | 0.0000000 | 0.9801587 | 10199.786 |
| | 0.0000000 | 0.0491143 | 17653.98849 | | 0.0000000 | 0.9801587 | 10247.31042 |
| | 0.0000001 | 0.0813205 | 45254.93201 | | 0.0000000 | 0.9801587 | 10308.99693 |
| F6CSL | 0.0000001 | 0.8965517 | 2990.018687 | | 0.0000000 | 0.9801587 | 10324.98028 |
| | 0.0000016 | 0.8544061 | 3360.866902 | | 0.0000000 | 0.9801587 | 10351.28255 |
| | 0.0000002 | 0.8965517 | 4410.72305 | | 0.0000000 | 0.9801587 | 10382.71287 |
| | 0.0000000 | 0.8965517 | 6487.935491 | | 0.0000000 | 0.9801587 | 10437.59021 |
| | 0.0000000 | 0.9386973 | 6689.164478 | | 0.0000000 | 0.9801587 | 10456.27805 |
| | 0.0000000 | 0.9386973 | 8842.966359 | | 0.0000000 | 0.9365079 | 10586.5933 |
| | 0.0000000 | 0.9527458 | 9046.727666 | | 0.0000000 | 0.9656085 | 10639.37916 |
| | 0.0000001 | 0.0740741 | 12480.3404 | | 0.0000000 | 0.9801587 | 10659.7841 |
| | 0.0000001 | 0.0881226 | 12651.6673 | | 0.0000000 | 0.9656085 | 10714.34361 |
| | 0.0000000 | 0.9527458 | 29021.87789 | | 0.0000000 | 0.9365079 | 10736.08599 |
| | 0.0000000 | 0.9246488 | 30269.89108 | | 0.0000001 | 0.9365079 | 10910.64978 |
| F6CSH | 0.0000000 | 0.9814815 | 10034.41899 | | 0.0000000 | 0.0925926 | 11623.61757 |
| | 0.0000000 | 0.9814815 | 10134.16987 | | 0.0000001 | 0.0780423 | 11673.12834 |
| | 0.0000000 | 0.9814815 | 10164.38262 | | 0.0000000 | 0.0343915 | 11750.83857 |
| | 0.0000000 | 0.9814815 | 10179.25579 | | 0.0000000 | 0.0634921 | 11779.63819 |
| | 0.0000000 | 0.9814815 | 10199.786 | | 0.0000000 | 0.0343915 | 11824.74215 |
| | 0.0000000 | 0.9814815 | 10247.31042 | | 0.0000000 | 0.0634921 | 11886.09971 |
| | 0.0000000 | 0.9814815 | 10308.99693 | | 0.0000000 | 0.0343915 | 11982.74557 |
| | 0.0000000 | 0.9814815 | 10324.98028 | | 0.0000000 | 0.0489418 | 12017.01197 |
| | 0.0000000 | 0.9814815 | 10351.28255 | | 0.0000000 | 0.0343915 | 12050.07302 |
| | 0.0000000 | 0.9814815 | 10382.71287 | | 0.0000000 | 0.0198413 | 12170.65112 |
| | 0.0000000 | 0.9814815 | 10437.59021 | | 0.0000000 | 0.0634921 | 12200.58095 |
| | 0.0000000 | 0.9814815 | 10456.27805 | | 0.0000000 | 0.0634921 | 12889.83948 |
| | 0.0000000 | 0.9543210 | 10586.5933 | | 0.0000000 | 0.0198413 | 13157.2016 |
| | 0.0000000 | 0.9679012 | 10639.37916 | | 0.0000000 | 0.0780423 | 13373.91926 |
| | 0.0000000 | 0.9814815 | 10659.7841 | | 0.0000000 | 0.0198413 | 14029.68761 |
| | 0.0000000 | 0.9679012 | 10714.34361 | | 0.0000000 | 0.0198413 | 14160.13964 |
| | 0.0000000 | 0.9679012 | 10736.08599 | | 0.0000000 | 0.0198413 | 14336.01183 |
| | 0.0000000 | 0.9271605 | 10910.64978 | | 0.0000000 | 0.0489418 | 14434.26849 |
| | 0.0000002 | 0.9000000 | 10929.45696 | | 0.0000000 | 0.0198413 | 15420.52874 |
| | 0.0000001 | 0.0907407 | 11623.61757 | | 0.0000002 | 0.9074074 | 16555.54127 |
| | 0.0000000 | 0.0419753 | 11750.83857 | | 0.0000001 | 0.9074074 | 16719.92602 |
| | 0.0000000 | 0.0419753 | 11779.63819 | | 0.0000000 | 0.0634921 | 17530.97848 |
| | 0.0000000 | 0.0283951 | 11824.74215 | | 0.0000000 | 0.0343915 | 17656.32521 |
| | 0.0000000 | 0.0419753 | 11886.09971 | | 0.0000000 | 0.9801587 | 18041.10658 |
| | 0.0000000 | 0.0283951 | 11982.74557 | | 0.0000000 | 0.9365079 | 18241.15243 |
| | 0.0000000 | 0.0827160 | 12017.01197 | | 0.0000004 | 0.0925926 | 33564.57869 |
| | 0.0000000 | 0.0283951 | 12050.07302 | | | | |
| | 0.0000000 | 0.0283951 | 12170.65112 | | | | |
| | 0.0000000 | 0.0555556 | 12200.58095 | | | | |
| | 0.0000000 | 0.0148148 | 13157.2016 | | | | |
| | 0.0000000 | 0.0691358 | 13373.91926 | | | | |
| | 0.0000000 | 0.0283951 | 14029.68761 | | | | |
| | 0.0000000 | 0.0148148 | 14160.13964 | | | | |
| | 0.0000000 | 0.0148148 | 14336.01183 | | | | |
| | 0.0000000 | 0.0283951 | 14434.26849 | | | | |
| | 0.0000000 | 0.0148148 | 15420.52874 | | | | |
| | 0.0000003 | 0.9000000 | 16555.54127 | | | | |
| | 0.0000002 | 0.9000000 | 16719.92602 | | | | |
| | 0.0000000 | 0.0555556 | 17530.97848 | | | | |
| | 0.0000000 | 0.0148148 | 17656.32521 | | | | |
| | 0.0000000 | 0.9814815 | 18041.10658 | | | | |
| | 0.0000000 | 0.9135802 | 18241.15243 | | | | |
| | 0.0000000 | 0.0555556 | 33564.57869 | | | | |
| | 0.0000000 | 0.0962963 | 33807.33449 | | | | |
| | 0.0000000 | 0.0827160 | 44709.3435 | | | | |

TABLE 24
Biomarkers identified by SELDI technology with a p-value smaller or equal to 0.05 that can
discriminate primary DENV from secondary DENV infection. Grouped according to fraction
it was found in.

| | 1DF1_2 vs 2DF1_2 | | | | 1DHF1_2 vs 2DHF1_2 | | |
|---|---|---|---|---|---|---|---|
| | p-value | roc value | m/z average | | p-value | roc value | m/z average |
| | 0.0000000 | 0.9833333 | 2667.845783 | F1CSL | 0.0000011 | 0.9611111 | 2625.181487 |
| | 0.0000000 | 0.9833333 | 2856.727191 | | 0.0000005 | 0.9833333 | 2667.845783 |
| | 0.0000000 | 0.9833333 | 2872.154934 | | 0.0000096 | 0.9388889 | 2741.725854 |
| | 0.0000000 | 0.9666667 | 2921.681011 | | 0.0000008 | 0.9833333 | 2856.727191 |
| | 0.0000003 | 0.9333333 | 2990.831829 | | 0.0000006 | 0.9833333 | 2872.154934 |
| | 0.0000020 | 0.9000000 | 3043.041845 | | 0.0000065 | 0.9472222 | 2897.527715 |
| | 0.0000000 | 0.9833333 | 3145.264181 | | 0.0000019 | 0.9611111 | 2921.681011 |
| | 0.0000000 | 0.9666667 | 3175.340726 | | 0.0000029 | 0.9611111 | 2938.078868 |
| | 0.0000000 | 0.9833333 | 3209.081505 | | 0.0000015 | 0.9611111 | 3175.340726 |
| | 0.0000000 | 0.9833333 | 3262.112346 | | 0.0000038 | 0.9611111 | 3209.081505 |
| | 0.0000001 | 0.9500000 | 3280.760876 | | 0.0000013 | 0.9833333 | 3262.112346 |
| | 0.0000001 | 0.9500000 | 3307.659966 | | 0.0000011 | 0.9611111 | 3280.760876 |
| | 0.0000010 | 0.9166667 | 3358.521584 | | 0.0000124 | 0.9166667 | 3307.659966 |
| | 0.0000000 | 0.9833333 | 3420.037403 | | 0.0004513 | 0.8444444 | 3358.521584 |
| | 0.0000000 | 0.9833333 | 3437.42944 | | 0.0000074 | 0.9166667 | 3420.037403 |
| | 0.0000003 | 0.9333333 | 3459.797001 | | 0.0000084 | 0.9166667 | 3437.42944 |
| | 0.0000000 | 0.9833333 | 3511.538012 | | 0.0000038 | 0.9611111 | 3459.786786 |
| | 0.0000000 | 0.9833333 | 3589.060244 | | 0.0000038 | 0.9611111 | 3459.797001 |
| | 0.0000000 | 0.9833333 | 3631.061336 | | 0.0000010 | 0.9611111 | 3511.538012 |
| | 0.0000001 | 0.9333333 | 3680.115892 | | 0.0000013 | 0.9833333 | 3589.060244 |
| | 0.0000000 | 0.9666667 | 3799.639473 | | 0.0000033 | 0.9611111 | 3680.115892 |
| | 0.0000000 | 0.9833333 | 3814.303638 | | 0.0000029 | 0.9833333 | 3799.639473 |
| | 0.0000000 | 0.9833333 | 3863.107595 | | 0.0000006 | 0.9833333 | 3814.303638 |
| | 0.0000000 | 0.9666667 | 3884.602302 | | 0.0000015 | 0.9833333 | 3863.107595 |
| | 0.0000001 | 0.9500000 | 3923.916704 | | 0.0000232 | 0.9166667 | 3923.916704 |
| | 0.0000010 | 0.9166667 | 3949.327603 | | 0.0000007 | 0.9833333 | 4063.241745 |
| | 0.0000001 | 0.9666667 | 4063.241745 | | 0.0000159 | 0.9166667 | 4182.768879 |
| | 0.0000003 | 0.9333333 | 4143.916106 | | 0.0000006 | 0.9833333 | 4299.724384 |
| | 0.0000003 | 0.9333333 | 4182.768879 | | 0.0000084 | 0.9388889 | 4417.386857 |
| | 0.0000001 | 0.9666667 | 4279.033423 | | 0.0000038 | 0.9388889 | 4468.900331 |
| | 0.0000000 | 0.9666667 | 4299.724384 | | 0.0000043 | 0.9611111 | 4492.301874 |
| | 0.0000001 | 0.9333333 | 4417.386857 | | 0.0000065 | 0.9611111 | 4524.063737 |
| | 0.0000002 | 0.9333333 | 4435.802022 | | 0.0000050 | 0.9472222 | 4646.271132 |
| | 0.0000003 | 0.9333333 | 4450.645147 | | 0.0000043 | 0.0444444 | 5675.016336 |
| | 0.0000000 | 0.9500000 | 4468.900331 | | 0.0000013 | 0.0222222 | 5689.765089 |
| | 0.0000001 | 0.9500000 | 4492.301874 | | 0.0004513 | 0.1333333 | 6013.779325 |
| | 0.0000001 | 0.9388889 | 4524.063737 | | 0.0000232 | 0.0888889 | 7415.948421 |
| | 0.0000013 | 0.9166667 | 4573.192933 | | 0.0000074 | 0.0666667 | 7484.032788 |
| | 0.0000018 | 0.9166667 | 4588.379469 | | 0.0000022 | 0.0666667 | 7495.290949 |
| | 0.0000001 | 0.9388889 | 4646.271132 | | 0.0000480 | 0.9166667 | 8459.519265 |
| | 0.0000028 | 0.1000000 | 5689.765089 | | 0.0000019 | 0.0222222 | 10289.18681 |
| | 0.0000013 | 0.0944444 | 5767.809439 | | 0.0000232 | 0.0888889 | 11743.17361 |
| | 0.0000023 | 0.1000000 | 6487.509564 | | 0.0000140 | 0.0888889 | 11902.07203 |
| | 0.0000002 | 0.0666667 | 6591.475901 | | 0.0000480 | 0.0888889 | 12083.60453 |
| | 0.0000052 | 0.1000000 | 6642.439305 | | 0.0000029 | 0.0666667 | 13375.41938 |
| | 0.0000005 | 0.0833333 | 6652.107044 | | 0.0000096 | 0.0666667 | 14757.7248 |
| | 0.0000001 | 0.0500000 | 6683.82645 | | 0.0000038 | 0.0444444 | 44810.68881 |
| | 0.0000001 | 0.0500000 | 6805.698908 | | 0.0000065 | 0.0444444 | 45184.10294 |
| | 0.0000001 | 0.0333333 | 6860.453871 | | 0.0000074 | 0.0444444 | 45616.26741 |
| | 0.0000004 | 0.0833333 | 7415.948421 | | 0.0000084 | 0.0666667 | 46357.92645 |
| | 0.0000003 | 0.0833333 | 7484.032788 | | 0.0008932 | 0.2277778 | 10130.28134 |
| | 0.0000001 | 0.0333333 | 7495.290949 | | 0.0000570 | 0.1444444 | 10171.11128 |
| | 0.0000017 | 0.1000000 | 7865.986549 | | 0.0000140 | 0.1277778 | 10196.44234 |
| | 0.0000002 | 0.0666667 | 11491.70855 | | 0.0000063 | 0.1111111 | 10351.51024 |
| | 0.0000001 | 0.0666667 | 11677.83181 | | 0.0000017 | 0.0944444 | 10369.2482 |
| | 0.0000001 | 0.0666667 | 11743.17361 | | 0.0000007 | 0.0777778 | 10413.34138 |
| | 0.0000002 | 0.0666667 | 11902.07203 | | 0.0000009 | 0.0777778 | 10444.68912 |
| | 0.0000005 | 0.0833333 | 12590.76335 | | 0.0000010 | 0.0777778 | 10461.79685 |
| | 0.0000007 | 0.0833333 | 13115.34777 | | 0.0000011 | 0.0944444 | 10481.71491 |
| | 0.0000000 | 0.0166667 | 13375.41938 | | 0.0000008 | 0.0777778 | 10490.47668 |
| | 0.0000008 | 0.0833333 | 14757.7248 | | 0.0000012 | 0.0944444 | 10507.40716 |
| | 0.0000023 | 0.0833333 | 15198.38517 | | 0.0000003 | 0.9333333 | 10755.48072 |
| | 0.0000047 | 0.1000000 | 22937.26034 | | 0.0000001 | 0.9555556 | 10783.37546 |
| | 0.0000012 | 0.0666667 | 23544.76704 | | 0.0000001 | 0.9555556 | 10808.95097 |
| | 0.0000011 | 0.9222222 | 35405.0346 | | 0.0000001 | 0.9666667 | 10835.54836 |
| | 0.0000001 | 0.0333333 | 44810.68881 | | 0.0000001 | 0.9555556 | 10882.15141 |
| | 0.0000001 | 0.0333333 | 45184.10294 | | 0.0000000 | 0.9833333 | 10908.0378 |
| | 0.0000001 | 0.0333333 | 45616.26741 | | 0.0000000 | 0.9833333 | 10926.28045 |
| | 0.0000001 | 0.0333333 | 46357.92645 | | 0.0000000 | 0.9833333 | 10951.30804 |
| | 0.0270506 | 0.3138889 | 10130.28134 | | 0.0000007 | 0.0833333 | 11383.78669 |
| | 0.0020619 | 0.2625000 | 10171.11128 | | 0.0000000 | 0.9666667 | 11823.52687 |
| | 0.0016282 | 0.2666667 | 10196.44234 | | 0.0000001 | 0.9666667 | 11853.93279 |
| | 0.0003589 | 0.2250000 | 10241.85866 | | 0.0000000 | 0.9833333 | 12009.62084 |
| | 0.0000001 | 0.0861111 | 10490.47668 | | 0.0000000 | 0.9833333 | 12080.71023 |
| | 0.0000001 | 0.0722222 | 10507.40716 | | 0.0000000 | 0.9833333 | 12112.68049 |
| | 0.0000002 | 0.1000000 | 10514.61735 | | 0.0000000 | 0.9833333 | 12132.64107 |
| | 0.0000001 | 0.9166667 | 10808.95097 | | 0.0000000 | 0.9500000 | 12163.0594 |
| | 0.0000000 | 0.9444444 | 10835.54836 | | 0.0000000 | 0.9833333 | 12268.93847 |
| | 0.0000001 | 0.9305556 | 10882.15141 | | 0.0000001 | 0.9500000 | 12358.50727 |
| | 0.0000001 | 0.9027778 | 10908.0378 | | 0.0000000 | 0.9833333 | 12493.48295 |
| | 0.0000000 | 0.9305556 | 10926.28045 | | 0.0000000 | 0.0333333 | 12954.81113 |
| | 0.0000000 | 0.9861111 | 10951.30804 | | 0.0000013 | 0.1000000 | 13063.59409 |
| | 0.0000005 | 0.9027778 | 11078.55501 | | 0.0000001 | 0.0500000 | 13086.15535 |
| | 0.0000003 | 0.9027778 | 11725.21956 | | 0.0000000 | 0.9833333 | 13930.38141 |
| | 0.0000002 | 0.9027778 | 11823.52687 | | 0.0000003 | 0.9333333 | 14140.38344 |
| | 0.0000000 | 0.9305556 | 11853.93279 | | 0.0000001 | 0.0333333 | 14449.85539 |
| | 0.0000000 | 0.9722222 | 12009.62084 | | 0.0000002 | 0.0666667 | 14639.78045 |
| | 0.0000000 | 0.9861111 | 12080.71023 | | 0.0000000 | 0.0166667 | 14934.23152 |
| | 0.0000000 | 0.9583333 | 12112.68049 | | 0.0000002 | 0.9333333 | 100409.516 |
| | 0.0000000 | 0.9861111 | 12132.64107 | | 0.0000018 | 0.9166667 | 101137.8334 |
| | 0.0000000 | 0.9861111 | 12163.0594 | | 0.0000002 | 0.9500000 | 102198.3507 |
| | 0.0000000 | 0.9861111 | 12268.93847 | | 0.0000011 | 0.9166667 | 107068.7099 |
| | 0.0000000 | 0.9861111 | 12358.50727 | | 0.0000002 | 0.9500000 | 113333.4714 |
| | 0.0000000 | 0.9861111 | 12493.48295 | | 0.0000004 | 0.9333333 | 174369.2245 |
| | 0.0000001 | 0.9069444 | 12562.71997 | | 0.0000000 | 0.9666667 | 192541.7062 |
| | 0.0000000 | 0.9583333 | 13930.38141 | F1ISL | 0.0000003 | 0.0400000 | 2503.154582 |
| | 0.0000000 | 0.9583333 | 14140.38344 | | 0.0000010 | 0.0600000 | 2513.073032 |
| | 0.0000006 | 0.9027778 | 192541.7062 | | 0.0000053 | 0.1000000 | 2517.656451 |
| F1ISL | 0.0000000 | 0.0166667 | 2503.154582 | | 0.0000060 | 0.0800000 | 2522.859861 |
| | 0.0000005 | 0.1000000 | 2534.62918 | | 0.0000012 | 0.0600000 | 2523.669165 |
| | 0.0000000 | 0.0722222 | 2565.111611 | | 0.0000007 | 0.0600000 | 2524.520651 |
| | 0.0000001 | 0.0861111 | 2595.854473 | | 0.0000030 | 0.0800000 | 2549.242272 |
| | 0.0000000 | 0.0583333 | 2601.511801 | | 0.0000067 | 0.1000000 | 2565.111611 |
| | 0.0000002 | 0.0861111 | 2614.032211 | | 0.0000008 | 0.0600000 | 2570.087202 |
| | 0.0000000 | 0.0444444 | 2617.583867 | | 0.0000021 | 0.0800000 | 2595.854473 |
| | 0.0000004 | 0.1000000 | 2621.696749 | | 0.0000053 | 0.1000000 | 2601.511801 |
| | 0.0000001 | 0.0722222 | 2634.20847 | | 0.0000005 | 0.0400000 | 2614.032211 |
| | 0.0000001 | 0.0861111 | 2635.700585 | | 0.0000006 | 0.0400000 | 2617.583867 |
| | 0.0000000 | 0.0166667 | 2637.169955 | | 0.0000013 | 0.0600000 | 2621.696749 |
| | 0.0000000 | 0.0444444 | 2638.060903 | | 0.0000024 | 0.0800000 | 2634.20847 |
| | 0.0000000 | 0.0444444 | 2639.173428 | | 0.0000005 | 0.0400000 | 2635.700585 |
| | 0.0000000 | 0.0166667 | 2641.926515 | | 0.0000003 | 0.0400000 | 2637.169955 |
| | 0.0000000 | 0.0166667 | 2642.885878 | | 0.0000003 | 0.0400000 | 2637.618569 |
| | 0.0000000 | 0.0444444 | 2643.949918 | | 0.0000004 | 0.0400000 | 2638.060903 |
| | 0.0000000 | 0.0583333 | 2658.967508 | | 0.0000004 | 0.0400000 | 2638.54484 |
| | 0.0000000 | 0.0444444 | 2659.814331 | | 0.0000001 | 0.0200000 | 2639.173428 |
| | 0.0000000 | 0.0166667 | 2661.84737 | | 0.0000001 | 0.0200000 | 2639.795493 |
| | 0.0000000 | 0.0166667 | 2662.858635 | | 0.0000001 | 0.0200000 | 2640.467576 |
| | 0.0000000 | 0.0305556 | 2666.570765 | | 0.0000001 | 0.0200000 | 2641.164374 |
| | 0.0000000 | 0.0861111 | 2674.971433 | | 0.0000001 | 0.0200000 | 2641.926515 |
| | 0.0000000 | 0.0583333 | 2679.146726 | | 0.0000001 | 0.0200000 | 2642.885878 |
| | 0.0000000 | 0.0166667 | 2681.308781 | | 0.0000030 | 0.0600000 | 2643.949918 |
| | 0.0000000 | 0.0166667 | 2684.692433 | | 0.0000053 | 0.1000000 | 2646.227988 |
| | 0.0000000 | 0.0166667 | 2686.45453 | | 0.0000005 | 0.0400000 | 2657.390501 |
| | 0.0000000 | 0.0166667 | 2704.270371 | | 0.0000105 | 0.1000000 | 2658.351659 |
| | 0.0000000 | 0.0305556 | 2709.71622 | | 0.0000013 | 0.0600000 | 2658.967508 |
| | 0.0000000 | 0.0583333 | 2726.112884 | | 0.0000010 | 0.0600000 | 2659.814331 |
| | 0.0000004 | 0.1000000 | 2738.579429 | | 0.0000053 | 0.0800000 | 2661.194232 |
| | 0.0000001 | 0.0861111 | 2749.363653 | | 0.0000007 | 0.0600000 | 2661.84737 |
| | 0.0000000 | 0.0166667 | 2752.680443 | | 0.0000015 | 0.0800000 | 2662.858635 |
| | 0.0000000 | 0.0166667 | 2753.648132 | | 0.0000009 | 0.0600000 | 2666.570765 |
| | 0.0000000 | 0.0722222 | 2772.763638 | | 0.0000021 | 0.0600000 | 2679.146726 |
| | 0.0000000 | 0.0305556 | 2775.410216 | | 0.0000001 | 0.0200000 | 2681.308781 |
| | 0.0000001 | 0.0722222 | 2796.034541 | | 0.0000021 | 0.0600000 | 2684.692433 |
| | 0.0000002 | 0.0861111 | 2803.236487 | | 0.0000003 | 0.0600000 | 2686.45453 |
| | 0.0000000 | 0.0166667 | 2811.436125 | | 0.0000002 | 0.0400000 | 2704.270371 |
| | 0.0000000 | 0.0583333 | 2817.188037 | | 0.0000004 | 0.0600000 | 2709.71622 |
| | 0.0000000 | 0.0166667 | 2824.989455 | | 0.0000131 | 0.1000000 | 2738.579429 |
| | 0.0000010 | 0.1000000 | 2866.751136 | | 0.0000002 | 0.0400000 | 2749.363653 |
| | 0.0000000 | 0.0305556 | 2879.703192 | | 0.0000053 | 0.0800000 | 2752.680443 |
| | 0.0000000 | 0.0166667 | 2883.890105 | | 0.0000182 | 0.1000000 | 2753.648132 |
| | 0.0000000 | 0.0166667 | 2887.453423 | | 0.0000182 | 0.1000000 | 2753.857675 |
| | 0.0000000 | 0.0583333 | 2894.062647 | | 0.0000182 | 0.1000000 | 2753.946851 |
| | 0.0000000 | 0.0583333 | 2904.031336 | | 0.0000005 | 0.0400000 | 2755.192724 |
| | 0.0000000 | 0.0444444 | 2906.91955 | | 0.0000008 | 0.0600000 | 2772.763638 |
| | 0.0000000 | 0.0583333 | 2908.899563 | | 0.0000021 | 0.0800000 | 2788.581536 |
| | 0.0000000 | 0.0166667 | 2911.83993 | | 0.0000030 | 0.0800000 | 2791.185756 |
| | 0.0000001 | 0.0861111 | 2915.763212 | | 0.0000024 | 0.0800000 | 2796.034541 |
| | 0.0000001 | 0.0722222 | 2930.543416 | | 0.0000024 | 0.0800000 | 2803.236487 |
| | 0.0000000 | 0.0583333 | 2933.649099 | | 0.0000003 | 0.0400000 | 2811.436125 |
| | 0.0000000 | 0.0583333 | 2948.917414 | | 0.0000038 | 0.0600000 | 2817.188037 |
| | 0.0000000 | 0.0305556 | 2951.608271 | | 0.0000001 | 0.0200000 | 2819.017882 |
| | 0.0000000 | 0.0166667 | 2953.403838 | | 0.0000021 | 0.0600000 | 2824.989455 |
| | 0.0000000 | 0.0166667 | 2956.963771 | | 0.0000012 | 0.0600000 | 2883.890105 |
| | 0.0000000 | 0.0444444 | 2976.912269 | | 0.0000034 | 0.0800000 | 2887.453423 |
| | 0.0000000 | 0.0444444 | 2993.494244 | | 0.0000002 | 0.0200000 | 2933.649099 |
| | 0.0000000 | 0.0166667 | 3015.428516 | | 0.0000012 | 0.0600000 | 2948.917414 |
| | 0.0000000 | 0.0444444 | 3020.653111 | | 0.0000005 | 0.0400000 | 2951.608271 |
| | 0.0000003 | 0.0861111 | 3042.639369 | | 0.0000053 | 0.0800000 | 2953.403838 |
| | 0.0000000 | 0.0583333 | 3066.155713 | | 0.0000024 | 0.0800000 | 2976.912269 |
| | 0.0000000 | 0.0583333 | 3083.362119 | | 0.0000048 | 0.0800000 | 2993.494244 |
| | 0.0000000 | 0.0583333 | 3092.389989 | | 0.0000003 | 0.0400000 | 2997.743006 |
| | 0.0000000 | 0.0444444 | 3113.412549 | | 0.0000146 | 0.1000000 | 3015.428516 |
| | 0.0000001 | 0.1000000 | 3149.522222 | | 0.0000034 | 0.1000000 | 3066.155713 |
| | 0.0000000 | 0.0305556 | 3158.254319 | | 0.0000006 | 0.0800000 | 3092.389989 |
| | 0.0000000 | 0.0305556 | 3177.822062 | | 0.0000005 | 0.0600000 | 3129.459825 |
| | 0.0000001 | 0.0861111 | 3213.068347 | | 0.0000010 | 0.0800000 | 3158.254319 |
| | 0.0000000 | 0.0305556 | 3221.413524 | | 0.0000043 | 0.1000000 | 3197.777988 |
| | 0.0000004 | 0.1000000 | 3235.296597 | | 0.0000053 | 0.1000000 | 3213.068347 |
| | 0.0000003 | 0.1000000 | 3314.079782 | | 0.0000060 | 0.1000000 | 3221.413524 |
| | 0.0000000 | 0.0444444 | 3318.297755 | | 0.0000034 | 0.1000000 | 3235.296597 |
| | 0.0000000 | 0.0722222 | 3324.2993 | | 0.0000009 | 0.0600000 | 3276.379562 |
| | 0.0000001 | 0.1000000 | 3397.259967 | | 0.0000251 | 0.1000000 | 3285.606735 |
| | 0.0000000 | 0.0444444 | 3468.46665 | | 0.0000060 | 0.0800000 | 3290.515874 |
| | 0.0000000 | 0.0444444 | 3515.357908 | | 0.0000006 | 0.0600000 | 3315.115414 |
| | 0.0000000 | 0.0444444 | 3528.345658 | | 0.0000015 | 0.0600000 | 3315.909084 |
| | 0.0000003 | 0.0861111 | 3536.763944 | | 0.0000024 | 0.0800000 | 3316.53443 |
| | 0.0000002 | 0.1000000 | 3588.453952 | | 0.0000001 | 0.0200000 | 3318.297755 |
| | 0.0000003 | 0.1000000 | 3604.347348 | | 0.0000251 | 0.1000000 | 3321.260411 |
| | 0.0000000 | 0.0583333 | 3644.486854 | | 0.0000038 | 0.0800000 | 3324.2993 |
| | 0.0000000 | 0.0583333 | 3687.780782 | | 0.0000048 | 0.1000000 | 3369.373189 |
| | 0.0000000 | 0.0583333 | 3699.672565 | | 0.0000013 | 0.0600000 | 3397.259967 |
| | 0.0000009 | 0.1000000 | 3739.751205 | | 0.0000004 | 0.0400000 | 3445.720567 |
| | 0.0000000 | 0.0305556 | 3772.107649 | | 0.0000004 | 0.0600000 | 3468.46665 |
| | 0.0000000 | 0.0305556 | 3827.712827 | | 0.0000004 | 0.0600000 | 3515.357908 |
| | 0.0000000 | 0.0444444 | 3891.091076 | | 0.0000118 | 0.1000000 | 3528.345658 |
| | 0.0000003 | 0.1000000 | 48592.66902 | | 0.0000038 | 0.1000000 | 3536.763944 |
| F1ISH | 0.0000000 | 0.0129630 | 10011.99352 | | 0.0000008 | 0.0600000 | 3644.486854 |
| | 0.0000000 | 0.0129630 | 10020.52117 | | 0.0000021 | 0.0800000 | 3670.116515 |
| | 0.0000000 | 0.0129630 | 10025.43594 | | 0.0000021 | 0.0600000 | 3687.780782 |
| | 0.0000000 | 0.0129630 | 10028.64467 | | 0.0000251 | 0.1000000 | 3699.672565 |
| | 0.0000000 | 0.0129630 | 10036.97216 | | 0.0000038 | 0.0800000 | 3772.107649 |
| | 0.0000000 | 0.0129630 | 10051.63047 | | 0.0000009 | 0.0600000 | 3972.21957 |
| | 0.0000000 | 0.0129630 | 10061.98167 | | 0.0000311 | 0.1000000 | 4301.196114 |
| | 0.0000000 | 0.0129630 | 10072.44679 | F1ISH | 0.0000008 | 0.0154321 | 10011.99352 |
| | 0.0000000 | 0.0129630 | 10081.58862 | | 0.0000008 | 0.0154321 | 10020.52117 |
| | 0.0000000 | 0.0129630 | 10090.2363 | | 0.0000008 | 0.0154321 | 10025.43594 |
| | 0.0000000 | 0.0129630 | 10098.45601 | | 0.0000008 | 0.0154321 | 10028.64467 |
| | 0.0000000 | 0.0129630 | 10105.92173 | | 0.0000008 | 0.0154321 | 10036.97216 |
| | 0.0000000 | 0.0129630 | 10114.78187 | | 0.0000008 | 0.0154321 | 10051.63047 |
| | 0.0000000 | 0.0129630 | 10135.6053 | | 0.0000008 | 0.0154321 | 10061.98167 |
| | 0.0000000 | 0.0129630 | 10147.36272 | | 0.0000008 | 0.0154321 | 10072.44679 |
| | 0.0000000 | 0.0296296 | 10162.00816 | | 0.0000008 | 0.0154321 | 10081.58862 |
| | 0.0000000 | 0.0296296 | 10175.58746 | | 0.0000008 | 0.0154321 | 10090.2363 |
| | 0.0000000 | 0.0296296 | 10187.91964 | | 0.0000010 | 0.0154321 | 10098.45601 |
| | 0.0000000 | 0.0462963 | 10198.46953 | | 0.0000015 | 0.0154321 | 10105.92173 |
| | 0.0000000 | 0.0462963 | 10208.17418 | | 0.0000010 | 0.0154321 | 10114.78187 |
| | 0.0000000 | 0.0462963 | 10217.54983 | | 0.0000010 | 0.0154321 | 10135.6053 |
| | 0.0000000 | 0.0462963 | 10230.57425 | | 0.0000021 | 0.0154321 | 10147.36272 |
| | 0.0000004 | 0.0629630 | 10239.94928 | | 0.0000018 | 0.0154321 | 10162.00816 |
| | 0.0000002 | 0.0796296 | 10262.82559 | | 0.0000032 | 0.0370370 | 10175.58746 |
| | 0.0000001 | 0.0462963 | 10294.56991 | | 0.0000077 | 0.0586420 | 10187.91964 |
| | 0.0000000 | 0.0462963 | 10306.64326 | | 0.0000118 | 0.0802469 | 10198.46953 |
| | 0.0000001 | 0.0462963 | 10324.32336 | | 0.0000067 | 0.0586420 | 10208.17418 |
| | 0.0000002 | 0.0629630 | 10346.11123 | | 0.0000089 | 0.0586420 | 10217.54983 |
| | 0.0000001 | 0.0629630 | 10363.09118 | | 0.0000399 | 0.0802469 | 10521.88611 |
| | 0.0000001 | 0.0462963 | 10383.28509 | | 0.0000350 | 0.0586420 | 11087.90879 |
| | 0.0000002 | 0.0629630 | 10402.18844 | | 0.0000021 | 0.0370370 | 11105.68287 |
| | 0.0000007 | 0.0796296 | 10408.85968 | | 0.0000118 | 0.0586420 | 11118.40053 |
| | 0.0000003 | 0.0629630 | 10414.59628 | | 0.0000089 | 0.9475309 | 11508.64295 |
| | 0.0000007 | 0.0962963 | 10425.2516 | | 0.0000067 | 0.9521605 | 11533.00585 |
| | 0.0000076 | 0.0962963 | 10434.47733 | | 0.0000021 | 0.9691358 | 11553.43803 |
| | 0.0000025 | 0.0962963 | 10521.88611 | | 0.0000037 | 0.9691358 | 11576.23632 |
| | 0.0000007 | 0.0796296 | 10890.43701 | | 0.0000118 | 0.9475309 | 11596.70327 |
| | 0.0000056 | 0.0962963 | 10909.77131 | | 0.0000032 | 0.9475309 | 11607.15919 |
| | 0.0000008 | 0.0796296 | 10994.43525 | | 0.0000015 | 0.9907407 | 11623.79327 |
| | 0.0000004 | 0.0629630 | 11014.94996 | | 0.0000011 | 0.9907407 | 11642.6909 |
| | 0.0000001 | 0.0462963 | 11035.719 | | 0.0000011 | 0.9907407 | 11662.86838 |
| | 0.0000001 | 0.0462963 | 11046.66769 | | 0.0000018 | 0.9907407 | 11682.71261 |
| | 0.0000002 | 0.0796296 | 11057.41916 | | 0.0000455 | 0.9043210 | 11810.11077 |
| | 0.0000000 | 0.0462963 | 11071.04109 | | 0.0000028 | 0.9691358 | 11849.00542 |
| | 0.0000000 | 0.0129630 | 11087.90879 | | 0.0000032 | 0.9691358 | 11881.99843 |
| | 0.0000000 | 0.0296296 | 11105.68287 | | 0.0000032 | 0.9691358 | 11909.00114 |
| | 0.0000000 | 0.0296296 | 11118.40053 | | 0.0000032 | 0.9691358 | 11918.54028 |
| | 0.0000002 | 0.0462963 | 11123.52137 | | 0.0000043 | 0.9691358 | 11925.28834 |
| | 0.0000000 | 0.0296296 | 11130.42377 | | 0.0000178 | 0.9259259 | 11930.35684 |
| | 0.0000003 | 0.0796296 | 11147.3563 | | 0.0000155 | 0.9043210 | 11946.65943 |
| | 0.0000003 | 0.0796296 | 11163.91984 | | 0.0000178 | 0.9089506 | 12009.23838 |
| | 0.0000001 | 0.0462963 | 11178.66084 | | 0.0000204 | 0.9259259 | 12043.45183 |
| | 0.0000001 | 0.0629630 | 11191.97145 | | 0.0000018 | 0.9907407 | 12063.7875 |
| | 0.0000003 | 0.0962963 | 11212.36119 | | 0.0000067 | 0.9475309 | 12080.17934 |
| | 0.0000000 | 0.0462963 | 11226.91495 | | 0.0000102 | 0.0586420 | 12526.96894 |
| | 0.0000016 | 0.0962963 | 11244.4887 | | 0.0000037 | 0.0370370 | 12586.1786 |
| | 0.0000000 | 0.9666667 | 11533.00585 | | 0.0000204 | 0.0802469 | 12616.77768 |
| | 0.0000000 | 0.9500000 | 11553.43803 | | 0.0000008 | 0.9907407 | 13517.3522 |
| | 0.0000005 | 0.9231481 | 11576.23632 | | 0.0000050 | 0.9475309 | 13572.89265 |
| | 0.0000001 | 0.9500000 | 11596.70327 | | 0.0000178 | 0.9043210 | 13664.61109 |
| | 0.0000002 | 0.9333333 | 11607.15919 | | 0.0000018 | 0.9691358 | 13743.15877 |
| | 0.0000001 | 0.9333333 | 11623.79327 | | 0.0000234 | 0.9259259 | 14868.45486 |
| | 0.0000000 | 0.9666667 | 11642.6909 | | 0.0000008 | 0.9907407 | 15277.38043 |
| | 0.0000000 | 0.9666667 | 11662.86838 | | 0.0000268 | 0.9259259 | 23238.98573 |
| | 0.0000000 | 0.9500000 | 11682.71261 | | 0.0000043 | 0.9475309 | 23638.15252 |
| | 0.0000004 | 0.9166667 | 11706.67327 | F5CSL | 0.0000009 | 0.9176245 | 3439.012926 |
| | 0.0000004 | 0.9166667 | 11737.66458 | | 0.0302687 | 0.3103448 | 6466.54012 |
| | 0.0000002 | 0.9166667 | 11758.16873 | | 0.0003051 | 0.1724138 | 6490.498273 |
| | 0.0000004 | 0.9166667 | 11881.99843 | | 0.0000427 | 0.1551724 | 6661.944 |
| | 0.0000004 | 0.9166667 | 11909.00114 | | 0.0000009 | 0.1034483 | 6689.07332 |
| | 0.0000004 | 0.9231481 | 11925.28834 | | 0.0003319 | 0.2068966 | 6863.56499 |
| | 0.0000003 | 0.9231481 | 11930.35684 | | 0.0036071 | 0.2586207 | 33593.85227 |
| | 0.0000002 | 0.9333333 | 12043.45183 | | 0.0050912 | 0.2758621 | 34007.46796 |
| | 0.0000001 | 0.9333333 | 12063.7875 | | 0.0000037 | 0.0706522 | 10146.3839 |
| | 0.0000002 | 0.9333333 | 12080.17934 | | 0.0000074 | 0.0706522 | 10162.43733 |
| | 0.0000002 | 0.9333333 | 12095.12377 | | 0.0000064 | 0.0706522 | 10178.08813 |
| | 0.0000003 | 0.9166667 | 12101.64446 | | 0.0000306 | 0.0896739 | 10215.263 |
| | 0.0000000 | 0.9833333 | 13517.3522 | | 0.0000125 | 0.0896739 | 10254.36842 |
| | 0.0000002 | 0.9333333 | 13572.89265 | | 0.0000006 | 0.0135870 | 10281.47372 |
| | 0.0000000 | 1.0000000 | 13743.15877 | | 0.0000005 | 0.0326087 | 10296.18987 |
| | 0.0000007 | 0.9166667 | 14868.45486 | | 0.0000012 | 0.0706522 | 10308.69901 |
| | 0.0000000 | 0.9666667 | 15277.38043 | | 0.0000006 | 0.0326087 | 10341.43948 |
| | 0.0000001 | 0.9666667 | 75269.22784 | | 0.0000006 | 0.0326087 | 10359.22541 |
| | 0.0000002 | 0.9333333 | 150365.8563 | | 0.0000005 | 0.0135870 | 10373.50565 |
| F5CSL | 0.0000006 | 0.8979885 | 3439.012926 | | 0.0000010 | 0.0516304 | 10388.7689 |
| | 0.0000627 | 0.1896552 | 6490.498273 | | 0.0000021 | 0.0516304 | 10402.74887 |
| | 0.0000499 | 0.1896552 | 6661.944 | | 0.0000043 | 0.0516304 | 10418.95812 |
| | 0.0000000 | 0.0603448 | 6689.07332 | | 0.0000096 | 0.0896739 | 10433.30772 |
| | 0.0000102 | 0.1321839 | 6863.56499 | | 0.0000185 | 0.0896739 | 10452.21013 |
| | 0.0000034 | 0.1321839 | 25619.17312 | | 0.0000096 | 0.0706522 | 10465.90587 |
| | 0.0062592 | 0.3045977 | 34736.3154 | | 0.0000125 | 0.0896739 | 10481.18415 |
| | 0.0000009 | 0.0731225 | 10281.47372 | | 0.0000270 | 0.0896739 | 10498.6017 |
| | 0.0000007 | 0.0731225 | 10296.18987 | | 0.0000096 | 0.0706522 | 10906.58841 |
| | 0.0000007 | 0.0909091 | 10308.69901 | | 0.0000162 | 0.0896739 | 10927.60682 |
| | 0.0000005 | 0.0909091 | 10341.43948 | | 0.0000008 | 0.9483696 | 12052.53166 |
| | 0.0000001 | 0.0553360 | 10359.22541 | | 0.0000110 | 0.9144022 | 14019.70586 |
| | 0.0000001 | 0.0553360 | 10373.50565 | | 0.0000010 | 0.9524457 | 14214.24986 |
| | 0.0000007 | 0.0909091 | 10388.7689 | | 0.0000005 | 0.9673913 | 14344.33114 |
| | 0.0000003 | 0.0731225 | 10402.74887 | | 0.0000001 | 0.9864130 | 17653.98849 |
| | 0.0000002 | 0.0731225 | 10418.95812 | | 0.0000005 | 0.9483696 | 17809.61765 |
| | 0.0000002 | 0.0731225 | 10433.30772 | | 0.0000059 | 0.0926724 | 3360.866902 |
| | 0.0000001 | 0.0553360 | 10452.21013 | | 0.0000115 | 0.0926724 | 4198.411686 |
| | 0.0000001 | 0.0731225 | 10465.90587 | | 0.0104430 | 0.7327586 | 5014.908804 |
| | 0.0000002 | 0.0553360 | 10481.18415 | | 0.0166282 | 0.6939655 | 5017.339281 |
| | 0.0000005 | 0.0731225 | 10508.92986 | | 0.0051433 | 0.2284483 | 6487.935491 |
| | 0.0000011 | 0.0909091 | 10575.10057 | | 0.0000410 | 0.1508621 | 6689.164478 |
| | 0.0000006 | 0.9268775 | 12052.53166 | | 0.0007600 | 0.7909483 | 10733.07419 |
| | 0.0000040 | 0.8913043 | 13356.70436 | | 0.0000679 | 0.1314655 | 12910.82457 |
| | 0.0000000 | 0.9802372 | 14019.70586 | | 0.0000042 | 0.0926724 | 17426.66866 |
| | 0.0000000 | 0.9624506 | 14214.24986 | | 0.0000074 | 0.1120690 | 18048.90648 |
| | 0.0000000 | 0.9802372 | 14344.33114 | | 0.0000093 | 0.0926724 | 30269.89108 |
| | 0.0000001 | 0.9446640 | 15566.82603 | | 0.0000038 | 0.0926724 | 31162.97802 |
| | 0.0000002 | 0.9446640 | 15645.08235 | | 0.0000016 | 0.0357143 | 10134.16987 |
| | 0.0000002 | 0.9446640 | 17653.98849 | | 0.0000003 | 0.0357143 | 10164.38262 |
| | 0.0000002 | 0.9446640 | 17809.61765 | | 0.0000005 | 0.0357143 | 10179.25579 |
| | 0.0000145 | 0.8557312 | 79145.35138 | | 0.0000006 | 0.0357143 | 10199.786 |
| F6CSL | 0.0000007 | 0.1222571 | 2990.018687 | | 0.0000084 | 0.0765306 | 10247.31042 |
| | 0.0000001 | 0.0595611 | 3360.866902 | | 0.0000005 | 0.0357143 | 10308.99693 |
| | 0.0000009 | 0.1065831 | 4252.48155 | | 0.0000003 | 0.0357143 | 10324.98028 |
| | 0.0000050 | 0.1379310 | 6487.935491 | | 0.0000003 | 0.0357143 | 10351.28255 |
| | 0.0008161 | 0.2319749 | 6544.667343 | | 0.0000003 | 0.0357143 | 10382.71287 |
| | 0.0000000 | 0.0438871 | 6689.164478 | | 0.0000003 | 0.0357143 | 10437.59021 |
| | 0.0000016 | 0.0909091 | 6855.504433 | | 0.0000003 | 0.0357143 | 10456.27805 |
| | 0.0000066 | 0.1379310 | 6871.100138 | | 0.0000084 | 0.0969388 | 10586.5933 |
| | 0.0000001 | 0.0752351 | 12910.82457 | | 0.0000005 | 0.0153061 | 10639.37916 |
| | 0.0000003 | 0.0752351 | 13920.46348 | | 0.0000002 | 0.0153061 | 10659.7841 |
| | 0.0000003 | 0.0909091 | 14102.40615 | | 0.0000008 | 0.0357143 | 10714.34361 |
| | 0.0000004 | 0.0909091 | 17426.66866 | | 0.0000095 | 0.0969388 | 10736.08599 |
| | 0.0000008 | 0.0909091 | 18048.90648 | | 0.0000003 | 0.9795918 | 11623.61757 |
| | 0.0000005 | 0.1222571 | 28178.79099 | | 0.0000005 | 0.9795918 | 11673.12834 |
| | 0.0000000 | 0.0595611 | 29021.87789 | | 0.0000002 | 0.9795918 | 11750.83857 |
| | 0.0000000 | 0.0595611 | 30269.89108 | | 0.0000010 | 0.9591837 | 11779.63819 |
| | 0.0000006 | 0.0909091 | 31162.97802 | | 0.0000003 | 0.9795918 | 11824.74215 |
| F6CSH | 0.0000000 | 0.0388889 | 10034.41899 | | 0.0000003 | 0.9795918 | 11886.09971 |
| | 0.0000000 | 0.0071429 | 10134.16987 | | 0.0000002 | 0.9795918 | 11982.74557 |
| | 0.0000000 | 0.0071429 | 10164.38262 | | 0.0000002 | 0.9795918 | 12017.01197 |
| | 0.0000000 | 0.0071429 | 10179.25579 | | 0.0000002 | 0.9795918 | 12050.07302 |
| | 0.0000000 | 0.0071429 | 10199.786 | | 0.0000002 | 0.9795918 | 12170.65112 |
| | 0.0000000 | 0.0230159 | 10247.31042 | | 0.0000002 | 0.9795918 | 12200.58095 |
| | 0.0000000 | 0.0071429 | 10308.99693 | | 0.0000002 | 0.9795918 | 12243.19154 |
| | 0.0000000 | 0.0071429 | 10324.98028 | | 0.0000003 | 0.9795918 | 13157.2016 |
| | 0.0000000 | 0.0071429 | 10351.28255 | | 0.0000095 | 0.9183673 | 13373.91926 |
| | 0.0000000 | 0.0071429 | 10382.71287 | | 0.0000002 | 0.9795918 | 14029.68761 |
| | 0.0000000 | 0.0071429 | 10437.59021 | | 0.0000002 | 0.9795918 | 14160.13964 |
| | 0.0000000 | 0.0071429 | 10456.27805 | | 0.0000002 | 0.9795918 | 14336.01183 |
| | 0.0000000 | 0.0230159 | 10639.37916 | | 0.0000005 | 0.9795918 | 14434.26849 |
| | 0.0000000 | 0.0230159 | 10659.7841 | | 0.0000005 | 0.0357143 | 14665.79315 |
| | 0.0000000 | 0.0230159 | 10714.34361 | | 0.0000030 | 0.9591837 | 15420.52874 |
| | 0.0000000 | 0.0230159 | 10736.08599 | | 0.0000002 | 0.9795918 | 17530.97848 |
| | 0.0000000 | 0.0388889 | 10910.64978 | | 0.0000002 | 0.9795918 | 17656.32521 |
| | 0.0000001 | 0.0706349 | 10929.45696 | | 0.0000002 | 0.0153061 | 18041.10658 |
| | 0.0000001 | 0.9523810 | 11824.74215 | | 0.0000121 | 0.0765306 | 18241.15243 |
| | 0.0000000 | 0.9523810 | 11982.74557 | | 0.0000006 | 0.9591837 | 23184.82846 |
| | 0.0000004 | 0.9047619 | 12017.01197 | | 0.0000005 | 0.9591837 | 39178.65241 |
| | 0.0000000 | 0.9841270 | 12050.07302 | | 0.0000007 | 0.9591837 | 39783.20564 |
| | 0.0000000 | 0.9365079 | 12170.65112 | | | | |
| | 0.0000000 | 0.9841270 | 13157.2016 | | | | |
| | 0.0000000 | 0.9682540 | 13373.91926 | | | | |
| | 0.0000002 | 0.9047619 | 13859.59685 | | | | |
| | 0.0000000 | 1.0000000 | 14029.68761 | | | | |
| | 0.0000000 | 1.0000000 | 14160.13964 | | | | |
| | 0.0000000 | 1.0000000 | 14336.01183 | | | | |
| | 0.0000000 | 0.9841270 | 14434.26849 | | | | |
| | 0.0000000 | 0.9523810 | 15244.10632 | | | | |
| | 0.0000000 | 1.0000000 | 15420.52874 | | | | |
| | 0.0000000 | 0.9841270 | 15617.44859 | | | | |
| | 0.0000007 | 0.9047619 | 16254.77706 | | | | |
| | 0.0000001 | 0.9365079 | 16335.46786 | | | | |
| | 0.0000000 | 0.9841270 | 17530.97848 | | | | |
| | 0.0000000 | 1.0000000 | 17656.32521 | | | | |
| | 0.0000000 | 0.0230159 | 18041.10658 | | | | |
| | 0.0000003 | 0.9047619 | 39178.65241 | | | | |
| | 0.0000001 | 0.9365079 | 39783.20564 | | | | |

### REFERENCES

1. 2002. DengueNet—WHO's Internet-based System for the global surveillance of dengue fever and dengue haemorrhagic fever (dengue/DHF). Dengue/DHF—global public health burden. Weekly Epidemiological Record. 77:300-304.
2. Burke, D. S., A. Nisalak, D. E. Johnson, and R. M. Scott. 1988. A prospective study of dengue infections in Bangkok. American Journal of Tropical Medicine & Hygiene. 38:172-80.
3. Chang, G. J. 1997. The molecular biology of dengue virus, p. 175-198. In D. J. Gubler and G. Kuno (ed.), Dengue and dengue haemorrhagic fever. CAB International, New York.
4. Chaturvedi, U., R. Nagar, and R. Shrivastava. 2006. Dengue and dengue haemorrhagic fever: implications of host genetics. FEMS Immunology & Medical Microbiology. 47:155-166.
5. Cobra, C., J. G. Rigau-Perez, G. Kuno, and V. Vorndam. 1995. Symptoms of dengue fever in relation to host immunologic response and virus serotype, Puerto Rico, 1990-1991. American Journal of Epidemiology. 142:1204-1211.
6. Dietz, V., D. J. Gubler, S. Ortiz, G. Kuno, A. Casta-Velez, G. E. Sather, I. Gomez, and E. Vergne. 1996. The 1986 dengue and dengue hemorrhagic fever epidemic in Puerto Rico: epidemiologic and clinical observations. Puerto Rico Health Sciences Journal. 15:201-210.
7. Fink, J., F. Gu, and S. G. Vasudevan. 2006. Role of T cells, cytokines and antibody in dengue fever and dengue haemorrhagic fever. Reviews in Medical Virology. 16:263-275.
8. Green, S. and A. Rothman. 2006. Immunopathological mechanisms in dengue and dengue hemorrhagic fever. Current Opinion in Infectious Diseases. 19:429-436.
9. Guzman, M. G. and G. Kouri. 2002. Dengue: an update. The Lancet Infectious Diseases. 2:33-42.
10. Kalayanarooj, S., D. W. Vaughn, S. Nimmannitya, S. Green, S. Suntayakorn, N. Kunentrasai, W. Viramitrachai, S. Ratanachu-eke, S. Kiatpolpoj, B. L. Innis, A. L. Rothman, A. Nisalak, and F. A. Ennis. 1997. Early clinical and laboratory indicators of acute dengue illness. Journal of Infectious Diseases. 176:313-321.
11. Kao, C. L., C. C. King, D. Y. Chao, H. L. Wu, and G. J. Chang. 2005. Laboratory diagnosis of dengue virus infection: current and future perspectives in clinical diagnosis and public health. Journal of Microbiology, Immunology & Infection. 38:5-16.
12. Kliks, S. C., A. Nisalak, W. E. Brandt, L. Wahl, and D. S. Burke. 1989. Antibody-dependent enhancement of dengue virus growth in human monocytes as a risk factor for dengue hemorrhagic fever. American Journal of Tropical Medicine & Hygiene. 40:444-451.
13. Kuberski, T., L. Rosen, D. Reed, and J. Mataika. 1977. Clinical and laboratory observations on patients with primary and secondary dengue type 1 infections with hemorrhagic manifestations in Fiji. American Journal of Tropical Medicine & Hygiene. 26:775-783.
14. Kuhn, R. J., W. Zhang, M. G. Rossmann, S. V. Pletnev, J. Corver, E. Lenches, C. T. Jones, S. Mukhopadhyay, P. R. Chipman, E. G. Strauss, T. S. Baker, and J. H. Strauss. 2002. Structure of dengue virus: implications for flavivirus organization, maturation, and fusion. Cell. 108:717-725.
15. Lei, H. Y., T. M. Yeh, H. S. Liu, Y. S. Lin, S. H. Chen, and C. C. Liu. 2001. Immunopathogenesis of dengue virus infection. Journal of Biomedical Science. 8:377-388.
16. Oishi, K., S. Inoue, M. T. Cinco, E. M. Dimaano, M. T. Alera, J. A. Alfon, F. Abanes, D. J. Cruz, R. R. Matias, H. Matsuura, F. Hasebe, S. Tanimura, A. Kumatori, K. Morita, F. F. Natividad, and T. Nagatake. 2003. Correlation between increased platelet-associated IgG and thrombocytopenia in secondary dengue virus infections. Journal of Medical Virology. 71:259-264.
17. Rigau-Perez, J. G., G. G. Clark, D. J. Gubler, P. Reiter, E. J. Sanders, and A. V. Vorndam. 1998. Dengue and dengue haemorrhagic fever. Lancet. 352:971-977.
18. Senanayake, S. 2006. Dengue fever and dengue haemorrhagic fever—a diagnostic challenge. [Review] [27 refs]. Australian Family Physician. 35:609-612.
19. Simmons, C. P., T. N. Chau, T. T. Thuy, N. M. Tuan, D. M. Hoang, N. T. Thien, L. B. Lien, N. T. Quy, N. T. Hieu, T. T. Hien, C. McElnea, P. Young, S. Whitehead, N. T. Hung, and J. Farrar. 2007. Maternal antibody and viral factors in the pathogenesis of dengue virus in infants. Journal of Infectious Diseases. 196:416-424.
20. Stephenson, J. R. 2005. Understanding dengue pathogenesis: implications for vaccine design. [Review] [49 refs]. Bulletin of the World Health Organization. 83:308-314.
21. Teles, F. R., D. M. Prazeres, and J. L. Lima-Filho. 2005. Trends in dengue diagnosis. Reviews in Medical Virology. 15:287-302.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

1. A method for qualifying dengue status in a subject comprising:
(a) measuring at least one biomarker in a biological sample from the subject, wherein the at least one biomarker is selected from the group consisting of the biomarkers of Table 1, Table 2, Table 3, Table 4, Table 5, Table 17, Table 21, and Table 24; and
(b) correlating the measurement with dengue status.

2. (canceled)

3. The method of claim 1, wherein the at least one biomarker is selected from the group consisting of biomarkers of molecular masses of about 2.5, 2.6, 2.7, 2.8, 3.0, 3.2, 3.4, 3.5, 3.8, 3.9, 4.0, 4.1, 4.2, 4.3, 4.4, 4.5, 4.6, 5.0, 5.1, 5.2, 5.3, 5.5, 5.6, 5.7, 6.0, 6.5, 6.6, 6.7, 6.8, 6.9, 7.0, 7.4, 7.5, 7.6, 8.2, 8.8, 9.0, 9.3, 9.5, 9.6, 10.0, 10.1, 10.2, 10.3, 10.4, 10.5, 10.6, 10.7, 10.8, 10.9, 11.0, 11.1, 11.3, 11.5, 11.7, 11.8, 11.9, 12.1, 12.2, 12.4, 12.5, 12.6, 12.7, 12.9, 13.0, 13.1, 13.2, 13.3, 13.4, 13.5, 13.8, 14.0, 14.1, 14.2, 14.3, 14.4, 15.3, 17.4, 17.7, 17.8, 18.0, 21.5, 22.4, 23.1, 23.3, 23.6, 23.7, 23.8, 25.4, 25.6, 26.5, 26.7, 28.2, 28.4, 29.0, 30.3, 30.8, 31.2, 32.3, 33.5, 33.6, 34.0, 34.2, 34.5, 34.7, 36.5, 36.6, 38.7, 39.5, 39.9, 42.3, 43.5, 44.0, 44.6, 44.7, 45.0, 45.4, 45.5, 45.6, 46.2, 46.6, 46.7, 49.7, 50.5, 51.7, 52.4, 52.6, 53.4, 53.6, 54.3, 54.4, 54.6, 54.8, 55.1, 55.3, 55.8, 56.6, 59.4, 59.5, 61.4, 63.1, 66.6, 66.7, 66.8, 67.1, 69.0, 70.9, 71.3, 71.5, 75.1, 75.2, 75.3, 77.1, 79.1, 79.3, 88.3, 95.7, 109.0, 111.3, 117.2, 123.0, 125.4, 133.4, 133.7, 150.1, 164.6, 188.6, 194.2, and 198.3 kDa.

4-28. (canceled)

29. The method of claim 1, wherein the correlating is performed by a software classification algorithm.

30. The method of claim 1, wherein dengue status is selected from chronic symptomatic, chronic asymptomatic, acute, and uninfected.

31. The method of claim 1, wherein dengue status is selected from dengue versus non-dengue.

32. The method of claim 1, wherein dengue status is selected from dengue fever (DF), dengue hemorrhagic fever (DHF), and dengue shock syndrome (DSS).

33. The method of claim 1, wherein dengue status is selected from primary dengue infection and secondary dengue infection.

34. The method of claim 1, further comprising: (c) managing subject treatment based on the status.

35-37. (canceled)

38. The method of claim 30, wherein, if the measurement correlates with dengue, then managing subject treatment comprises administering one or more drugs selected from the group consisting of paracetamol and antipyretics.

39. The method of claim 30, further comprising: (d) measuring the at least one biomarker after subject management and correlating the measurement with disease progression.

40-41. (canceled)

42. A method for determining the course of dengue comprising:
(a) measuring, at a first time, at least one biomarker in a biological sample from the subject, wherein the at least one biomarker is selected from the group consisting of the biomarkers of Table 1, Table 2, Table 3, Table 4, Table 5, Table 17, Table 21, and Table 24;
(b) measuring, at a second time, the at least one biomarker in a biological sample from the subject; and
(c) comparing the first measurement and the second measurement; wherein the comparative measurements determine the course of dengue.

43-45. (canceled)

46. The method of claim 42, wherein the at least one biomarker is selected from the group of biomarkers of molecular masses of about 2.5, 2.6, 2.7, 2.8, 3.0, 3.2, 3.4, 3.5, 3.8, 3.9, 4.0, 4.1, 4.2, 4.3, 4.4, 4.5, 4.6, 5.0, 5.1, 5.2, 5.3, 5.5, 5.6, 5.7, 6.0, 6.5, 6.6, 6.7, 6.8, 6.9, 7.0, 7.4, 7.5, 7.6, 8.2, 8.8, 9.0, 9.3, 9.5, 9.6, 10.0, 10.1, 10.2, 10.3, 10.4, 10.5, 10.6, 10.7, 10.8, 10.9, 11.0, 11.1, 11.3, 11.5, 11.7, 11.8, 11.9, 12.1, 12.2, 12.4, 12.5, 12.6, 12.7, 12.9, 13.0, 13.1, 13.2, 13.3, 13.4, 13.5, 13.8, 14.0, 14.1, 14.2, 14.3, 14.4, 15.3, 17.4, 17.7, 17.8, 18.0, 21.5, 22.4, 23.1, 23.3, 23.6, 23.7, 23.8, 25.4, 25.6, 26.5, 26.7, 28.2, 28.4, 29.0, 30.3, 30.8, 31.2, 32.3, 33.5, 33.6, 34.0, 34.2, 34.5, 34.7, 36.5, 36.6, 38.7, 39.5, 39.9, 42.3, 43.5, 44.0, 44.6, 44.7, 45.0, 45.4, 45.5, 45.6, 46.2, 46.6, 46.7, 49.7, 50.5, 51.7, 52.4, 52.6, 53.4, 53.6, 54.3, 54.4, 54.6, 54.8, 55.1, 55.3, 55.8, 56.6, 59.4, 59.5, 61.4, 63.1, 66.6, 66.7, 66.8, 67.1, 69.0, 70.9, 71.3, 71.5, 75.1, 75.2, 75.3, 77.1, 79.1, 79.3, 88.3, 95.7, 109.0, 111.3, 117.2, 123.0, 125.4, 133.4, 133.7, 150.1, 164.6, 188.6, 194.2, and 198.3 kDa.

47-51. (canceled)

52. A kit comprising:
(a) a solid support comprising at least one capture reagent attached thereto, wherein the capture reagent binds at least one biomarker from a first group consisting of the Biomarkers of Table 1, Table 2, Table 3, Table 4, Table 5, Table 17, Table 21, and Table 24; and
(b) instructions for using the solid support to detect a biomarker of Table 1, Table 2, Table 3, Table 4, Table 5, Table 17, Table 21, or Table 24.

53. The kit of claim 52 comprising instructions for using the solid support to detect at least one biomarker of molecular mass of about 2.5, 2.6, 2.7, 2.8, 3.0, 3.2, 3.4, 3.5, 3.8, 3.9, 4.0, 4.1, 4.2, 4.3, 4.4, 4.5, 4.6, 5.0, 5.1, 5.2, 5.3, 5.5, 5.6, 5.7, 6.0, 6.5, 6.6, 6.7, 6.8, 6.9, 7.0, 7.4, 7.5, 7.6, 8.2, 8.8, 9.0, 9.3, 9.5, 9.6, 10.0, 10.1, 10.2, 10.3, 10.4, 10.5, 10.6, 10.7, 10.8, 10.9, 11.0, 11.1, 11.3, 11.5, 11.7, 11.8, 11.9, 12.1, 12.2, 12.4, 12.5, 12.6, 12.7, 12.9, 13.0, 13.1, 13.2, 13.3, 13.4, 13.5, 13.8, 14.0, 14.1, 14.2, 14.3, 14.4, 15.3, 17.4, 17.7, 17.8, 18.0, 21.5, 22.4, 23.1, 23.3, 23.6, 23.7, 23.8, 25.4, 25.6, 26.5, 26.7, 28.2, 28.4, 29.0, 30.3, 30.8, 31.2, 32.3, 33.5, 33.6, 34.0, 34.2, 34.5, 34.7, 36.5, 36.6, 38.7, 39.5, 39.9, 42.3, 43.5, 44.0, 44.6, 44.7, 45.0, 45.4, 45.5, 45.6, 46.2, 46.6, 46.7, 49.7, 50.5, 51.7, 52.4, 52.6, 53.4, 53.6, 54.3, 54.4, 54.6, 54.8, 55.1, 55.3, 55.8, 56.6, 59.4, 59.5, 61.4, 63.1, 66.6, 66.7, 66.8, 67.1, 69.0, 70.9, 71.3, 71.5, 75.1, 75.2, 75.3, 77.1, 79.1, 79.3, 88.3, 95.7, 109.0, 111.3, 117.2, 123.0, 125.4, 133.4, 133.7, 150.1, 164.6, 188.6, 194.2, and 198.3 kDa.

54-55. (canceled)

56. The kit of claim 52, wherein the solid support comprising a capture reagent is a SELDI probe or a cation exchange adsorbent.

57. (canceled)

58. The kit of claim 56, wherein the adsorbent is a metal chelate adsorbent.

59. The kit of claim 52, additionally comprising: (c) a container containing at least one of the biomarkers of Table 1, Table 2, Table 3, Table 4, Table 5, Table 17, Table 21, or Table 24.

60-68. (canceled)

69. A software product comprising:
(a) code that accesses data attributed to a sample, the data comprising measurement of at least one biomarker in the sample, the biomarker selected from the group consisting of the biomarkers of Table 1, Table 2, Table 3, Table 4, Table 5, Table 17, Table 21, and Table 24; and
(b) code that executes a classification algorithm that classifies the disease status of the sample as a function of the measurement.

70-76. (canceled)

77. A method for qualifying dengue status in a subject in comparison to the status of a different infection, the method comprising:
(a) measuring at least one biomarker in a biological sample from the subject, wherein the at least one biomarker specifically indicates the presence of dengue and does not indicate the presence of a different viral infection, wherein the at least one biomarker is selected from the group of the biomarkers of Table 1, Table 2, Table 3, Table 4, Table 5, Table 17, Table 21, or Table 24; and
(b) correlating the measuring with dengue status in comparison to the status of the different viral infection.

78. (canceled)

79. The method of claim 77, wherein said viral infection comprises another febrile illness.
